(19) United States
(12) Patent Application Publication
(10) Pub. No.: US 2023/0357364 A1
(43) Pub. Date: Nov. 9, 2023

(54) COVID-19 ANTIBODIES AND USES THEREOF

(71) Applicants: International AIDS Vaccine Initiative, Inc., New York, NY (US); The Scripps Research Institute, La Jolla, CA (US)

(72) Inventors: Devin Sok, New York, NY (US); Joseph Jardine, New York, NY (US); Elise Landais, New York, NY (US); Dennis Burton, La Jolla, CA (US); Tom Rogers, La Jolla, CA (US); Fangzhu Zhao, La Jolla, CA (US); Deli Huang, La Jolla, CA (US)

(21) Appl. No.: 17/923,313

(22) PCT Filed: May 6, 2021
(86) PCT No.: PCT/US2021/031195

§ 371 Date: Nov. 4, 2022

Related U.S. Application Data
(60) Provisional application No. 63/021,086, filed on May 6, 2020.
(60) Provisional application No. 63/021,676, filed on May 7, 2020.
(60) Provisional application No. 63/024,512, filed on May 13, 2020.
(60) Provisional application No. 63/035,554, filed on Jun. 5, 2020.
(60) Provisional application No. 63/036,405, filed on Jun. 8, 2020.
(60) Provisional application No. 63/038,093, filed on Jun. 11, 2020.
(60) Provisional application No. 63/073,603, filed on Sep. 2, 2020.

(51) Int. Cl. C07K 16/10

(52) U.S. Cl. CPC C07K 16/10; C07K 2317/76; C07K 2317/92; C07K 2317/33; A61K 2039/505

(57) ABSTRACT

The present application is directed to recombinant monoclonal antibodies, or antigen fragments thereof that bind a Spike protein of SARS-CoV-2. Methods of using the antibodies to treat or prevent SARS-CoV-2 (COVID-19) are also disclosed.

### CROSS-REFERENCE TO RELATED APPLICATIONS

This application claims the benefit of priority from U.S. Provisional Application No. 63/021,086, filed on May 6, 2020; from U.S. Provisional Application No. 63/021,676, filed on May 7, 2020; from U.S. Provisional Application No. 63/024,512, filed on May 13, 2020; from U.S. Provisional Application No. 63/035,554, filed on Jun. 5, 2020; from U.S. Provisional Application No. 63/036,405, filed on Jun. 8, 2020; from U.S. Provisional Application No. 63/038,093, filed on Jun. 11, 2020; and from U.S. Provisional Application No. 63/073,603, filed on Sep. 2, 2020. The foregoing applications are incorporated herein by reference in their entirety.

### BACKGROUND OF THE INVENTION

Coronavirus disease 19 (COVID-19) is an illness caused by the zoonotic SARS-CoV-2 virus and its strains, which have caused a worldwide pandemic. The majority of infected individuals remain asymptomatic or experience mild symptoms, such as cough, fever, fatigue, or loss of smell. However, a significant percent of the infected, particularly elderly, population may manifest more severe disease symptoms and experience complications, leading to acute respiratory distress syndrome, pneumonia, and even death. The time from exposure to onset of symptoms is typically around five days, but may range from two to fourteen days.

Currently, there is no known vaccine or anti-viral treatment available.

Thus, there is a high, unmet need for effective therapies for preventing COVID-19. Accordingly, it is an object of the present disclosure to provide methods for treating, preventing, or reducing the progression rate and/or severity of COVID-19, particularly treating, preventing or reducing the progression rate and/or severity of one or more COVID-19-associated complications.

### SUMMARY OF THE INVENTION

In one aspect, the present application provides neutralizing monoclonal antibodies or an antigen-binding fragments thereof that bind a Spike protein of SARS-CoV-2 and/or SARS-CoV-1 and variants thereof. In certain embodiments, the antibodies and antigen binding fragments disclosed herein are cross-reactive and are capable of binding both the Spike protein of SARS-CoV-2, SARS-CoV-1, and variants thereof. In other embodiments, the antibodies and antigen binding fragments disclosed herein are not cross-reactive and bind the Spike protein of SARS-CoV-2 but not SARS-CoV-1.

In some embodiments, the neutralizing monoclonal antibody or antigen-binding fragment thereof binds a Spike protein of SARS-CoV-2, SARS-CoV-1, that comprises an amino acid sequence of SEQ ID Nos: 1 or 194, and variants thereof. In some embodiments, the neutralizing monoclonal antibody or antigen-binding fragment thereof binds the S1 region of the Spike protein. In some embodiments, the S1 region targeted by the neutralizing monoclonal antibody or antigen-binding fragment thereof comprises an amino acid sequence within SEQ ID Nos: 1 or 194, and variants thereof. In some embodiments, the neutralizing monoclonal antibody or antigen-binding fragment thereof binds a receptor binding domain (RBD) of the Spike protein. In some embodiments, the RBD, to which the neutralizing monoclonal antibody or antigen-binding fragment thereof binds, comprises an amino acid sequence of SEQ ID No: 2, 3, 195 or 196. In some of the above embodiments, the neutralizing monoclonal antibody or antigen-binding fragment thereof inhibits binding of SARS-CoV-2 and/or SARS-CoV-1 to an ACE-2 receptor.

In some embodiments, the neutralizing monoclonal antibody or antigen-binding fragment thereof has a binding affinity of 10⁻⁶ to 10⁻⁹ kD to the Spike protein of SARS-CoV-2 and/or SARS-CoV-1. In some embodiments, the antibody or antigen-binding fragment thereof has a binding affinity of 10⁻⁹ to 10⁻¹² kD.

In some of the above embodiments, the neutralizing monoclonal antibody or antigen-binding fragment thereof inhibits binding of SARS-CoV-2 to an ACE-2 receptor. In certain embodiments, the neutralizing monoclonal antibodies or antigen-binding fragments thereof disclosed herein do not cross-react and/or inhibit binding of SARS-CoV-1 to an ACE-2 receptor.

In some embodiments, the neutralizing monoclonal antibodies or antigen-binding fragments thereof do not cross-react with a human antigen.

In some embodiments, the neutralizing monoclonal antibody or antigen-binding fragment thereof of the present application comprises:
(a) a variable heavy chain (VH) selected from the group comprising:
   1) a VH-CDR1 comprising an amino acid sequence of SEQ ID NO: 6, a VH-CDR2 comprising an amino acid sequence of SEQ ID NO: 7, and a VH-CDR3 comprising an amino acid sequence of SEQ ID NO: 8;
   2) a VH-CDR1 comprising an amino acid sequence of SEQ ID NO: 16, a VH-CDR2 comprising an amino acid sequence of SEQ ID NO: 17, and a VH-CDR3 comprising an amino acid sequence of SEQ ID NO: 18;
   3) a VH-CDR1 comprising an amino acid sequence of SEQ ID NO: 26, a VH-CDR2 comprising an amino acid sequence of SEQ ID NO: 27, and a VH-CDR3 comprising an amino acid sequence of SEQ ID NO: 28;
   4) a VH-CDR1 comprising an amino acid sequence of SEQ ID NO: 36, a VH-CDR2 comprising an amino acid sequence of SEQ ID NO: 37, and a VH-CDR3 comprising an amino acid sequence of SEQ ID NO: 38;
   5) a VH-CDR1 comprising an amino acid sequence of SEQ ID NO: 46, a VH-CDR2 comprising an amino acid sequence of SEQ ID NO: 47, and a VH-CDR3 comprising an amino acid sequence of SEQ ID NO: 48;
   6) a VH-CDR1 comprising an amino acid sequence of SEQ ID NO: 56, a VH-CDR2 comprising an amino acid sequence of SEQ ID NO: 57, and a VH-CDR3 comprising an amino acid sequence of SEQ ID NO: 58;
   7) a VH-CDR1 comprising an amino acid sequence of SEQ ID NO: 66, a VH-CDR2 comprising an amino acid sequence of SEQ ID NO: 67, and a VH-CDR3 comprising an amino acid sequence of SEQ ID NO: 68;
   8) a VH-CDR1 comprising an amino acid sequence of SEQ ID NO: 76, a VH-CDR2 comprising an amino acid sequence of SEQ ID NO: 77, and a VH-CDR3 comprising an amino acid sequence of SEQ ID NO: 78;
   9) a VH-CDR1 comprising an amino acid sequence of SEQ ID NO: 86, a VH-CDR2 comprising an amino acid sequence of SEQ ID NO: 87, and a VH-CDR3 comprising an amino acid sequence of SEQ ID NO: 88;
   10) a VH-CDR1 comprising an amino acid sequence of SEQ ID NO: 96, a VH-CDR2 comprising an amino acid sequence of SEQ ID NO: 97, and a VH-CDR3 comprising an amino acid sequence of SEQ ID NO: 98;
   11) a VH-CDR1 comprising an amino acid sequence of SEQ ID NO: 106, a VH-CDR2 comprising an amino acid sequence of SEQ ID NO: 107, and a VH-CDR3 comprising an amino acid sequence of SEQ ID NO: 108;
   12) a VH-CDR1 comprising an amino acid sequence of SEQ ID NO: 116, a VH-CDR2 comprising an amino acid sequence of SEQ ID NO: 117, and a VH-CDR3 comprising an amino acid sequence of SEQ ID NO: 118;
   13) a VH-CDR1 comprising an amino acid sequence of SEQ ID NO: 126, a VH-CDR2 comprising an amino acid sequence of SEQ ID NO: 127, and a VH-CDR3 comprising an amino acid sequence of SEQ ID NO: 128;
   14) a VH-CDR1 comprising an amino acid sequence of SEQ ID NO: 136, a VH-CDR2 comprising an amino acid sequence of SEQ ID NO: 137, and a VH-CDR3 comprising an amino acid sequence of SEQ ID NO: 138;
   15) a VH-CDR1 comprising an amino acid sequence of SEQ ID NO: 146, a VH-CDR2 comprising an amino acid sequence of SEQ ID NO: 147, and a VH-CDR3 comprising an amino acid sequence of SEQ ID NO: 148;
   16) a VH-CDR1 comprising an amino acid sequence of SEQ ID NO: 156, a VH-CDR2 comprising an amino acid sequence of SEQ ID NO: 157, and a VH-CDR3 comprising an amino acid sequence of SEQ ID NO: 158;
   17) a VH-CDR1 comprising an amino acid sequence of SEQ ID NO: 166, a VH-CDR2 comprising an amino acid sequence of SEQ ID NO: 167, and a VH-CDR3 comprising an amino acid sequence of SEQ ID NO: 168;
   18) a VH-CDR1 comprising an amino acid sequence of SEQ ID NO: 176, a VH-CDR2 comprising an amino acid sequence of SEQ ID NO: 177, and a VH-CDR3 comprising an amino acid sequence of SEQ ID NO: 178;
   19) a VH-CDR1 comprising an amino acid sequence of SEQ ID NO: 186, a VH-CDR2 comprising an amino acid sequence of SEQ ID NO: 187, and a VH-CDR3 comprising an amino acid sequence of SEQ ID NO: 188;
   20) a VH-CDR1 comprising an amino acid sequence of SEQ ID NO: 199, a VH-CDR2 comprising an amino acid sequence of SEQ ID NO: 200, and a VH-CDR3 comprising an amino acid sequence of SEQ ID NO: 201;
   21) a VH-CDR1 comprising an amino acid sequence of SEQ ID NO: 209, a VH-CDR2 comprising an amino acid sequence of SEQ ID NO: 210, and a VH-CDR3 comprising an amino acid sequence of SEQ ID NO: 211;
   22) a VH-CDR1 comprising an amino acid sequence of SEQ ID NO: 219, a VH-CDR2 comprising an amino acid sequence of SEQ ID NO: 220, and a VH-CDR3 comprising an amino acid sequence of SEQ ID NO: 221.
   23) a VH-CDR1 comprising an amino acid sequence of SEQ ID NO: 229, a VH-CDR2 comprising an amino acid sequence of SEQ ID NO: 230, and a VH-CDR3 comprising an amino acid sequence of SEQ ID NO: 231;
   24) a VH-CDR1 comprising an amino acid sequence of SEQ ID NO: 239, a VH-CDR2 comprising an amino acid sequence of SEQ ID NO: 240, and a VH-CDR3 comprising an amino acid sequence of SEQ ID NO: 241;
   25) a VH-CDR1 comprising an amino acid sequence of SEQ ID NO: 249, a VH-CDR2 comprising an amino acid sequence of SEQ ID NO: 250, and a VH-CDR3 comprising an amino acid sequence of SEQ ID NO: 251;
   26) a VH-CDR1 comprising an amino acid sequence of SEQ ID NO: 259, a VH-CDR2 comprising an amino acid sequence of SEQ ID NO: 260, and a VH-CDR3 comprising an amino acid sequence of SEQ ID NO: 261;
   27) a VH-CDR1 comprising an amino acid sequence of SEQ ID NO: 269, a VH-CDR2 comprising an amino acid sequence of SEQ ID NO: 270, and a VH-CDR3 comprising an amino acid sequence of SEQ ID NO: 271;
   28) a VH-CDR1 comprising an amino acid sequence of SEQ ID NO: 279, a VH-CDR2 comprising an amino acid sequence of SEQ ID NO: 280, and a VH-CDR3 comprising an amino acid sequence of SEQ ID NO: 281;
   29) a VH-CDR1 comprising an amino acid sequence of SEQ ID NO: 289, a VH-CDR2 comprising an amino acid sequence of SEQ ID NO: 290, and a VH-CDR3 comprising an amino acid sequence of SEQ ID NO: 291;
   30) a VH-CDR1 comprising an amino acid sequence of SEQ ID NO: 299, a VH-CDR2 comprising an amino acid sequence of SEQ ID NO: 300, and a VH-CDR3 comprising an amino acid sequence of SEQ ID NO: 301;
   31) a VH-CDR1 comprising an amino acid sequence of SEQ ID NO: 309, a VH-CDR2 comprising an amino acid sequence of SEQ ID NO: 310, and a VH-CDR3 comprising an amino acid sequence of SEQ ID NO: 311;
   32) a VH-CDR1 comprising an amino acid sequence of SEQ ID NO: 319, a VH-CDR2 comprising an amino acid sequence of SEQ ID NO: 320, and a VH-CDR3 comprising an amino acid sequence of SEQ ID NO: 321;
   33) a VH-CDR1 comprising an amino acid sequence of SEQ ID NO: 329, a VH-CDR2 comprising an amino acid sequence of SEQ ID NO: 330, and a VH-CDR3 comprising an amino acid sequence of SEQ ID NO: 331;
   34) a VH-CDR1 comprising an amino acid sequence of SEQ ID NO: 339, a VH-CDR2 comprising an amino acid sequence of SEQ ID NO: 340, and a VH-CDR3 comprising an amino acid sequence of SEQ ID NO: 341;
   35) a VH-CDR1 comprising an amino acid sequence of SEQ ID NO: 349, a VH-CDR2 comprising an amino acid sequence of SEQ ID NO: 350, and a VH-CDR3 comprising an amino acid sequence of SEQ ID NO: 351;
   36) a VH-CDR1 comprising an amino acid sequence of SEQ ID NO: 359, a VH-CDR2 comprising an amino acid sequence of SEQ ID NO: 360, and a VH-CDR3 comprising an amino acid sequence of SEQ ID NO: 361;
   37) a VH-CDR1 comprising an amino acid sequence of SEQ ID NO: 369, a VH-CDR2 comprising an amino acid sequence of SEQ ID NO: 370, and a VH-CDR3 comprising an amino acid sequence of SEQ ID NO: 371;
   38) a VH-CDR1 comprising an amino acid sequence of SEQ ID NO: 379, a VH-CDR2 comprising an amino acid sequence of SEQ ID NO: 380, and a VH-CDR3 comprising an amino acid sequence of SEQ ID NO: 381;
   39) a VH-CDR1 comprising an amino acid sequence of SEQ ID NO: 389, a VH-CDR2 comprising an amino acid sequence of SEQ ID NO: 390, and a VH-CDR3 comprising an amino acid sequence of SEQ ID NO: 391;
   40) a VH-CDR1 comprising an amino acid sequence of SEQ ID NO: 399, a VH-CDR2 comprising an amino acid sequence of SEQ ID NO: 400, and a VH-CDR3 comprising an amino acid sequence of SEQ ID NO: 401;
   41) a VH-CDR1 comprising an amino acid sequence of SEQ ID NO: 409, a VH-CDR2 comprising an amino acid sequence of SEQ ID NO: 410, and a VH-CDR3 comprising an amino acid sequence of SEQ ID NO: 411;
   42) a VH-CDR1 comprising an amino acid sequence of SEQ ID NO: 419, a VH-CDR2 comprising an amino acid sequence of SEQ ID NO: 420, and a VH-CDR3 comprising an amino acid sequence of SEQ ID NO: 421;
   43) a VH-CDR1 comprising an amino acid sequence of SEQ ID NO: 429, a VH-CDR2 comprising an amino acid sequence of SEQ ID NO: 430, and a VH-CDR3 comprising an amino acid sequence of SEQ ID NO: 431;
   44) a VH-CDR1 comprising an amino acid sequence of SEQ ID NO: 439, a VH-CDR2 comprising an amino acid sequence of SEQ ID NO: 440, and a VH-CDR3 comprising an amino acid sequence of SEQ ID NO: 441;
   45) a VH-CDR1 comprising an amino acid sequence of SEQ ID NO: 449, a VH-CDR2 comprising an amino acid sequence of SEQ ID NO: 450, and a VH-CDR3 comprising an amino acid sequence of SEQ ID NO: 451;
   46) a VH-CDR1 comprising an amino acid sequence of SEQ ID NO: 459, a VH-CDR2 comprising an amino acid sequence of SEQ ID NO: 460, and a VH-CDR3 comprising an amino acid sequence of SEQ ID NO: 461;
   47) a VH-CDR1 comprising an amino acid sequence of SEQ ID NO: 469, a VH-CDR2 comprising an amino acid sequence of SEQ ID NO: 470, and a VH-CDR3 comprising an amino acid sequence of SEQ ID NO: 471;
   48) a VH-CDR1 comprising an amino acid sequence of SEQ ID NO: 479, a VH-CDR2 comprising an amino acid sequence of SEQ ID NO: 480, and a VH-CDR3 comprising an amino acid sequence of SEQ ID NO: 481;
wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody; and
(b) a variable light chain (VL)) selected from the group comprising:
   (1) a VL-CDR1 comprising an amino acid sequence of SEQ ID NO: 11, a VL-CDR2 comprising an amino acid sequence of SEQ ID NO: 12, and a VL-CDR3 comprising an amino acid sequence of SEQ ID NO: 13;
   (2) a VL-CDR1 comprising an amino acid sequence of SEQ ID NO: 21, a VL-CDR2 comprising an amino acid sequence of SEQ ID NO: 22, and a VL-CDR3 comprising an amino acid sequence of SEQ ID NO: 23;
   (3) a VL-CDR1 comprising an amino acid sequence of SEQ ID NO: 31, a VL-CDR2 comprising an amino acid sequence of SEQ ID NO: 32, and a VL-CDR3 comprising an amino acid sequence of SEQ ID NO: 33;
   (4) a VL-CDR1 comprising an amino acid sequence of SEQ ID NO: 41, a VL-CDR2 comprising an amino acid sequence of SEQ ID NO: 42, and a VL-CDR3 comprising an amino acid sequence of SEQ ID NO: 43;
   (5) a VL-CDR1 comprising an amino acid sequence of SEQ ID NO: 51, a VL-CDR2 comprising an amino acid sequence of SEQ ID NO: 52, and a VL-CDR3 comprising an amino acid sequence of SEQ ID NO: 53;
   (6) a VL-CDR1 comprising an amino acid sequence of SEQ ID NO: 61, a VL-CDR2 comprising an amino acid sequence of SEQ ID NO: 62, and a VL-CDR3 comprising an amino acid sequence of SEQ ID NO: 63;
   (7) a VL-CDR1 comprising an amino acid sequence of SEQ ID NO: 71, a VL-CDR2 comprising an amino acid sequence of SEQ ID NO: 72, and a VL-CDR3 comprising an amino acid sequence of SEQ ID NO: 73;
   (8) a VL-CDR1 comprising an amino acid sequence of SEQ ID NO: 81, a VL-CDR2 comprising an amino acid sequence of SEQ ID NO: 82, and a VL-CDR3 comprising an amino acid sequence of SEQ ID NO: 83;
   (9) a VL-CDR1 comprising an amino acid sequence of SEQ ID NO: 91, a VL-CDR2 comprising an amino acid sequence of SEQ ID NO: 92, and a VL-CDR3 comprising an amino acid sequence of SEQ ID NO: 93;
   (10) a VL-CDR1 comprising an amino acid sequence of SEQ ID NO: 101, a VL-CDR2 comprising an amino acid sequence of SEQ ID NO: 102, and a VL-CDR3 comprising an amino acid sequence of SEQ ID NO: 103;
   (11) a VL-CDR1 comprising an amino acid sequence of SEQ ID NO: 111, a VL-CDR2 comprising an amino acid sequence of SEQ ID NO: 112, and a VL-CDR3 comprising an amino acid sequence of SEQ ID NO: 113;
   (12) a VL-CDR1 comprising an amino acid sequence of SEQ ID NO: 121, a VL-CDR2 comprising an amino acid sequence of SEQ ID NO: 122, and a VL-CDR3 comprising an amino acid sequence of SEQ ID NO: 123;
   (13) a VL-CDR1 comprising an amino acid sequence of SEQ ID NO: 131, a VL-CDR2 comprising an amino acid sequence of SEQ ID NO: 132, and a VL-CDR3 comprising an amino acid sequence of SEQ ID NO: 133;
   (14) a VL-CDR1 comprising an amino acid sequence of SEQ ID NO: 141, a VL-CDR2 comprising an amino acid sequence of SEQ ID NO: 142, and a VL-CDR3 comprising an amino acid sequence of SEQ ID NO: 143;
   (15) a VL-CDR1 comprising an amino acid sequence of SEQ ID NO: 151, a VL-CDR2 comprising an amino acid sequence of SEQ ID NO: 152, and a VL-CDR3 comprising an amino acid sequence of SEQ ID NO: 153;
   (16) a VL-CDR1 comprising an amino acid sequence of SEQ ID NO: 161, a VL-CDR2 comprising an amino acid sequence of SEQ ID NO: 162, and a VL-CDR3 comprising an amino acid sequence of SEQ ID NO: 163.
   (17) a VL-CDR1 comprising an amino acid sequence of SEQ ID NO: 171, a VL-CDR2 comprising an amino acid sequence of SEQ ID NO: 172, and a VL-CDR3 comprising an amino acid sequence of SEQ ID NO: 173;
   (18) a VL-CDR1 comprising an amino acid sequence of SEQ ID NO: 181, a VL-CDR2 comprising an amino acid sequence of SEQ ID NO: 182, and a VL-CDR3 comprising an amino acid sequence of SEQ ID NO: 183;
   (19) a VL-CDR1 comprising an amino acid sequence of SEQ ID NO: 191, a VL-CDR2 comprising an amino acid sequence of SEQ ID NO: 192, and a VL-CDR3 comprising an amino acid sequence of SEQ ID NO: 193;
   (20) a VL-CDR1 comprising an amino acid sequence of SEQ ID NO: 204, a VL-CDR2 comprising an amino acid sequence of SEQ ID NO: 205, and a VL-CDR3 comprising an amino acid sequence of SEQ ID NO: 206;
   (21) a VL-CDR1 comprising an amino acid sequence of SEQ ID NO: 214, a VL-CDR2 comprising an amino acid sequence of SEQ ID NO: 215, and a VL-CDR3 comprising an amino acid sequence of SEQ ID NO: 216;
   (22) a VL-CDR1 comprising an amino acid sequence of SEQ ID NO: 224, a VL-CDR2 comprising an amino acid sequence of SEQ ID NO: 225, and a VL-CDR3 comprising an amino acid sequence of SEQ ID NO: 226;
   (23) a VL-CDR1 comprising an amino acid sequence of SEQ ID NO: 234, a VL-CDR2 comprising an amino acid sequence of SEQ ID NO: 235, and a VL-CDR3 comprising an amino acid sequence of SEQ ID NO: 236;
   (24) a VL-CDR1 comprising an amino acid sequence of SEQ ID NO: 244, a VL-CDR2 comprising an amino acid sequence of SEQ ID NO: 245, and a VL-CDR3 comprising an amino acid sequence of SEQ ID NO: 246;
   (25) a VL-CDR1 comprising an amino acid sequence of SEQ ID NO: 254, a VL-CDR2 comprising an amino acid sequence of SEQ ID NO: 255, and a VL-CDR3 comprising an amino acid sequence of SEQ ID NO: 256;
   (26) a VL-CDR1 comprising an amino acid sequence of SEQ ID NO: 264, a VL-CDR2 comprising an amino acid sequence of SEQ ID NO: 265, and a VL-CDR3 comprising an amino acid sequence of SEQ ID NO: 266;
   (27) a VL-CDR1 comprising an amino acid sequence of SEQ ID NO: 274, a VL-CDR2 comprising an amino acid sequence of SEQ ID NO: 275, and a VL-CDR3 comprising an amino acid sequence of SEQ ID NO: 276;
   (28) a VL-CDR1 comprising an amino acid sequence of SEQ ID NO: 284, a VL-CDR2 comprising an amino acid sequence of SEQ ID NO: 285, and a VL-CDR3 comprising an amino acid sequence of SEQ ID NO: 286;
   (29) a VL-CDR1 comprising an amino acid sequence of SEQ ID NO: 294, a VL-CDR2 comprising an amino acid sequence of SEQ ID NO: 295, and a VL-CDR3 comprising an amino acid sequence of SEQ ID NO: 296;
   (30) a VL-CDR1 comprising an amino acid sequence of SEQ ID NO: 304, a VL-CDR2 comprising an amino acid sequence of SEQ ID NO: 305, and a VL-CDR3 comprising an amino acid sequence of SEQ ID NO: 306;
   (31) a VL-CDR1 comprising an amino acid sequence of SEQ ID NO: 314, a VL-CDR2 comprising an amino acid sequence of SEQ ID NO: 315, and a VL-CDR3 comprising an amino acid sequence of SEQ ID NO: 316;
   (32) a VL-CDR1 comprising an amino acid sequence of SEQ ID NO: 324, a VL-CDR2 comprising an amino acid sequence of SEQ ID NO: 325, and a VL-CDR3 comprising an amino acid sequence of SEQ ID NO: 326;
   (33) a VL-CDR1 comprising an amino acid sequence of SEQ ID NO: 334, a VL-CDR2 comprising an amino acid sequence of SEQ ID NO: 335, and a VL-CDR3 comprising an amino acid sequence of SEQ ID NO: 336;
   (34) a VL-CDR1 comprising an amino acid sequence of SEQ ID NO: 344, a VL-CDR2 comprising an amino acid sequence of SEQ ID NO: 345, and a VL-CDR3 comprising an amino acid sequence of SEQ ID NO: 346;
   (35) a VL-CDR1 comprising an amino acid sequence of SEQ ID NO: 354, a VL-CDR2 comprising an amino acid sequence of SEQ ID NO: 355, and a VL-CDR3 comprising an amino acid sequence of SEQ ID NO: 356;
   (36) a VL-CDR1 comprising an amino acid sequence of SEQ ID NO: 364, a VL-CDR2 comprising an amino acid sequence of SEQ ID NO: 365, and a VL-CDR3 comprising an amino acid sequence of SEQ ID NO: 366;
   (37) a VL-CDR1 comprising an amino acid sequence of SEQ ID NO: 374, a VL-CDR2 comprising an amino acid sequence of SEQ ID NO: 375, and a VL-CDR3 comprising an amino acid sequence of SEQ ID NO: 376;
   (38) a VL-CDR1 comprising an amino acid sequence of SEQ ID NO: 384, a VL-CDR2 comprising an amino acid sequence of SEQ ID NO: 385, and a VL-CDR3 comprising an amino acid sequence of SEQ ID NO: 386;
   (39) a VL-CDR1 comprising an amino acid sequence of SEQ ID NO: 394, a VL-CDR2 comprising an amino acid sequence of SEQ ID NO: 395, and a VL-CDR3 comprising an amino acid sequence of SEQ ID NO: 396;
   (40) a VL-CDR1 comprising an amino acid sequence of SEQ ID NO: 404, a VL-CDR2 comprising an amino acid sequence of SEQ ID NO: 405, and a VL-CDR3 comprising an amino acid sequence of SEQ ID NO: 406;
   (41) a VL-CDR1 comprising an amino acid sequence of SEQ ID NO: 414, a VL-CDR2 comprising an amino acid sequence of SEQ ID NO: 415, and a VL-CDR3 comprising an amino acid sequence of SEQ ID NO: 416;
   (42) a VL-CDR1 comprising an amino acid sequence of SEQ ID NO: 424, a VL-CDR2 comprising an amino acid sequence of SEQ ID NO: 425, and a VL-CDR3 comprising an amino acid sequence of SEQ ID NO: 426;
   (43) a VL-CDR1 comprising an amino acid sequence of SEQ ID NO: 434, a VL-CDR2 comprising an amino acid sequence of SEQ ID NO: 435, and a VL-CDR3 comprising an amino acid sequence of SEQ ID NO: 436;
   (44) a VL-CDR1 comprising an amino acid sequence of SEQ ID NO: 444, a VL-CDR2 comprising an amino acid sequence of SEQ ID NO: 445, and a VL-CDR3 comprising an amino acid sequence of SEQ ID NO: 446;
   (45) a VL-CDR1 comprising an amino acid sequence of SEQ ID NO: 454, a VL-CDR2 comprising an amino acid sequence of SEQ ID NO: 455, and a VL-CDR3 comprising an amino acid sequence of SEQ ID NO: 456;
   (46) a VL-CDR1 comprising an amino acid sequence of SEQ ID NO: 464, a VL-CDR2 comprising an amino acid sequence of SEQ ID NO: 465, and a VL-CDR3 comprising an amino acid sequence of SEQ ID NO: 466;
   (47) a VL-CDR1 comprising an amino acid sequence of SEQ ID NO: 474, a VL-CDR2 comprising an amino acid sequence of SEQ ID NO: 475, and a VL-CDR3 comprising an amino acid sequence of SEQ ID NO: 476;
   (48) a VL-CDR1 comprising an amino acid sequence of SEQ ID NO: 484, a VL-CDR2 comprising an amino acid sequence of SEQ ID NO: 485, and a VL-CDR3 comprising an amino acid sequence of SEQ ID NO: 486;
wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody. In some these embodiments, the amino acid substitutions, deletions or insertions comprises a homologous substitution.

In some embodiments of the neutralizing monoclonal antibody or antigen-binding fragment thereof as described herein, the VH chain comprises an amino acid sequence that is at least 80%, 85%, 90%, 92%, 93%, 95%, 97%, 98%, 99% or 100% identical to a sequence selected from the group consisting of: SEQ ID NOs: 4, 14, 24, 34, 44, 54, 64, 74, 84, 94, 104, 114, 124, 134, 144, 154, 164, 174, 184, 197, 207, 217, 227, 237, 247, 257, 267, 277, 287, 297, 307, 317, 327, 337, 347, 357, 367, 377, 387, 397, 407, 417, 427, 437, 447, 457, 467, and 477.

In some embodiments of the neutralizing monoclonal antibody or antigen-binding fragment thereof as described herein, the VL chain comprises an amino acid sequence that is at least 80%, 85%, 90%, 92%, 93%, 95%, 97%, 98%, 99% or 100% identical to a sequence selected from the group consisting of: SEQ ID NOs: 9, 19, 29, 39, 49, 59, 69, 79, 89, 99, 109, 119, 129, 139, 149, 159, 169, 179, 189, 202, 212, 222, 232, 242, 252, 262, 272, 282, 292, 302, 312, 322, 332, 342, 352, 362, 372, 382, 392, 402, 412, 422, 432, 442, 452, 462, 472, and 482.

In some embodiments, the neutralizing monoclonal antibody or antigen-binding fragment thereof of the present application is an antigen-binding fragment. In some embodiments, the antigen-binding fragment is a scFv. In some embodiments, the antigen-binding fragment is a Fab′. In some embodiments, the neutralizing monoclonal antibody or antigen-binding fragment of the present application is an antibody. In some embodiments, the antibody is an IgG antibody.

In another aspect, this application provides a composition comprising a neutralizing monoclonal antibody or antigen-binding fragment thereof as described herein and a pharmaceutically acceptable carrier.

In another aspect, this application provides a nucleic acid encoding a neutralizing monoclonal antibody or antigen-binding fragment thereof as described herein. In some embodiments, the present application provides a nucleic acid molecule encoding a VH chain comprising an amino acid sequence that is at least 80%, 85%, 90%, 92%, 93%, 95%, 97%, 98%, 99% or 100% identical to an amino acid sequence selected from the group consisting of: SEQ ID NOs: 4, 14, 24, 34, 44, 54, 64, 74, 84, 94, 104, 114, 124, 134, 144, 154, 164, 174, 184, 197, 207, 217, 227, 237, 247, 257, 267, 277, 287, 297, 307, 317, 327, 337, 347, 357, 367, 377, 387, 397, 407, 417, 427, 437, 447, 457, 467, and 477. In some embodiments, the nucleic acid sequence is at least 80%, 85%, 90%, 92%, 93%, 95%, 97%, 98%, 99% or 100% identical to a nucleic acid sequence selected from the group consisting of SEQ ID NOs: 5, 15, 25, 35, 45, 55, 65, 75, 85, 95, 105, 115, 125, 135, 145, 155, 165, 175, 185, 198, 208, 218, 228, 238, 248, 258, 268, 278, 288, 298, 308, 318, 328, 338, 348, 358, 368, 378, 388, 398, 408, 418, 428, 438, 448, 458, 468, and 478.

In some embodiments, the present application provides a nucleic acid molecule that encodes a VL chain comprising an amino acid sequence that is at least 80%, 85%, 90%, 92%, 93%, 95%, 97%, 98%, 99% or 100% identical to an amino acid sequence selected from the group consisting of: SEQ ID NOs: 9, 19, 29, 39, 49, 59, 69, 79, 89, 99, 109, 119, 129, 139, 149, 159, 169, 179, 189, 202, 212, 222, 232, 242, 252, 262, 272, 282, 292, 302, 312, 322, 332, 342, 352, 362, 372, 382, 392, 402, 412, 422, 432, 442, 452, 462, 472, and 482. In some embodiments, the nucleic acid sequence is at least 80%, 85%, 90%, 92%, 93%, 95%, 97%, 98%, 99% or 100% identical to a nucleic acid sequence selected from the group consisting of SEQ ID NOs: 10, 20, 30, 40, 50, 60, 70, 80, 90, 100, 110, 120, 130, 140, 150, 160, 170, 180, 190, 203, 213, 223, 233, 243, 253, 263, 273, 283, 293, 303, 313, 323, 333, 343, 353, 363, 373, 383, 393, 403, 413, 423, 433, 443, 453, 463, 473, and 483.

In another aspect, this application provides a vector comprising a nucleic acid as described herein. In another aspect, this application provides a host cell comprising a vector as described herein.

In some embodiments, the present application provides a lyophilized composition comprising a neutralizing monoclonal antibody or antigen-binding fragment thereof as described herein. In some embodiments, the present application provides a reconstituted lyophilized composition comprising a neutralizing monoclonal antibody or antigen-binding fragment thereof as described herein.

In some embodiment, the composition of the present application is formulated for administration by lozenge, spray, oral administration, delayed release or sustained 25 release, transmucosal administration, syrup, mucoadhesive, buccal formulation, mucoadhesive tablet, topical administration, parenteral administration, injection, subdermal administration, oral solution, rectal administration, buccal administration or transdermal administration.

In another aspect, the present application provides a method of treating SARS-CoV-2 infections comprising administering a therapeutically effective amount of a neutralizing monoclonal antibody or antigen-binding fragment thereof as described herein.

For the treatment of Covid-19 viral infection, the appropriate dosage of the antibodies, or antibody fragments (e.g., antigen binding fragments), depend on various factors, such as the type of infection to be treated, the severity and course of the infection, the responsiveness of the infection, the generation of viral resistance to therapy, previous therapy, patient's clinical history, and so on. The antibody can be administered one time or over a series of treatments lasting from several days to several months, or until a cure is effected or a diminution of the infection is achieved (e.g., reduction in viruria or viral damage to the kidney). Optimal dosing schedules can be calculated from measurements of drug accumulation in the body of the patient and will vary depending on the relative potency of an individual antibody or antibody fragment (e.g., antigen binding fragment). In certain aspects, dosage is from 0.01 mg to 10 mg (e.g., 0.01 mg, 0.05 mg, 0.1 mg, 0.5 mg, 1 mg, 2 mg, 3 mg, 4 mg, 5 mg, 7 mg, 8 mg, 9 mg, or 10 mg) per kg of body weight, and can be given once or more daily, weekly, monthly or yearly. In certain aspects, the antibody or antibody fragment (e.g., antigen binding fragment), of the present disclosure is given once every two weeks or once every three weeks. The treating physician can estimate repetition rates for dosing based on measured half-life and concentrations of the antibody in bodily fluids or tissues.

In certain embodiments, the antibodies or antigen binding fragments thereof, disclosed herein have a half-life anywhere from 1 day to 5 weeks. In some embodiments, the antibodies or antigen binding fragments thereof have a half-life of 1 week to 3 weeks. In certain embodiments, the antibodies or antigen binding fragments thereof, disclosed herein have a half-life anywhere from 2 weeks to 3 weeks.

In another aspect, the present application provides a method of producing a neutralizing monoclonal antibody or antigen-binding fragment thereof as described herein, the method comprising the steps of: expressing the nucleic acid or set of nucleic acids encoding the antibody or antigen-binding fragment as described herein in a cultured cell, purifying the antibody or antigen-binding fragment.

### BRIEF DESCRIPTION OF THE DRAWINGS

The file of this patent application contains at least one drawing/photograph executed in color. Copies of this patent application with color drawing(s)/photograph(s) will be provided by the Office upon request and payment of the necessary fee.

FIG. 1A depicts a graph illustrating the binding affinity of neutralizing monoclonal anti-SARS-CoV-2 antibodies against the Spike protein of SARS-CoV-2 virus. FIG. 1B depicts a graph illustrating the binding affinity of neutralizing monoclonal anti-SARS-CoV-2 antibodies against the RBD of SARS-CoV-2 virus. FIG. 1C depicts a graph illustrating the binding affinity of neutralizing monoclonal anti-SARS-CoV-2 antibodies against the RBD of the SARS-CoV-1 virus.

FIG. 2A depicts a graph illustrating the binding affinity of neutralizing monoclonal anti-SARS-CoV-2 antibodies against the Spike protein of SARS-CoV-1 virus. FIG. 2B depicts a graph illustrating the binding affinity of neutralizing monoclonal anti-SARS-CoV-2 antibodies against the Spike protein of SARS-CoV-2 virus.

FIG. 3A depicts a graph illustrating that neutralizing monoclonal anti-SARS-CoV-2 antibodies are not polyreactive against CHO SMP (solubilized membrane preps). FIG. 3B depicts a graph illustrating that neutralizing monoclonal anti-SARS-CoV-2 antibodies are not polyreactive against ssDNA. FIG. 3C depicts a graph illustrating that neutralizing monoclonal anti-SARS-CoV-2 antibodies are not polyreactive against Insulin.

FIG. 4 depicts a graph illustrating a large cluster of potent SARS-CoV-2 neutralizing antibodies with competing epitopes bin into 6 non-competing (distinct) epitopes, which are called RBD-A, RBD-B, RBD-C, Spike-A, Spike-B. and Spike-C. The antibodies that bind to RBD-A, RBD-B, or RBD-C can bind to their epitopes on RBD by itself as well as to RBD that is present on the full-length spike. The antibodies that bind to Spike-A, Spike-B, or Spike-C bind only to spike and do not appear to bind to RBD. RBD-A is the most important epitope as all the most potent antibodies target this epitope.

FIG. 5A depicts a graph illustrating the SARS-CoV-2 neutralizing monoclonal antibodies with a neutralization K_{D} value of 50 ng/mL using Huh7 reporter cells. FIG. 5B depicts a graph from group illustrating the SARS-CoV-2 neutralizing monoclonal antibodies with a neutralization K_{D} value of 100 ng/mL using VeroE6-ACE2 reporter cells. FIG. 5C depicts a graph illustrating the SARS-CoV-2 neutralizing monoclonal antibodies K_{D} value of 8-16 ng/mL using HeLa-ACE2 reporter cells. FIG. 5D depicts a graph illustrating the SARS-CoV-2 neutralizing monoclonal antibodies neutralization (represented as infection percent) K_{D} value of 1000 ng/mL using VeroE6 cells.

FIG. 6 depicts all antibodies that expressed at significant levels and were tested for binding to S protein and RBD to determine their specificity, and then screened for neutralization. FIG. 6A depicts a diagram illustrating that a small proportion of the binding antibodies showed neutralization activity and that activity was equally distributed between RBD+/S+ and S+ only binders, despite a much larger number of S+ only binding supernatants.

FIG. 6B and FIG. 6C depict charts of sequencing results that reveal that these antibodies identified 25 distinct lineages, with 23 containing a single member. VH1 and VH3-gene families were notably prominent in these Abs and there was a diversity of CDR3 lengths.

FIG. 6D depicts a dotplot illustrating that there was one prominent example of a clonally expanded lineage, with 8 recovered clonal members that averaged 4.3% and 2.8% mutations from germline at the nucleotide level in the heavy chain and light chain, respectively.

FIG. 7A depicts bubble plots of antibody functional activity by epitope specificities. Monoclonal antibody epitope binning was completed using RBD and SARS-CoV-2 S protein as target antigens. A total of three non-competing epitopes for RBD (RBD-A, RBD-B, and RBD-C) and three non-competing epitopes for S (S-A, S-B, and S-C) were identified. FIG. 7B depicts a dotplot of MAbs were evaluated for binding to different target antigens (Spike. N-terminal domain (NTD). RBD, RBD-SD1, and RBD-SD1-2) by ELISA and apparent EC₅₀s are reported in μg/ml. FIG. 7C depicts a dotplot of MAbs were evaluated for neutralization on SARS-CoV-2 pseudovirus and HeLA-ACE2 target cells in reference to epitope binning. Antibodies are grouped according to epitope specificities and neutralization IC₅₀ values are reported in μg/ml. FIG. 7D depicts a dotplot of maximum plateaus of neutralization (MPN) are reported for each mAb and grouped by epitope specificity. FIG. 7E depicts a dotplot of measurements of a mixture of MAbs and S binding to HeLA-ACE2 target cells as a measure of competition to the cell surface ACE-2 receptor. FIG. 7F depicts a dotplot of measurements of a mixture of MAbs and RBD binding to HeLA-ACE2 target cells as a measure of competition to the cell surface ACE-2 receptor. FIG. 7G depicts a dotplot of monoclonal antibody neutralization potencies (IC₅₀, μg/ml), which are plotted compared to dissociation constants (K_{D}, M) measured by surface plasmon resonance (SPR) to RBD target antigen.

FIG. 8A depicts a schemata illustrating of the strategy for the intraperitoneal injection of SARS-CoV-2-specific human neutralizing mAb CC12.1, which was isolated from natural infection, into Syrian hamsters at a starting dose of 2 mg/animal (on average 16.5 mg/kg) and subsequent serial 4-fold dilutions. Control animals received 2 mg of a dengue-specific human IgG1 (Den3). Each group of 6 animals were challenged intranasally 12 h post infusion with 1×10⁶ PFU of SARS-CoV-2. Serum was collected at the time of challenge Day 0, and their weight monitored as an indicator of disease progression. On day 5, lung tissue was collected for viral burden assessment. FIG. 8B depicts dotplots illustrating the percentage weight change, which was calculated from day 0 for all animals at all time points. The upper panel illustrates the percentage weight change at day 5 for each animal of each group. The lower panel illustrates the average weight loss in each group over time.

FIG. 9 depicts a schemata illustrating the neutralization against all circulating COV-2 variants for antibodies L6.dP03H10 (FIG. 9A) and L6.dP4E05 (FIG. 9B). Reduced neutralization was observed for L6.dP2E5 (FIG. 9C) on variant G476S. Reduced neutralization was also observed for antibody L6.P4A3 (FIG. 9D) on variant V367F. The tables underneath each graph lists the neutralization IC₅₀ against those indicated viruses at ng/ml concentrations. Additionally, monoclonal antibodies were evaluated for neutralization against the USA-WA1/2020 (WT) strain of SARS-CoV-2. Mutants of these viruses were generated by site-directed mutagenesis. The mutations were chosen based on a published manuscript (Korber et al. Biorxiv, 2020).

FIG. 10 depicts a graph illustrating the neutralization against the SARS-CoV-1 by the monoclonal antibodies. None could neutralize except for L6.P4A3. The SARS-CoV-1 neutralizing antibody CR3022 was included as a positive control.

FIG. 11 depicts graphs illustrating the polyreactivity of the antibodies using single stranded DNA (ssDNA) (FIG. 11A), insulin (FIG. 11B), and CHO solubilized membrane preps (FIG. 11C). The licensed antibody Bococizumab, which elicited ADA in the clinic, was included as a positive control. None of the neutralizing antibodies to SARS-CoV-2 were polyreactive in these assays.

FIG. 12A-F depicts histograms illustrating antibody aggregation, which was purified by SEC-HPLC using a TOSHO TSKgel SuperSQ mAb HTP column.

FIG. 13A-B depicts SDS-PAGE for evidence of aggregation or clipping of the heavy or variable genes of the antibodies. The antibodies were run as non-reduced (−DTT) and reduced (+DTT) for each antibody. The P04E05 on the gel was shown in with a box (FIG. 13B).

FIG. 14A depicts a dotplot of viral N RNA copies per lung at differing antibody doses of antibody CC12.1 (L12.bP11A6). FIG. 14B depicts a dotplot correlating the viral N RNA copies per lung at day 5 to the weight change.

FIG. 15A depicts a dotplot correlating serum neutralizing antibody titer at Day 0 to the dosage of antibody CC12.1 (L12.bP11A6). FIG. 15B depicts a dotplot correlating the percent weight change to the antibody titer.

FIG. 16 depicts neutralization assay development. FIG. 16A depicts a graph illustrating the comparison between Vero and HeLa-ACE2 cells. HeLa-ACE2 cell line showed 100% infection at a dilution factor of 2. FIG. 16B depicts a diagram of the alignment of SARS-CoV-1 and SARS-CoV-2 transmembrane domain and cytoplasmic tail. FIG. 16C depicts a graph of the infectious efficiency in relative luminescence units (RLU) of MLV viral particles pseudotyped with the indicated spike (S) proteins: FL indicates full-length spike. D18 and D28 denote C-terminal truncations in the indicated cells. FIG. 16D depicts a graph correlating relative luciferase and fold-dilution.

FIG. 17 depicts functional screening of cloned Ab H+L pairs rescued from SARS-CoV-2-specific single B-cell sorting. FIG. 17A depicts histograms illustrating the presence of IgG, binding to recombinant SARS-CoV-2 S-protein, and RBD subunit as well as for pseudotyped SARS-CoV-2 neutralization. ELISA considered positive when OD405 nm was >0.5 (dotted line). Results are plotted to show the proportion of expressed, binding and neutralizing pairs. FIG. 17B depicts a histogram illustrating the correlation between ELISA binding signal (OD 405 nm) and corresponding sorted cell staining level (MFI) for each antigenic bait (SARS-CoV-2 S-protein or RBD).

FIG. 18 depicts graphs illustrating ELISA data for evaluation of SARS-CoV-2 specific mAbs for polyreactivity and autoreactivity and assessed for binding to several polyspecificity reagents (PSR): CHO-cell soluble membrane protein extracts (SMP) (FIG. 18A), single stranded DNA (ssDNA) (FIG. 18B), and Insulin (FIG. 18C).

FIG. 19A-D depicts histograms in which monoclonal antibodies were evaluated for epitope competition using an Octet RED384 platform.

FIG. 20 depicts graphs illustrating the functional characterization of SARS-CoV-2 specific mAbs selected from the HTP screening. FIG. 20A depicts graphs illustrating ELISA data in which antibodies were tested for binding to several truncated versions of the recombinant SARS-CoV-1 and SARS-CoV-2 S proteins. FIG. 20B depicts graphs illustrating monoclonal antibodies binding to cell surface binding to SARS-CoV-1 and CoV-2 Spike. FIG. 20C depicts graphs illustrating neutralization of pseudotyped SARS-CoV-2 on HeLa-ACE2 or Vero cells. FIG. 20D depicts graphs illustrating neutralization of replicating live SARS-CoV-2 on HeLa-ACE2 or Vero cells.

FIG. 21 depict diagrams illustrating the functional characterization of SARS-CoV-2 specific mAbs selected from the HTP screening as assessed by flow cytometry.

FIG. 22 depicts a histogram illustrating SARS-CoV-2 nAb affinities. FIG. 22A depicts a graph illustrating that mAb CC12.1 binding inhibits the interaction of SARS-CoV-2-RBD with ACE2 (Top). Representative data from a SPR competition experiment is shown, in which the effect of varying concentrations of ACE2 on the interaction of 50 nM SARS-CoV-2-RBD with CC12.1 was examined. A legend showing the identity of each sensorgram is inset, in which the ACE2 injection alone was subtracted from the SARS-CoV-2-RBD+ACE2 injection series. The residual SARS-CoV-2-RBD binding in the presence of multiple ACE2 concentrations is shown in varying colors, while the sensorgram for the same concentration of SARS-CoV-2-RBD in the absence of ACE2 is shown as the darkest black line (Bottom). Residual SARS-CoV-2-RBD binding in the presence of various concentrations of ACE2, normalized to 50 nM CoV-2-RBD (100%) and buffer (0%), and fit to a dose-response curve (IC₅₀=120 nM). FIG. 22B depicts histograms illustrating SPR sensorgrams for SARS-CoV-2-RBD binding. Recombinant antibodies were captured via Fc-capture to an anti-human IgG (Fc) antibody and varying concentrations of SARS-CoV-2-RBD were injected using a multi-cycle method. Representative sensorgrams in resonance units (RUs) plotted against time of injection are shown. Black lines are the experimental trace obtained from the SPR experiments and red are the best global fits (1:1 Langmuir binding model) to the data used to calculate the association (kₐ) and dissociation (k_{d}) rate constants.

FIG. 23 depicts a dotplot illustrating Plasma and nAb neutralization abilities against global SARS-CoV-2 variants. Potent SARS-CoV-2 nAbs were tested neutralization activities against pseudotyped (PSV) SARS-CoV-2 virus and its escaped variants. Fold decrease of plasma neutralization ID₅₀ as compared to wildtype SARS-CoV-2 pseudovirus.

FIG. 24 depicts dotplots illustrating animal passive immunization studies. Syrian hamsters received various doses of SARS-CoV-2-specific human mAbs CC12.1 or CC12.23 or 2 mg dengue-specific human mAb Den3 i.p 12 h before i.n. challenge with SARS-CoV-2. FIG. 24A depicts dotplots illustrating CC12.1 protection experiment. From top to bottom, left to right: Weights of animals at time of challenge (Day 0): CC12.1 serum concentration in each animal as measured by ELISA at time of challenge (12 h post administration, Day 0); Viral load in lung tissue for each animal at day 5-post challenge; linear correlation between serum human IgG concentration at time of termination (Day 5) and % weight loss at day 5 (95% confidence intervals indicated in grey shade, R-square value is also indicated); Weight change (%) in each animal at Day 1, 3, 4 and 5 post challenge. FIG. 24B depicts dotplots illustrating CC12.23 protection experiment. From top to bottom, left to right: Weights of animals at time of challenge (Day 0); Weight change (%) in each animal at Day 3, 4 and 5 post challenge (Day 0); average weight loss in each group over time; linear correlation between serum human IgG concentration at time of challenge (Day 0) and % weight loss at Day 5 (95% confidence intervals indicated in grey shade, R-square value is also indicated). Significance of the difference between the groups was evaluated with Mann-Whitney U-tests using a 95% confidence interval. P-values are indicated (ns: non-significant; (*) <0.0332; (**)<0.0021; (***)<0.0002; (****)<0.00001).

FIG. 25A depicts a graph illustrating the binding affinity of eL6.P4A3 enhanced IgG1 neutralizing monoclonal anti-SARS-CoV-2 antibodies against the Spike protein of SARS-CoV-2 virus. FIG. 25B depicts a graph illustrating the binding affinity of enhanced IgG1 neutralizing monoclonal anti-SARS-CoV-2 antibodies against the Spike of SARS-CoV virus. FIG. 25C depicts a graph illustrating the binding affinity of enhanced IgG1 neutralizing monoclonal anti-SARS-CoV-2 antibodies against the RBD of the SARS-CoV-2 virus. FIG. 25D depicts a graph illustrating the binding affinity of enhanced IgG1 neutralizing monoclonal anti-SARS-CoV-2 antibodies against the RBD of the SARS-CoV-1 virus. Data indicate that all eL6.P4A3 variants bind with higher affinity to SARS-CoV2 spike protein than parental (L6.P4A3), which is highlighted as a black square.

FIG. 26A depicts a graph illustrating the neutralization of eL6.P4A3 enhanced IgG1 neutralizing monoclonal anti-SARS-CoV-2 antibodies against SARS-CoV-2 pseudovirus. FIG. 26B depicts a graph illustrating the neutralization of enhanced IgG1 neutralizing monoclonal anti-SARS-CoV-2 antibodies against SARS-CoV-1 pseudovirus. Data indicate that all the enhanced antibodies neutralize SARS-Cov and SARS-Cov-2 virus at a higher potency than (P4A3), which is highlighted as a black circle.

FIG. 27A-B depicts SDS-PAGE for visualizing 5 ug of IgG recombinant antibodies, which were run as non-reduced (−DTI) and reduced (+DTT) for each antibody and stained with brilliant blue coomassie.

FIG. 28A-K depicts histograms illustrating size exclusion chromatography using 5 μg of IgG recombinant antibodies, which were run as non-reduced (−DTI) and reduced (+DTT) for each antibody and stained with brilliant blue coomassie.

FIG. 29A-M depicts images of from autoreactivity staining assay performed on human epithelial 2 (HEp-2) cells using the eL6.P4A3 enhanced IgG1 monoclonal antibodies. Antibodies 4E10 and Bococizumab were included as a positive control. Data indicate antibodies are not polyreactive in the HEp-2 assay relative to 4E10 positive controls.

FIG. 30 depicts a graph illustrating that the eL6.P4A3 enhanced IgG1 from the neutralizing monoclonal anti-SARS-CoV-2 antibodies are not polyreactive against CHO SMP.

FIG. 31A-E depicts graphs and a table of the binding affinity and neutralization potency of enhanced SARS-CoV-2 neutralizing antibodies. (A) Enhanced and parental nAbs binding affinity against SARS-CoV-2 RBD by surface plasmon resonance. Parental nAbs were highlighted in black. RBD binding to antibodies via an Fc-capture, multi-cycle method. Association and dissociation rate constants were calculated through a 1:1 Langmuir binding model using the BIAevaluation software. (B) Neutralization IC₅₀ against pseudotyped SARS-CoV-2 and SARS-CoV viruses. (C) SARS-CoV-2 neutralization curves of parental CC12.1 and eCC12.1.1 to eCC12.1.12. The eCC12.1.6 and eCC12.1.7 nAbs were highlighted in yellow and orange respectively while other enhanced nAbs were grey. (D) SARS-CoV-2 and SARS-CoV pseudovirus neutralization curves of parental CC6.33 and eCC6.33.1 to eCC6.33.12. eCC6.33.1, eCC6.33.3, and eCC6.33.8 were highlighted according to the key while other enhanced nAbs were grey. Assays were run in duplicate. Error bars represent standard deviation. Data were representative for at least two independent experiments. (E) Summary table of nAb neutralization IC₅₀ against pseudotyped SARS-CoV and SARS-CoV2, as well as replicating SARS-CoV-2.

FIG. 32A-C depicts graphs and a table of SARS-CoV-2 RBD and spike binding affinity of parental and enhanced nAbs. (A) RBD binding to antibodies via a Fc-capture, multi-cycle method. (B) Fabs binding to SARS-CoV-2 spike via a His-capture, multi-cycle method. Association and dissociation rate constants for both IgG and Fab were calculated through a 1:1 Langmuir binding model using the BIAevaluation software. (C) Summary table of association constant, dissociation constant, and equilibrium dissociation constant measured from (A) and (B).

FIG. 33A-B depicts graphs and images of the polyreactivity of parental and enhanced nAbs. (A) ELISA of eCC6.30, eCC6.33, eCC12.1 variants and parental clones to CHO solubilized membrane proteins, human insulin, and ssDNA. Bococizumab serves as positive control while Den3 serves as negative control. Error bars represent standard deviations. (B) HEp2 epithelial cells staining with mAbs at 100 ug/mL. 4E10 and Bococizumab serve as positive control.

FIG. 34A-C depicts graphs and a table of monoclonal antibody IgG and Fab neutralization of pseudotyped SARS-CoV-2. Neutralization curves of (A) parental CC6.33 and eCC6.33 variants and (B) parental CC6.30 and eCC6.30 variants in both IgG and Fab formats. Error bars represent standard deviations. (B) Summary table of neutralization potency of IgG and Fab variants against SARS-CoV-2 pseudovirus.

FIG. 35A-G depicts graphs of antibody neutralization activities against circulating variants. (A) Prevalence of circulating SARS-CoV-2 mutations relative to WIV04 strain from Wuhan (EPI_ISL_402124). Fold decrease of antibody neutralization potency against (B) 7 most common circulating variants with single mutation at RBD, (C) B.1.1.7 strain, 501Y.V2 strain, E484K and K417N variants, relative to wild type. (D) SARS-CoV-2 neutralization curves of parental CC12.1 and eCC12.1.1 to eCC12.1.12. eCC12.1.6 and eCC12.1.7 were highlighted in yellow and orange respectively while other enhanced nAbs were grey. (D) Neutralization curves of antibodies against SARS-CoV-2 variants B.1.1.7, 501Y.V2, K417N and (E) E484K. Fold decrease of antibody neutralization potency against SARS-CoV-2 mutations at (F) CC6.33 and (G) CC6.30 key binding sites relative to wild type. Assays were run in duplicate. Error bars represent standard deviation. Data were representative for at least two independent experiments.

FIG. 36 depicts a graph of in vivo neutralizing activities of enhanced nAbs in a Syrian hamster model.

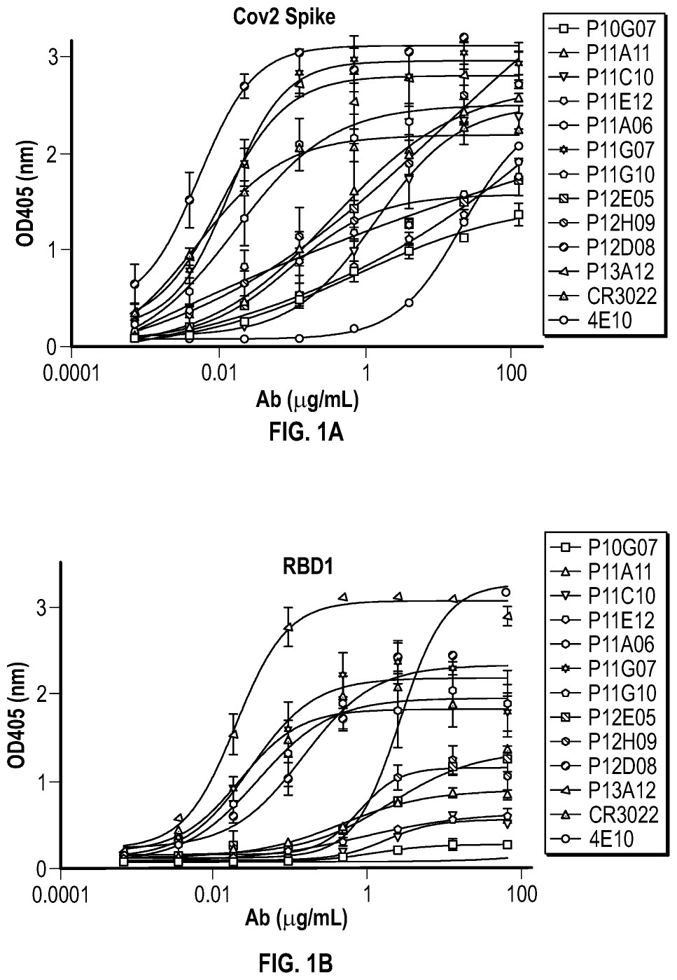

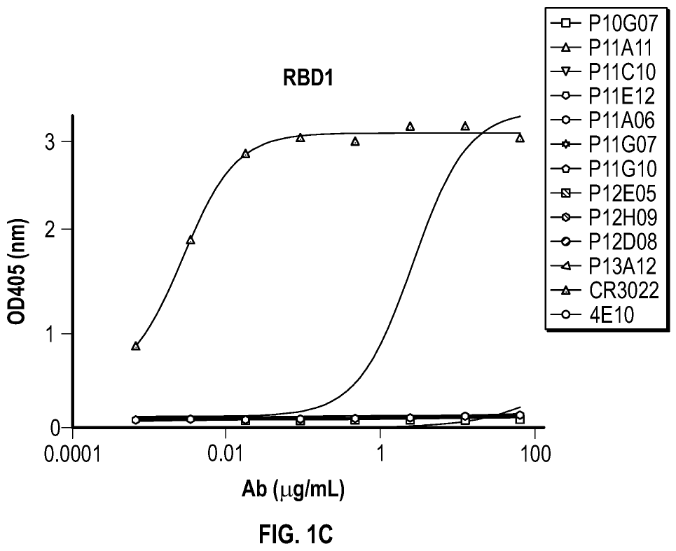

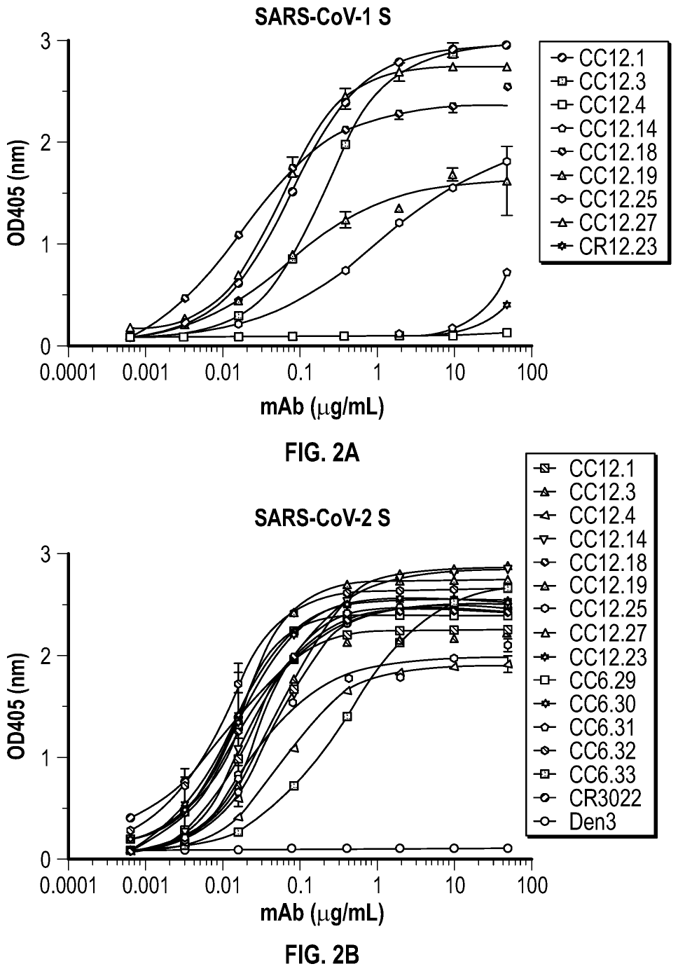

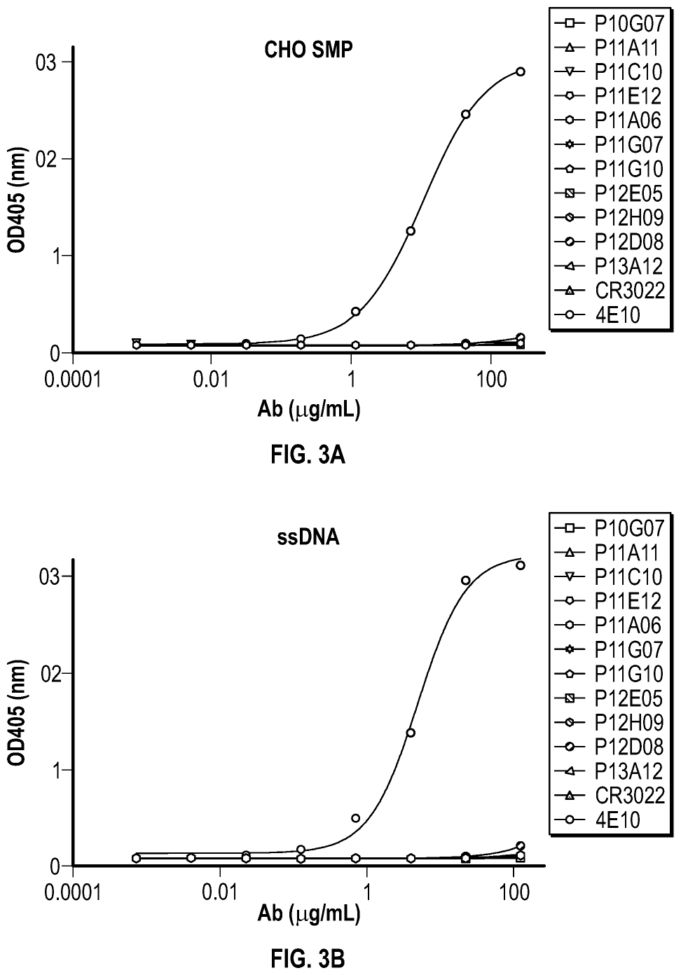

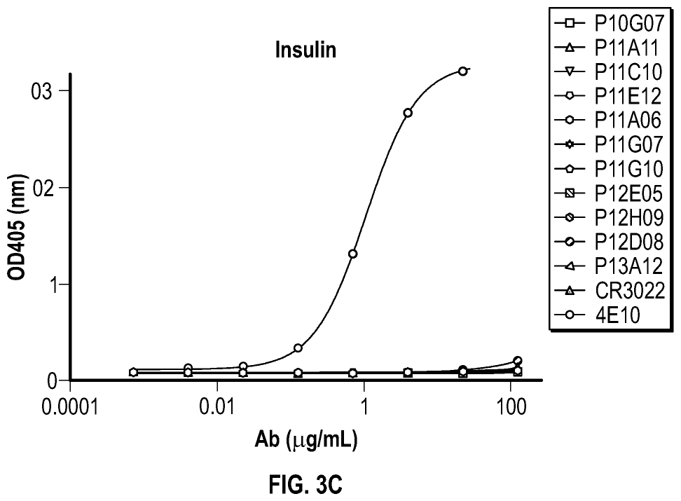

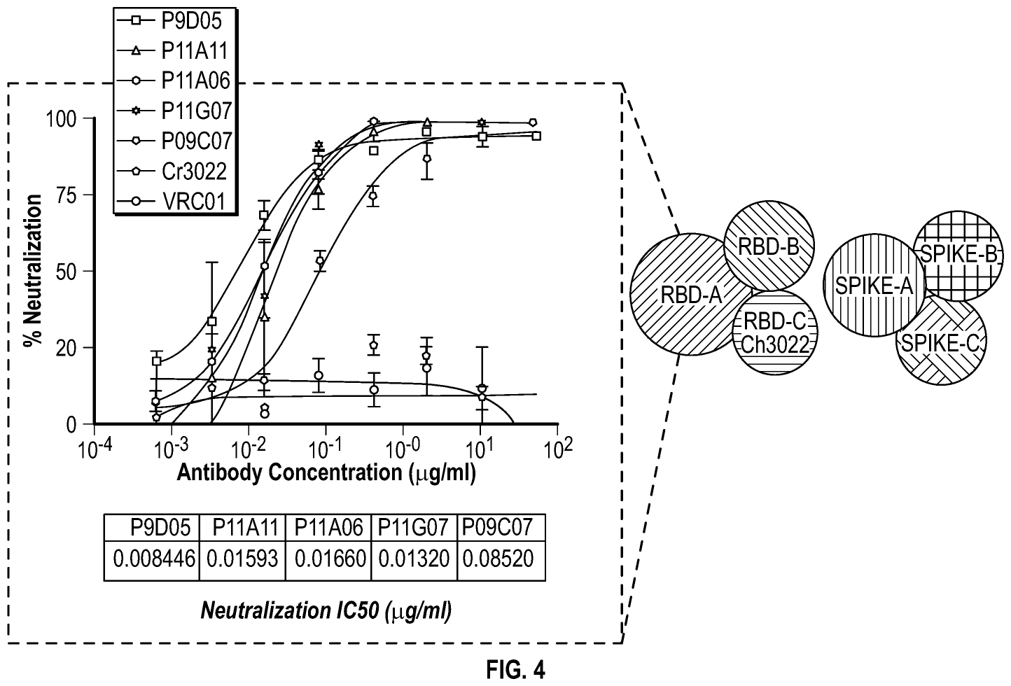

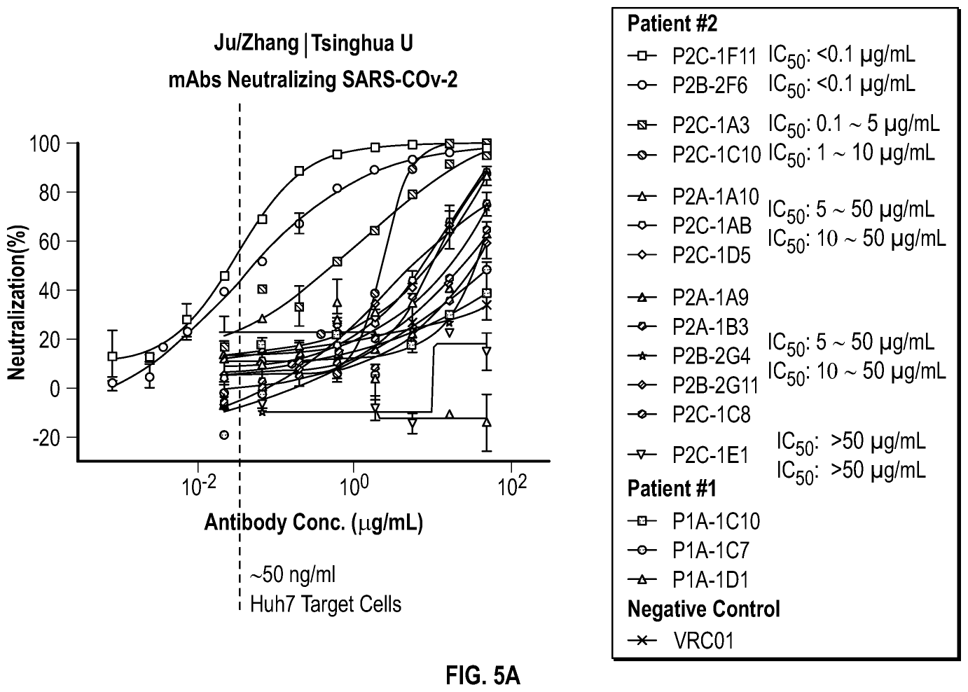

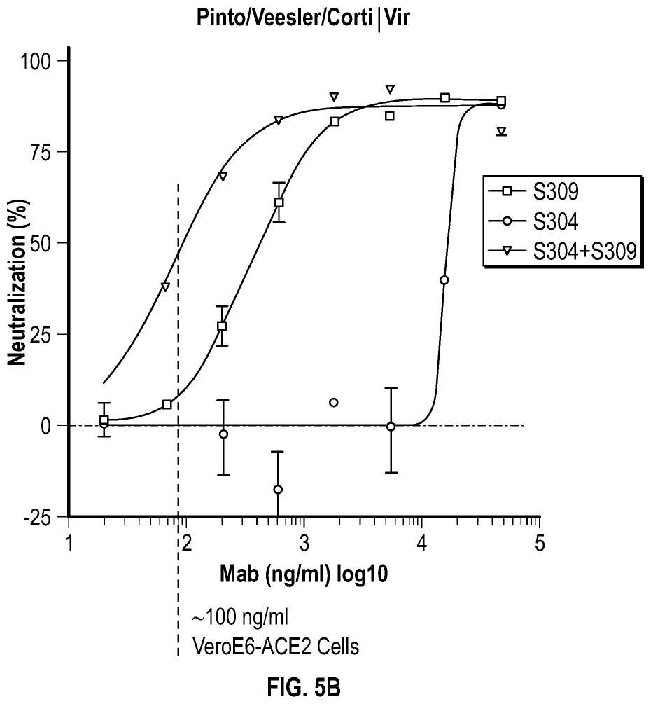

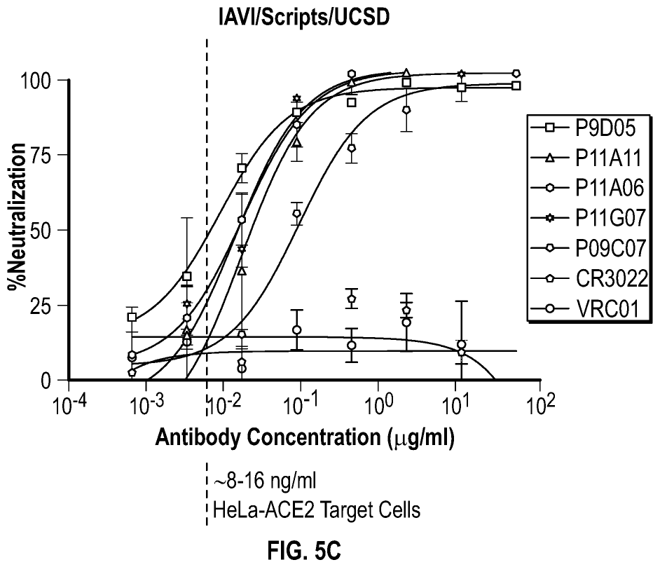

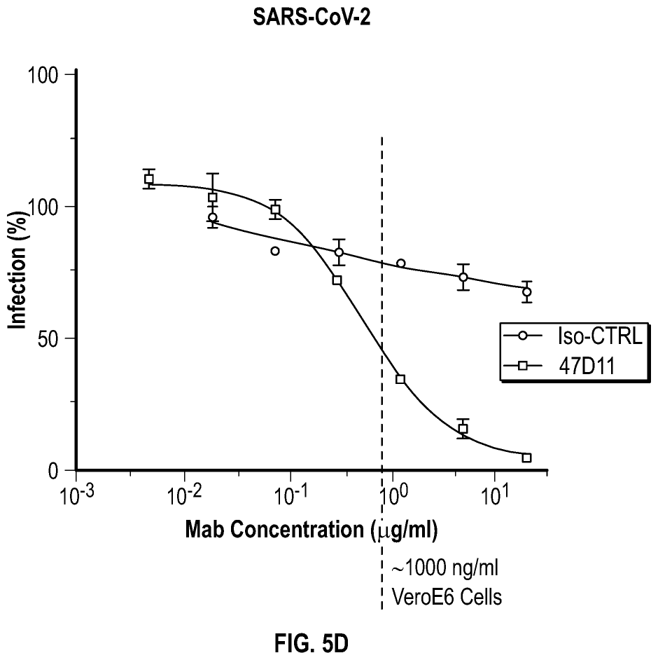

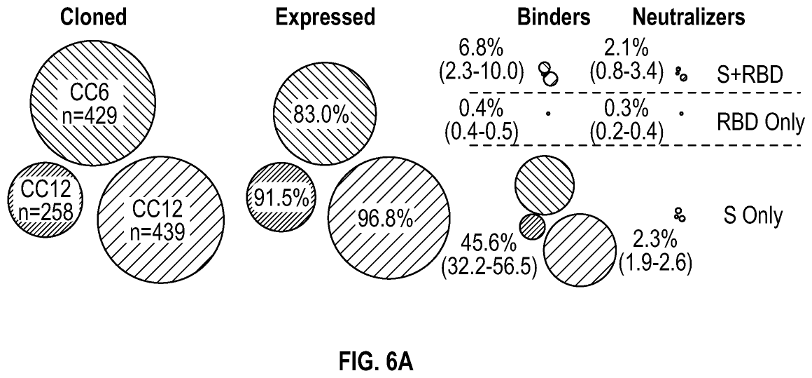

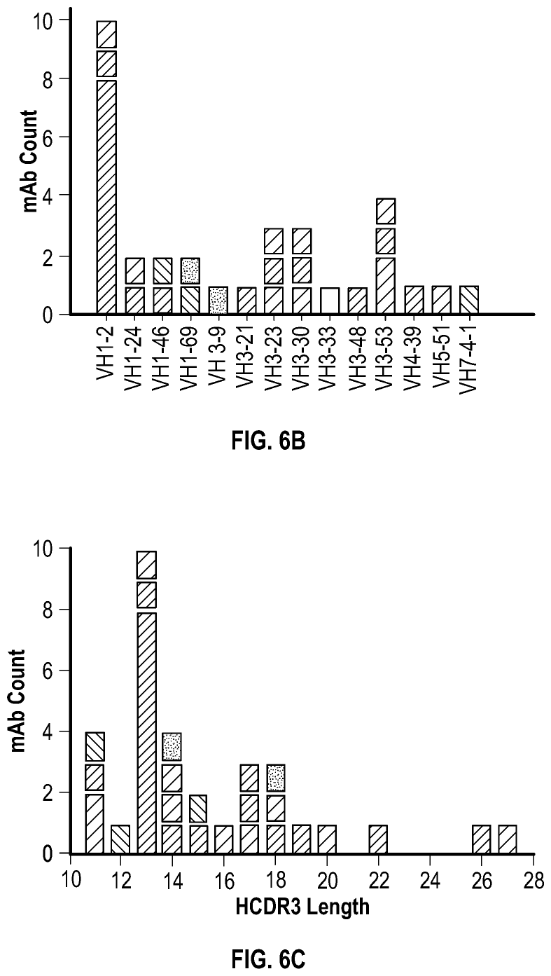

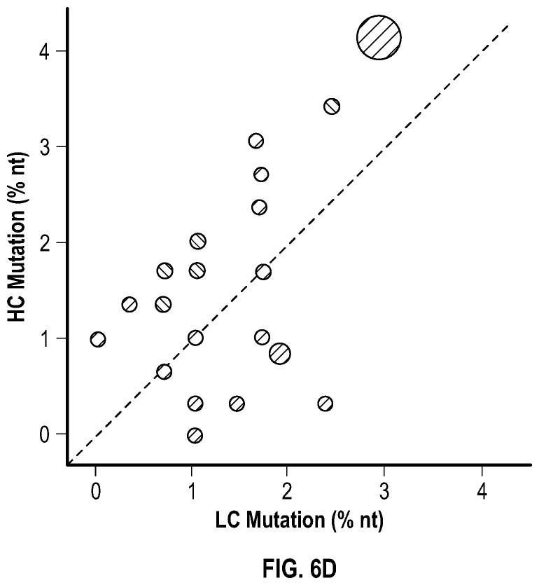

### DETAILED DESCRIPTION

### Overview/Definitions

In late 2019, a distinctive coronavirus (CoV) was determined to be responsible for an outbreak of potentially fatal atypical pneumonia, ultimately referred to as Severe Acute Respiratory Syndrome CoV-2 or COVID-19. This novel CoV, SARS-CoV-2, was found to be similar to the CoV that was responsible for the SARS pandemic that occurred in 2002.

CoVs are a large family of enveloped, positive-sense, single-stranded RNA viruses that infect a broad range of vertebrates. They are extensive in bats but are also found in many other birds and mammals including humans. CoVs can cause a variety of diseases such as enteritis in pigs and cows and upper respiratory disease in chickens. In humans, CoVs tend to cause mild to moderate upper respiratory tract infections such as the common cold. In the past couple of decades, there have been outbreaks of severe, and sometimes fatal, respiratory illnesses that are caused by these novel, human pathogenic CoVs. These CoV strains are extremely contagious, exhibit strong virulence and quickly transfer from human to human.

Accordingly, it is an object of the present disclosure to provide methods for treating, preventing, or reducing the progression rate and/or severity of SARS-CoV-2 infections or COVID-19, particularly treating, preventing or reducing the progression rate and/or severity of one or more SARS-CoV-2 or COVID-19-associated complications. In particular, this application discloses antibodies that are useful in treating, preventing, or reducing the progression rate and/or severity of SARS-CoV-2 or COVID-19 infections. In particular, treating, preventing or reducing the progression rate and/or severity of one or more COVID-19-associated complications.

As used in this specification and the appended claims, the singular form “a”, “an” and “the” include plural referents unless the context clearly dictates otherwise.

Amino acids may be referred to herein by either their commonly known three letter symbols or by the one-letter symbols recommended by the IUPAC-IUB Bio-chemical Nomenclature Commission. Nucleotides, likewise, may be referred to by their commonly accepted single-letter codes.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The terms “polypeptide,” “peptide” and “protein” are used interchangeably herein to refer to a polymer of amino acid residues. The terms apply to amino acid poly-mers in which one or more amino acid residue is an artificial chemical mimetic of a corresponding naturally occurring amino acid, as well as to naturally occurring amino acid polymers and non-naturally occurring amino acid polymer.

The term “SARS-CoV-2”, also called as “COVID-19”, refers to the newly-emerged Severe Acute Respiratory Syndrome, which was first identified in Wuhan, China in 2019 (World Health Organization 2020). It belongs to the betacoronavirus lineage B and causes severe respiratory disease, similar to the Severe Acute Respiratory Syndrome coronavirus (SARS-CoV) that emerged in China in 2002. The SARS coronavirus 2 has been found to be closely related to coronaviruses found in bats (Perlman et al 2020. New England Journal of Medicine 382: 760-762) and pangolins (Zhang et al 2020, Current Biology. 30: 1346-1351). It appears that SARS-CoV-2 binds via the viral spike protein to the human host cell. The host cell receptor is the Angiotensin Converting Enzyme 2 (ACE-2) receptor. SARS-CoV-2 spike protein has been found to bind to ACE-2 receptor of other species, especially bats and pandolins (Hoffman et al 2020, Cell. 181: 271-280).

As used herein, the term “Severe Acute Respiratory Syndrome-Coronavirus-2 Spike”, “SARS-CoV-2-S,” refers to the viral spike protein. The term “SARS-CoV-2-S” includes protein variants of the SARS-CoV-2 spike protein isolated from different SARS-CoV-2 isolates (shown in, recombinant SARS-CoV-2 spike protein or fragments thereof. The term also encompasses SARS-CoV-2 spike protein or a fragment thereof coupled to various tags, such as for example, histidine tag, mouse or human Fc, or a signal sequence such as ROR1. The SARS-CoV-2 spike protein is as set forth in SEQ ID Nos: 1 and 194. The Spike protein is a type I membrane glycoprotein which assembles into trimers that constitute the spikes or peplomers on the surface of the enveloped MERS coronavirus particle. The protein has two essential functions, host receptor binding and membrane fusion, which are attributed to the N-terminal (S1) and C-terminal (S2) halves of the S protein.

The term “Severe Acute Respiratory Syndrome-Coronavirus-2 Receptor Binding Domain”, “SARS-CoV-2-RBD.” as used herein, refers to a viral receptor binding domain of the Spike protein that is present in the S1 subunit of the Spike protein and comprises the sequence set forth in SEQ ID NO: 2 or 195, or biologically active fragments thereof.

As used herein, the ACE-2 receptor refers to a type I transmembrane metallocarboxypeptidase with homology to ACE, an enzyme that plays a role in the Renin-Angiotensin system (RAS) and is generally considered to be a target for the treatment of hypertension. The ACE-2 receptor is mainly expressed in vascular endothelial cells, the renal tubular epithelium, and in Leydig cells in the testes. ACE-2 is also expressed in the lung, kidney, and gastrointestinal tract, tissues shown to harbor SARS-CoV-2.

The term “SARS-CoV-2 infection” as used herein, refers to the respiratory illness caused by the SARS-CoV-2 coronavirus. The term includes respiratory tract infection, often in the lower respiratory tract. The symptoms include high fever, cough, shortness of breath pneumonia, gastro intestinal symptoms such as diarrhea, organ failure (kidney failure and renal dysfunction), septic shock and death in certain cases.

The term “SARS-CoV-1” refers to Severe Acute Respiratory Syndrome, which was first identified in southern China in 2002 (World Health Organization 2020). The SARS coronavirus (SARS-CoV) is a member of the Coronaviridae family of enveloped, positive-Stranded RNA viruses, which as a group, have a broad host range. It contains three major structural proteins: spike (S), membrane (M), nucleocapsid (N). Though it has been shown that passive protection from murine hepatitis virus (MHV, aintensively investigated coronavirus), infection has been achieved by administration of MAb specific for all major structural proteins of the virus, the Spike protein (S) is the major antigenic determinant for coronaviruses. The serological response in the host is typically raised against the S protein (see Moore et al., Arch. Virol. 142 (11):2249-56 (1997); Talbot et al., J. Virol. 62:3032 (1988); Gallagher et al., Virology 279(2):371-74 (2001): Song et al., J. Gen. Virol. 79(4):719-23 (1998); and Lamarre et al., Eur. J. Immunol. 27:3447-55 (1997),

As used herein, the term “Severe Acute Respiratory Syndrome-Coronavirus-1 Spike”, “SARS-CoV-1-S,” refers to the viral spike protein. The term “SARS-CoV-1-S” includes protein variants of the SARS-CoV-1 spike protein isolated from different SARS-CoV-1 isolates (shown in, recombinant SARS-CoV-1 spike protein or fragments thereof. The term also encompasses SARS-CoV-1 spike protein or a fragment thereof coupled to various tags, such as for example, histidine tag, mouse or human Fc, or a signal sequence such as ROR1. The Spike protein is a type I membrane glycoprotein which assembles into trimers that constitute the spikes or peplomers on the surface of the enveloped MERS coronavirus particle. The protein has two essential functions, host receptor binding and membrane fusion, which are attributed to the N-terminal (S1) and C-terminal (S2) halves of the S protein.

The term “Severe Acute Respiratory Syndrome-Coronavirus-1 Receptor Binding Domain”. “SARS-CoV-1-RBD,” as used herein, refers to a viral receptor binding domain of the Spike protein that is present in the S1 subunit of the Spike protein and comprises the sequence set forth in SEQ ID NOs: 3 and 196, or biologically active fragments thereof.

Antibodies and Antigen-Binding Fragments Thereof As used herein, “antibodies or antigen binding fragments of the disclosure” refer to any one or more of the antibodies and antigen binding fragments provided herein. Antibodies and antigen binding fragments of the disclosure comprise a heavy chain (VH) comprising a heavy chain variable domain and a light chain (VL) comprising a light chain variable domain. A VH domain comprises three CDRs, such as any of the CDRs provided herein and as defined or identified by the Chothia, Kabat or IMGT systems. These CDRs are typically interspersed with frame-work regions (FR), and together comprise the VH domain. Similarly, a VL comprises three CDRs, such as any of the CDRs provided herein and as defined by the Chothia, Kabat or IMGT systems. These CDRs are typically interspersed with framework regions (FR), and together comprise the VL domain. The FR regions, such as FR1, FR2, FR3, and/or FR4 can similarly be defined or identified by the Chothia. Kabat or IMGT systems. Throughout the application, when CDRs are indicated as being, as identified or as defined by the Chothia, Kabat or IMGT systems, what is meant is that the CDRs are in accordance with that system (e.g., the Chothia CDRs, Kabat CDRs or the IMGT CDRs). Any of these terms can be used to indicate whether the Chothia, Kabat or IMGT CDRs are being referred to.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suit-able standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including. e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

The antibody name designations as used herein follow the formats: P0XA0Y or PXAY, P0XE0Y or PXEY, P0XD0Y or PXDY, P0XF0Y or PXFY, P0XH0Y or PXHY, P0XC0Y or PXCY, P0XG0Y or PXGY, P0XB0Y or PXBY, each denotes the same antibody. For example, the antibody name designation P04A05 is being used interchangeably with the designation P4A5, both designations denote the same antibody.

In some embodiments, the disclosure provides for antibodies or antigen-binding fragments thereof that bind SARS-CoV-2 and/or SARS-CoV-1. In certain embodiments, the antibodies or antigen-binding fragments thereof bind the spike protein of SARS-CoV-2 and/or SARS-CoV-1. In certain embodiments, the antibodies or antigen-binding fragments thereof bind the spike protein having an amino acid sequence that is at least 90%, 91%, 92%, 93%, 94%, 95%, 96%, 97%, 98%, 99%, or 100% identical to the amino acid sequence of SEQ ID NOs: 1, 3, 194 or 196 or biologically active fragments thereof. In certain embodiments, the antibodies or antigen-binding fragments thereof bind the S1 region of the spike protein. In other embodiments, the antibodies or antigen binding fragments thereof bind the receptor-binding domain (RBD) of the spike protein. In yet other embodiments, the antibodies or antigen binding fragments thereof bind an RBD protein having an amino acid sequence that is at least 90%, 91%, 92%, 93%, 94%, 95%, 96%, 97%, 98%, 99%, or 100% identical to the amino acid sequence of SEQ ID NO: 2, 3, 195, or 196. In certain embodiments the antibodies and antigen binding fragments are neutralizing antibodies.

In certain embodiments, the antibodies and antigen binding fragments thereof do not cross react with human antigens. In yet other embodiments, the antibodies and antigen binding fragments thereof do not cross-react the RBD of the SARS-CoV-1 RBD having an amino acid sequence as set forth in SEQ ID No: 3 or 196.

In certain embodiments, the recombinant antibody or antigen-binding fragment thereof in any one of the preceding claims, wherein in the antibody or antigen-binding fragment is capable of binding and neutralizing one or more of SARS-CoV-2 variants designated as: D614G, N501Y, E484K, E484Q, K417N and/or L452R. In other embodiments, the recombinant antibody or antigen-binding fragment thereof in any one of the preceding claims, wherein in the antibody or antigen-binding fragment is capable of binding and neutralizing one or more of SARS-CoV-2 variants designated as: B.1.1.7, B.1.351, 501YV2

In certain embodiments, the antibodies or antigen binding fragments thereof bind the RBD of the spike protein, such as for example, RBD-A or RBD-B. Examples of antibodies or antigen binding fragments thereof that bind RBD-A include but are not limited to the following antibodies P09D05, P11A11, P11A06, P11G07, P09D07, or P04E05.

In certain embodiments, the neutralizing antibodies or antigen-binding fragments comprise a variable heavy chain (VH) and variable light chain (VL). In some embodiments, the VH chain is selected from the group comprising:
1) a VH-CDR1 comprising an amino acid sequence of SEQ ID NO: 6, a VH-CDR2 comprising an amino acid sequence of SEQ ID NO: 7, and a VH-CDR3 comprising an amino acid sequence of SEQ ID NO: 8;
2) a VH-CDR1 comprising an amino acid sequence of SEQ ID NO: 16, a VH-CDR2 comprising an amino acid sequence of SEQ ID NO: 17, and a VH-CDR3 comprising an amino acid sequence of SEQ ID NO: 18;
3) a VH-CDR1 comprising an amino acid sequence of SEQ ID NO: 26, a VH-CDR2 comprising an amino acid sequence of SEQ ID NO: 27, and a VH-CDR3 comprising an amino acid sequence of SEQ ID NO: 28;
4) a VH-CDR1 comprising an amino acid sequence of SEQ ID NO: 36, a VH-CDR2 comprising an amino acid sequence of SEQ ID NO: 37, and a VH-CDR3 comprising an amino acid sequence of SEQ ID NO: 38;
5) a VH-CDR1 comprising an amino acid sequence of SEQ ID NO: 46, a VH-CDR2 comprising an amino acid sequence of SEQ ID NO: 47, and a VH-CDR3 comprising an amino acid sequence of SEQ ID NO: 48;
6) a VH-CDR1 comprising an amino acid sequence of SEQ ID NO: 56, a VH-CDR2 comprising an amino acid sequence of SEQ ID NO: 57, and a VH-CDR3 comprising an amino acid sequence of SEQ ID NO: 58;
7) a VH-CDR1 comprising an amino acid sequence of SEQ ID NO: 66, a VH-CDR2 comprising an amino acid sequence of SEQ ID NO: 67, and a VH-CDR3 comprising an amino acid sequence of SEQ ID NO: 68;
8) a VH-CDR1 comprising an amino acid sequence of SEQ ID NO: 76, a VH-CDR2 comprising an amino acid sequence of SEQ ID NO: 77, and a VH-CDR3 comprising an amino acid sequence of SEQ ID NO: 78;
9) a VH-CDR1 comprising an amino acid sequence of SEQ ID NO: 86, a VH-CDR2 comprising an amino acid sequence of SEQ ID NO: 87, and a VH-CDR3 comprising an amino acid sequence of SEQ ID NO: 88;
10) a VH-CDR1 comprising an amino acid sequence of SEQ ID NO: 96, a VH-CDR2 comprising an amino acid sequence of SEQ ID NO: 97, and a VH-CDR3 comprising an amino acid sequence of SEQ ID NO: 98;
11) a VH-CDR1 comprising an amino acid sequence of SEQ ID NO: 106, a VH-CDR2 comprising an amino acid sequence of SEQ ID NO: 107, and a VH-CDR3 comprising an amino acid sequence of SEQ ID NO: 108;
12) a VH-CDR1 comprising an amino acid sequence of SEQ ID NO: 116, a VH-CDR2 comprising an amino acid sequence of SEQ ID NO: 117, and a VH-CDR3 comprising an amino acid sequence of SEQ ID NO: 118;
13) a VH-CDR1 comprising an amino acid sequence of SEQ ID NO: 126, a VH-CDR2 comprising an amino acid sequence of SEQ ID NO: 127, and a VH-CDR3 comprising an amino acid sequence of SEQ ID NO: 128;
14) a VH-CDR1 comprising an amino acid sequence of SEQ ID NO: 136, a VH-CDR2 comprising an amino acid sequence of SEQ ID NO: 137, and a VH-CDR3 comprising an amino acid sequence of SEQ ID NO: 138;
15) a VH-CDR1 comprising an amino acid sequence of SEQ ID NO: 146, a VH-CDR2 comprising an amino acid sequence of SEQ ID NO: 147, and a VH-CDR3 comprising an amino acid sequence of SEQ ID NO: 148;
16) a VH-CDR1 comprising an amino acid sequence of SEQ ID NO: 156, a VH-CDR2 comprising an amino acid sequence of SEQ ID NO: 157, and a VH-CDR3 comprising an amino acid sequence of SEQ ID NO: 158;
17) a VH-CDR1 comprising an amino acid sequence of SEQ ID NO: 166, a VH-CDR2 comprising an amino acid sequence of SEQ ID NO: 167, and a VH-CDR3 comprising an amino acid sequence of SEQ ID NO: 168.
18) a VH-CDR1 comprising an amino acid sequence of SEQ ID NO: 176, a VH-CDR2 comprising an amino acid sequence of SEQ ID NO: 177, and a VH-CDR3 comprising an amino acid sequence of SEQ ID NO: 178;
19) a VH-CDR1 comprising an amino acid sequence of SEQ ID NO: 186, a VH-CDR2 comprising an amino acid sequence of SEQ ID NO: 187, and a VH-CDR3 comprising an amino acid sequence of SEQ ID NO: 188.
20) a VH-CDR1 comprising an amino acid sequence of SEQ ID NO: 199, a VH-CDR2 comprising an amino acid sequence of SEQ ID NO: 200, and a VH-CDR3 comprising an amino acid sequence of SEQ ID NO: 201.
21) a VH-CDR1 comprising an amino acid sequence of SEQ ID NO: 209, a VH-CDR2 comprising an amino acid sequence of SEQ ID NO: 210, and a VH-CDR3 comprising an amino acid sequence of SEQ ID NO: 211.
22) a VH-CDR1 comprising an amino acid sequence of SEQ ID NO: 219, a VH-CDR2 comprising an amino acid sequence of SEQ ID NO: 220, and a VH-CDR3 comprising an amino acid sequence of SEQ ID NO: 221.
23) a VH-CDR1 comprising an amino acid sequence of SEQ ID NO: 229, a VH-CDR2 comprising an amino acid sequence of SEQ ID NO: 230, and a VH-CDR3 comprising an amino acid sequence of SEQ ID NO: 231.
24) a VH-CDR1 comprising an amino acid sequence of SEQ ID NO: 239, a VH-CDR2 comprising an amino acid sequence of SEQ ID NO: 240, and a VH-CDR3 comprising an amino acid sequence of SEQ ID NO: 241.
25) a VH-CDR1 comprising an amino acid sequence of SEQ ID NO: 249, a VH-CDR2 comprising an amino acid sequence of SEQ ID NO: 250, and a VH-CDR3 comprising an amino acid sequence of SEQ ID NO: 251.
26) a VH-CDR1 comprising an amino acid sequence of SEQ ID NO: 259, a VH-CDR2 comprising an amino acid sequence of SEQ ID NO: 260, and a VH-CDR3 comprising an amino acid sequence of SEQ ID NO: 261.
27) a VH-CDR1 comprising an amino acid sequence of SEQ ID NO: 269, a VH-CDR2 comprising an amino acid sequence of SEQ ID NO: 270, and a VH-CDR3 comprising an amino acid sequence of SEQ ID NO: 271.
28) a VH-CDR1 comprising an amino acid sequence of SEQ ID NO: 279, a VH-CDR2 comprising an amino acid sequence of SEQ ID NO: 280, and a VH-CDR3 comprising an amino acid sequence of SEQ ID NO: 281.
29) a VH-CDR1 comprising an amino acid sequence of SEQ ID NO: 289, a VH-CDR2 comprising an amino acid sequence of SEQ ID NO: 290, and a VH-CDR3 comprising an amino acid sequence of SEQ ID NO: 291.
30) a VH-CDR1 comprising an amino acid sequence of SEQ ID NO: 299, a VH-CDR2 comprising an amino acid sequence of SEQ ID NO: 300, and a VH-CDR3 comprising an amino acid sequence of SEQ ID NO: 301.
31) a VH-CDR1 comprising an amino acid sequence of SEQ ID NO: 309, a VH-CDR2 comprising an amino acid sequence of SEQ ID NO: 310, and a VH-CDR3 comprising an amino acid sequence of SEQ ID NO: 311.
32) a VH-CDR1 comprising an amino acid sequence of SEQ ID NO: 319, a VH-CDR2 comprising an amino acid sequence of SEQ ID NO: 320, and a VH-CDR3 comprising an amino acid sequence of SEQ ID NO: 321.
33) a VH-CDR1 comprising an amino acid sequence of SEQ ID NO: 329, a VH-CDR2 comprising an amino acid sequence of SEQ ID NO: 330, and a VH-CDR3 comprising an amino acid sequence of SEQ ID NO: 331.
34) a VH-CDR1 comprising an amino acid sequence of SEQ ID NO: 339, a VH-CDR2 comprising an amino acid sequence of SEQ ID NO: 340, and a VH-CDR3 comprising an amino acid sequence of SEQ ID NO: 341.
35) a VH-CDR1 comprising an amino acid sequence of SEQ ID NO: 349, a VH-CDR2 comprising an amino acid sequence of SEQ ID NO: 350, and a VH-CDR3 comprising an amino acid sequence of SEQ ID NO: 351.
36) a VH-CDR1 comprising an amino acid sequence of SEQ ID NO: 359, a VH-CDR2 comprising an amino acid sequence of SEQ ID NO: 360, and a VH-CDR3 comprising an amino acid sequence of SEQ ID NO: 361.
37) a VH-CDR1 comprising an amino acid sequence of SEQ ID NO: 369, a VH-CDR2 comprising an amino acid sequence of SEQ ID NO: 370, and a VH-CDR3 comprising an amino acid sequence of SEQ ID NO: 371;
38) a VH-CDR1 comprising an amino acid sequence of SEQ ID NO: 379, a VH-CDR2 comprising an amino acid sequence of SEQ ID NO: 380, and a VH-CDR3 comprising an amino acid sequence of SEQ ID NO: 381;
39) a VH-CDR1 comprising an amino acid sequence of SEQ ID NO: 389, a VH-CDR2 comprising an amino acid sequence of SEQ ID NO: 390, and a VH-CDR3 comprising an amino acid sequence of SEQ ID NO: 391.
40) a VH-CDR1 comprising an amino acid sequence of SEQ ID NO: 399, a VH-CDR2 comprising an amino acid sequence of SEQ ID NO: 400, and a VH-CDR3 comprising an amino acid sequence of SEQ ID NO: 401,
41) a VH-CDR1 comprising an amino acid sequence of SEQ ID NO: 409, a VH-CDR2 comprising an amino acid sequence of SEQ ID NO: 410, and a VH-CDR3 comprising an amino acid sequence of SEQ ID NO: 411;
42) a VH-CDR1 comprising an amino acid sequence of SEQ ID NO: 419, a VH-CDR2 comprising an amino acid sequence of SEQ ID NO: 420, and a VH-CDR3 comprising an amino acid sequence of SEQ ID NO: 421;
43) a VH-CDR1 comprising an amino acid sequence of SEQ ID NO: 429, a VH-CDR2 comprising an amino acid sequence of SEQ ID NO: 430, and a VH-CDR3 comprising an amino acid sequence of SEQ ID NO: 431;
44) a VH-CDR1 comprising an amino acid sequence of SEQ ID NO: 439, a VH-CDR2 comprising an amino acid sequence of SEQ ID NO: 440, and a VH-CDR3 comprising an amino acid sequence of SEQ ID NO: 441;
45) a VH-CDR1 comprising an amino acid sequence of SEQ ID NO: 449, a VH-CDR2 comprising an amino acid sequence of SEQ ID NO: 450, and a VH-CDR3 comprising an amino acid sequence of SEQ ID NO: 451;
46) a VH-CDR1 comprising an amino acid sequence of SEQ ID NO: 459, a VH-CDR2 comprising an amino acid sequence of SEQ ID NO: 460, and a VH-CDR3 comprising an amino acid sequence of SEQ ID NO: 461.
47) a VH-CDR1 comprising an amino acid sequence of SEQ ID NO: 469, a VH-CDR2 comprising an amino acid sequence of SEQ ID NO: 470, and a VH-CDR3 comprising an amino acid sequence of SEQ ID NO: 471;
48) a VH-CDR1 comprising an amino acid sequence of SEQ ID NO: 479, a VH-CDR2 comprising an amino acid sequence of SEQ ID NO: 480, and a VH-CDR3 comprising an amino acid sequence of SEQ ID NO: 481;

wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody.

In some embodiments, the VL chain is selected from the group comprising:
(1) a VL-CDR1 comprising an amino acid sequence of SEQ ID NO: 11, a VL-CDR2 comprising an amino acid sequence of SEQ ID NO: 12, and a VL-CDR3 comprising an amino acid sequence of SEQ ID NO: 13;
(2) a VL-CDR1 comprising an amino acid sequence of SEQ ID NO: 21, a VL-CDR2 comprising an amino acid sequence of SEQ ID NO: 22, and a VL-CDR3 comprising an amino acid sequence of SEQ ID NO: 23;
(3) a VL-CDR1 comprising an amino acid sequence of SEQ ID NO: 31, a VL-CDR2 comprising an amino acid sequence of SEQ ID NO: 32, and a VL-CDR3 comprising an amino acid sequence of SEQ ID NO: 33;
(4) a VL-CDR1 comprising an amino acid sequence of SEQ ID NO: 41, a VL-CDR2 comprising an amino acid sequence of SEQ ID NO: 42, and a VL-CDR3 comprising an amino acid sequence of SEQ ID NO: 43;
(5) a VL-CDR1 comprising an amino acid sequence of SEQ ID NO: 51, a VL-CDR2 comprising an amino acid sequence of SEQ ID NO: 52, and a VL-CDR3 comprising an amino acid sequence of SEQ ID NO: 53;
(6) a VL-CDR1 comprising an amino acid sequence of SEQ ID NO: 61, a VL-CDR2 comprising an amino acid sequence of SEQ ID NO: 62, and a VL-CDR3 comprising an amino acid sequence of SEQ ID NO: 63;
(7) a VL-CDR1 comprising an amino acid sequence of SEQ ID NO: 71, a VL-CDR2 comprising an amino acid sequence of SEQ ID NO: 72, and a VL-CDR3 comprising an amino acid sequence of SEQ ID NO: 73;
(8) a VL-CDR1 comprising an amino acid sequence of SEQ ID NO: 81, a VL-CDR2 comprising an amino acid sequence of SEQ ID NO: 82, and a VL-CDR3 comprising an amino acid sequence of SEQ ID NO: 83;
(9) a VL-CDR1 comprising an amino acid sequence of SEQ ID NO: 91, a VL-CDR2 comprising an amino acid sequence of SEQ ID NO: 92, and a VL-CDR3 comprising an amino acid sequence of SEQ ID NO: 93;
(10) a VL-CDR1 comprising an amino acid sequence of SEQ ID NO: 101, a VL-CDR2 comprising an amino acid sequence of SEQ ID NO: 102, and a VL-CDR3 comprising an amino acid sequence of SEQ ID NO: 103;
(11) a VL-CDR1 comprising an amino acid sequence of SEQ ID NO: 111, a VL-CDR2 comprising an amino acid sequence of SEQ ID NO: 112, and a VL-CDR3 comprising an amino acid sequence of SEQ ID NO: 113;
(12) a VL-CDR1 comprising an amino acid sequence of SEQ ID NO: 121, a VL-CDR2 comprising an amino acid sequence of SEQ ID NO: 122, and a VL-CDR3 comprising an amino acid sequence of SEQ ID NO: 123;
(13) a VL-CDR1 comprising an amino acid sequence of SEQ ID NO: 131, a VL-CDR2 comprising an amino acid sequence of SEQ ID NO: 132, and a VL-CDR3 comprising an amino acid sequence of SEQ ID NO: 133;
(14) a VL-CDR1 comprising an amino acid sequence of SEQ ID NO: 141, a VL-CDR2 comprising an amino acid sequence of SEQ ID NO: 142, and a VL-CDR3 comprising an amino acid sequence of SEQ ID NO: 143;
(15) a VL-CDR1 comprising an amino acid sequence of SEQ ID NO: 151, a VL-CDR2 comprising an amino acid sequence of SEQ ID NO: 152, and a VL-CDR3 comprising an amino acid sequence of SEQ ID NO: 153;
(16) a VL-CDR1 comprising an amino acid sequence of SEQ ID NO: 161, a VL-CDR2 comprising an amino acid sequence of SEQ ID NO: 162, and a VL-CDR3 comprising an amino acid sequence of SEQ ID NO: 163;
(17) a VL-CDR1 comprising an amino acid sequence of SEQ ID NO: 171, a VL-CDR2 comprising an amino acid sequence of SEQ ID NO: 172, and a VL-CDR3 comprising an amino acid sequence of SEQ ID NO: 173;
(18) a VL-CDR1 comprising an amino acid sequence of SEQ ID NO: 181, a VL-CDR2 comprising an amino acid sequence of SEQ ID NO: 182, and a VL-CDR3 comprising an amino acid sequence of SEQ ID NO: 183.
(19) a VL-CDR1 comprising an amino acid sequence of SEQ ID NO: 191, a VL-CDR2 comprising an amino acid sequence of SEQ ID NO: 192, and a VL-CDR3 comprising an amino acid sequence of SEQ ID NO: 193;
(20) a VL-CDR1 comprising an amino acid sequence of SEQ ID NO: 204, a VL-CDR2 comprising an amino acid sequence of SEQ ID NO: 205, and a VL-CDR3 comprising an amino acid sequence of SEQ ID NO: 206;
(21) a VL-CDR1 comprising an amino acid sequence of SEQ ID NO: 214, a VL-CDR2 comprising an amino acid sequence of SEQ ID NO: 215, and a VL-CDR3 comprising an amino acid sequence of SEQ ID NO: 216;
(22) a VL-CDR1 comprising an amino acid sequence of SEQ ID NO: 224, a VL-CDR2 comprising an amino acid sequence of SEQ ID NO: 225, and a VL-CDR3 comprising an amino acid sequence of SEQ ID NO: 226;
(23) a VL-CDR1 comprising an amino acid sequence of SEQ ID NO: 234, a VL-CDR2 comprising an amino acid sequence of SEQ ID NO: 235, and a VL-CDR3 comprising an amino acid sequence of SEQ ID NO: 236;
(24) a VL-CDR1 comprising an amino acid sequence of SEQ ID NO: 244, a VL-CDR2 comprising an amino acid sequence of SEQ ID NO: 245, and a VL-CDR3 comprising an amino acid sequence of SEQ ID NO: 246;
(25) a VL-CDR1 comprising an amino acid sequence of SEQ ID NO: 254, a VL-CDR2 comprising an amino acid sequence of SEQ ID NO: 255, and a VL-CDR3 comprising an amino acid sequence of SEQ ID NO: 256;
(26) a VL-CDR1 comprising an amino acid sequence of SEQ ID NO: 264, a VL-CDR2 comprising an amino acid sequence of SEQ ID NO: 265, and a VL-CDR3 comprising an amino acid sequence of SEQ ID NO: 266;
(27) a VL-CDR1 comprising an amino acid sequence of SEQ ID NO: 274, a VL-CDR2 comprising an amino acid sequence of SEQ ID NO: 275, and a VL-CDR3 comprising an amino acid sequence of SEQ ID NO: 276;
(28) a VL-CDR1 comprising an amino acid sequence of SEQ ID NO: 284, a VL-CDR2 comprising an amino acid sequence of SEQ ID NO: 285, and a VL-CDR3 comprising an amino acid sequence of SEQ ID NO: 286;
(29) a VL-CDR1 comprising an amino acid sequence of SEQ ID NO: 294, a VL-CDR2 comprising an amino acid sequence of SEQ ID NO: 295, and a VL-CDR3 comprising an amino acid sequence of SEQ ID NO: 296;
(30) a VL-CDR1 comprising an amino acid sequence of SEQ ID NO: 304, a VL-CDR2 comprising an amino acid sequence of SEQ ID NO: 305, and a VL-CDR3 comprising an amino acid sequence of SEQ ID NO: 306;
(31) a VL-CDR1 comprising an amino acid sequence of SEQ ID NO: 314, a VL-CDR2 comprising an amino acid sequence of SEQ ID NO: 315, and a VL-CDR3 comprising an amino acid sequence of SEQ ID NO: 316;
(32) a VL-CDR1 comprising an amino acid sequence of SEQ ID NO: 324, a VL-CDR2 comprising an amino acid sequence of SEQ ID NO: 325, and a VL-CDR3 comprising an amino acid sequence of SEQ ID NO: 326;
(33) a VL-CDR1 comprising an amino acid sequence of SEQ ID NO: 334, a VL-CDR2 comprising an amino acid sequence of SEQ ID NO: 335, and a VL-CDR3 comprising an amino acid sequence of SEQ ID NO: 336;
(34) a VL-CDR1 comprising an amino acid sequence of SEQ ID NO: 344, a VL-CDR2 comprising an amino acid sequence of SEQ ID NO: 345, and a VL-CDR3 comprising an amino acid sequence of SEQ ID NO: 346;
(35) a VL-CDR1 comprising an amino acid sequence of SEQ ID NO: 354, a VL-CDR2 comprising an amino acid sequence of SEQ ID NO: 355, and a VL-CDR3 comprising an amino acid sequence of SEQ ID NO: 356;
(36) a VL-CDR1 comprising an amino acid sequence of SEQ ID NO: 364, a VL-CDR2 comprising an amino acid sequence of SEQ ID NO: 365, and a VL-CDR3 comprising an amino acid sequence of SEQ ID NO: 366;
(37) a VL-CDR1 comprising an amino acid sequence of SEQ ID NO: 374, a VL-CDR2 comprising an amino acid sequence of SEQ ID NO: 375, and a VL-CDR3 comprising an amino acid sequence of SEQ ID NO: 376;
(38) a VL-CDR1 comprising an amino acid sequence of SEQ ID NO: 384, a VL-CDR2 comprising an amino acid sequence of SEQ ID NO: 385, and a VL-CDR3 comprising an amino acid sequence of SEQ ID NO: 386;
(39) a VL-CDR1 comprising an amino acid sequence of SEQ ID NO: 394, a VL-CDR2 comprising an amino acid sequence of SEQ ID NO: 395, and a VL-CDR3 comprising an amino acid sequence of SEQ ID NO: 396;
(40) a VL-CDR1 comprising an amino acid sequence of SEQ ID NO: 404, a VL-CDR2 comprising an amino acid sequence of SEQ ID NO: 405, and a VL-CDR3 comprising an amino acid sequence of SEQ ID NO: 406;
(41) a VL-CDR1 comprising an amino acid sequence of SEQ ID NO: 414, a VL-CDR2 comprising an amino acid sequence of SEQ ID NO: 415, and a VL-CDR3 comprising an amino acid sequence of SEQ ID NO: 416;
(42) a VL-CDR1 comprising an amino acid sequence of SEQ ID NO: 424, a VL-CDR2 comprising an amino acid sequence of SEQ ID NO: 425, and a VL-CDR3 comprising an amino acid sequence of SEQ ID NO: 426;
(43) a VL-CDR1 comprising an amino acid sequence of SEQ ID NO: 434, a VL-CDR2 comprising an amino acid sequence of SEQ ID NO: 435, and a VL-CDR3 comprising an amino acid sequence of SEQ ID NO: 436;
(44) a VL-CDR1 comprising an amino acid sequence of SEQ ID NO: 444, a VL-CDR2 comprising an amino acid sequence of SEQ ID NO: 445, and a VL-CDR3 comprising an amino acid sequence of SEQ ID NO: 446;
(45) a VL-CDR1 comprising an amino acid sequence of SEQ ID NO: 454, a VL-CDR2 comprising an amino acid sequence of SEQ ID NO: 455, and a VL-CDR3 comprising an amino acid sequence of SEQ ID NO: 456;
(46) a VL-CDR1 comprising an amino acid sequence of SEQ ID NO: 464, a VL-CDR2 comprising an amino acid sequence of SEQ ID NO: 465, and a VL-CDR3 comprising an amino acid sequence of SEQ ID NO: 466;
(47) a VL-CDR1 comprising an amino acid sequence of SEQ ID NO: 474, a VL-CDR2 comprising an amino acid sequence of SEQ ID NO: 475, and a VL-CDR3 comprising an amino acid sequence of SEQ ID NO: 476;
(48) a VL-CDR1 comprising an amino acid sequence of SEQ ID NO: 484, a VL-CDR2 comprising an amino acid sequence of SEQ ID NO: 485, and a VL-CDR3 comprising an amino acid sequence of SEQ ID NO: 486;

wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody.

In some embodiments, the disclosure provides for an antibody or antigen-binding fragment thereof comprising a heavy chain variable domain (VH) and a light chain variable domain (VL), wherein the VH comprises: i) a VH-CDR1 having the amino acid sequence of SEQ ID NO: 26, but wherein 1, 2, 3, 4, or 5 amino acid substitutions, deletions or insertions are optionally present in the sequence of SEQ ID NO: 26; ii) a VH-CDR2 having the amino acid sequence of SEQ ID NO: 27, but wherein 1, 2, 3, 4, or 5 amino acid substitutions, deletions or insertions are optionally present in the sequence of SEQ ID NO: 27; and iii) a VH-CDR3 having the amino acid sequence of SEQ ID NO: 28, but wherein 1, 2, 3, 4, or 5 amino acid substitutions, deletions or insertions are optionally present in the sequence of SEQ ID NO: 28; and wherein the VL comprises: i) a VL-CDR1 having the amino acid sequence of SEQ ID NO: 31; but wherein 1, 2, 3, 4, or 5 amino acid substitutions, deletions or insertions are optionally present in the sequence of SEQ ID NO: 31; ii) a VL-CDR2 having the amino acid sequence of SEQ ID NO: 32, but wherein 1, 2, 3, 4, or 5 amino acid substitutions, deletions or insertions are optionally present in the sequence of SEQ ID NO: 32; and iii) a VL-CDR3 having the amino acid sequence of SEQ ID NO: 33: but wherein 1, 2, 3, 4, or 5 amino acid substitutions, deletions or insertions are optionally present in the sequence of SEQ ID NO: 33; wherein the amino acid substitutions, deletions or insertions reduce the binding affinity of the antibody or antigen-binding fragment thereof for the spike protein of SARS-CoV-2 affinity by no more than 2, 3, 4, 5, or 10-fold as compared to a reference antibody.

The present disclosure includes anti-SARS2-CoV-2 and/or SARS-CoV-1 antibodies and antigen-binding fragments thereof that bind the SARS2-CoV-2-S and/or SARS-CoV-1-S or spike protein. In some embodiments, the antibody is a neutralizing and/or blocking anti-SARS2-CoV-2 and/or SARS-CoV-1 antibody or antigen-binding fragment. A “neutralizing” or “blocking” antibody or antigen-binding fragment, as used herein, is intended to refer to an antibody or antigen-binding fragment whose binding to the SARS2-CoV-2-S and/or SARS-CoV-1 or spike protein: (i) interferes with and/or blocks the interaction between the SARS-CoV-2 and/or SARS-CoV-1 with the ACE receptor, such as the human ACE-2 receptor and/or (ii) inhibits the rate of infection and or disease progression.

In one embodiment, the neutralizing monoclonal antibody and antigen-binding fragments thereof bind SARS-CoV-2-S or Spike protein or fragments thereof. In some embodiments, an anti-SARS-CoV-2 antibody or antigen binding fragment thereof binds to the S1 portion of SARS-CoV-2 Spike protein. In some embodiments, an anti-SARS-CoV-2 antibody or antigen binding fragment thereof binds RBD portion of SARS-CoV-2 Spike protein.

The inhibition caused by an anti-SARS-CoV-2 neutralizing or blocking antibody may or may not be complete so long as it is detectable using an appropriate assay. Some examples of assays for detecting activity of a representative SARS-CoV-2 antibody or antigen-binding fragment are described in the Exemplification section. The skilled worker is aware of additional SARS-CoV-2 antibody activity assays.

In particular embodiments, the antibodies or antigen-binding fragments disclosed herein interferes with the interaction between SARS-CoV-2 and the RBD region of the SARS-CoV-2-S protein. In some embodiments, the anti-SARS-CoV2 antibodies or antigen-binding fragments block the interaction between SARS-CoV-2 and as ACE receptor, such as the human ACE-2 receptor, with an IC50 value of less than about 15 nM, as measured by the assay such as that described in the Exemplification section. In certain embodiments, the IC50 of the anti-SARS-CoV-2 antibody or fragment thereof is measured in an epitope competition assay, such as the epitope competition assay described in the Exemplification section provided herein.

In other embodiments, certain the antibodies or antigen-binding fragments disclosed herein interferes with the interaction between SARS-CoV-2 and/or SARS-CoV-land the RBD region of the SARS-CoV-2-S/SARS-CoV-1-S protein. In some embodiments, the anti-SARS-CoV2, and/or SARS-CoV-1 antibodies or antigen-binding fragments block the interaction between SARS-CoV-2 and/or SARS-CoV-1 with an ACE receptor, such as the human ACE-2 receptor, with an IC50 value of less than about 15 nM, as measured by the assay such as that described in the Exemplification section. In certain embodiments, the IC50 of the anti-SARS-CoV-2 antibody or fragment thereof is measured in an epitope competition assay, such as the epitope competition assay described in the Exemplification section provided herein.

The antibodies or antigen-binding fragments of the present disclosure may possess one or more of the aforementioned biological characteristics, or any combinations thereof. Other biological characteristics of the antibodies of the present disclosure will be evident to a person of ordinary skill in the art from a review of the present disclosure including the Exemplification section provided herein As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In some embodiments, any of the antibodies or antigen-binding fragments disclosed herein comprise 1, 2, 3, 4, 5, 6, 7, 8, 9, 10, 11, 12, 13, 14, 15, 16, 17, 18, 19, or 20 conservative amino acid substitutions as compared to a reference sequence. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2.

The heavy chain constant domains that correspond to the different classes of immunoglobulins are called a, S, e, y, and p, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

Non-limiting examples of antigen-binding fragments include: (i) Fab fragments; (ii) Fab′ fragments; (iii) F(ab′)2 fragments; (iv) Fd fragments; (v) Fv fragments; (vi) single-chain Fv (scFv) molecules; (vii) dAb fragments; and (viii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, cameliid antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), adnectins, small modular immunopharmaceuticals (SMIPs), and shark variable IgNAR domains, are also encompassed within the expression “antigen-binding fragment,” as used herein.

An antigen-binding fragment of an antibody will typically comprise at least one variable domain (e.g., at least one of a VH or VL). The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. In some embodiments, the hinge region comprises a glycine-serine linker.

Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present disclosure using routine techniques available in the art.

In certain embodiments of the disclosure, the anti-SARS-CoV-2 antibodies of the disclosure are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs, and in some embodiments, CDR3. However, the term “human antibody”, as used herein, is not intended to include antibodies in which CDR sequences derived from the germline of another mammalian species, such as a mouse, have been grafted onto human framework sequences.

The antibodies of the disclosure may, in some embodiments, be recombinant human antibodies. The term “recombinant human antibody”, as used herein, is intended to include all human antibodies that are prepared, expressed, or created by recombinant means, such as antibodies expressed using a recombinant expression vector transfected into a host cell or other methods that are well known in the art. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The current disclosure contemplates antibodies having one or more mutations in the hinge, CH2 or CH3 region, which may be desirable, for example, in production, to improve the yield of the desired antibody form.

The antibodies of the disclosure may be isolated antibodies or isolated antigen-binding fragments. An “isolated antibody” or “isolated antigen-binding fragment,” as used herein, means an antibody or antigen-binding fragment that has been identified and separated and/or recovered from at least one component of its natural environment. For example, an antibody or antigen-binding fragment that has been separated or removed from at least one component of an organism, or from a tissue or cell in which the antibody naturally exists or is naturally produced, is an “isolated antibody” or an “isolated antigen-binding fragment” for purposes of the present disclosure. An isolated antibody also includes an antibody in situ within a recombinant cell. Isolated antibodies or antigen-binding fragments are antibodies or antigen-binding fragments that have been subjected to at least one purification or isolation step. According to certain embodiments, an isolated antibody or antigen-binding fragment may be substantially free of other cellular material and/or chemicals.

The anti-SARS-CoV-2 and/or SARS-CoV-1 antibodies or antigen-binding fragments disclosed herein may comprise one or more amino acid substitutions, insertions and/or deletions in the framework and/or CDR regions of the heavy and light chain variable domains as compared to the corresponding germline sequences from which the antibodies were derived. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody or antigen-binding fragment was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments, which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence.

Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes anti-SARS-CoV-2 and/or SARS-CoV-1 antibodies (cross-reactive and non-cross reactive) comprising variants of any of the VH, VL, and/or CDR amino acid sequences disclosed herein having one or more conservative substitutions. For example, the present disclosure includes anti-SARS-CoV-2 and/or SARS-CoV-1 (cross-reactive and non-cross reactive) antibodies having VH, VL, and/or CDR amino acid sequences with, e.g., 10, 9, 8, 7, 6, 5, 4, 3, 2, or 1 conservative amino acid substitutions relative to any of the VH, VL, and/or CDR amino acid sequences disclosed herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

It should be noted that any portion of any of the antibodies or antigen-binding fragments of the disclosure may be similarly modified, such as with an epitope tag, a PEG moiety or moieties, and the like. Moreover, the antibodies or antigen-binding fragments may comprise more than one epitope tags, such as 2 epitope tags, or may include 0 epitope tags.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402. In some embodiments, the sequences are compared using EMBOSS Needle pairwise sequence alignment.

Two antibodies or antigen-binding fragments are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies or antigen-binding fragments will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In some embodiments, two antibodies or antigen-binding fragments are bioequivalent if there are no clinically meaningful differences in their safety, purity, and potency.

In some embodiments, two antibodies or antigen-binding fragments are bioequivalent if a patient can be switched one or more times between the reference product and the biological product without an expected increase in the risk of adverse effects, including a clinically significant change in immunogenicity, or diminished effectiveness, as compared to continued therapy without such switching.

In some embodiments, two antibodies or antigen-binding fragments are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-SARS-CoV-2 and/or SARS-CoV-1 antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies or antigen-binding fragments may include anti-SARS-CoV-2 and/or SARS-CoV-1 antibody variants comprising amino acid changes which modify the glycosylation characteristics of the antibodies or antigen-binding fragments, e.g., mutations which eliminate or remove glycosylation.

The present disclosure, according to certain embodiments, provides anti-SARS-CoV-2 antibodies or antigen-binding fragments that bind to SARS-CoV-2-S protein but not to the SARS-CoV-1-S protein. In some embodiments, the antibodies or antigen binding fragments thereof bind the RBD of SARS-CoV-2 but not the RBD of SARS-CoV-1.

The present disclosure also includes anti-SARS-CoV-2 antibodies that do not cross-react with human proteins.

The disclosure encompasses anti-SARS-CoV-2 and/or SARS-CoV-1 monoclonal antibodies conjugated to a therapeutic moiety (“immunoconjugate”), such as a cytotoxin or an antiviral agent.

In some embodiments, the antibodies of the present disclosure may be used in combination therapy.

In some embodiments, the antibodies of the present disclosure may be monospecific, bi-specific, or multispecific. Multispecific antibodies may be specific for different epitopes of one target polypeptide or may contain antigen-binding domains specific for more than one target polypeptide. See. e.g., Tutt et al., 1991, J. Immunol. 147:60-69; Kufer ei a/., 2004, Trends Biotechnol. 22:238-244. The anti-SARSCoV-2 antibodies or antigen-binding fragments of the present disclosure can be linked to or co-expressed with another functional molecule, e.g., another peptide or protein. For example, an antibody or antigen-binding fragment thereof can be functionally linked (e.g., by chemical coupling, genetic fusion, noncovalent association or otherwise) to one or more other molecular entities, such as another antibody or antigen-binding fragment to produce a bi-specific or a multispecific antibody with a second binding specificity. For example, the present disclosure includes bi-specific antibodies wherein one arm of an immunoglobulin is specific for SARS-CoV-2-S or a fragment thereof, such as the S1 region or the RBD region and the other arm of the immunoglobulin is specific for a second CoV such as SARS-CoV-1-S target or is conjugated to a therapeutic moiety.

An exemplary bi-specific antibody or antigen-binding fragment format that can be used in the context of the present disclosure involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to its antigen as compared to a bi-specific antibody lacking the amino acid difference. Variations on the bi-specific antibody format described above are contemplated within the scope of the present disclosure.

Other exemplary bispecific formats that can be used in the context of the present disclosure include, without limitation, e.g., scFv-based or diabody bispecific formats, IgG-scFv fusions, dual variable domain (DVD)-1g, Quadroma, knobs-into-holes, common light chain (e.g., common light chain with knobs-into-holes, etc.), CrossMab, CrossFab, (SEED)body, leucine zipper, Duobody, 1gG1/1gG2, dual acting Fab (DAF)-1gG, and Mab<2>bispecific formats (see, e.g., Klein et al. 2012, mAbs 4:6, 1 −1 1, and references cited therein, for a review of the foregoing formats). Bispecific antibodies or antigen-binding fragments can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. C em. Soc. [Epub: Dec. 4, 2012]).

### Nucleic Acids Encoding SARS-CoV-2 Antibodies

In some embodiments, the disclosure provides for a nucleic acid capable of expressing any of the antibodies of antigen-binding fragments disclosed herein. The nucleic acids may be single-stranded or double-stranded, DNA or RNA molecules. In further embodiments, the antibody or antigen-binding fragment nucleic acid sequences can be isolated, recombinant, and/or fused with a heterologous nucleotide sequence, or in a DNA library. In some embodiments, the nucleic acid comprises a nucleotide sequence that is at least 80%, 85/a, 90%, 91%, 92%, 93%, 94%, 95%, 96%, 97%, 98%, 99% or 100% identical to any one of SEQ ID NOs: 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 198, 203, 208, 213, 218, 223, 228, 233, 238, 243, 248, 253, 258, 263, 268, 273, 278, 283, 288, 293, 298, 303, 308, 313, 318, 323, 328, 333, 338, 343, 348, 353, 358, 363, 368, 373, 378, 383, 388, 393, 398, 403, 408, 413, 418, 423, 428, 433, 438, 443, 448, 453, 458, 463, 468, 473, 478, and/or 483.

In certain embodiments, nucleic acids encoding antibodies or antigen-binding fragments also include nucleotide sequences that hybridize under highly stringent conditions to a polynucleotide encoding any of the above-mentioned antibodies or antigen-binding fragments nucleotide sequence, or complement sequences thereof. In some embodiments, the nucleic acids hybridize under highly stringent conditions to a polynucleotide encoding an amino acid sequence that is at least 80%, 85%, 90%, 91%, 92%, 93%, 94%, 95%, 96%, 97%, 98%, 99% or 100% identical to any one of SEQ ID NOs: 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 198, 203, 208, 213, 218, 223, 228, 233, 238, 243, 248, 253, 258, 263, 268, 273, 278, 283, 288, 293, 298, 303, 308, 313, 318, 323, 328, 333, 338, 343, 348, 353, 358, 363, 368, 373, 378, 383, 388, 393, 398, 403, 408, 413, 418, 423, 428, 433, 438, 443, 448, 453, 458, 463, 468, 473, 478, and/or 483.

In some embodiments, the nucleic acids hybridize under highly stringent conditions to a polynucleotide encoding an amino acid sequence that is at least 80%, 85%, 90%, 91%, 92%, 93%, 94%, 95%, 96%, 97%, 98%, 99% or 100% identical to any one of SEQ ID NOs: 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 198, 203, 208, 213, 218, 223, 228, 233, 238, 243, 248, 253, 258, 263, 268, 273, 278, 283, 288, 293, 298, 303, 308, 313, 318, 323, 328, 333, 338, 343, 348, 353, 358, 363, 373, 378, 383, 388, 393, 398, 403, 408, 413, 418, 423, 428, 433, 438, 443, 448, 453, 458, 463, 468, 473, 478, and/or 483. One of ordinary skill in the art will understand readily that appropriate stringency conditions, which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids encoding the antibody or antigen-binding fragment thereof due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In some embodiments, the disclosure provides for a vector comprising any of the nucleic acids disclosed herein. In some embodiments, the disclosure provides for a host cell comprising any of the vectors disclosed herein.

Regardless of when an antibody of the disclosure is a full length antibody or an antigen binding fragment, antibodies and antigen binding fragments of the disclosure can be recombinantly expressed in cell lines. In these embodiments, sequences encoding particular antibodies or antigen binding fragments can be used for transformation of a suitable host cell, such as a mammalian host cell or yeast host cell. According to these embodiments, transformation can be achieved using any known method for introducing polynucleotides into a host cell, including, for example packaging the polynucleotide in a virus (or into a viral vector) and transducing a host cell with the virus (or vector) or by transfection procedures known in the art. Generally, the transformation procedure used may depend upon the host to be transformed. Methods for introducing heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

According to certain embodiments of the disclosure, a nucleic acid molecule encoding the amino acid sequence of a heavy chain constant region (all or a portion), a heavy chain variable region of the disclosure, a light chain constant region, or a light chain variable region of the disclosure is inserted into an appropriate expression vector using standard ligation techniques. In a preferred embodiment, the heavy or light chain constant region is appended to the C-terminus of the appropriate variable region and is ligated into an expression vector. The vector is typically selected to be functional in the particular host cell employed (i.e., the vector is compatible with the host cell machinery such that amplification of the gene and/or expression of the gene can occur). For a review of expression vectors, see, Goeddel (ed.), 1990, Meth. Enzymol. Vol. 185, Academic Press. N.Y. In the context of antibody expression, both the heavy and light chain may be expressed from the same vector (e.g., from the same or different promoters present on the same vector) or the heavy and light chains may be expressed from different vectors. In certain embodiments, the heavy and light chains are expressed from different vectors, which are transfected into the same host cell and co-expressed. Regardless of when the heavy and light chains are expressed in the same host cell from the same or a different vector, the chains can then associate to form an antibody (or antibody fragment, depending on the portions of the heavy and light chain being expressed).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. These portions of vectors are well known, and there are numerous generally available vectors that can be selected and used for the expression of proteins. One can readily select vectors based on the desired host cell and application.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly. Mass.) is suitable for most gram-negative bacteria and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

The expression and cloning vectors of the disclosure will typically contain a promoter that is recognized by the host organism and operably linked to the molecule encoding heavy and/or light chain. Promoters are untranscribed sequences located upstream (i.e., 5′) to the start codon of a structural gene (generally within about 100 to 1000 bp) that control the transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding the heavy chain or light chain comprising an antibody or antigen binding fragment of the disclosure. In certain embodiments, the same promoter is used for both the heavy and light chain. In other embodiments, different promoters (present on the same or different vectors) are used for each.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest include, but are not limited to: the SV40 early promoter region (Bemoist and Chambon, 1981, Nature 290:304-10); the CMV promoter; the promoter contained in the 3′ long terminal repeat of Rous sarcoma virus (Yamamoto et al., 1980, Cell 22:787-97); the herpes thymidine kinase promoter (Wagner et al., 1981, Proc. Natl. Acad. Sci. USA 78:1444-45); the regulatory sequences of the metallothionine gene (Brinster et al., 1982, Nature 296:39-42); prokaryotic expression vectors such as the beta-lactamase promoter (Villa-Kamaroff et al., 1978, Proc. Natl. Acad. Sci. USA 75:3727-31); or the tac promoter (DeBoer et al., 1983, Proc. Natl. Acad. Sci. USA 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-46; Omitz et al., 1986, Cold Spring Harbor Symp. Quant. Biol. 50:399-409 (1986); MacDonald, 1987, Hepatology 7:425-515); the insulin gene control region that is active in pancreatic beta cells (Hanahan, 1985, Nature 315:115-22); the immunoglobulin gene control region that is active in lymphoid cells (Grosschedl et al., 1984, Cell 38:647-58; Adames et al., 1985. Nature 318:533-38; Alexander et al., 1987, Mol. Cell. Biol. 7:1436-44); the mouse mammary tumor virus control region that is active in testicular, breast, lymphoid and mast cells (Leder et al., 1986, Cell 45:485-95); the albumin gene control region that is active in liver (Pinkert et al., 1987, Genes and Devel. 1:268-76); the alpha-feto-protein gene control region that is active in liver (Krumlauf et al., 1985, Mol. Cell. Biol. 5:1639-48; Hammer et al., 1987, Science 235:53-58); the alpha 1-antitrypsin gene control region that is active in liver (Kelsey et al., 1987, Genes and Devel. 1:161-71); the beta-globin gene control region that is active in myeloid cells (Mogram et al., 1985, Nature 315:338-40; Kollias et al., 1986, Cell 46:89-94); the myelin basic protein gene control region that is active in oligodendrocyte cells in the brain (Readhead et al., 1987, Cell 48:703-12); the myosin light chain-2 gene control region that is active in skeletal muscle (Sani, 1985, Nature 314:283-86); and the gonadotropic releasing hormone gene control region that is active in the hypothalamus (Mason et al., 1986, Science 234:1372-78).

The vector may also include an enhancer sequence to increase transcription of DNA encoding light chain or heavy chain.

Expression vectors of the disclosure may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding light chain or heavy chain or light chain and heavy chain comprising an antibody or antigen binding fragment of the disclosure has been inserted into the proper site of the vector, the completed vector may be inserted into a suitable host cell for amplification and/or polypeptide expression. The transformation of an expression vector into a selected host cell may be accomplished by well-known methods including transfection, infection, calcium phosphate co-precipitation, electroporation, microinjection, lipofection. DEAE-dextran mediated transfection, or other known techniques. The method selected will in part be a function of the type of host cell to be used. These methods and other suitable methods are well known to the skilled worker.

The host cell, when cultured under appropriate conditions, synthesizes the antibody or antigen binding fragment of the disclosure that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as host cells for expression are well known in the art and include, but are not limited to, many immortalized cell lines available from the American Type Culture Collection (A.T.C.C.), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. In another embodiment, one may select a cell line from the B cell lineage that does not make its own antibody but has a capacity to make and secrete a heterologous antibody (e.g., mouse myeloma cell lines NS0 and SP2/0). In other embodiments, a cell other than a mammalian cell is used, such as a yeast cell line (e.g., Pichia).

In certain embodiments, the cell line stably expresses an antibody or antigen binding fragment of the disclosure. In other embodiments, the cells transiently express an antibody or antigen binding fragment of the disclosure.

### Pharmaceutical Compositions and Modes of Administration

The antibodies or agents of the invention (also referred to herein as “active compounds”), and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent, which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Methods of Treatment and Therapeutic Uses

The antibodies (or antigen-binding fragments thereof) and compositions of the present application are useful for the treatment of a disease, disorder, or condition associated with COVID-19 (e.g., SARS-CoV-2 infections and/or SARS-CoV-1 infections). As used herein, “treatment,” “treat.” or “treating” is defined as the application or administration of a therapeutic agent to a patient, who has a disease or condition associated with COVID-19 (e.g., SARS-CoV-2 infections and/or SARS-CoV-1 infections); or a symptom of, or a predisposition towards such disease or condition associated with COVID-19 (e.g., SARS-CoV-2 infections and/or SARS-CoV-1 infections), with the purpose to cure, heal, alleviate, relieve, alter, remedy, ameliorate, improve or affect the disease, condition, symptoms thereof or the predisposition thereto. In some embodiments, the present application provides a method of treating one or more COVID-19-associated complications (e.g., SARS-CoV-2 infections and/or SARS-CoV-1 infections) by administrating an antibody or antigen-binding fragment thereof or a composition as described herein to a patient under conditions that generate a beneficial therapeutic response in the patient. In some embodiments, an antibody or antigen-binding fragment thereof as described herein may be administered at a therapeutically effective dose or amount to a patient with COVID-19 infection (e.g., SARS-CoV-2 infection and/or SARS-CoV-1 infections).

In some embodiments, the antibodies or antigen-binding fragments thereof, or compositions comprising any of the foregoing, as described herein are useful to treat subjects suffering from the severe and acute respiratory infection caused by COVID-19 (e.g., SARS-CoV-2). In some embodiments, the antibodies or antigen-binding fragments thereof, or compositions comprising any of the foregoing, as described herein are useful in decreasing viral titer or reducing viral load in the host. In some embodiments, the antibodies or antigen-binding fragments thereof, or compositions comprising any of the foregoing, as described herein are useful in preventing or reducing inflammation in the lung of a subject with COVID-19 infection (e.g., SARS-CoV-2 infection and/or SARS-CoV-1 infections). In some embodiments, the antibodies or antigen-binding fragments thereof, or compositions comprising any of the foregoing, as described herein are useful in preventing or reducing interstitial, peribronchiolar or perivascular inflammation, alveolar damage and pleural changes in a subject with COVID-19 infection (e.g., SARS-CoV-2 infection and/or SARS-CoV-1 infections).

In some embodiments, the antibodies or antigen-binding fragments thereof, or compositions comprising any of the foregoing, as described herein may be used in or administered to a subject in need thereof to relieve or prevent or ameliorate or decrease the severity of one or more of the symptoms or conditions of the disease or disorder. The antibodies or antigen-bind fragments thereof, or compositions comprising any of the foregoing, may be used to ameliorate or reduce the severity of at least one symptom of COVID-19 infection (e.g., SARS-CoV-2 infection and/or SARS-CoV-1 infections), including, but not limited to fever, cough, shortness of breath, pneumonia, diarrhea, organ failure (e.g., kidney failure and renal dysfunction), septic shock, and death.

In some embodiments, the antibodies or antigen-binding fragments thereof, or compositions comprising any of the foregoing, as described in the present applicant may be used prophylactically in subjects at risk for developing COVID-19 infection (e.g., SARS-CoV-2 and/or SARS-CoV-1 infections), such as immunocompromised individuals, elderly adults (more than 65 years of age), children younger than 2 years of age, travelers, healthcare workers, family members in close proximity to a COVID-19 infection (e.g., SARS-CoV-2 and/or SARS-CoV-1 infections) patient, adults or children with contact with persons with confirmed or suspected COVID-19 infection (e.g., SARS-CoV-2 infection and/or SARS-CoV-1 infections), and patients with a medical history (e.g., increased risk of pulmonary infection, heart disease or diabetes).

In some embodiments, the antibodies or antigen-binding fragments thereof, or compositions comprising any of the foregoing, as described in the present applicant may be used in the preparation of a medicament for treating patients suffering from COVID-19 infection (e.g., SARS-CoV-2 and/or SARS-CoV-1 infections). In some embodiments, the antibodies or antigen-binding fragments thereof, or compositions comprising any of the foregoing, as described in the present application may be used as adjunct therapy with any other agent or any other therapy known to those skilled in the art useful for the treatment of COVID-19 infection (e.g., SARS-CoV-2 and/or SARS-CoV-1 infections).

TABLE 1
Sequence ID NOs. Key

| | | SEQ ID |
|---|---|---|
| Name | Description | NO: |
| SARS-COV-2 | MFVFLVLLPLVSSQCVNLTTRTQLPPAYTNSFTRGVYYPDKVFR | 1 |
| Spike | SSVLHSTQDLFLPFFSNVTWFHAIHVSGTNGTKRFDNPVLPFND | |
| (This includes | GVYFASTEKSNIIRGWIFGTTLDSKTQSLLIVNNATNVVIKVCEF | |
| a linker) | QFCNDPFLGVYYHKNNKSWMESEFRVYSSANNCTFEYVSQPFL | |
| | MDLEGKQGNFKNLREFVFKNIDGYFKIYSKHTPINLVRDLPQGF | |
| | SALEPLVDLPIGINITRFQTLLALHRSYLTPGDSSSGWTAGAAAY | |
| | YVGYLQPRTFLLKYNENGTITDAVDCALDPLSETKCTLKSFTVE | |
| | KGIYQTSNFRVQPTESIVRFPNITNLCPFGEVFNATRFASVYAW | |
| | NRKRISNCVADYSVLYNSASFSTFKCYGVSPTKLNDLCFTNVY | |
| | ADSFVIRGDEVRQIAPGQTGKIADYNYKLPDDFTGCVIAWNSN | |
| | NLDSKVGGNYNYLYRLFRKSNLKPFERDISTEIYQAGSTPCNGV | |
| | EGFNCYFPLQSYGFQPTNGVGYQPYRVVVLSFELLHAPATVCG | |
| | PKKSTNLVKNKCVNFNFNGLTGTGVLTESNKKFLPFQQFGRDI | |
| | ADTTDAVRDPQTLEILDITPCSFGGVSVITPGTNTSNQVAVLYQ | |
| | DVNCTEVPVAIHADQLTPTWRVYSTGSNVFQTRAGCLIGAEHV | |
| | NNSYECDIPIGAGICASYQTQTNSPGSASSVASQSIIAYTMSLGA | |
| | ENSVAYSNNSIAIPTNFTISVTTEILPVSMTKTSVDCTMYICGDST | |
| | ECSNLLLQYGSFCTQLNRALTGIAVEQDKNTQEVFAQVKQIYK | |
| | TPPIKDFGGFNFSQILPDPSKPSKRSFIEDLLFNKVTLADAGFIKQ | |
| | YGDCLGDIAARDLICAQKFNGLTVLPPLLTDEMIAQYTSALLAG | |
| | TITSGWTFGAGAALQIPFAMQMAYRENGIGVTQNVLYENQKLI | |
| | ANQFNSAIGKIQDSLSSTASALGKLQDVVNQNAQALNTLVKQL | |
| | SSNFGAISSVLNDILSRLDPPEAEVQIDRLITGRLQSLQTYVTQQL | |
| | IRAAEIRASANLAATKMSECVLGQSKRVDFCGKGYHLMSFPQS | |
| | APHGVVFLHVTYVPAQEKNFTTAPAICHDGKAHFPREGVFVSN | |
| | GTHWFVTQRNFYEPQIITTDNTFVSGNCDVVIGIVNNTVYDPLQ | |
| | PELDSFKEELDKYFKNHTSPDVDLGDISGINASVVNIQKEIDRLN | |
| | EVAKNLNESLIDLQELGKYEQGSGYIPEAPRDGQAYVRKDGEW | |
| | VLLSTFLGRSLEVLFQGPGHHHHHHHHSAWSHPQFEKGGGSGG | |
| | GGSGGSAWSHPQFEK | |
| SARS-COV-2 | RVQPTESIVRFPNITNLCPFGEVFNATRFASVYAWNRKRISNCV | 2 |
| RBD2 | ADYSVLYNSASFSTFKCYGVSPTKLNDLCFTNVYADSFVIRGDE | |
| (This includes | VRQIAPGQTGKIADYNYKLPDDFTGCVIAWNSNNLDSKVGGNY | |
| a linker) | NYLYRLFRKSNLKPFERDISTEIYQAGSTPCNGVEGFNCYFPLQS | |
| | YGFQPTNGVGYQPYRVVVLSFELLHAPATVCGPKKSTNLVKNK | |
| | CVNFNFNGLTGTGVLTESNKKFLPFQQFGRDIADTTDAVRDPQ | |
| | TLEILDITPCSGSGHHHHHHHHHHGSGGLNDIFEAQKIEWHE | |
| SARS-COV-1 | RVVPSGDVVRFPNITNLCPFGEVFNATKFPSVYAWERKKISNCV | 3 |
| RBD1 | ADYSVLYNSTFFSTFKCYGVSATKLNDLCFSNVYADSFVVKGD | |
| (This includes | DVRQIAPGQTGVIADYNYKLPDDFMGCVLAWNTRNIDATSTG | |
| a linker) | NYNYKYRYLRHGKLRPFERDISNVPFSPDGKPCTPPALNCYWP | |
| | LNDYGFYTTTGIGYQPYRVVVLSFELLNAPATVCGPKLSTDLIK | |
| | NQCVNFNFNGLTGTGVLTPSSKRFQPFQQFGRDVSDFTDSVRD | |
| | PKTSEILDISPCSGSGHHHHHHHHHHGSGGLNDIFEAQKIEWHE | |
| L12.bP11A06 | EVQLVESGGGLIQPGGSLRLSCAASGLTVSSNYMSWVRQAPGK | 4 |
| | GLEWVSVIYSGGSTFYADSVKGRFTISRDNSKNTLYLQMNSLR | |
| | AEDTAVYYCARDLDVYGLDVWGQGTTVTVSS | |
| | GAAGTGCAGCTGGTGGAGTCTGGAGGAGGCTTGATCCAGCC | 5 |
| | TGGGGGGTCCCTGAGACTCTCCTGTGCAGCCTCTGGGTTAAC | |
| | CGTCAGTAGCAACTACATGAGCTGGGTCCGCCAGGCTCCAG | |
| | GGAAGGGGCTGGAGTGGGTCTCAGTTATTTATAGCGGTGGT | |
| | AGCACATTCTACGCAGACTCCGTGAAGGGCCGATTCACCATC | |
| | TCCAGAGACAATTCCAAGAACACGCTGTATCTTCAAATGAAC | |
| | AGCCTGAGAGCCGAGGACACGGCCGTGTATTACTGTGCGAG | |
| | AGATTTAGATGTCTACGGITTGGACGTCTGGGGCCAAGGGAC | |
| | CACGGTCACCGTCTCCTCA | |
| | GLTVSSNY | 6 |
| | IYSGGST | 7 |
| | ARDLDVYGLDV | 8 |
| | DIVMTQSPSFLSASVGDRVTITCRASQGISSYLAWYQQKPGKAP | 9 |
| | KLLIYAASTLQSGVPSRFSGSGSGTEFTLTISSLQPEDFATYYCQ | |
| | QLNSYPPKFTFGPGTKVEIK | |
| | GACATCGTGATGACCCAGTCTCCATCCTTCCTGTCTGCATCT | 10 |
| | GTAGGAGACAGAGTCACCATCACTTGCCGGGCCAGTCAGGG | |
| | CATTAGCAGTTATTTAGCCTGGTATCAGCAAAAACCAGGGA | |
| | AAGCCCCTAAGCTCCTGATCTATGCTGCATCCACTTTGCAAA | |
| | GTGGGGTCCCATCAAGGTTCAGCGGCAGTGGATCTGGGACA | |
| | GAATTCACTCTCACAATCAGCAGCCTGCAGCCTGAAGATTTT | |
| | GCAACTTATTACTGTCAACAGCTTAATAGTTACCCTCCAAAA | |
| | TTCACTTTCGGCCCTGGGACCAAGGIGGAAATCAAA | |
| | QGISSY | 11 |
| | AAS | 12 |
| | QQLNSYPPKFT | 13 |
| L12.bP11A11 | QVQLVQSGAEVKKPGASVKVSCKASGYTFTGYYMHWVRQAP | 14 |
| | GQGLEWMGWISPNSGGTNYAQKFQGWVTMTRDTSVSTAYME | |
| | LSRLRFDDTAVYYCATESWVYGSGSYSSGAFDIWGQGTMVTV | |
| | SS | |
| | CAGGTGCAGCTGGTGCAGTCTGGGGCTGAGGTGAAGAAGCC | 15 |
| | TGGGGCCTCAGTGAAGGTCTCCTGCAAGGCTTCTGGATACAC | |
| | CTTCACCGGCTACTATATGCACTGGGTGCGACAGGCCCCTGG | |
| | ACAAGGGCTTGAGTGGATGGGATGGATCAGCCCTAATAGTG | |
| | GTGGCACAAACTATGCACAGAAGTTTCAGGGCTGGGTCACC | |
| | ATGACCAGGGACACGTCCGTCAGCACAGCCTACATGGAACT | |
| | GAGCAGGCTGAGATTTGACGACACGGCCGTGTATTACTGTGC | |
| | GACTGAATCCTGGGTATATGGTTCGGGGAGTTATTCTTCGGG | |
| | TGCTTTTGATATCTGGGGCCAAGGGACAATGGTCACCGTCTC | |
| | TTCA | |
| | GYTFTGYY | 16 |
| | ISPNSGGT | 17 |
| | ATESWVYGSGSYSSGAFDI | 18 |
| | QSVLTQPPSASGSPGQSVTISCTGTSSDVGGYDYVSWYQQHPG | 19 |
| | KAPKLMIYEVSKRPSGVPDRFSGSKSGNTASLTVPGLQAEDEA | |
| | DYYCTSYAGSNNFVFGGGTKLIVL | |
| | CAGTCTGTGCTGACTCAGCCTCCCTCCGCGTCCGGGTCTCCT | 20 |
| | GGACAGTCAGTCACCATCTCCTGCACTGGAACCAGCAGTGA | |
| | CGTTGGTGGTTATGACTATGTCTCCTGGTACCAACAGCACCC | |
| | AGGCAAAGCCCCCAAACTCATGATTTATGAGGTCAGTAAGC | |
| | GGCCCTCAGGGGTCCCTGATCGCTTCTCTGGCTCCAAGTCTG | |
| | GCAACACGGCCTCCCTGACCGTCCCTGGGCTCCAGGCTGAG | |
| | GATGAGGCTGATTATTACTGCACCTCATATGCAGGCAGCAAC | |
| | AATTTTGTATTCGGCGGAGGGACCAAGCTGATCGTCCTA | |
| | SSDVGGYDY | 21 |
| | EVS | 22 |
| | TSYAGSNNFV | 23 |
| L12.bP11G07 | QVQLVESGGGLIQPGGSLRLSCAASGFTVSSNYMSWVRQAPGK | 24 |
| | GLEWVSVIYSGGSTFYADSVKGRFTISRDNSKSTLYLQMNSLRV | |
| | EDTAVYYCARDFGDFYFDYWGQGTLVTVSS | |
| | CAGGTGCAGCTGGTGGAGTCTGGAGGAGGCTTGATCCAGCC | 25 |
| | TGGGGGGTCCCTGAGACTCTCCTGTGCAGCCTCTGGGTTCAC | |
| | CGTCAGTAGCAACTACATGAGCTGGGTCCGCCAGGCTCCAG | |
| | GGAAGGGGCTGGAGTGGGTCTCAGTTATTTATAGCGGTGGT | |
| | AGCACATTCTACGCAGACTCCGTGAAGGGCCGATTCACCATC | |
| | TCCAGAGACAATTCCAAGAGCACGCTGTATCTTCAAATGAAC | |
| | AGCCTGAGAGTCGAGGACACGGCCGTGTATTACTGTGCGAG | |
| | AGACTTCGGTGACTTCTACTTTGACTACTGGGGCCAGGGAAC | |
| | CCTGGTCACCGTCTCCTCA | |
| | GFTVSSNY | 26 |
| | IYSGGST | 27 |
| | ARDFGDFYFDY | 28 |
| | EIVLTQSPGTLSLSPGERATLSCRASQSVSSYLAWYQQKPGQAP | 29 |
| | RLLIYGASSRATGIPDRFSGSGSGTDFTLTISRLEPEDFAVYYCQ | |
| | QYGSSPRTFGQGTKLEIK | |
| | GAAATTGTGTTGACGCAGTCTCCAGGCACCCTGTCTTTGTCT | 30 |
| | CCAGGGGAAAGAGCCACCCTCTCCTGCAGGGCCAGTCAGAG | |
| | TGTTAGTAGCTACTTAGCCTGGTACCAGCAGAAACCTGGCCA | |
| | GGCTCCCAGGCTCCTCATCTATGGTGCATCCAGCAGGGCCAC | |
| | TGGCATCCCAGACAGGTTCAGTGGCAGTGGGTCTGGGACAG | |
| | ACTTCACTCTCACCATCAGCAGACTGGAGCCTGAAGATTTTG | |
| | CAGTGTATTACTGTCAGCAGTATGGTAGCTCACCTCGAACGT | |
| | TCGGCCAAGGGACCAAGCTGGAGATCAAA | |
| | QSVSSY | 31 |
| | GAS | 32 |
| | QQYGSSPRT | 33 |
| L12.bP12D08 | EVQLVESGGGVVQPGRSLRLSCAASGFTFRNYGMHWVRQAPG | 34 |
| | KGLEGVAVISYDGSNKYYADSVKGRFTISRDNSKNTLYLQMNS | |
| | LRAEDTAVYYCAKSSGSYYYYYYGMDVWGQGTTVTVSS | |
| | GAGGTGCAGCTGGTGGAGTCTGGGGGAGGCGTGGTCCAGCC | 35 |
| | TGGGAGGTCCCTGAGACTCTCCTGTGCAGCCTCTGGATTCAC | |
| | CTTCAGAAACTATGGCATGCACTGGGTCCGCCAGGCTCCAGG | |
| | CAAGGGGCTGGAGGGGGTGGCAGTTATATCATATGATGGAA | |
| | GTAATAAATACTATGCAGACTCCGTGAAGGGCCGATTCACC | |
| | ATCTCCAGAGACAATTCCAAGAACACGCTGTATCTGCAAATG | |
| | AACAGCCTGAGAGCTGAGGACACGGCTGTGTATTACTGTGC | |
| | GAAGTCGAGTGGGAGCTACTACTACTACTACTACGGTATGG | |
| | ACGTCTGGGGCCAAGGGACCACGGTCACCGTCTCCTCA | |
| | GFTFRNYG | 36 |
| | ISYDGSNK | 37 |
| | AKSSGSYYYYYYGMDV | 38 |
| | SYELTQPPSVSVAPGKTARITCGGNNIGSKSVHWYQQKPGQAP | 39 |
| | VLVVYDDTDRPSGIPERFSGSSSGNTATLIISRVEAGDEADYYC | |
| | QVWDSSSDHPVVFGGGTKLTVL | |
| | TCCTATGAGCTGACACAGCCACCTTCGGTGTCAGTGGCCCCA | 40 |
| | GGAAAGACGGCCAGGATTACCTGTGGGGGAAACAACATTGG | |
| | AAGTAAAAGTGTGCACTGGTACCAGCAGAAGCCAGGCCAGG | |
| | CCCCTGTGCTGGTCGTCTATGATGATACCGACCGGCCCTCAG | |
| | GGATCCCTGAGCGATTCTCTGGCTCCAGCTCTGGGAACACGG | |
| | CCACCCTGACCATCAGCAGGGTCGAAGCCGGGGATGAGGCC | |
| | GACTATTACTGTCAGGTGTGGGATAGTAGTAGTGATCATCCT | |
| | GTGGTATTCGGCGGAGGGACCAAGCTGACCGTCCTA | |
| | NIGSKS | 41 |
| | DDT | 42 |
| | QVWDSSSDHPVV | 43 |
| L12.bP10G07 | EVQLVQSGAEVKKPGASVKVSCKASGYIYSGYYMHWVRQAPG | 44 |
| | QGLEWMGWISPDSGGTNYAQRFQGRVTMTRDTSTTTAYMELS | |
| | RLRSDDTAMYYCARGPRYSGTYFDYWGQGTLVTVSS | |
| | GAGGTGCAGCTGGTGCAGTCTGGGGCCGAGGTGAAGAAGCC | 45 |
| | TGGGGCCTCAGTGAAGGTCTCCTGCAAGGCTTCTGGGTACAT | |
| | CTACAGCGGCTACTATATGCACTGGGTGCGACAGGCCCCTGG | |
| | ACAAGGGCTTGAGTGGATGGGATGGATCAGCCCTGACAGTG | |
| | GAGGCACAAACTATGCGCAGAGGTTTCAGGGCAGGGTCACC | |
| | ATGACCAGGGACACGTCCACCACCACAGCCTACATGGAGCT | |
| | GAGCAGGCTGAGATCTGACGACACGGCCATGTATTACTGTG | |
| | CGAGAGGTCCGCGGTATAGTGGGACCTACTTTGACTACTGGG | |
| | GCCAGGGAACCCTGGTCACCGTCTCCTCA | |
| | GYIYSGYY | 46 |
| | ISPDSGGT | 47 |
| | ARGPRYSGTYFDY | 48 |
| | QTVVTQPASVSGSPGQSITISCTGTSSDVGGYNYVSWYQQHPD | 49 |
| | KAPKLIIYDVSNRPSGVSNRFSGSKSGNTASLTISGLQAEDEAAY | |
| | YCSSYTSSSTQVFGGGTKLTVL | |
| | CAGACTGTGGTGACTCAGCCTGCCTCCGTGTCTGGGTCTCCT | 50 |
| | GGACAGTCGATCACCATCTCCTGCACTGGAACCAGCAGTGA | |
| | CGTTGGTGGTTATAACTATGTCTCCTGGTACCAACAACACCC | |
| | AGACAAAGCCCCCAAACTCATCATTTATGATGTCAGTAATCG | |
| | GCCCTCAGGGGTTTCTAATCGCTTCTCTGGCTCCAAGTCTGG | |
| | CAACACGGCCTCCCTGACCATCTCTGGACTCCAGGCTGAGGA | |
| | CGAGGCTGCTTATTACTGCAGCTCATATACAAGCAGCAGCAC | |
| | TCAGGTGTTCGGCGGAGGGACCAAGCTGACCGTCCTA | |
| | SSDVGGYNY | 51 |
| | DVS | 52 |
| | SSYTSSSTQV | 53 |
| L12.bP11C10 | QVQLVQSGAEVKKPGASVKVSCKASGYIFSGYYMHWVRQAPG | 54 |
| | QGLEWMGWISPDSGGTNYAQKFQGRVTMTRDTSITTGYMELS | |
| | GLRSDDTAVYYCARGPRYSGTYFDYWGQGTLVTISS | |
| | CAGGTGCAGCTGGTGCAGTCTGGGGCTGAGGTGAAGAAGCC | 55 |
| | TGGGGCCTCAGTGAAGGTCTCCTGCAAGGCTTCTGGATACAT | |
| | CTTCAGCGGCTACTATATGCACTGGGTGCGACAGGCCCCTGG | |
| | ACAAGGACTTGAGTGGATGGGGTGGATCAGCCCTGACAGTG | |
| | GTGGCACGAACTATGCACAGAAGTTTCAGGGCAGGGTCACC | |
| | ATGACCAGGGACACGTCCATCACCACAGGCTACATGGAGTT | |
| | GAGCGGGCTGAGATCTGACGACACGGCCGTCTATTACTGTGC | |
| | GAGAGGTCCGCGGTATAGTGGGACCTACTTTGACTACTGGG | |
| | GCCAGGGAACCCTGGTCACCATCTCCTCA | |
| | GYIFSGYY | 56 |
| | ISPDSGGT | 57 |
| | ARGPRYSGTYFDY | 58 |
| | QSALTQPTSVSGSPGQSITISCTGTSSDVGGYNYVSWYQQHPGK | 59 |
| | APKLIIYDVSNRPSGVSNRFSGSKSGNTASLTISGLQAEDEADYY | |
| | CSSYTVSSTQVFGGGTKLTVL | |
| | CAGTCTGCCCTGACTCAGCCTACCTCCGTGTCTGGGTCTCCT | 60 |
| | GGACAGTCGATCACCATCTCCTGCACTGGAACCAGCAGTGA | |
| | CGTTGGTGGTTATAACTATGTCTCCTGGTACCAACAGCACCC | |
| | AGGCAAAGCCCCCAAACTCATTATTTATGATGTCAGTAATCG | |
| | GCCCTCAGGGGTTTCTAATCGCTTCTCTGGCTCCAAGTCTGG | |
| | CAACACGGCCTCCCTGACCATCTCTGGGCTCCAGGCTGAGGA | |
| | CGAGGCTGATTATTACTGCAGCTCATATACAGTCAGCAGCAC | |
| | TCAGGTGTTCGGCGGAGGGACCAAGCTGACCGTCCTA | |
| | SSDVGGYNY | 61 |
| | DVS | 62 |
| | SSYTVSSTQV | 63 |
| L12.bP12E05 | EVQLVQSGAEVKKPGASVKVTCKTSGYIFSGYYMHWVRQVPG | 64 |
| | QGLEWMGWISPDSGATNYAQKFQGRVTMTRDTSITTSYVELT | |
| | WLKSDDTAVYYCARGPRYSGTYFDFWGQGTLVTVSS | |
| | GAGGTGCAGCTGGTGCAGTCTGGGGCTGAGGTGAAGAAGCC | 65 |
| | TGGGGCCTCAGTGAAGGTCACCTGCAAGACTTCTGGGTACAT | |
| | CTTCAGCGGCTACTATATGCACTGGGTGCGACAGGTCCCCGG | |
| | ACAAGGGCTTGAGTGGATGGGTTGGATCAGCCCTGACAGTG | |
| | GTGCCACAAACTACGCACAGAAGTTTCAGGGCAGGGTCACC | |
| | ATGACCAGGGACACGTCCATTACTACGTCCTACGTGGAGCTG | |
| | ACCTGGCTTAAATCTGACGACACGGCCGTGTATTACTGTGCG | |
| | AGAGGCCCTCGATACAGTGGGACCTACTTTGACTTCTGGGGC | |
| | CAGGGAACCCTGGTCACCGTCTCCTCA | |
| | GYIFSGYY | 66 |
| | ISPDSGAT | 67 |
| | ARGPRYSGTYFDF | 68 |
| | QAVVTQPASVSGSPGQSLTISCTGTSSDIGGFNYVSWYQQHPGK | 69 |
| | APKLMIFDVSKRPSGVPNRFSGSKSGNTASLTISGLQAEDEGDY | |
| | YCSSYTISSAQVFGGGTKLTVL | |
| | CAGGCTGTGGTGACTCAGCCTGCCTCCGTGTCTGGGTCTCCT | 70 |
| | GGACAGTCGCTCACCATCTCCTGCACTGGAACCAGCAGTGAC | |
| | ATTGGTGGTTTTAATTATGTCTCCTGGTATCAACAACACCCA | |
| | GGCAAAGCCCCCAAACTCATGATTTTCGATGTCAGTAAACGG | |
| | CCCTCAGGGGTTCCTAATCGGTTCTCTGGCTCCAAGTCTGGC | |
| | AACACGGCCTCCCTGACCATCTCTGGGCTCCAGGCTGAGGAC | |
| | GAGGGTGATTATTATTGCAGCTCATATACAATCAGCAGTGCT | |
| | CAGGTGTTCGGCGGAGGGACGAAGCTGACCGTCCTA | |
| | SSDIGGFNY | 71 |
| | DVS | 72 |
| | SSYTISSAQV | 73 |
| L12.bP12H09 | QVQLVQSGAEVKKPGASVKVSCKASGYIFSGYYTHWVRQAPG | 74 |
| | QGLEWMGWISPDSGGTNYAQKFQGRVTMTRDTSITTAYVELS | |
| | GLRSDDTAVYYCARGPRYSGTYFDYWGQGTLVTVSS | |
| | ACAGGCCCCTGGACAAGGGCTTGAGTGGATGGGATGGATCA | 75 |
| | GCCCTGACAGTGGTGGCACAAACTACGCACAGAAGTTTCAG | |
| | GGCAGGGTCACCATGACCAGGGACACGTCCATCACCACAGC | |
| | CTACGTGGAGCTGAGCGGGCTGAGATCTGACGACACGGCCG | |
| | TGTATTACTGTGCGAGAGGTCCGCGGTATAGTGGGACCTACT | |
| | TTGACTACTGGGGCCAGGGAACCCTGGTCACCGTCTCCTCA | |
| | GYIFSGYY | 76 |
| | ISPDSGGT | 77 |
| | ARGPRYSGTYFDY | 78 |
| | QSALTQPASVSGSPGQSITISCTGTSSDVGGYNHVSWYQQHPGK | 79 |
| | APKLMIYDVSNRPSGVSNRFSGSKSGNTASLTISGLQAEDEADY | |
| | YCSSYTSSSAQLFGGGTKLTVL | |
| | CAGTCTGCCCTGACTCAGCCTGCCTCCGTGTCTGGGTCTCCT | 80 |
| | GGACAGTCGATCACCATCTCCTGCACTGGAACCAGCAGTGA | |
| | CGTTGGTGGTTATAACCATGTCTCCTGGTACCAACAGCACCC | |
| | AGGCAAAGCCCCCAAACTCATGATTTATGATGTCAGTAATCG | |
| | GCCCTCAGGGGTTTCTAATCGCTTCTCTGGTTCCAAGTCTGG | |
| | CAACACGGCCTCCCTGACCATCTCTGGGCTCCAGGCTGAGGA | |
| | CGAGGCTGATTATTACTGCAGCTCATATACAAGCAGCAGCGC | |
| | TCAGTTGTTCGGCGGAGGGACCAAGCTGACCGTCCTA | |
| | SSDVGGYNH | 81 |
| | DVS | 82 |
| | SSYTSSSAQL | 83 |
| L12.bP11E12 | QVQLVQSGAEVKKPGASVKVSCKVSGYTLTELSIHWVRQAPG | 84 |
| | KGLEWMGGFDPEDAETIYAQKFQGRVTMTEDTSTDTAYMELS | |
| | SLRSEDTAVYYCATGFAQNMVLLTPYWGQGTLVTVSS | |
| | CAGGTGCAGCTGGTACAGTCTGGGGCTGAGGTGAAGAAGCC | 85 |
| | TGGGGCCTCAGTGAAGGTCTCCTGCAAGGTTTCCGGATACAC | |
| | CCTCACTGAATTATCCATTCACTGGGTGCGACAGGCTCCTGG | |
| | AAAAGGGCTTGAGTGGATGGGAGGTTTTGATCCTGAAGATG | |
| | CTGAAACAATCTACGCACAGAAGTTCCAGGGCAGAGTCACC | |
| | ATGACCGAGGACACATCTACAGACACAGCCTACATGGAGCT | |
| | GAGCAGCCTGAGATCTGAGGACACGGCCGTGTATTACTGTG | |
| | CAACAGGATTCGCCCAAAATATGGTTTTGTTGACTCCTTACT | |
| | GGGGCCAGGGTACCCTGGTCACCGTCTCCTCA | |
| | GYTLTELS | 86 |
| | FDPEDAET | 87 |
| | ATGFAQNMVLLTPY | 88 |
| | EIVMTQSPATLSVSPGERATLSCRASQSVSSNLAWYQQKPGQA | 89 |
| | PRLLIYGASTRATGIPARFSGSGSGTEFTLTISSLQSEDFAVYYCQ | |
| | QYNNWPPITFGQGTRLEIK | |
| | GAAATAGTGATGACGCAGTCTCCAGCCACCCTGTCTGTGTCT | 90 |
| | CCAGGGGAAAGAGCCACCCTCTCCTGCAGGGCCAGTCAGAG | |
| | TGTTAGCAGCAACTTAGCCTGGTACCAGCAGAAACCTGGCC | |
| | AGGCTCCCAGGCTCCTCATCTATGGTGCATCCACCAGGGCCA | |
| | CTGGTATCCCAGCCAGGTTCAGTGGCAGTGGGTCTGGGACA | |
| | GAGTTCACTCTCACCATCAGCAGCCTGCAGTCTGAAGATTTT | |
| | GCAGTTTATTACTGTCAGCAGTATAATAACTGGCCTCCGATC | |
| | ACCTTCGGCCAAGGGACACGACTGGAGATCAAA | |
| | QSVSSN | 91 |
| | GAS | 92 |
| | QQYNNWPPIT | 93 |
| L12.bP13A12 | EVQLVESGGGLVQPGGSLRPSCAASGFTFSSYAMSWVRQAPGK | 94 |
| | GLEWVPAISGSGGSTYYADSVKGRFTISRDNSKNTLYLQMNSL | |
| | RAEDTAVYYCAKGSGSGSYPNYYYYYGMDVWGQGTTVTVSS | |
| | GAGGTGCAGCTGGTGGAGTCTGGGGGAGGCTTGGTACAGCC | 95 |
| | TGGGGGGTCCCTGAGACCCTCCTGTGCAGCCTCTGGATTCAC | |
| | CTTTAGCAGCTATGCCATGAGCTGGGTCCGCCAGGCTCCAGG | |
| | GAAGGGGCTGGAGTGGGTCCCAGCTATTAGTGGTAGTGGTG | |
| | GTAGTACATACTACGCAGACTCCGTGAAGGGCCGGTTCACC | |
| | ATCTCCAGAGACAATTCCAAGAACACGCTGTATCTGCAAATG | |
| | AACAGCCTGAGAGCCGAGGACACGGCCGTATATTACTGTGC | |
| | GAAAGGTAGTGGTTCGGGGAGTTACCCTAATTACTACTACTA | |
| | CTACGGTATGGACGTCTGGGGCCAAGGGACCACGGTCACCG | |
| | TCTCTTCA | |
| | GFTFSSYA | 96 |
| | ISGSGGST | 97 |
| | AKGSGSGSYPNYYYYYGMDV | 98 |
| | SYELTQSPSVSVAPGQTARITCGGNNIGSKSVHWYQRKPGQAP | 99 |
| | VLVVYDDSDRPSGIPERFSGSNSGNTATLTISRVEAGDEADYYC | |
| | QVWDNNSDHLVFGGGTKLTVL | |
| | TCCTATGAGCTGACTCAGTCACCCTCGGTGTCAGTGGCCCCA | 100 |
| | GGACAGACGGCCAGGATTACCTGTGGGGGAAACAACATTGG | |
| | AAGTAAAAGTGTGCACTGGTACCAGCGGAAGCCAGGCCAGG | |
| | CCCCTGTGCTGGTCGTCTATGATGATAGCGACCGGCCCTCAG | |
| | GGATCCCTGAGCGATTCTCTGGCTCCAACTCTGGGAACACGG | |
| | CCACCCTGACCATCAGCAGGGTCGAAGCCGGGGATGAGGCC | |
| | GACTATTACTGICAGGTGTGGGATAATAATAGTGATCATCTG | |
| | GTATTCGGCGGAGGGACCAAGCTGACCGTCCTA | |
| | NIGSKS | 101 |
| | DDS | 102 |
| | QVWDNNSDHLV | 103 |
| L12.cP07E10 | EVQLVESGGGLIQPGGSLRLSCAASGFTVSSNYMSWVRQAPGK | 104 |
| | GLEWVSVIYSGGSTYYADSVKGRFTISRDNSKNTLYLQMNSLR | |
| | AEDTAVYYCARDPYGYSSIWDGQGGHWGQGTLVTVSS | |
| | GAGGTGCAGCTGGTGGAGTCTGGAGGAGGCTTGATCCAGCC | 105 |
| | TGGGGGGTCCCTGAGACTCTCCTGTGCAGCCTCTGGGTTCAC | |
| | CGTCAGTAGCAACTACATGAGCTGGGTCCGCCAGGCTCCAG | |
| | GGAAGGGGCTGGAGTGGGTCTCAGTTATTTATAGCGGTGGT | |
| | AGCACATACTACGCAGACTCCGTGAAGGGCCGATTTACCATC | |
| | TCCAGAGACAATTCCAAGAACACGCTGTATCTTCAAATGAAC | |
| | AGCCTGAGAGCCGAGGACACGGCCGTGTATTACTGTGCGAG | |
| | AGATCCGTACGGGTATAGTAGCATCTGGGACGGACAAGGCG | |
| | GCCACTGGGGCCAGGGAACCCTGGTCACCGTCTCCTCA | |
| | GFTVSSNY | 106 |
| | IYSGGST | 107 |
| | ARDPYGYSSIWDGQGGH | 108 |
| | DVVMTQSPSSLSASVGDRVTITCQASQDISNYLNWYQQKPGKA | 109 |
| | PKLLIYDASNFATGVPSRFSGTGSGTDFTFTISSLQPEDIATYYCQ | |
| | QYDNLPITFGQGTRLEIK | |
| | GATGTTGTGATGACTCAGTCTCCATCCTCCCTGTCTGCATCTG | 110 |
| | TAGGAGACAGAGTCACCATCACTTGCCAGGCGAGTCAGGAC | |
| | ATTAGCAACTATTTAAATTGGTATCAGCAGAAACCAGGGAA | |
| | AGCCCCTAAGCTCCTGATCTACGATGCATCCAATTTTGCAAC | |
| | AGGGGTCCCATCAAGGTTCAGTGGAACTGGATCTGGGACAG | |
| | ATTTTACTTTCACCATCAGCAGCCTGCAGCCTGAAGATATTG | |
| | CAACATATTACTGTCAACAGTATGATAATCTCCCGATCACCT | |
| | TCGGCCAAGGGACACGACTGGAGATCAAA | |
| | QDISNY | 111 |
| | DAS | 112 |
| | QQYDNLPIT | 113 |
| L12.cP08D07 | QVQLQESGPGLVKPSETLSLTCTVSGGSISSSSYYWGWIRQPPG | 114 |
| | KGLEWIGSIYYSGSTYYNPSLKSRVTISVDTSKNQFSLKLSSVTA | |
| | ADTAVYYCARQGDCSTTSCAYDYWGQGTLVTVSS | |
| | CAGGTACAGCTGCAGGAGTCGGGCCCAGGACTGGTGAAGCC | 115 |
| | TTCGGAGACCCTGTCCCTCACCTGCACTGTCTCTGGTGGCTC | |
| | CATCAGCAGTAGTAGTTACTACTGGGGCTGGATCCGCCAGCC | |
| | CCCAGGGAAGGGGCTGGAGTGGATTGGGAGTATCTATTATA | |
| | GTGGGAGCACCTACTACAACCCGTCCCTCAAGAGTCGAGTC | |
| | ACCATATCCGTAGACACGTCCAAGAACCAGTTCTCCCTGAAG | |
| | CTGAGCTCTGTGACCGCCGCAGACACGGCTGTGTATTACTGT | |
| | GCGAGACAAGGGGATTGTAGTACTACCAGCTGCGCCTACGA | |
| | CTACTGGGGCCAGGGAACCCTGGTCACCGTCTCCTCA | |
| | GGSISSSSYY | 116 |
| | IYYSGST | 117 |
| | ARQGDCSTTSCAYDY | 118 |
| | SYELTQPPSVSVSPGQTARITCSGDALPKQYAYWYQQKPGQAP | 119 |
| | VLVIYKDSERPSGIPERFSGSSSGTTATLTISGVQAEDEADYYCQ | |
| | SADSSGTYLVVFGGGTKLAVL | |
| | TCCTATGAGCTGACACAGCCACCCTCGGTGTCAGTGTCCCCA | 120 |
| | GGACAGACGGCCAGGATCACCTGCTCTGGAGATGCATTGCC | |
| | AAAGCAATATGCTTATTGGTACCAGCAGAAGCCAGGCCAGG | |
| | CCCCTGTGCTGGTGATATATAAAGACAGTGAGAGGCCCTCA | |
| | GGGATCCCTGAGCGATTCTCTGGCTCCAGCTCAGGGACAACA | |
| | GCCACGTTGACCATCAGTGGAGTCCAGGCAGAAGACGAGGC | |
| | TGACTATTACTGTCAATCAGCAGACAGCAGTGGTACTTATCT | |
| | TGTGGTATTCGGCGGAGGGACCAAGCTGGCCGTCCTA | |
| | ALPKQY | 121 |
| | KDS | 122 |
| | QSADSSGTYLVV | 123 |
| L12.cP09B02 | EVQLVESGGGLIQPGGSLRLSCAASGFTVSSNYMSWVRQAPGK | 124 |
| | GLEWVSVFYSGGSTYYADSVKGRFTISRDNSKNTLYLQMNSLR | |
| | AEDTAVYYCARDYGDLYFDYWGQGTLVTVSS | |
| | GAAGTGCAGCTGGTGGAGTCTGGAGGAGGCTTGATCCAGCC | 125 |
| | TGGGGGGTCCCTGAGACTCTCCTGTGCAGCCTCTGGGTTCAC | |
| | CGTCAGTAGCAACTACATGAGCTGGGTCCGCCAGGCTCCAG | |
| | GGAAGGGGCTGGAGTGGGTCTCAGTTTTTTATAGCGGTGGTA | |
| | GCACATACTACGCAGACTCCGTGAAGGGCCGATTCACCATCT | |
| | CCAGAGACAATTCCAAGAACACGCTGTATCTTCAAATGAAC | |
| | AGCCTGAGAGCCGAGGACACGGCCGTGTATTACTGTGCGAG | |
| | AGACTACGGTGACCTCTACTTTGACTACTGGGGCCAGGGAAC | |
| | GCTGGTCACCGTCTCCTCA | |
| | GFTVSSNY | 126 |
| | FYSGGST | 127 |
| | ARDYGDLYFDY | 128 |
| | EIVMTQSPGTLSLSPGERATLSCRASQSVSSSYLAWYQQKPGQA | 129 |
| | PRLLIYGVSSRATGIPDRESGSGSETDFTLTISRLEPEEFAVYYCQ | |
| | QYGSSPRTFGQGTKLEIK | |
| | GAAATAGTGATGACGCAGTCTCCAGGCACCCTGTCTTTGTCT | 130 |
| | CCAGGGGAAAGAGCCACCCTCTCCTGCAGGGCCAGCCAGAG | |
| | CGTTAGCAGCAGCTACTTAGCCTGGTACCAGCAGAAACCTG | |
| | GCCAGGCCCCCAGGCTCCTCATTTATGGTGTATCCAGTAGGG | |
| | CCACTGGCATCCCAGACAGGTTCAGTGGCAGTGGGTCTGAG | |
| | ACAGACTTCACTCTCACCATCAGCAGACTGGAGCCTGAAGA | |
| | GTTTGCAGTGTATTACTGTCAGCAGTATGGTAGCTCACCTCG | |
| | GACGTTCGGCCAAGGGACCAAGCTGGAGATCAAA | |
| | QSVSSSY | 131 |
| | GVS | 132 |
| | QQYGSSPRT | 133 |
| L12.cP08A09 | QVQLVQSGAEVKKPGASVKVSCKASGYIYSGYYMHWVRQAP | 134 |
| | GQGLEWMGWISPDSGGTNYAQRFQGRVTMTRDTSTTTAYMEL | |
| | SRLRSDDTAVYYCARGPRYSGTYFDYWGQGTLVTVSS | |
| | CAGGTGCAGCTGGTGCAGTCTGGGGCCGAGGTGAAGAAGCC | 135 |
| | TGGGGCCTCAGTGAAGGTCTCCTGCAAGGCTTCTGGGTACAT | |
| | CTACAGCGGCTACTATATGCACTGGGTGCGACAGGCCCCTGG | |
| | ACAAGGGCTTGAGTGGATGGGATGGATCAGCCCTGACAGTG | |
| | GTGGCACAAACTATGCACAGAGGTTTCAGGGCAGGGTCACC | |
| | ATGACCAGGGACACGTCCACCACCACAGCCTACATGGAGCT | |
| | GAGCAGGCTGAGATCTGACGACACGGCCGTGTATTACTGTG | |
| | CGAGAGGTCCGCGGTATAGTGGGACCTACTTTGACTACTGGG | |
| | GCCAGGGAACCCTGGTCACCGTCTCCTCA | |
| | GYIYSGYY | 136 |
| | ISPDSGGT | 137 |
| | ARGPRYSGTYFDY | 138 |
| | QSALTQPASVSGSPGQSITISCTGTSSDVGGYNYVSWYQQHPDK | 139 |
| | APKLMIYDVSNRPSGVSNRFSGSKSGNTASLTISGLQAEDEADY | |
| | YCSSYTSGSTQVFGGGTKLTVL | |
| | CAGTCTGCCCTGACTCAGCCTGCCTCTGTGTCTGGGTCTCCTG | 140 |
| | GACAGTCGATCACCATCTCCTGCACTGGAACCAGCAGTGAC | |
| | GTTGGTGGTTATAACTATGTCTCCTGGTACCAACAACACCCA | |
| | GACAAAGCCCCCAAACTCATGATTTATGATGTCAGTAATCGG | |
| | CCCTCAGGGGTTTCTAATCGCTTCTCTGGCTCCAAGTCTGGC | |
| | AACACGGCCTCCCTGACCATCTCTGGACTCCAGGCTGAGGAC | |
| | GAGGCTGATTATTACTGCAGCTCATATACAAGCGGCAGCACT | |
| | CAGGTGTTCGGCGGAGGGACCAAGCTGACCGTCCTA | |
| | SSDVGGYNY | 141 |
| | DVS | 142 |
| | SSYTSGSTQV | 143 |
| L12.cP08D10 | EVQLVQSGAEVKKPGASVKVSCKASGYIFSGYYMHWVRQAPG | 144 |
| | QGLEWMGWISPDSGGTNYAQNFQGRVTMTRDTSISTGYMELS | |
| | RLRSDDTAMYYCARGPRYSGTYFDYWGQGVLVTVSS | |
| | GAGGTGCAGCTGGTGCAGTCTGGGGCTGAGGTGAAGAAGCC | 145 |
| | TGGGGCCTCAGTGAAGGTCTCCTGCAAGGCTTCTGGATACAT | |
| | CTTCAGCGGCTACTATATGCACTGGGTGCGACAGGCCCCTGG | |
| | ACAAGGGCTTGAGTGGATGGGATGGATCAGCCCTGACAGTG | |
| | GTGGCACAAACTATGCACAGAATTTTCAGGGCAGGGTCACC | |
| | ATGACCAGGGACACGTCCATCAGCACAGGTTACATGGAACT | |
| | GAGCAGGCTGAGATCTGACGACACGGCCATGTATTACTGTG | |
| | CGAGAGGTCCGCGATATAGTGGGACATACTTTGACTACTGG | |
| | GGCCAGGGAGTCCTGGTCACCGTCTCCTCA | |
| | GYIFSGYY | 146 |
| | ISPDSGGT | 147 |
| | ARGPRYSGTYFDY | 148 |
| | QSVLTQPASVSGSPGQSITISCTGTSSDVGGYNYVSWYQQHPGK | 149 |
| | VPKLMIYDVSNRPSGVSNRFSGSKSGNTASLTISGLQAEDEADY | |
| | YCSSYTSSSAQVFGGGTKLTVL | |
| | CAGTCTGTGCTGACTCAGCCTGCCTCCGTGTCTGGGTCTCCT | 150 |
| | GGACAGTCGATCACCATCTCCTGCACTGGAACCAGCAGTGA | |
| | CGTTGGTGGTTATAACTATGTCTCCTGGTACCAACAGCACCC | |
| | AGGCAAAGTCCCCAAACTCATGATTTATGATGTCAGTAATCG | |
| | GCCCTCAGGGGTTTCTAATCGCTTCTCTGGCTCCAAGTCTGG | |
| | CAACACGGCCTCCCTGACCATCTCTGGGCTCCAGGCTGAGGA | |
| | CGAGGCTGATTATTACTGCAGCTCATATACAAGCAGCAGCGC | |
| | TCAGGTGTTCGGCGGAGGGACCAAGCTGACCGTCCTA | |
| | SSDVGGYNY | 151 |
| | DVS | 152 |
| | SSYTSSSAQV | 153 |
| L12.cP09C07 | QVQLVQSGAEVKKPGASVKVSCKASGYIYSGYFMHWVRQAPG | 154 |
| | QGLEWMGWISPDSGGANYAQTFQGRVTMTRDTSTTTAHMELS | |
| | RLRSDDTAVYYCARGPRYSGTHFDYWGQGTLVTVSS | |
| | CAGGTGCAGCTGGTGCAGTCTGGGGCCGAGGTGAAGAAGCC | 155 |
| | TGGGGCCTCAGTGAAGGTCTCCTGCAAGGCTTCTGGGTACAT | |
| | CTACAGCGGCTACTTTATGCACTGGGTGCGACAGGCCCCTGG | |
| | ACAAGGGCTTGAGTGGATGGGATGGATCAGCCCTGACAGTG | |
| | GTGGCGCAAACTATGCACAGACGTTTCAGGGCAGGGTCACC | |
| | ATGACCAGGGACACGTCCACCACCACAGCCCACATGGAGCT | |
| | GAGCAGGCTGAGATCTGACGACACGGCCGTGTATTACTGTG | |
| | CGAGAGGTCCGCGGTATAGTTGGGACCCACTTTGACTACTGG | |
| | GGCCAGGGAACCCTGGTCACCGTCTCCTCA | |
| | GYIYSGYF | 156 |
| | ISPDSGGA | 157 |
| | ARGPRYSGTHFDY | 158 |
| | QSALTQPASVSGSPGQSLTISCTGTSSDVGGYNYVSWYQQHPD | 159 |
| | KAPKLMIYDVNNRPSGVSNRFSGSKSGSTASLTISGLQAEDEAD | |
| | YYCSSYAGSSTQVFGGGTKLIVL | |
| | CAGTCTGCCCTGACTCAGCCTGCCTCCGTGTCTGGGTCTCCT | 160 |
| | GGACAGTCGCTCACCATCTCCTGCACTGGAACCAGCAGTGAC | |
| | GTTGGTGGTTACAACTATGTCTCCTGGTACCAGCAACACCCA | |
| | GACAAAGCCCCCAAACTCATGATTTATGATGTCAATAATCGG | |
| | CCCTCAGGGGTTTCTAATCGCTTCTCTGGCTCCAAGTCTGGC | |
| | AGCACGGCCTCCCTGACCATCTCTGGCCTCCAGGCTGAGGAC | |
| | GAGGCCGATTATTACTGCAGCTCATATGCAGGCAGCAGCACT | |
| | CAGGTGTTCGGCGGAGGGACCAAGCTGATCGTCCTA | |
| | SSDVGGYNY | 161 |
| | DVN | 162 |
| | SSYAGSSTQV | 163 |
| L12.cP09D03 | QVQLVQSGAEVKKPGASVKVSCKASGYIFSGYYTHWVRQAPG | 164 |
| | QGLEWMGWISPDSGGTNYAQKFQGRVTMTRDTSITTAYVELS | |
| | GLRSDDTAVYYCARGPRYSGTYFDYWGQGTLVTVSS | |
| | CAGGTTCAGCTGGTGCAGTCTGGGGCTGAGGTGAAGAAGCC | 165 |
| | TGGGGCCTCAGTGAAGGTCTCCTGCAAGGCTTCTGGATACAT | |
| | CTTCAGCGGCTACTATACGCACTGGGTGCGACAGGCCCCTGG | |
| | ACAAGGGCTTGAGTGGATGGGATGGATCAGCCCTGACAGTG | |
| | GTGGCACAAACTACGCACAGAAGTTTCAGGGCAGGGTCACC | |
| | ATGACCAGGGACACGTCCATCACCACAGCCTACGTGGAGCT | |
| | GAGCGGGCTGAGATCTGACGACACGGCCGTGTATTACTGTG | |
| | CGAGAGGTCCGCGGTATAGTGGGACCTACTTTGACTACTGGG | |
| | GCCAGGGAACCCTGGTCACCGTCTCCTCA | |
| | GYIFSGYY | 166 |
| | ISPDSGGT | 167 |
| | ARGPRYSGTYFDY | 168 |
| | QSVLTQPASVSGSPGQSITISCTGTSSDVGGYNYVSWYQQHPGK | 169 |
| | VPKLMIYDVSNRPSGVSNRFSGSKSGNTASLTISGLQAEDEADY | |
| | YCSSYTSSSAQLFGGGTKLTVL | |
| | CAGTCTGTGCTGACTCAGCCTGCCTCCGTGTCTGGGTCTCCT | 170 |
| | GGACAGTCGATCACCATCTCCTGCACTGGAACCAGCAGTGA | |
| | CGTTGGTGGTTATAACTATGTCTCCTGGTACCAACAGCACCC | |
| | AGGCAAAGTCCCCAAACTCATGATTTATGATGICAGTAATCG | |
| | GCCCTCAGGGGTTTCTAATCGCTTCTCTGGTTCCAAGTCTGG | |
| | CAACACGGCCTCCCTGACCATCTCTGGGCTCCAGGCTGAGGA | |
| | CGAGGCTGATTATTACTGCAGCTCATATACAAGCAGCAGCGC | |
| | TCAGTTGTTCGGCGGAGGGACCAAGCTGACCGTCCTA | |
| | SSDVGGYNY | 171 |
| | DVS | 172 |
| | SSYTSSSAQL | 173 |
| L12.cP09D05 | QVQLVESGGGLVKPGGSLRLSCAASGFTFNYYSMNWVRQAPG | 174 |
| | KGLEWISSISTSSSFVYYADSVKGRFTISRDNAKTSLYLQMNSLR | |
| | AEDTAVYFCARGGYCSDGSCYVQDRLIYYYSGLDVWGQGTTV | |
| | TVSS | |
| | CAGGTGCAGCTGGTGGAGTCTGGGGGAGGCCTGGTCAAGCC | 175 |
| | TGGGGGGTCCCTGAGACTCTCCTGTGCAGCCTCTGGATTCAC | |
| | CTTCAATTACTATAGCATGAACTGGGTCCGCCAGGCTCCAGG | |
| | GAAGGGGCTGGAGTGGATCTCATCCATTAGTACTAGTAGTA | |
| | GTTTCGTATACTACGCAGACTCAGTGAAGGGCCGATTCACCA | |
| | TCTCCAGAGACAACGCCAAGACCTCACTGTATCTGCAAATGA | |
| | ACAGCCTGAGAGCCGAGGACACGGCTGTGTATTTCTGTGCG | |
| | AGAGGTGGATATTGTAGTGATGGTAGCTGCTACGTTCAAGAT | |
| | CGCCTCATCTACTACTACTCCGGTTTGGACGTCTGGGGCCAA | |
| | GGGACCACGGTCACCGTCTCCTCA | |
| | GFTFNYYS | 176 |
| | ISTSSSFV | 177 |
| | ARGGYCSDGSCYVQDRLIYYYSGLDV | 178 |
| | DIVMTQSPLSLPVTLGQPASISCRSSQSLVYSDGNTYLNWFQQR | 179 |
| | PGQSPRRLIYKVSNRDSGVPDRFSGSGSGTDFTLKISRVEAEDV | |
| | GVFYCMQGTHWPPTFGQGTKVDIK | |
| | GATATTGTGATGACTCAGTCTCCACTCTCCCTGCCCGTCACC | 180 |
| | CTTGGACAGCCGGCCTCCATCTCCTGCAGGTCTAGTCAAAGC | |
| | CTCGTATACAGTGATGGAAACACCTACTTGAATTGGTTTCAG | |
| | CAGAGGCCAGGCCAATCTCCAAGGCGCCTTATTTATAAGGTT | |
| | TCTAATAGGGACTCTGGGGTCCCAGACAGATTCAGCGGCAG | |
| | TGGGTCAGGCACTGATTTCACACTGAAAATCAGCAGGGTGG | |
| | AGGCTGAGGATGTTGGGGTTTTTTACTGCATGCAAGGTACAC | |
| | ACTGGCCTCCGACGTTCGGCCAAGGGACCAAAGTGGATATC | |
| | AAA | |
| | QSLVYSDGNTY | 181 |
| | KVS | 182 |
| | MQGTHWPPT | 183 |
| L12.bP13A09 | EVQLVESGGGLVQPGGSLRLSCAASGFTFSSYAMSWVRQAPGK | 184 |
| | GLEWVSAISGSGDSTYYADSVKGRFTISRDNSKNTLYLQMNSL | |
| | RAEDTAVYYCAKDRYYEFWSGYSNWFDPWGQGTLVTISS | |
| | GAGGTGCAGCTGGTGGAGTCTGGGGGAGGCTTGGTACAGCC | 185 |
| | TGGGGGGTCCCTGAGACTCTCCTGTGCAGCCTCTGGATTCAC | |
| | CTTTAGCAGCTATGCCATGAGCTGGGTCCGCCAGGCTCCAGG | |
| | GAAGGGGCTGGAGTGGGTCTCAGCTATTAGTGGTAGTGGTG | |
| | ATAGCACATACTACGCAGACTCCGTGAAGGGCCGGTTCACC | |
| | ATCTCCAGAGACAATTCCAAGAACACGCTGTATCTGCAAATG | |
| | AACAGCCTGAGAGCCGAGGACACGGCCGTATATTACTGTGC | |
| | GAAAGATCGTTATTACGAGTTTTGGAGTGGTTACTCGAACTG | |
| | GTTCGACCCCTGGGGCCAGGGAACCCTGGTCACCATCTCCTC | |
| | A | |
| | GFTFSSYA | 186 |
| | ISGSGDST | 187 |
| | AKDRYYEFWSGYSNWFDP | 188 |
| | QSALTQPPSASGTPGQRVTISCSGSSSNIGSNTVNWYQQLPGTA | 189 |
| | PKVLVYSNDQRPSGVPDRFSGSKSGTSASLAISGLQSEDEADYY | |
| | CAAWDDSLNGPVFGGGTKLTVL | |
| | CAGTCTGCCCTGACTCAGCCACCCTCAGCGTCTGGGACCCCC | 190 |
| | GGGCAGAGGGTCACCATCTCTTGTTCTGGAAGCAGCTCCAAC | |
| | ATCGGAAGTAATACTGTAAACTGGTACCAGCAGCTCCCAGG | |
| | AACGGCCCCCAAAGTCCTCGTCTATAGCAATGATCAGCGGCC | |
| | CTCAGGGGTCCCTGACCGATTCTCTGGCTCCAAGTCTGGCAC | |
| | CTCAGCCTCCCTGGCCATCAGTGGGCTCCAGTCTGAGGATGA | |
| | GGCTGATTACTACTGTGCAGCATGGGATGACAGCCTGAATG | |
| | GTCCGGTGTTCGGCGGAGGGACCAAGCTGACCGTCCTA | |
| | SSNIGSNT | 191 |
| | SND | 192 |
| | AAWDDSLNGPV | 193 |
| Spike | MFVFLVLLPLVSSQCVNLTTRTQLPPAYTNSFTRGVYYPDKVFR | 194 |
| (This does not | SSVLHSTQDLFLPFFSNVTWFHAIHVSGTNGTKRFDNPVLPFND | |
| include a | GVYFASTEKSNIIRGWIFGTTLDSKTQSLLIVNNATNVVIKVCEF | |
| linker) | QFCNDPFLGVYYHKNNKSWMESEFRVYSSANNCTFEYVSQPFL | |
| | MDLEGKQGNFKNLREFVFKNIDGYFKIYSKHTPINLVRDLPQGF | |
| | SALEPLVDLPIGINITRFQTLLALHRSYLTPGDSSSGWTAGAAAY | |
| | YVGYLQPRTFLLKYNENGTITDAVDCALDPLSETKCTLKSFTVE | |
| | KGIYQTSNFRVQPTESIVRFPNITNLCPFGEVFNATRFASVYAW | |
| | NRKRISNCVADYSVLYNSASFSTFKCYGVSPTKLNDLCFTNVY | |
| | ADSFVIRGDEVRQIAPGQTGKIADYNYKLPDDFTGCVIAWNSN | |
| | NLDSKVGGNYNYLYRLFRKSNLKPFERDISTEIYQAGSTPCNGV | |
| | EGFNCYFPLQSYGFQPINGVGYQPYRVVVLSFELLHAPATVCG | |
| | PKKSTNLVKNKCVNFNFNGLTGTGVLTESNKKFLPFQQFGRDI | |
| | ADTTDAVRDPQTLEILDITPCSFGGVSVITPGTNTSNQVAVLYQ | |
| | DVNCTEVPVAIHADQLTPTWRVYSTGSNVFQTRAGCLIGAEHV | |
| | NNSYECDIPIGAGICASYQTQTNSPGSASSVASQSIIAYTMSLGA | |
| | ENSVAYSNNSIAIPTNFTISVTTEILPVSMTKTSVDCTMYICGDST | |
| | ECSNLLLQYGSFCTQLNRALTGIAVEQDKNTQEVFAQVKQIYK | |
| | TPPIKDFGGFNFSQILPDPSKPSKRSFIEDLLFNKVTLADAGFIKQ | |
| | YGDCLGDIAARDLICAQKFNGLTVLPPLLTDEMIAQYTSALLAG | |
| | TITSGWTFGAGAALQIPFAMQMAYRENGIGVTQNVLYENQKLI | |
| | ANQFNSAIGKIQDSLSSTASALGKLQDVVNQNAQALNTLVKQL | |
| | SSNFGAISSVLNDILSRLDPPEAEVQIDRLITGRLQSLQTYVTQQL | |
| | IRAAEIRASANLAATKMSECVLGQSKRVDFCGKGYHLMSFPQS | |
| | APHGVVFLHVTYVPAQEKNFTTAPAICHDGKAHFPREGVFVSN | |
| | GTHWFVTQRNFYEPQIITTDNTFVSGNCDVVIGIVNNTVYDPLQ | |
| | PELDSFKEELDKYFKNHTSPDVDLGDISGINASVVNIQKEIDRLN | |
| | EVAKNLNESLIDLQELGKYEQ | |
| RBD-2 | RVQPTESIVRFPNITNLCPFGEVFNATRFASVYAWNRKRISNCV | 195 |
| (This does not | ADYSVLYNSASFSTFKCYGVSPTKLNDLCFTNVYADSFVIRGDE | |
| include a | VRQIAPGQTGKIADYNYKLPDDFTGCVIAWNSNNLDSKVGGNY | |
| linker) | NYLYRLFRKSNLKPFERDISTEIYQAGSTPCNGVEGFNCYFPLQS | |
| | YGFQPTNGVGYQPYRVVVLSFELLHAPATVCGPKKSTNLVKNK | |
| | CVNFNFNGLTGTGVLTESNKKFLPFQQFGRDIADTTDAVRDPQ | |
| | TLEILDITPCS | |
| RBD-1 | RVVPSGDVVRFPNITNLCPFGEVFNATKFPSVYAWERKKISNCV | 196 |
| (This does not | ADYSVLYNSTFFSTFKCYGVSATKLNDLCFSNVYADSFVVKGD | |
| include a | DVRQIAPGQTGVIADYNYKLPDDFMGCVLAWNTRNIDATSTG | |
| linker) | NYNYKYRYLRHGKLRPFERDISNVPFSPDGKPCTPPALNCYWP | |
| | LNDYGFYTTTGIGYQPYRVVVLSFELLNAPATVCGPKLSTDLIK | |
| | NQCVNFNFNGLTGTGVLTPSSKRFQPFQQFGRDVSDFTDSVRD | |
| | PKTSEILDISPCS | |
| L12.cP08E01 | EVQLLESGGGLVQPGGSLRLSCAASGFTFSNYAMTWVRQAPG | 197 |
| | KGLEWVSAISSGSGSTYYADSVKGRFTISRDNSKNTVYLQMNS | |
| | LRAEDTAIYYCAKANKYSSSEFDFWGQGTLVTISS | |
| | GAGGTGCAGCTGTTGGAGTCTGGGGGAGGCTTGGTACAGCC | 198 |
| | TGGGGGGTCCCTGAGACTCTCCTGTGCAGCCTCTGGATTCAC | |
| | CTTTAGCAACTATGCCATGACCTGGGTCCGCCAGGCTCCAGG | |
| | GAAGGGGCTGGAGTGGGTCTCAGCTATTAGTAGTGGTAGTG | |
| | GTAGCACATACTACGCAGACTCCGTGAAGGGCCGGTTCACC | |
| | ATCTCCAGAGACAATTCCAAGAACACGGTGTATTTGCAAATG | |
| | AACAGCCTGAGAGCCGAGGACACGGCCATATATTACTGTGC | |
| | GAAAGCTAATAAGTATAGCAGCTCGGAATTTGACTTCTGGG | |
| | GCCAGGGAACCCTGGTCACCATCTCCTCA | |
| | GFTFSNYA | 199 |
| | ISSGSGST | 200 |
| | AKANKYSSSEFDF | 201 |
| | QSALTQPPSASGTPGQRVTISCSGSSSNIGSNYVYWYQQLPGAA | 202 |
| | PKLLIYRNDQRPSGVPDRFSGSKSGTSVSLAISGLRSEDEADYYC | |
| | AAWDDSLSGWVFGGGTKLTVL | |
| | CAGTCTGCCCTGACTCAGCCACCCTCAGCGTCTGGGACCCCC | 203 |
| | GGGCAGAGGGTCACCATCTCTTGTTCTGGAAGCAGCTCCAAC | |
| | ATCGGAAGTAATTATGTATACTGGTACCAGCAGCTCCCAGGA | |
| | GCGGCCCCCAAACTCCTCATCTATAGGAATGATCAGCGGCCC | |
| | TCAGGGGTCCCTGACCGATTCTCTGGCTCCAAGTCTGGCACC | |
| | TCAGTCTCCCTGGCCATCAGTGGGCTCCGGTCCGAGGATGAG | |
| | GCTGATTATTACTGTGCAGCATGGGATGACAGCCTGAGTGGC | |
| | TGGGTGTTCGGCGGAGGGACCAAACTGACCGTCCTA | |
| | SSNIGSNY | 204 |
| | RND | 205 |
| | AAWDDSLSGWV | 206 |
| L6.dP02E05 | QVQLVQSGSELKKPGASVKVSCKASGYTFATYALNWVRQAPG | 207 |
| | QGLEWMGWVNTNTGSPTYAQGFTGRFVFSFDTSVSTAYLQIRT | |
| | LKAEDTAVYYCAVYYYDSGSPGWFDPWGQGTLVTVSS | |
| | CAGGTGCAGCTGGTGCAATCTGGGTCTGAGTTGAAGAAGCC | 208 |
| | TGGGGCCTCAGTGAAGGTTTCCTGCAAGGCTTCTGGATACAC | |
| | CTTCGCTACGTATGCTTTGAATTGGGTGCGGCAGGCCCCTGG | |
| | ACAAGGGCTTGAGTGGATGGGATGGGTCAACACCAACACTG | |
| | GGAGCCCAACGTATGCCCAGGGCTTCACAGGACGGTTTGTCT | |
| | TCTCCTTCGACACCTCTGTCAGCACGGCATATCTGCAGATCC | |
| | GCACCCTAAAGGCTGAGGACACTGCCGTGTATTACTGTGCGG | |
| | TGTATTACTATGATTCGGGGAGTCCGGGCTGGTTCGACCCCT | |
| | GGGGCCAGGGAACCCTGGTCACCGTCTCCTCA | |
| | GYTFATYA | 209 |
| | VNTNTGSP | 210 |
| | AVYYYDSGSPGWFDP | 211 |
| | DIQLTQSPSSLSASVGDRVTITCRASQTASSYLNWYQQKPGKAP | 212 |
| | NLLIYAASSLQSGVPSRFSGSGSVTDFTLTISSLQPEDFATYYCQ | |
| | QSYSTPPTFGQGTKVDIK | |
| | GACATCCAGTTGACCCAGTCTCCATCCTCCCTGTCTGCATCT | 213 |
| | GTAGGAGACAGAGTCACCATCACTTGCCGGGCAAGTCAGAC | |
| | CGCTAGCAGCTATTTAAATTGGTATCAGCAGAAACCAGGGA | |
| | AAGCCCCTAACCTCCTGATCTATGCTGCATCCAGTTTGCAAA | |
| | GTGGGGTCCCATCAAGGTTCAGTGGCAGTGGATCTGTAACA | |
| | GATTTCACTCTCACCATCAGCAGTCTGCAACCTGAAGATTTT | |
| | GCAACTTACTACTGTCAACAGAGTTACAGTACCCCTCCAACG | |
| | TTCGGCCAAGGGACCAAAGTGGATATCAAA | |
| | QTASSY | 214 |
| | AAS | 215 |
| | QQSYSTPPT | 216 |
| L6.dP03H10 | QVQLVQSGAEVKKPGSSVKVSCKASGGTFSIYAITWVRQAPGQ | 217 |
| | GLEWMGGIIPIIGTANYAQKFQGRVTITADKSTSTAYMELSSLRS | |
| | EDTAVYYCARDFRYCSSTRCYFWFDPWGQGTLVTVSS | |
| | GACATCCAGATGACCCAGTCTCCATCCTCCCTGTCTGCATCT | 218 |
| | GTAGGAGACAGAGTCACCATCACTTGCCGGGCAAGTCAGAA | |
| | CATTAGCAGCTATTTAAATTGGTATCAGCAGGAAGCAGGGA | |
| | AAGCCCCTAAGCTCCTGATCTATGCTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGICAACAGAGTTACAGTACCCCTCGGACG | |
| | TTCGGCCAAGGGACCAAAGTGGATATCAAA | |
| | GGTFSIYA | 219 |
| | IIPIIGTA | 220 |
| | ARDFRYCSSTRCYFWFDP | 221 |
| | DIQMTQSPSSLSASVGDRVTITCRASQNISSYLNWYQQEAGKAP | 222 |
| | KLLIYAASSLQSGVPSRFSGSGSGTDFTLTISSLQPEDFATYYCQ | |
| | QSYSTPRTFGQGTKVDIK | |
| | GACATCCAGATGACCCAGTCTCCATCCTCCCTGTCTGCATCT | 223 |
| | GTAGGAGACAGAGTCACCATCACTTGCCGGGCAAGTCAGAA | |
| | CATTAGCAGCTATTTAAATTGGTATCAGCAGGAAGCAGGGA | |
| | AAGCCCCTAAGCTCCTGATCTATGCTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAACAGAGTTACAGTACCCCTCGGACG | |
| | TTCGGCCAAGGGACCAAAGTGGATATCAAA | |
| | QNISSY | 224 |
| | AAS | 225 |
| | QQSYSTPRT | 226 |
| L6.dP02A05 | EVQLVQSGAEVKKPGASVKVSCMASGYTFTSYYMHWVRQAP | 227 |
| | GQGLEWMGIISPSGGGTSYAQKFQGRVTLTRDTSTSTVYMELSS | |
| | LRSEDTAVYYCARWYDSTGSIDYWGQGTLVTVSS | |
| | GAAGTGCAGCTGGTGCAGTCTGGGGCTGAGGTGAAGAAGCC | 228 |
| | TGGGGCCTCAGTGAAGGTTTCCTGCATGGCATCTGGATACAC | |
| | CTTCACCAGCTACTATATGCACTGGGTGCGACAGGCCCCTGG | |
| | ACAAGGGCTTGAGTGGATGGGAATAATCAGCCCTAGTGGTG | |
| | GTGGCACAAGCTACGCACAGAAGTTCCAGGGCAGAGTCACC | |
| | CTGACCAGGGACACGTCCACGAGCACAGTCTACATGGAGCT | |
| | GAGCAGCCTGAGATCTGAGGACACGGCCGTGTATTACTGTG | |
| | CGAGATGGTATGATAGTACTGGTAGTATTGACTACTGGGGCC | |
| | AGGGAACCCTGGTCACCGTCTCTTCA | |
| | GYTFTSYY | 229 |
| | ISPSGGGT | 230 |
| | ARWYDSTGSIDY | 231 |
| | DIVMTQSPSSLSASVGDRVTITCRASQGIRNDLGWYQQKPGKA | 232 |
| | PKRLIYAASSLQSGVPSRFSGSGSGTEFTLTISSLQPEDFATYYCL | |
| | QHNSYPILTFGGGTKVEIK | |
| | GACATCGTGATGACCCAGTCTCCATCCTCCCTGTCTGCATCT | 233 |
| | GTAGGAGACAGAGTCACCATCACTTGCCGGGCAAGTCAGGG | |
| | CATTAGAAATGATTTAGGCTGGTATCAGCAGAAACCAGGGA | |
| | AAGCCCCTAAGCGCCTGATCTATGCTGCATCCAGTTTGCAAA | |
| | GTGGGGTCCCATCAAGGTTCAGCGGCAGTGGATCTGGGACA | |
| | GAATTCACTCTCACAATCAGCAGCCTGCAGCCTGAAGATTTT | |
| | GCAACTTATTACTGTCTACAGCATAATAGTTACCCTATTCTC | |
| | ACTTTCGGCGGAGGGACCAAGGTGGAAATCAAA | |
| | QGIRND | 234 |
| | AAS | 235 |
| | LQHNSYPILT | 236 |
| L6.dP02F01 | EVQLVESGGGLVQPGRSLRLSCAASGFTFDDYAMHWVRQAPG | 237 |
| | KGLEWVSGISWNSGSIAFAGSVKGRFTISRDNAKNSLYLQMNS | |
| | LRAEDTALYYCAKDQGYSYGNYFDYWGQGTLVTVSS | |
| | GAAGTGCAGCTGGTGGAGTCTGGGGGAGGCTTGGTACAGCC | 238 |
| | TGGCAGGTCCCTGAGACTCTCCTGTGCAGCCTCTGGATTCAC | |
| | CTTTGATGATTATGCCATGCACTGGGTCCGGCAAGCTCCAGG | |
| | GAAGGGCCTGGAATGGGTCTCAGGTATTAGTTGGAATAGTG | |
| | GTAGCATAGCCTTTGCGGGCTCTGTGAAGGGCCGATTCACCA | |
| | TCTCCAGAGACAACGCCAAGAACTCCCTGTATCTGCAAATGA | |
| | ACAGTCTGAGAGCTGAGGACACGGCCTTGTATTACTGTGCAA | |
| | AAGATCAAGGATACAGCTATGGAAACTACTTTGACTACTGG | |
| | GGCCAGGGAACCCTGGTCACCGTCTCCTCA | |
| | GFTFDDYA | 239 |
| | ISWNSGSI | 240 |
| | AKDQGYSYGNYFDY | 241 |
| | QLVLTQPPSVSVAPGKTARITCGGNNIGSKSVYWYQQKPGQAP | 242 |
| | VLVVYDDSDRPSGIPERFSGSNSGNTATLTISRVEAGDEADYYC | |
| | QVWDSSSDHPYVFGSGTKVTVL | |
| | CAGCTTGTGCTGACTCAGCCACCCTCGGTGTCAGTGGCCCCA | 243 |
| | GGAAAGACGGCCAGGATTACCTGTGGGGGAAACAACATTGG | |
| | AAGTAAAAGTGTGTACTGGTACCAGCAGAAGCCAGGCCAGG | |
| | CCCCTGTGCTGGTCGTCTATGATGATAGCGACCGGCCCTCAG | |
| | GGATCCCTGAGCGATTCTCTGGCTCCAACTCTGGGAACACGG | |
| | CCACCCTGACCATCAGCAGGGTCGAAGCCGGGGATGAGGCC | |
| | GACTATTACTGTCAGGTGTGGGATAGTAGTAGTGATCACCCA | |
| | TATGTCTTCGGAAGTGGGACCAAGGTCACCGTCCTA | |
| | NIGSKS | 244 |
| | DDS | 245 |
| | QVWDSSSDHPYV | 246 |
| L6.dP04A03 | QVQLVQSGAEVKKPGSSVKVSCKASGGTFSSSAISWVRQAPGQ | 247 |
| | GLEWMGGIIPILDITNYAQKFQGRVTITADKSTSTAFMELSSLRS | |
| | EDTAVYYCALRNQWDLLVYWGQGTLVTVSS | |
| | CAGGTGCAGCTGGTGCAGTCTGGGGCTGAGGTGAAGAAGCC | 248 |
| | TGGGTCCTCAGTGAAGGTCTCCTGCAAGGCTTCTGGAGGCAC | |
| | CTTCAGCAGCTCTGCTATCAGCTGGGTGCGACAGGCCCCTGG | |
| | ACAAGGGCTTGAGTGGATGGGAGGGATCATCCCTATCCTTG | |
| | ATATAACAAACTACGCACAGAAGTTCCAGGGCAGAGTCACG | |
| | ATTACCGCGGACAAATCCACGAGCACAGCCTTCATGGAGCT | |
| | GAGCAGCCTGAGATCTGAGGACACGGCCGTGTATTACTGTG | |
| | CGTTACGTAACCAGTGGGACCTCCTAGTCTACTGGGGCCAGG | |
| | GAACCCTGGTCACCGTCTCCTCA | |
| | GGTFSSSA | 249 |
| | IIPILDIT | 250 |
| | ALRNQWDLLVY | 251 |
| | EIVLTQSPGTLSLSPGERATLSCRASQSVSSSYLAWYQQKPGQA | 252 |
| | PRLLIYGASSRATGIPDRFSGSGSGTDFTLTISRLEPEDFAVYYCQ | |
| | HYGSSLWTFGQGTKLEIK | |
| | GAAATTGTGTTGACACAGTCTCCAGGCACCCTGTCTTTGTCT | 253 |
| | CCAGGGGAAAGAGCCACCCTCTCCTGCAGGGCCAGTCAGAG | |
| | TGTTAGCAGCAGCTACTTAGCCTGGTACCAGCAGAAACCTGG | |
| | CCAGGCTCCCAGGCTCCTCATCTATGGTGCATCCAGCAGGGC | |
| | CACTGGCATCCCAGACAGGTTCAGTGGCAGTGGGTCTGGGA | |
| | CAGACTTCACTCTCACCATCAGCAGACTGGAGCCTGAAGATT | |
| | TTGCAGTGTATTACTGTCAGCATTATGGTAGCTCACTTTGGA | |
| | CGTTCGGCCAAGGGACCAAGCTGGAGATCAAA | |
| | QSVSSSY | 254 |
| | GAS | 255 |
| | QHYGSSLWT | 256 |
| L6.dP02C11 | EVQLVQSGAEVKKPGASVKVSCKASGYIFTGYYMHWVRQAPG | 257 |
| | QGLEWMGWINPISGGTNYAQKFQGRVTMTRDTSISTTSMELSR | |
| | LRSDDTAVYYCARDTTFSRVSPQDWFDPWGQGTLVTVSS | |
| | GAGGTTCAGCTGGTGCAGTCTGGGGCTGAGGTGAAGAAGCC | 258 |
| | TGGGGCCTCAGTGAAGGTCTCCTGCAAGGCTTCTGGATACAT | |
| | TTTCACCGGCTACTATATGCACTGGGTGCGACAGGCCCCTGG | |
| | ACAAGGGCTTGAGTGGATGGGATGGATCAACCCTATCAGTG | |
| | GTGGCACAAACTATGCACAGAAGTTTCAGGGCAGGGTCACC | |
| | ATGACCAGGGACACGTCCATCAGCACAACCTCCATGGAGCT | |
| | GAGCAGGCTGAGATCTGACGACACGGCCGTGTATTACTGTG | |
| | CGAGAGATACGACCTTTTCTCGAGTCTCCCCGCAGGACTGGT | |
| | TCGACCCCTGGGGCCAGGGAACCCTGGTCACCGTCTCCTCA | |
| | GYIFTGYY | 259 |
| | INPISGGT | 260 |
| | ARDTTFSRVSPQDWFDP | 261 |
| | EIVLTQSPGTLSLSPGERATLSCRASQSVSSSYLAWYQQKPGQA | 262 |
| | PRLLIYGASSRATGIPDRFSGSGSGTDFTLTISRLEPEDFAVYYCQ | |
| | HYGSSLWTFGQGTKLEIK | |
| | CAGTCTGTGCTGACGCAGCCGCCCTCAGTGTCTGGGGCCCCA | 263 |
| | GGGCAGAGGGTCACCATCTCCTGCACTGGGAGCAGCTCCAA | |
| | CATCGGGGCAGGTTATGATGTACACTGGTACCAGCAGCTCCC | |
| | AGGAACAGCCCCCAAACTCCTCATCTATGGTAACAGCAATC | |
| | GGCCCTCAGGGGTCCCTGACCGATTCTCTGGCTCCAAGTCTG | |
| | GCACCTCAGCCTCCCTGGCCATCACTGGGCTCCAGGCTGAGG | |
| | ATGAGGCTGATTATTACTGCCAGTCCTATGACAGCAGCCTGA | |
| | GTGGTTGGGTGTTCGGCGGAGGGACCAAGCTGACCGTCCTA | |
| | SSNIGAGYD | 264 |
| | GNS | 265 |
| | QSYDSSLSGWV | 266 |
| L6.dP04C08 | QVQLVQSGAEVKKPGASVKVSCKASGYTFTGYYIHWVRQAPG | 267 |
| | QGLEWMGWINPNSGGTNYAQKFQGRVTMTRDTSISTAYMELS | |
| | RLRSDDTAVYYCARFFGGNYYGSETKNWEFDYWGQGTLVTVS | |
| | S | |
| | CAGGTTCAGCTGGTGCAGTCTGGGGCTGAGGTGAAGAAGCC | 268 |
| | TGGGGCCTCAGTGAAGGTCTCCTGCAAGGCTTCTGGATACAC | |
| | CTTCACCGGCTACTATATACACTGGGTGCGACAGGCCCCTGG | |
| | ACAAGGGCTTGAGTGGATGGGATGGATCAACCCTAACAGTG | |
| | GTGGCACAAACTATGCACAGAAATTTCAGGGCAGGGTCACC | |
| | ATGACCAGGGACACGTCCATCAGCACAGCCTACATGGAGCT | |
| | GAGCAGGCTGAGATCTGACGACACGGCCGTGTATTACTGTG | |
| | CGAGATTCTTCGGGGGAAATTACTATGGTTCGGAGACCAAA | |
| | AACTGGGAATTTGACTACTGGGGCCAGGGAACCCTGGTCAC | |
| | CGTCTCCTCA | |
| | GYTFTGYY | 269 |
| | INPNSGGT | 270 |
| | ARFFGGNYYGSETKNWEFDY | 271 |
| | QSVLTQPPSVSGAPGQRVTISRTGSSSNIGAGYDVQWYQQLPGT | 272 |
| | APKLLIYGNSNRPSGVPDRFSGSKSGTSASLAITGLQAEDEADY | |
| | YCQSYDSSLSGSWVFGGGTKLTVL | |
| | CAGTCTGTGCTGACGCAGCCGCCCTCAGTGTCTGGGGCCCCA | 273 |
| | GGGCAGAGGGTCACCATCTCCCGCACTGGGAGCAGCTCCAA | |
| | CATCGGGGCAGGTTATGATGTACAGTGGTACCAGCAGCTTCC | |
| | AGGAACAGCCCCCAAACTCCTCATCTATGGTAACAGCAATC | |
| | GGCCCTCAGGGGTCCCTGACCGATTCTCTGGCTCCAAGTCTG | |
| | GCACCTCAGCCTCCCTGGCCATCACTGGGCTCCAGGCTGAGG | |
| | ATGAGGCTGATTATTACTGCCAGTCCTATGACAGCAGCCTGA | |
| | GTGGTTCTTGGGTGTTCGGCGGAGGGACCAAGCTGACCGTCC | |
| | TA | |
| | SSNIGAGYD | 274 |
| | GNS | 275 |
| | QSYDSSLSGSWV | 276 |
| L25.eP04C02 | QVQLVQSGAEVKKPGASVKVSCKASGYTFTDYFMHWVRQAP | 277 |
| | GQGLEWMGWINPNSGGTNYAQKFQGRVTMTRDTSISTAYMEL | |
| | SRLRSDDTAVYYCARDPPLYSNGFFGFDFWGQGTLVTVSS | |
| | CAGGTGCAGCTGGTGCAGTCCGGGGCTGAGGTGAAGAAGCC | 278 |
| | TGGGGCCTCAGTGAAGGTCTCCTGCAAGGCTTCTGGATACAC | |
| | CTTCACCGACTACTTTATGCACTGGGTTCGACAGGCCCCTGG | |
| | ACAAGGGCTTGAGTGGATGGGATGGATCAACCCTAACAGTG | |
| | GTGGCACAAACTATGCACAGAAGTTTCAGGGCAGGGTCACC | |
| | ATGACCAGGGACACGTCCATCAGCACAGCCTACATGGAGCT | |
| | GAGCAGGCTGAGATCTGACGACACGGCCGTGTATTACTGTG | |
| | CGAGAGATCCCCCCCTCTACAGCAACGGTTTCTTCGGCTTTG | |
| | ACTTCTGGGGCCAGGGAACCCTGGTCACCGTCTCCTCA | |
| | GYTFTDYF | 279 |
| | INPNSGGT | 280 |
| | ARDPPLYSNGFFGFDF | 281 |
| | QSVLTQPASVSGSPGQSITISCTGTSSDVGGYNYVSWYQQHPGK | 282 |
| | APKLMIYDVSNRPSGVSNRFSGSKSGNTASLTISGLQAEDEADY | |
| | YCSSYTSSSTVVFGGGTKLTVL | |
| | CAGTCTGTGCTGACTCAGCCTGCCTCCGTGTCTGGGTCTCCT | 283 |
| | GGACAGTCGATCACCATCTCCTGCACTGGAACCAGCAGTGA | |
| | CGTTGGTGGTTATAACTATGTCTCCTGGTACCAACAACACCC | |
| | AGGCAAAGCCCCCAAACTCATGATTTATGATGTCAGTAATCG | |
| | GCCCTCAGGGGTTTCTAATCGCTTCTCTGGCTCCAAGTCTGG | |
| | CAACACGGCCTCCCTGACCATCTCTGGGCTCCAGGCTGAGGA | |
| | CGAGGCTGATTATTACTGCAGCTCATATACAAGCAGCAGCAC | |
| | TGTGGTATTCGGCGGAGGGACCAAGCTGACCGTCCTA | |
| | SSDVGGYNY | 284 |
| | DVS | 285 |
| | SSYTSSSTVV | 286 |
| L25.dP06E11 | QVQLVESGGGLVQPGRSLRLSCAASGFTFDDYAMHWVRQAPG | 287 |
| | KGLEWVSGISWNSGSIGYADSVKGRFTISRDNAKNSLYLQMNG | |
| | LRAEDTALYYCAKVPQRSRYLVGWFDPWGQGTLVTVSS | |
| | CAGGTGCAGCTGGTGGAGTCTGGGGGAGGCTTGGTACAGCC | 288 |
| | TGGCAGGTCCCTGAGACTCTCCTGTGCAGCCTCTGGATTCAC | |
| | CTTTGATGATTATGCCATGCACTGGGTCCGGCAAGCTCCAGG | |
| | GAAGGGCCTGGAGTGGGTCTCAGGTATTAGTTGGAATAGTG | |
| | GTAGCATAGGCTATGCGGACTCTGTGAAGGGCCGATTCACC | |
| | ATCTCCAGAGACAACGCCAAGAACTCCCTGTATCTGCAAATG | |
| | AACGGTCTGAGAGCTGAGGACACGGCTTTGTATTACTGTGCA | |
| | AAAGTCCCACAAAGGAGTAGGTACTTAGTAGGATGGTTCGA | |
| | CCCCTGGGGCCAGGGAACCCTGGTCACCGTCTCCTCA | |
| | GFTFDDYA | 289 |
| | ISWNSGSI | 290 |
| | AKVPQRSRYLVGWFDP | 291 |
| | EIVLTQSPATLSVSPGERATLSCRASQSVSSNLAWYQQKPGQAP | 292 |
| | RLLIYGASTRATGIPARFSGSGSGTEFTLTISSLQSEDFAVYYCQ | |
| | QYNNWPPITFGQGTRLEIK | |
| | GAAATTGTGTTGACGCAGTCTCCAGCCACCCTGTCTGTGTCT | 293 |
| | CCAGGGGAAAGAGCCACCCTCTCCTGCAGGGCCAGTCAGAG | |
| | TGTTAGCAGCAACTTAGCCTGGTACCAGCAGAAACCTGGCC | |
| | AGGCTCCCAGGCTCCTCATCTATGGTGCATCCACCAGGGCCA | |
| | CTGGTATCCCAGCCAGGTTCAGTGGCAGTGGGTCTGGGACA | |
| | GAGTTCACTCTCACCATCAGCAGCCTGCAGTCTGAAGATTTT | |
| | GCAGTTTATTACTGTCAGCAGTATAATAACTGGCCTCCGATC | |
| | ACCTTCGGCCAAGGGACACGACTGGAGATCAAA | |
| | QSVSSN | 294 |
| | GAS | 295 |
| | QQYNNWPPIT | 296 |
| L11.bP10D06 | EVQLVESGGGLVQPGGSLRLSCAASGFTVSSNYMSWVRQAPG | 297 |
| | KGLEWASVIYSGGSTFYADSVKGRFTISRDNSKNTLYLQMNSL | |
| | RAEDTAVYYCARGLPAAVDYFDYWGRGTLVTVSS | |
| | GAGGTGCAGCTGGTGGAGTCTGGGGGAGGCTTGGTCCAGCC | 298 |
| | TGGGGGGTCCCTGAGACTCTCCTGTGCAGCCTCTGGATTCAC | |
| | CGTCAGTAGCAACTACATGAGCTGGGTCCGCCAGGCTCCAG | |
| | GGAAGGGGCTGGAGTGGGCCTCAGTTATTTATAGCGGTGGT | |
| | AGCACATTCTACGCAGACTCCGTGAAGGGCCGATTCACCATC | |
| | TCCAGAGACAATTCCAAGAACACGCTGTATCTTCAAATGAAC | |
| | AGCCTGAGAGCTGAGGACACGGCTGTGTATTACTGTGCGAG | |
| | GGGGCTTCCAGCAGCAGTGGACTACTTTGACTACTGGGGCCG | |
| | GGGAACCCTGGTCACCGTCTCTTCA | |
| | GFTVSSNY | 299 |
| | TYSGGST | 300 |
| | ARGLPAAVDYFDY | 301 |
| | QLVLTQPPSVSGAPGQRVTISCTGSSSNIGAGYDVHWYQQLPGT | 302 |
| | APKLLIFGNSNRPSGVPDRFSGSKSGTSASLAITGLQAEDEADY | |
| | YCQSYDSSLSDLYVFGTGTKVTVL | |
| | CAGCTTGTGCTGACGCAGCCGCCCTCAGTGTCTGGGGCCCCA | 303 |
| | GGGCAGAGGGTCACCATCTCCTGCACTGGGAGCAGCTCCAA | |
| | CATCGGGGCAGGTTATGATGTACACTGGTACCAGCAGCTTCC | |
| | AGGAACAGCCCCCAAACTCCTCATCTTTGGTAACAGCAATCG | |
| | GCCCTCAGGGGTCCCTGACCGATTCTCTGGCTCCAAGTCTGG | |
| | CACCTCAGCCTCCCTGGCCATCACTGGGCTCCAGGCTGAGGA | |
| | TGAGGCTGATTATTACTGCCAGTCCTATGACAGCAGCCTGAG | |
| | TGACCTTTATGTCTTCGGAACTGGGACCAAGGTCACCGTCCT | |
| | A | |
| | SSNIGAGYD | 304 |
| | GNS | 305 |
| | QSYDSSLSDLYV | 306 |
| L13.bP15C03 | QVQLQESGPGLVKPSETLSLTCTVSGGSISSTSYYWGWIRQPPG | 307 |
| | KGLEWIGTIYYSGNTYYNPSLKSRVTISVDTSKNQFSLKLNSVT | |
| | AADTTVYYCARAAYWGNSNPGFDYWGQGTLVTVSS | |
| | CAGGTACAGCTGCAGGAGTCGGGCCCAGGACTGGTGAAGCC | 308 |
| | TTCGGAGACCCTGTCCCTCACCTGCACTGTCTCTGGTGGCTC | |
| | CATCAGCAGTACTAGTTACTACTGGGGCTGGATCCGCCAGCC | |
| | CCCAGGGAAGGGGCTGGAGTGGATTGGGACTATCTATTATA | |
| | GTGGGAACACCTACTACAACCCGTCCCTCAAGAGTCGAGTC | |
| | ACCATATCCGTAGACACGTCCAAGAACCAGTTCTCCCTGAAG | |
| | CTGAATTCTGTGACCGCCGCAGACACGACTGTGTATTACTGT | |
| | GCGAGGGCGGCTTATTGGGGCAATTCAAACCCAGGCTTTGA | |
| | CTACTGGGGCCAGGGAACCCTGGTCACCGTCTCTTCA | |
| | GGSISSTSYY | 309 |
| | IYYSGNT | 310 |
| | ARAAYWGNSNPGFDY | 311 |
| | QTVVTQPASVSGSPGQSITISCTGTSNDVGSYNLVSWYQQHPGK | 312 |
| | APKLMIYEGSKRPSGVSNRFSGSKSGNTASLTISGLQAEDEADY | |
| | YCCSYAGSSTWVFGGGTKLTVL | |
| | CAGACTGTGGTGACTCAGCCTGCCTCCGTGTCTGGGTCTCCT | 313 |
| | GGACAGTCGATCACCATCTCCTGCACTGGAACCAGCAATGAT | |
| | GTTGGGAGTTATAACCTTGTCTCCTGGTACCAACAGCACCCA | |
| | GGCAAAGCCCCCAAACTCATGATTTATGAGGGCAGTAAGCG | |
| | GCCCTCAGGGGTTTCTAATCGCTTCTCTGGCTCCAAGTCTGG | |
| | CAACACGGCCTCCCTGACAATCTCTGGGCTCCAGGCTGAGGA | |
| | CGAGGCTGATTATTACTGCTGCTCATATGCAGGTAGTAGCAC | |
| | TTGGGTGTTCGGCGGAGGGACCAAGCTGACCGTCCTA | |
| | SNDVGSYNL | 314 |
| | EGS | 315 |
| | CSYAGSSTWV | 316 |
| L13.bP16B10 | EVQLQESGPGLVKPSETLSLTCTVSGYSISSAYYWGWIRQPPGK | 317 |
| | GLEWIGSIYHSGSTYYNPSLRSRVTISVDTSKNQFSLKLTSVTAA | |
| | DTAVYCCARVSRGSAVPMNWFDPWGQGTLVTVSS | |
| | GAGGTGCAGCTGCAGGAGTCGGGCCCAGGACTGGTGAAGCC | 318 |
| | TTCGGAGACCCTGTCCCTCACGTGCACTGTCTCTGGTTACTCC | |
| | ATCAGCAGTGCTTACTACTGGGGCTGGATCCGGCAGCCCCCA | |
| | GGGAAGGGGCTGGAGTGGATTGGGAGTATCTATCATAGTGG | |
| | GAGCACCTACTACAACCCGTCCCTCAGGAGTCGAGTCACCAT | |
| | ATCAGTAGACACGTCCAAGAACCAGTTCTCCCTGAAGCTGAC | |
| | CTCTGTGACCGCCGCAGACACGGCCGTATATTGCTGTGCGAG | |
| | AGTTTCCCGTGGTTCAGCTGTCCCAATGAACTGGTTCGACCC | |
| | CTGGGGCCAGGGAACCCTGGTCACCGTCTCCTCA | |
| | GYSISSAYY | 319 |
| | IYHSGST | 320 |
| | ARVSRGSAVPMNWFDP | 321 |
| | QSVLTQPPSVSGAPGQRVTISCTGSSSNIGAGYDVHWYQQLPGT | 322 |
| | APKLLIYVNSNRPSGVPDRFSGSKSGTSASLAITGLQAEDEADY | |
| | YCQSYDSSLSWGVFGGGTKLTVL | |
| | CAGTCTGTGCTGACGCAGCCGCCCTCAGTGTCTGGGGCCCCA | 323 |
| | GGGCAGAGGGTCACCATCTCCTGCACTGGGAGCAGCTCCAA | |
| | CATCGGGGCAGGTTATGATGTACACTGGTACCAGCAGCTTCC | |
| | AGGAACAGCCCCCAAACTCCTCATCTATGTTAACAGCAATCG | |
| | GCCCTCAGGGGTCCCTGACCGATTCTCTGGCTCCAAGTCTGG | |
| | CACCTCAGCCTCCCTGGCCATCACTGGGCTCCAGGCTGAGGA | |
| | TGAGGCTGATTATTACTGCCAGTCCTATGACAGCAGCCTGAG | |
| | TTGGGGAGTGTTCGGCGGAGGGACCAAGCTGACCGTCCTA | |
| | SSNIGAGYD | 324 |
| | VNS | 325 |
| | QSYDSSLSWGV | 326 |
| L13.bP16G09 | QVQLVQSGAEVKKPGASVKVSCKASGYTFTDYYMHWVRQAP | 327 |
| | AQGLEWMGWINPNSGGTNYAQKFQGRVTMTRDTSISTAYMEL | |
| | SRLRSDDTAVYYCARDLPPYYYGSGILKPPYYYYGMDVWGQG | |
| | TTVTVSS | |
| | CAGGTGCAGCTGGTGCAGTCTGGGGCTGAGGTGAAGAAGCC | 328 |
| | TGGGGCCTCAGTGAAGGTCTCCTGCAAGGCTTCTGGATACAC | |
| | CTTCACCGACTACTATATGCACTGGGTGCGACAGGCCCCTGC | |
| | ACAAGGGCTTGAGTGGATGGGATGGATCAACCCTAACAGTG | |
| | GTGGCACAAACTATGCACAGAAGTTTCAGGGCAGGGTCACC | |
| | ATGACCAGGGACACGTCCATCAGCACAGCCTACATGGAGCT | |
| | GAGCAGGCTGAGATCTGACGACACGGCCGTGTATTACTGTG | |
| | CGAGAGATTTACCGCCCTATTACTATGGTTCGGGGATCTTGA | |
| | AACCCCCCTACTACTACTACGGTATGGACGTCTGGGGCCAAG | |
| | GGACCACGGTCACCGTCTCCTCA | |
| | GYTFTDYY | 329 |
| | INPNSGGT | 330 |
| | ARDLPPYYYGSGILKPPYYYYGMDV | 331 |
| | QSVLTQPASVSGSPGQSITISCTGTSSDVGSYNLVSWYQQHPGK | 332 |
| | APKLMIYEVSKRPSGVSNRFSGSKSGNTASLTISGLQAEDEADY | |
| | YCCSYASSFTVLYGGGTKLTVL | |
| | CAGTCTGTGCTGACTCAGCCTGCCTCCGTGTCTGGGTCTCCT | 333 |
| | GGACAGTCGATCACCATCTCCTGCACTGGAACCAGCAGTGAT | |
| | GTTGGGAGTTATAACCTTGTCTCCTGGTACCAACAGCACCCA | |
| | GGCAAAGCCCCCAAACTCATGATTTATGAGGTCAGTAAGCG | |
| | GCCCTCAGGGGTTTCTAATCGCTTCTCTGGCTCCAAGTCTGG | |
| | CAACACGGCCTCCCTGACAATCTCTGGGCTCCAGGCTGAGGA | |
| | CGAGGCTGATTATTACTGCTGCTCATATGCAAGTAGTTTCAC | |
| | CGTGCTATACGGCGGAGGGACCAAGCTGACCGTCCTA | |
| | SSDVGSYNL | 334 |
| | EVS | 335 |
| | CSYASSFTVL | 336 |
| L13.bP17E11 | QVQLVQSGAEVKKPGASVKVSCKASGYTFTSYFISWVRQAPGQ | 337 |
| | GLEWMGWISAYNGNTNYAQNLQGRVTMTTDTSTSTAYMELR | |
| | SLRSDDTAVYYCARGLMVYTGADYWGQGTLVTVSS | |
| | CAGGTTCAGCTGGTGCAGTCTGGAGCTGAGGTGAAGAAGCC | 338 |
| | TGGGGCCTCAGTGAAGGTCTCCTGCAAGGCTTCTGGTTACAC | |
| | CTTTACCAGCTACTTTATCAGCTGGGTGCGACAGGCCCCTGG | |
| | ACAAGGGCTTGAGTGGATGGGATGGATCAGCGCTTACAATG | |
| | GTAACACAAACTATGCACAGAACCTCCAGGGCAGAGTCACC | |
| | ATGACCACAGACACATCCACGAGCACAGCCTACATGGAGCT | |
| | GAGGAGCCTGAGATCTGACGACACGGCCGTGTATTACTGTG | |
| | CGAGAGGACTAATGGTGTATACGGGGGCTGACTACTGGGGC | |
| | CAGGGAACCCTGGTCACCGTCTCCTCA | |
| | GYTFTSYF | 339 |
| | ISAYNGNT | 340 |
| | ARGLMVYTGADY | 341 |
| | NFMLTQPHSVSESPGKTVTISCTGSSGSIASNYVQWYQQRPGSA | 342 |
| | PTTVIYEDNQRPSGVPDRFSGSIDSSSNSASLTISGLKTEDEADY | |
| | YCQSYDSSTWVFGGGTKLIVL | |
| | AATTTTATGCTGACTCAGCCCCACTCTGTGTCGGAGTCTCCG | 343 |
| | GGGAAGACGGTAACCATCTCCTGCACCGGCAGCAGTGGCAG | |
| | CATTGCCAGCAACTATGTGCAGTGGTACCAGCAGCGCCCGG | |
| | GCAGTGCCCCCACCACTGTGATCTATGAGGATAACCAAAGA | |
| | CCCTCTGGGGTCCCTGATCGGTTCTCTGGCTCCATCGACAGC | |
| | TCCTCCAACTCTGCCTCCCTCACCATCTCTGGACTGAAGACT | |
| | GAGGACGAGGCTGACTACTACTGTCAGTCTTATGATAGCAGC | |
| | ACTTGGGTGTTCGGCGGAGGGACCAAGCTGATCGTCCTA | |
| | SGSIASNY | 344 |
| | EDN | 345 |
| | QSYDSSTWV | 346 |
| L13.bP14F05 | EVQLVESGGGLVQPGGSLRLSCAASGFTFSSYWMSWVRQAPG | 347 |
| | KGLEWVANIKQDGSEKYYVDSVKGRFTISRDNAKNSLFLQMNS | |
| | LRAEDTAVYYCARVGVESWYFDYWGQGTLVTVSS | |
| | GAGGTGCAGCTGGTGGAGTCTGGGGGAGGCTTGGTCCAGCC | 348 |
| | TGGGGGGTCCCTGAGACTCTCCTGTGCAGCCTCTGGATTCAC | |
| | CTTCAGTAGCTATTGGATGAGCTGGGTCCGCCAGGCTCCAGG | |
| | GAAGGGGCTGGAGTGGGTGGCCAACATAAAGCAAGATGGA | |
| | AGTGAGAAATACTATGTGGACTCTGTGAAGGGCCGATTCAC | |
| | CATCTCCAGAGACAACGCCAAGAACTCACTCTTTCTGCAAAT | |
| | GAACAGCCTGAGAGCCGAGGACACGGCCGTGTATTACTGTG | |
| | CGAGAGTTGGAGTTGAGAGCTGGTACTTTGACTACTGGGGCC | |
| | AGGGAACCCTGGTCACCGTCTCCTCA | |
| | GFTFSSYW | 349 |
| | IKQDGSEK | 350 |
| | ARVGVESWYFDY | 351 |
| | SYELTQPPSVSVSPGQTASITCSGDKLGDKYACWYQQKPGQSP | 352 |
| | VLVIYQDNKRPSGIPERFSGSNSGNTATLTISGTQAMDEADYYC | |
| | QAWDSSTAVFGGGTKLTVL | |
| | TCCTATGAGCTGACTCAGCCACCCTCAGTGTCCGTGTCCCCA | 353 |
| | GGACAGACAGCCAGCATCACCTGCTCTGGAGATAAATTGGG | |
| | GGATAAATATGCTTGCTGGTATCAGCAGAAGCCAGGCCAGT | |
| | CCCCTGTGCTGGTCATCTATCAAGATAACAAGCGGCCCTCAG | |
| | GGATCCCTGAGCGATTCTCTGGCTCCAACTCTGGGAACACAG | |
| | CCACTCTGACCATCAGCGGGACCCAGGCTATGGATGAGGCT | |
| | GACTATTACTGTCAGGCGTGGGACAGCAGCACTGCGGTATTC | |
| | GGCGGAGGGACCAAGCTGACCGTCCTA | |
| | KLGDKY | 354 |
| | QDN | 355 |
| | QAWDSSTAV | 356 |
| L6.dP04E05 | EVQLVESGGGLVQPGRSLRLSCAASGFTFDDYAMHWVRQAPG | 357 |
| | KGLEWVSGISWNSDNIGYADSVKGRFTVSRDNAKNSLY | |
| | LQMNSLRAEDTALYYCTKDIHRKNDYYDILTGYYRVRYYYYA | |
| | MDVWGQGTTVTVSS | |
| | GAGGTGCAGCTGGTGGAGTCTGGGGGAGGCTTGGTACAGCC | 358 |
| | TGGCAGGTCCCTGAGACTCTCCTGTGCAGCCTCTGGATT | |
| | CACCTTTGATGATTATGCCATGCACTGGGTCCGGCAAGCTCC | |
| | AGGGAAGGGCCTGGAGTGGGTCTCAGGTATTAGTTGGA | |
| | ATAGTGATAACATAGGCTATGCGGACTCTGTGAAGGGCCGA | |
| | TTCACCGTCTCCAGAGACAACGCCAAGAACTCCCTGTAT | |
| | CTGCAAATGAACAGTCTGAGAGCTGAGGACACGGCCTTGTA | |
| | TTACTGTACAAAAGATATCCACCGAAAAAATGACTATTA | |
| | CGATATTTTGACTGGTTATTATAGGGTTCGGTACTACTACTA | |
| | CGCTATGGACGTCTGGGGCCAAGGGACCACGGTCACCG | |
| | TCTCCTCA | |
| | GFTFDDYA | 359 |
| | ISWNSDNI | 360 |
| | TKDIHRKNDYYDILTGYYRVRYYYYAMDV | 361 |
| | QSVLTQPPSASGSPGQSVTISCTGTSSDAGGYNYVSWYQQHPG | 362 |
| | KAPKLMIYEVIKRPSGVPDRFSGSKSGNTASLTVSGL | |
| | QAEDEADYYCSSFAGTSVVFGGGTKLTVL | |
| | CAGTCTGTGCTGACTCAGCCTCCCTCCGCGTCCGGGTCTCCT | 363 |
| | GGACAGTCAGTCACCATCTCCTGCACTGGAACCAGCAG | |
| | TGACGCTGGTGGTTATAACTATGTCTCCTGGTACCAACAGCA | |
| | CCCAGGCAAAGCCCCCAAACTCATGATTTATGAGGTCA | |
| | TTAAGCGGCCCTCAGGGGTCCCTGATCGCTTCTCTGGCTCCA | |
| | AGTCTGGCAACACGGCCTCCCTGACCGTCTCTGGGCTC | |
| | CAGGCTGAGGATGAGGCTGATTATTACTGCAGCTCATTTGCA | |
| | GGCACCAGCGTGGTATTCGGCGGAGGGACCAAGCTGAC | |
| | CGTCCTA | |
| | SSDAGGYNY | 364 |
| | EVI | 365 |
| | SSFAGTSVV | 366 |
| L6.P4A3.1 | QVQLVQSGAEVKKPGSSVKVSCKASGGIFSSSAISWVRQAPGQ | 367 |
| | GLEWMGGIIPILDISNYAQKFQGRVTITADKSTSTAFMELSSLRS | |
| | EDTAVYYCALTNQWDLLVYWGQGTLVTVSS | |
| | CAAGTTCAATTGGTTCAATCTGGTGCTGAAGTTAAAAAGCCA | 368 |
| | GGATCGTCTGTCAAGGTATCCTGCAAAGCTTCTGGTGGTATC | |
| | TTTTCTTCTTCTGCTATTTCTTGGGTCAGACAGGCTCCAGGTC | |
| | AGGGTTTGGAATGGATGGGTGGTATTATTCCAATTTTGGATA | |
| | TTAGCAATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGTTGAC | |
| | CAATCAATGGGATTTGTTGGTTTACTGGGGTCAGGGAACTTT | |
| | GGTTACTGTTTCTAGT | |
| | GGIFSSSA | 369 |
| | TIPILDIS | 370 |
| | ALTNQWDLLVY | 371 |
| | EIVLTQSPGTLSLSPGERATLSCRASQRVSSSYLAWYQQKPGQA | 372 |
| | PRLLIYGAESRATGIPDRFSGSGSGTDFTLTISRLEPEDFAVYYC | |
| | QHYGSSLWTFGQGTKLEIK | |
| | GAAATTGTACTTACTCAATCTCCAGGCACGTTGTCTTTGAGT | 373 |
| | CCAGGTGAGAGGGCTACGTTGTCGTGCAGAGCTTCTCAGCGT | |
| | GTTTCTTCCTCTTATTTGGCTTGGTATCAACAGAAGCCAGGTC | |
| | AAGCTCCGAGGTTGCTGATCTACGGTGCTGAATCTAGAGCTA | |
| | CAGGTATCCCAGACAGGTTCTCCGGTTCTGGATCTGGTACTG | |
| | ATTTTACTTTGACTATTTCTAGATTGGAACCAGAGGACTTCG | |
| | CTGTTTACTACTGCCAACATTATGGATCCTCTTTGTGGACTTT | |
| | CGGTCAGGGTACGAAGTTGGAAATTAAG | |
| | QRVSSSY | 374 |
| | GAE | 375 |
| | QHYGSSLWT | 376 |
| L6.P4A3.2 | QVQLVQSGAEVKKPGSSVKVSCKAPGGTFSSSAISWVRQAPGQ | 377 |
| | GLEWMGTIIPILDITNYAQKFQGRVTITADKSTSTAFMELSSLRS | |
| | EDTAVYYCAQRNQWDLLVYWGQGTLVTVSS | |
| | CAAGTTCAATTGGTTCAATCTGGTGCTGAAGTTAAAAAGCCA | 378 |
| | GGATCGTCTGTCAAGGTATCCTGCAAAGCTCCAGGTGGTACT | |
| | TTTTCTTCTTCTGCTATTTCTTGGGTCAGACAGGCTCCAGGTC | |
| | AGGGTTTGGAATGGATGGGTACCATTATTCCAATTTTGGATA | |
| | TTACTAATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGCAAAG | |
| | AAATCAATGGGATTTGTTGGTTTACTGGGGTCAGGGAACTTT | |
| | GGTTACTGTTTCTAGT | |
| | GGTFSSSA | 379 |
| | IIPILDIT | 380 |
| | AQRNQWDLLVY | 381 |
| | EIVLTQSPGTLSLSPGERATLSCRASQSVSASYLAWYQQKPGQA | 382 |
| | PRLLIYGAESRATGIPDRFSGSGSGTDFTLTISRLEPEDFAVYYC | |
| | QHYGSSRWTFGQGTKLEIK | |
| | GAAATTGTACTTACTCAATCTCCAGGCACGTTGTCTTTGAGT | 383 |
| | CCAGGTGAGAGGGCTACGTTGTCGTGCAGAGCTTCTCAGTCT | |
| | GTTTCTGCATCTTATTTGGCTTGGTATCAACAGAAGCCAGGT | |
| | CAAGCTCCGAGGTTGCTGATCTACGGTGCTGAATCTAGAGCT | |
| | ACAGGTATCCCAGACAGGTTCTCCGGTTCTGGATCTGGTACT | |
| | GATTTTACTTTGACTATTTCTAGATTGGAACCAGAGGACTTC | |
| | GCTGTTTACTACTGCCAACATTATGGATCATCTCGTTGGACTT | |
| | TCGGTCAGGGTACGAAGTTGGAAATTAAG | |
| | QSVSASY | 384 |
| | GAE | 385 |
| | QHYGSSRWT | 386 |
| L6.P4A3.3 | QVQLVQSGAEVKKPGSSVKVSCKAPGGTFSSSAISWVRQAPGQ | 387 |
| | GLEWMGGIIPILDITHYAQKFQGRVTITADKSTSTAFMELSSLRS | |
| | EDTAVYYCALRNQWDLLVYWGQGTLVTVSS | |
| | CAAGTTCAATTGGTTCAATCTGGTGCTGAAGTTAAAAAGCCA | 388 |
| | GGATCGTCTGTCAAGGTATCCTGCAAAGCTCCAGGTGGTACT | |
| | TTTTCTTCTTCTGCTATTTCTTGGGTCAGACAGGCTCCAGGTC | |
| | AGGGTTTGGAATGGATGGGTGGTATTATTCCAATTTTGGATA | |
| | TTACTCATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGTTGAG | |
| | AAATCAATGGGATCTGTTGGTTTACTGGGGTCAGGGAACTTT | |
| | GGTTACTGTTTCTAGT | |
| | GGTFSSSA | 389 |
| | IIPILDIT | 390 |
| | ALRNQWDLLVY | 391 |
| | EIVLTQSPGTLSLSPGERATLSCRASQDVSSSYLAWYQQKPGQA | 392 |
| | PRLLIYGAESRATGIPDRFSGSGSGTDFTLTISRLEPEDFAVYYC | |
| | QHYGSSEWTFGQGTKLEIK | |
| | GAAATTGTACTTACTCAATCTCCAGGCACGTTGTCTTTGAGT | 393 |
| | CCAGGTGAGAGGGCTACGTTGTCGTGCAGAGCTTCTCAGGAT | |
| | GTTTCTTCCTCTTATTTGGCTTGGTATCAACAGAAGCCAGGTC | |
| | AAGCTCCGAGGTTGCTGATCTACGGTGCTGAATCTAGAGCTA | |
| | CAGGTATCCCAGACAGGTTCTCCGGTTCTGGATCTGGTACTG | |
| | ATTTTACTTTGACTATTTCTAGATTGGAACCAGAGGACTTCG | |
| | CTGTTTACTACTGCCAACATTATGGATCCTCTGAATGGACTTT | |
| | CGGTCAGGGTACGAAGTTGGAAATTAAG | |
| | QDVSSSY | 394 |
| | GAE | 395 |
| | QHYGSSEWT | 396 |
| L6.P4A3.4 | QVQLVQSGAEVKKPGSSVKVSCKASGGIFSSSAISWVRQAPGQ | 397 |
| | GLEWMGGIIPILDITNYAQKFQGRVTITADKSTSTAFMELSSLRS | |
| | EDTAVYYCALINQWDLLVYWGQGTLVTVSS | |
| | CAAGTTCAATTGGTTCAATCTGGTGCTGAAGTTAAAAAGCCA | 398 |
| | GGATCGTCTGTCAAGGTATCCTGCAAAGCTTCTGGTGGTATC | |
| | TTTTCTTCTTCTGCTATTTCTTGGGTCAGACAGGCTCCAGGCC | |
| | AGGGTTTGGAATGGATGGGTGGTATTATTCCAATTTTGGATA | |
| | TTACTAATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGTTGAC | |
| | CAATCAATGGGATTTGTTGGTTTACTGGGGTCAGGGAACTTT | |
| | GGTTACTGTTTCTAGT | |
| | GGIFSSSA | 399 |
| | IIPILDIT | 400 |
| | ALTNQWDLLVY | 401 |
| | EIVLTQSPGTLSLSPGERATLSCRASQSVSSSYLAWYQQKPGQA | 402 |
| | PRLLIYGAESRATGIPDRFSGSGSGTDFTLTISRLEPEDFAVYYC | |
| | QHYGSSAWTFGQGTKLEIK | |
| | GAAATTGTACTTACTCAATCTCCAGGCACGTTGTCTTTGAGT | 403 |
| | CCAGGTGAGAGGGCTACGTTGTCGTGCAGAGCTTCTCAGTCT | |
| | GTTTCTTCCTCTTATTTGGCTTGGTATCAACAGAAGCCAGGTC | |
| | AAGCTCCGAGGTTGCTGATCTACGGTGCTGAATCTAGAGCTA | |
| | CAGGTATCCCAGACAGGTTCTCCGGTTCTGGATCTGGTACTG | |
| | ATTTTACTTTGACTATTTCTAGATTGGAACCAGAGGACTTCG | |
| | CTGTTTACTACTGCCAACATTATGGATCCTCTGCATGGACTTT | |
| | CGGTCAGGGTACGAAGTTGGAAATTAAG | |
| | QSVSSSY | 404 |
| | GAE | 405 |
| | QHYGSSAWT | 406 |
| L6.P4A3.5 | QVQLVQSGAEVKKPGSSVKVSCKAVGGTFSSSAIYWVRQAPG | 407 |
| | QGLEWMGGIIPILDIANYAQKFQGRVTITADKSTSTAFMELSSLR | |
| | SEDTAVYYCALRNQWDLVVYWGQGTLVTVSS | |
| | CAAGTTCAATTGGTTCAATCTGGTGCTGAAGTTAAAAAGCCA | 408 |
| | GGATCGTCTGTCAAGGTATCCTGCAAAGCTGTTGGTGGTACT | |
| | TTTTCTTCTTCTGCTATTTACTGGGTCAGACAGGCTCCAGGTC | |
| | AGGGTTTGGAATGGATGGGTGGTATTATTCCAATTTTGGATA | |
| | TTGCAAATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGTTGAG | |
| | AAATCAATGGGATTTGGTTGTTTACTGGGGTCAGGGAACTTT | |
| | GGTTACTGTTTCTAGT | |
| | GGTFSSSA | 409 |
| | IIPILDIA | 410 |
| | ALRNQWDLVVY | 411 |
| | EIVLTQSPGTLSLSPGERATLSCHASQSVSSSYLAWYQQKPGQA | 412 |
| | PRLLIYGAESRATGIPDRFSGSGSGTDFTLTISRLEPEDFAVYYC | |
| | QHYGSSTWTFGQGTKLEIK | |
| | GAAATTGTACTTACTCAATCTCCAGGCACGTTGTCTTTGAGT | 413 |
| | CCAGGTGAGAGGGCTACGTTGTCGTGCCATGCTTCTCAGTCT | |
| | GTTTCTTCCTCTTATTTGGCTTGGTATCAACAGAAGCCAGGTC | |
| | AAGCTCCGAGGTTGCTGATCTACGGTGCTGAATCTAGAGCTA | |
| | CAGGTATCCCAGACAGGTTCTCCGGTTCTGGATCTGGTACTG | |
| | ATTTTACTTTGACTATTTCTAGATTGGAACCAGAGGACTTCG | |
| | CTGTTTACTACTGCCAACATTATGGATCCTCTACCTGGACTTT | |
| | CGGTCAGGGTACGAAGTTGGAAATTAAG | |
| | QSVSSSY | 414 |
| | GAE | 415 |
| | QHYGSSTWT | 416 |
| L6.P4A3.b | QVQLVQSGAEVKKPGSSVKVSCKAPGGTFSSSAISWVRQAPGQ | 417 |
| | GLEWMGGIIPILDITTYAQKFQGRVTITADKSTSTAFMELSSLRS | |
| | EDTAVYYCALRNQWDLLIYWGQGTLVTVSS | |
| | CAAGTTCAATTGGTTCAATCTGGTGCTGAAGTTAAAAAGCCA | 418 |
| | GGATCGTCTGTCAAGGTATCCTGCAAAGCTCCAGGTGGTACT | |
| | TTTTCTTCTTCTGCTATTTCTTGGGTCAGACAGGCTCCAGGTC | |
| | AGGGTTTGGAATGGATGGGTGGTATTATTCCAATTTTGGATA | |
| | TTACTACCTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGTTGAG | |
| | AAATCAATGGGATTTGTTGATCTACTGGGGTCAGGGAACTTT | |
| | GGTTACTGTTTCTAGT | |
| | GGTFSSSA | 419 |
| | IIPILDIT | 420 |
| | ALRNQWDLLIY | 421 |
| | EIVLTQSPGTLSLSPGERATLSCRASQSVSSHYLAWYQQKPGQA | 422 |
| | PRLLIYGASSRYTGIPDRFSGSGSGTDFTLTISRLEPEDFAVYYCQ | |
| | HYGSSHWTFGQGTKLEIK | |
| | GAAATTGTACTTACTCAATCTCCAGGCACGTTGTCTTTGAGT | 423 |
| | CCAGGTGAGAGGGCTACGTTGTCGTGCAGAGCTTCTCAGTCT | |
| | GTTTCTTCCCATTATTTGGCTTGGTATCAACAGAAGCCAGGT | |
| | CAAGCTCCGAGGTTGCTGATCTACGGTGCTTCTTCTAGATAC | |
| | ACAGGTATCCCAGACAGGTTCTCCGGTTCTGGATCTGGTACT | |
| | GATTTTACTTTGACTATTTCTAGATTGGAACCAGAGGACTTC | |
| | GCTGTTTACTACTGCCAACATTATGGATCCTCTCATTGGACTT | |
| | TCGGTCAGGGTACGAAGTTGGAAATTAAG | |
| | QSVSSHY | 424 |
| | GAS | 425 |
| | QHYGSSHWT | 426 |
| L6.P4A3.7 | QVQLVQSGAEVKKPGSSVKVSCKASGGIFSSSAISWVRQAPGQ | 427 |
| | GLEWMGGIIPILDYTNYAQKFQGRVTITADKSTSTAFMELSSLR | |
| | SEDTAVYYCALRNQWDLLVYWGQGTLVTVSS | |
| | CAAGTTCAATTGGTTCAATCTGGTGCTGAAGTTAAAAAGCCA | 428 |
| | GGATCGTCTGTCAAGGTATCCTGCAAAGCTTCTGGTGGTATC | |
| | TTTTCTTCTTCTGCTATTTCTTGGGTCAGACAGGCTCCAGGTC | |
| | AGGGTTTGGAATGGATGGGTGGTATTATTCCAATTTTGGATT | |
| | ACACTAATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGTTGCG | |
| | TAATCAATGGGATTTGTTGGTTTACTGGGGTCAGGGAACTTT | |
| | GGTTACTGTTTCTAGT | |
| | GGIFSSSA | 429 |
| | IIPILDYT | 430 |
| | ALRNQWDLLVY | 431 |
| | EIVLTQSPGTLSLSPGERATLSCRASQEVSSSYLAWYQQKPGQA | 432 |
| | PRLLIYGAESRATGIPDRFSGSGSGTDFTLTISRLEPEDFAVYYC | |
| | QHYGSSAWTFGQGTKLEIK | |
| | GAAATTGTACTTACTCAATCTCCAGGCACGTTGTCTTTGAGT | 433 |
| | CCAGGTGAGAGGGCTACGTTGTCGTGCAGAGCTTCTCAGGA | |
| | AGTTTCTTCCTCTTATTTGGCTTGGTATCAACAGAAGCCAGG | |
| | TCAAGCTCCGAGGTTGCTGATCTACGGTGCTGAATCTAGAGC | |
| | TACAGGTATCCCAGACAGGTTCTCCGGTTCTGGATCTGGTAC | |
| | TGATTTTACTTTGACTATTTCTAGATTGGAACCAGAGGACTT | |
| | CGCTGTTTACTACTGCCAACATTATGGATCCTCTGCATGGAC | |
| | TTTCGGTCAGGGTACGAAGTTGGAAATTAAG | |
| | QEVSSSY | 434 |
| | GAE | 435 |
| | QHYGSSAWT | 436 |
| L6.P4A3.8 | QVQLVQSGAEVKKPGSSVKVSCKVSGGTFSSSAISWVRQAPGQ | 437 |
| | GLEWMGKIIPILDITNYAQKFQGRVTITADKSTSTAFMELSSLRS | |
| | EDTAVYYCALRNQWDLLVYWGQGTLVTVSS | |
| | CAAGTTCAATTGGTTCAATCTGGTGCTGAAGTTAAAAAGCCA | 438 |
| | GGATCGTCTGTCAAGGTATCCTGCAAAGTTTCTGGTGGTACT | |
| | TTTTCTTCTTCTGCTATTTCTTGGGTCAGACAGGCTCCAGGCC | |
| | AGGGTTTGGAATGGATGGGTAAGATTATTCCAATTTTGGATA | |
| | TTACTAATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGCTGAG | |
| | AAATCAATGGGATTTGTTGGTTTACTGGGGTCAGGGAACTTT | |
| | GGTTACTGTTTCTAGT | |
| | GGTFSSSA | 439 |
| | IIPILDIT | 440 |
| | ALRNQWDLLVY | 441 |
| | EIVLTQSPGTLSLSPGERATLSCRASQSVSSHYLAWYQQKPGQA | 442 |
| | PRLLIYGAESRATGIPDRFSGSGSGTDFTLTISRLEPEDFAVYYC | |
| | QHYGSSPWTFGQGTKLEIK | |
| | GAAATTGTACTTACTCAATCTCCAGGCACGTTGTCTTTGAGT | 443 |
| | CCAGGTGAGAGGGCTACGTTGTCGTGCAGAGCTTCTCAGTCT | |
| | GTTTCTTCCCATTATTTGGCTTGGTATCAACAGAAGCCAGGT | |
| | CAAGCTCCGAGGTTGCTGATCTACGGTGCTGAATCTAGAGCT | |
| | ACAGGTATCCCAGACAGGTTCTCCGGTTCTGGATCTGGTACT | |
| | GATTTTACTTTGACTATTTCTAGATTGGAACCAGAGGACTTC | |
| | GCTGTTTACTACTGCCAACATTATGGATCCTCTCCATGGACTT | |
| | TCGGTCAGGGTACGAAGTTGGAAATTAAG | |
| | QSVSSHY | 444 |
| | GAE | 445 |
| | QHYGSSPWT | 446 |
| L6.P4A3.9 | QVQLVQSGAEVKKPGSSVKVSCKSSGGTFSSSAYSWVRQAPGQ | 447 |
| | GLEWMGTIIPILDITNYAQKFQGRVTITADKSTSTAFMELSSLRS | |
| | EDTAVYYCAQRNQWDLIVYWGQGTLVTVSS | |
| | CAAGTTCAATTGGTTCAATCTGGTGCTGAAGTTAAAAAGCCA | 448 |
| | GGATCGTCTGTCAAGGTATCCTGCAAAAGCTCTGGTGGTACT | |
| | TTTTCTTCTTCTGCTTACTCTTGGGTCAGACAGGCTCCAGGCC | |
| | AGGGTTTGGAATGGATGGGTACCATTATTCCAATTTTGGATA | |
| | TTACTAATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGCAAAG | |
| | AAATCAATGGGATTTGATCGTTTACTGGGGTCAGGGAACTTT | |
| | GGTTACTGTTTCTAGT | |
| | GGTFSSSA | 449 |
| | IIPILDIT | 450 |
| | AQRNQWDLIVY | 451 |
| | EIVLTQSPGTLSLSPGERATLSCRASQWVSSSYIAWYQQKPGQA | 452 |
| | PRLLIYGAESRATGIPDRFSGSGSGTDFTLTISRLEPEDFAVYYC | |
| | QHYGSSSWTFGQGTKLEIK | |
| | GAAATTGTACTTACTCAATCTCCAGGCACGTTGTCTTTGAGT | 453 |
| | CCAGGTGAGAGGGCTACGTTGTCGTGCAGAGCTTCTCAGTGG | |
| | GTTTCTTCCTCTTATATCGCTTGGTATCAACAGAAGCCAGGT | |
| | CAAGCTCCGAGGTTGCTGATCTACGGTGCTGAATCTAGAGCA | |
| | ACAGGTATCCCAGACAGGTTCTCCGGTTCTGGATCTGGTACT | |
| | GATTTTACTTTGACTATTTCTAGATTGGAACCAGAGGACTTC | |
| | GCTGTTTACTACTGCCAACATTATGGATCCTCTAGCTGGACT | |
| | TTCGGTCAGGGTACGAAGTTGGAAATTAAG | |
| | QWVSSSY | 454 |
| | GAE | 455 |
| | QHYGSSSWT | 456 |
| L6.P4A3.10 | QVQLVQSGAEVKKPGSSVKVSCKAPGGTFSSSAISWVRQAPGQ | 457 |
| | GLEWMGGIIPILDITHYAQKFQGRVTITADKSTSTAFMELSSLRS | |
| | EDTAVYYCALRNQWDLLVYWGQGTLVTVSS | |
| | CAAGTTCAATTGGTTCAATCTGGTGCTGAAGTTAAAAAGCCA | 458 |
| | GGATCGTCTGTCAAGGTATCCTGCAAAGCTCCAGGTGGTACT | |
| | TTTTCTTCTTCTGCTATTTCTTGGGTCAGACAGGCTCCAGGTC | |
| | AGGGTTTGGAATGGATGGGIGGTATTATTCCAATTTTGGATA | |
| | TTACTCATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGTTGAG | |
| | AAATCAATGGGATCTGTTGGTTTACTGGGGTCAGGGAACTTT | |
| | GGTTACTGTTTCTAGT | |
| | GGTFSSSA | 459 |
| | IIPILDIT | 460 |
| | ALRNQWDLLVY | 461 |
| | EIVLTQSPGTLSLSPGERATLSCRASQSVSSSYLAWYQQKPGQA | 462 |
| | PRLLIYGAESRATGIPDRFSGSGSGTDFTLTISRLEPEDFAVYYC | |
| | QHYGSSVWTFGQGTKLEIK | |
| | GAAATTGTACTTACTCAATCTCCAGGCACGTTGTCTTTGAGT | 463 |
| | CCAGGTGAGAGGGCTACGTTGTCGTGCAGAGCTTCTCAGTCT | |
| | GTTTCTTCCTCTTATTTGGCTTGGTATCAACAGAAGCCAGGTC | |
| | AAGCTCCGAGGTTGCTGATCTACGGTGCTGAATCTAGAGCTA | |
| | CAGGTATCCCAGACAGGTTCTCCGGTTCTGGATCTGGTACTG | |
| | ATTTTACTTTGACTATTTCTAGATTGGAACCAGAGGACTTCG | |
| | CTGTTTACTACTGCCAACATTATGGATCCTCTGTTTGGACTTT | |
| | CGGTCAGGGTACGAAGTTGGAAATTAAG | |
| | QSVSSSY | 464 |
| | GAE | 465 |
| | QHYGSSVWT | 466 |
| L6.P4A3.11 | QVQLVQSGAEVKKPGSSVKVSCKISGGYFSSSAISWVRQAPGQ | 467 |
| | GLEWMGGIIPVLDITNYAQKFQGRVTITADKSTSTAFMELSSLR | |
| | SEDTAVYYCALINQWDLLVYWGQGTLVTVSS | |
| | CAAGTTCAATTGGTTCAATCTGGTGCTGAAGTTAAAAAGCCA | 468 |
| | GGATCGTCTGTCAAGGTATCCTGCAAAATCTCTGGTGGTTAC | |
| | TTTTCTTCTTCTGCTATTTCTTGGGTCAGACAGGCTCCAGGCC | |
| | AGGGTTTGGAATGGATGGGTGGTATTATTCCAGTTTTGGATA | |
| | TTACTAATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGTTGAC | |
| | CAATCAATGGGATTTGTTGGTTTACTGGGGTCAGGGAACTTT | |
| | GGTTACTGTTTCTAGT | |
| | GGYFSSSA | 469 |
| | IIPVLDIT | 470 |
| | ALTNQWDLLVY | 471 |
| | EIVLTQSPGTLSLSPGERATLSCSASQSVSSSYLAWYQQKPGQA | 472 |
| | PRLLIYGAESRATGIPDRFSGSGSGTDFTLTISRLEPEDFAVYYC | |
| | QHYGSSHWTFGQGTKLEIK | |
| | GAAATTGTACTTACTCAATCTCCAGGCACGTTGTCTTTGAGT | 473 |
| | CCAGGTGAGAGGGCTACGTTGTCGTGCAGCGCTTCTCAGTCT | |
| | GTTTCTTCCTCTTATTTGGCTTGGTATCAACAGAAGCCAGGTC | |
| | AAGCTCCGAGGTTGCTGATCTACGGTGCTGAATCTAGAGCTA | |
| | CAGGTATCCCAGACAGGTTCTCCGGTTCTGGATCTGGTACTG | |
| | ATTTTACTTTGACTATTTCTAGATTGGAACCAGAGGACTTCG | |
| | CTGTTTACTACTGCCAACATTATGGATCCTCTCATTGGACTTT | |
| | CGGTCAGGGTACGAAGTTGGAAATTAAG | |
| | QSVSSSY | 474 |
| | GAE | 475 |
| | QHYGSSHWT | 476 |
| L6.P4A3.12 | QVQLVQSGAEVKKPGSSVKVSCKASGGLFSSSAISWVRQAPGQ | 477 |
| | GLEWMGGIIPILDITNYAQKFQGRVTITADKSTSTAFMELSSLRS | |
| | EDTAVYYCALTNQWDLLVYWGQGTLVTVSS | |
| | CAAGTTCAATTGGTTCAATCTGGTGCTGAAGTTAAAAAGCCA | 478 |
| | GGATCGTCTGTCAAGGTATCCTGCAAAGCTTCTGGTGGTCTG | |
| | TTTTCTTCTTCTGCTATTTCTTGGGTCAGACAGGCTCCAGGCC | |
| | AGGGTTTGGAATGGATGGGTGGTATTATTCCAATTTTGGATA | |
| | TTACTAATTACGCTCAGAAGTTCCAGGGTAGAGTTACTATTA | |
| | CTGCTGATAAATCTACTTCTACTGCTTTTATGGAATTGTCTTC | |
| | TTTGAGATCTGAAGACACTGCCGTCTACTATTGTGCGTTGAC | |
| | CAATCAATGGGATTTGTTGGTTTACTGGGGTCAGGGAACTTT | |
| | GGTTACTGTTTCTAGT | |
| | GGLFSSSA | 479 |
| | IIPILDIT | 480 |
| | ALTNQWDLLVY | 481 |
| | EIVLTQSPGTLSLSPGERATLSCRASQSVSSSYLAWYQQKPGQA | 482 |
| | PRLLIYGAESRATGIPDRFSGSGSGTDFTLTISRLEPEDFAVYYC | |
| | QHYGSSRWTFGQGTKLEIK | |
| | GAAATTGTACTTACTCAATCTCCAGGCACGTTGTCTTTGAGT | 483 |
| | CCAGGTGAGAGGGCTACGTTGTCGTGCAGAGCTTCTCAGTCT | |
| | GTTTCTTCCTCTTATTTGGCTTGGTATCAACAGAAGCCAGGTC | |
| | AAGCTCCGAGGTTGCTGATCTACGGTGCTGAATCTAGAGCTA | |
| | CAGGTATCCCAGACAGGTTCTCCGGTTCTGGATCTGGTACTG | |
| | ATTTTACTTTGACTATTTCTAGATTGGAACCAGAGGACTTCG | |
| | CTGTTTACTACTGCCAACATTATGGATCCTCTCGTTGGACTTT | |
| | CGGTCAGGGTACGAAGTTGGAAATTAAG | |
| | QSVSSSY | 484 |
| | GAE | 485 |
| | QHYGSSRWT | 486 |

The disclosure above will be more readily understood by reference to the following examples, which are included merely for purposes of illustration of certain embodiments of the present invention, and are not intended to limiting.

### EXEMPLIFICATION

### Example: 1

Target Cell Line

HeLa-hACE2 and A549-hACE2 cells were generated through transduction of human ACE2 lentivirus, pBOB-hACE2 construct was co-transfected into HEK293T cells along with lentiviral packaging plasmids pMDL, pREV, and pVSV-G (Addgene) by Lipofectamine 2000 (ThermoFischer Scientific, 11668019) according to manufacturer's instructions. Supernatants were collected 32 h after transfection, and then were transducted to pre-seeded HeLa or A549 cells. 12 h after transduction, stable cell lines were collected, and stored for neutralization assay.

### Example: 2

Growing Virus

Vero E6 cells (ATCC CRL-1586) were plated in a T225 flask with complete DMEM (Corning 15-013-CV) containing 10% FBS, 1×PenStrep (Corning 20-002-CL), 2 mM L-Glutamine (Corning 25-005-CL) overnight at 37° CC 5% CO₂. The media in the flask was removed and 2 mL of SARS-CoV-2 strain USA-WA1/2020 (BEI Resources NR-52281) in complete DMEM was added to the flask at an MOI of 0.5 and was allowed to incubate for 30 minutes at 34° C. 5% CO₂. After incubation, 30 mL of complete DMEM was added to the flask. The flask was then placed in a 34° C. incubator at 5% CO₂ for 5 days. On day 5 post-infection the supernatant was harvested and centrifuged at 1,000×g for 5 minutes. The supernatant was filtered through a 0.22 μuM filter and stored at −80° C.

### Example: 3

Pseudovirus Assay

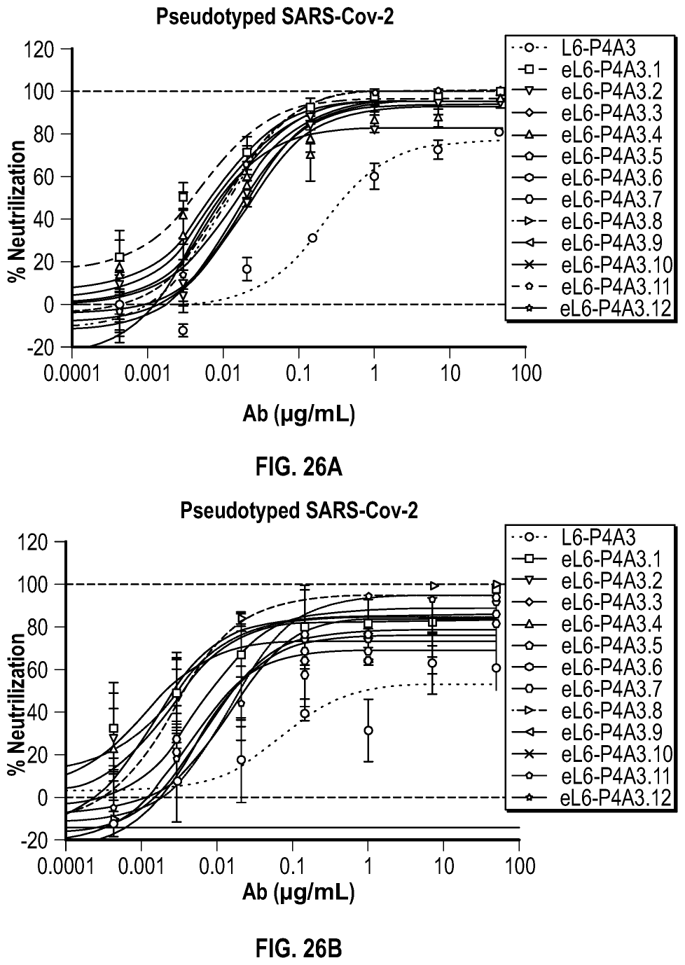

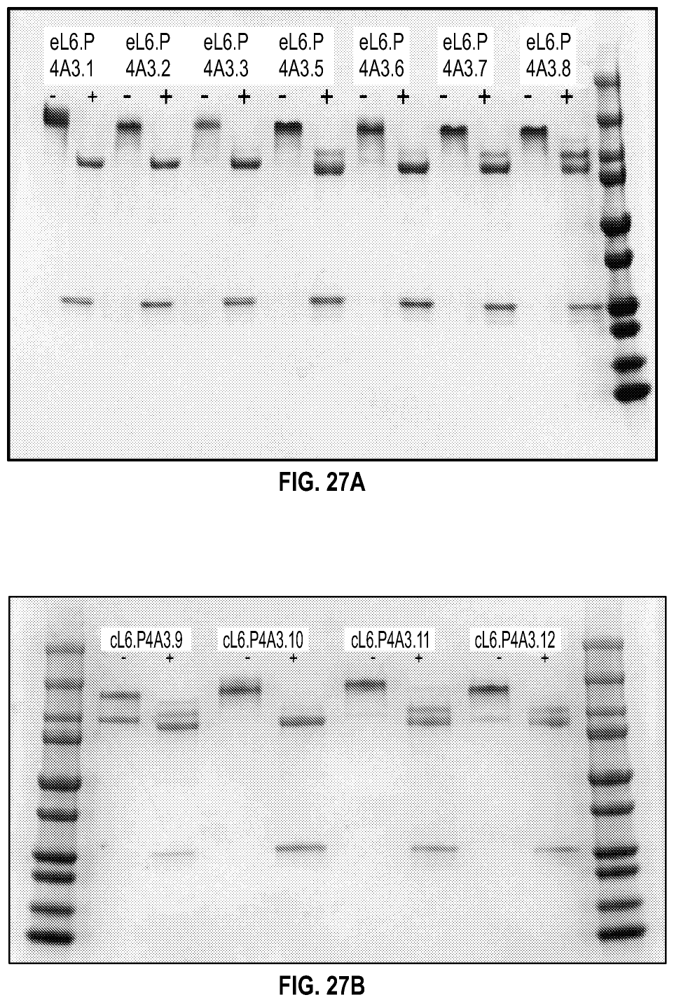

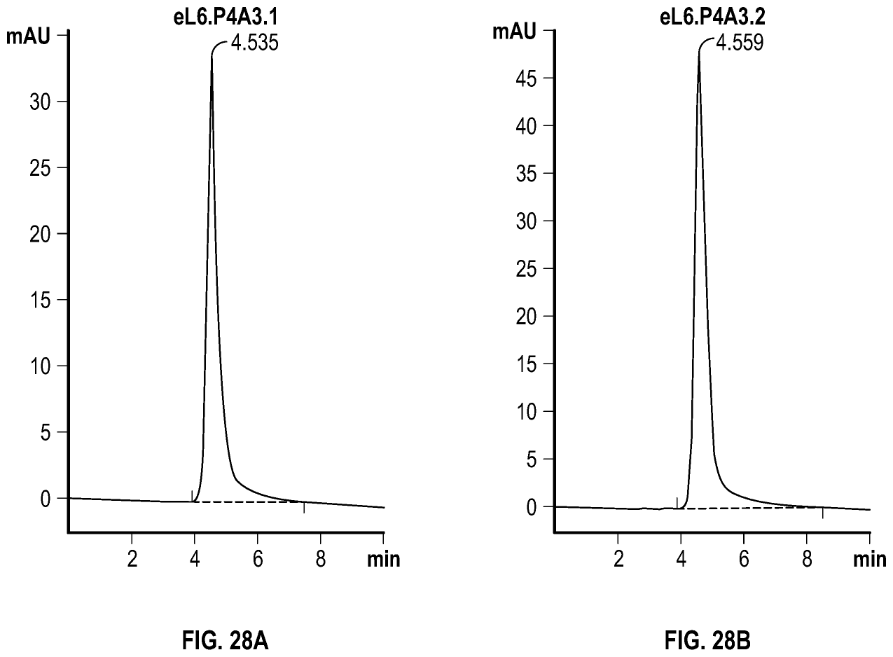

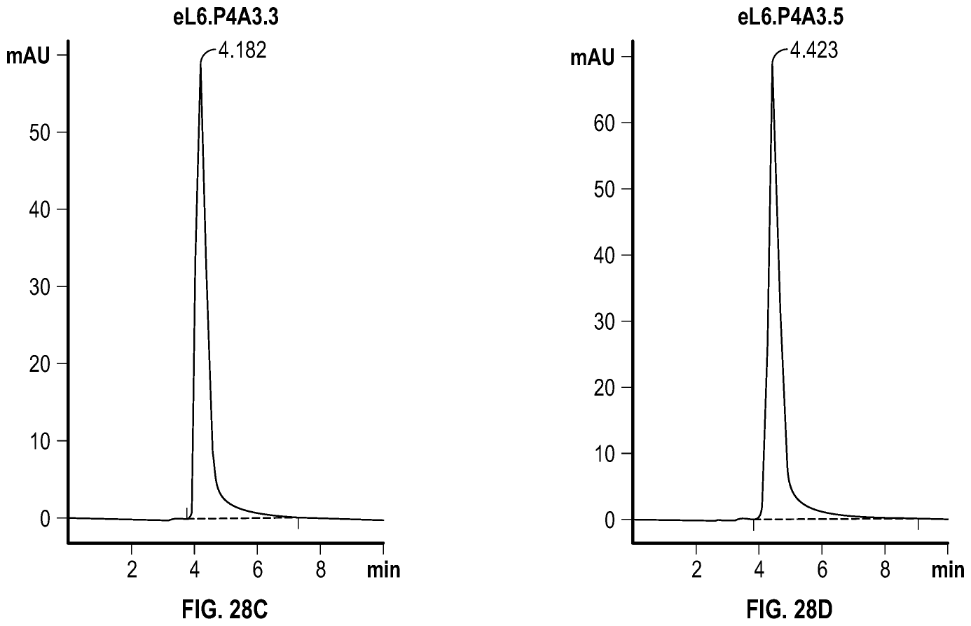

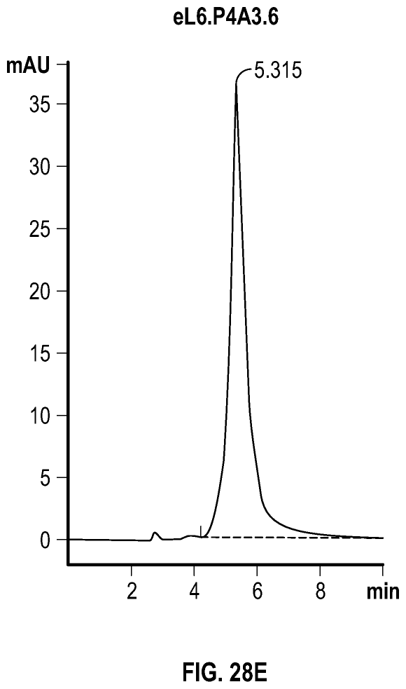

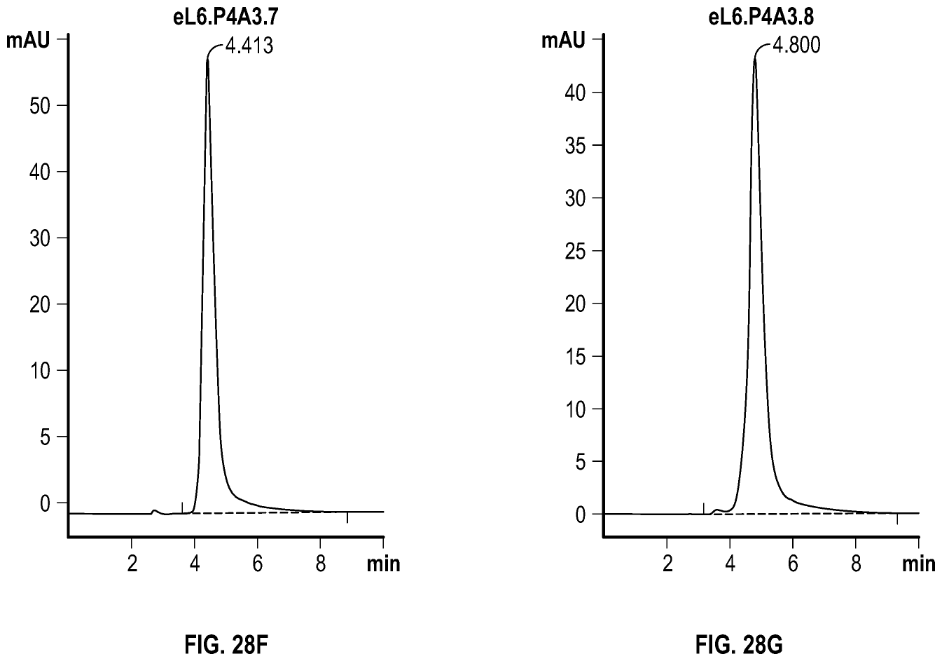

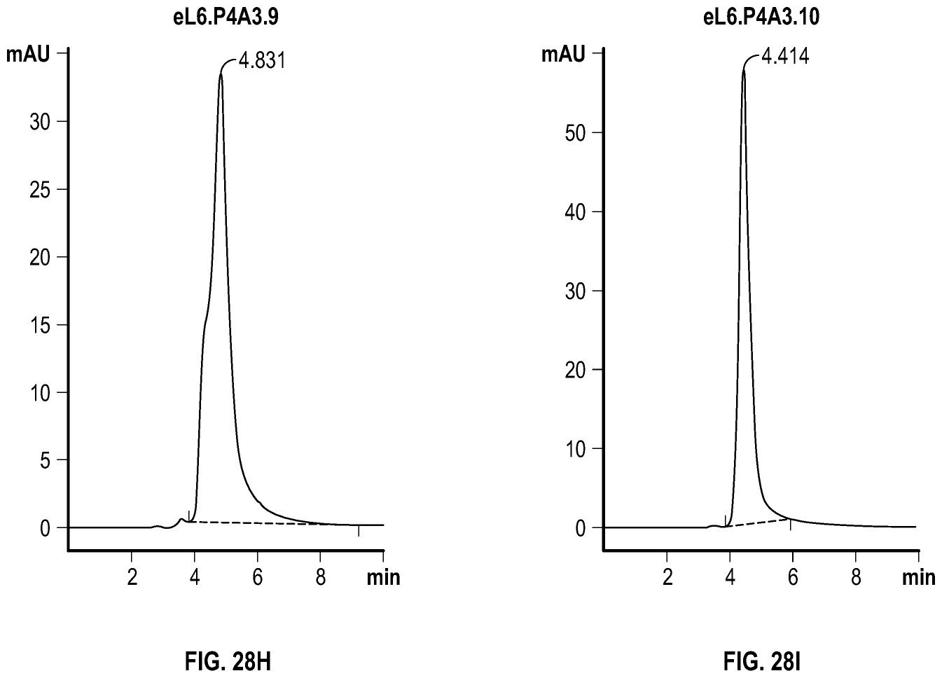

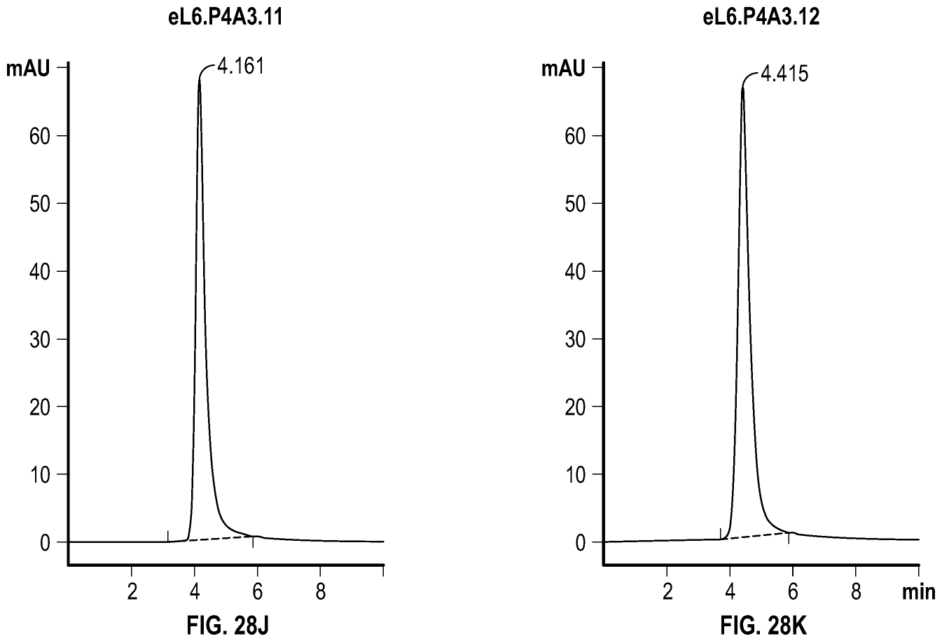

MLV-gag/pol and MLV-CMV plasmids were co-transfected with full-length or truncated SARS-Cov and SARS-Cov-2 plasmid respectively with transfection reagent Lipofectamine 2000 in HEK293T cells. After 48 h of transfection, pseudoviruses were harvested from cell culture supernatants and frozen at −80° C. for long-term storage. Serially diluted plasma or mAbs were incubated with pseudovirus at 37° C. for 1 h, then transferred onto HeLa-hACE2 cells in 96-well plates at 10,000 cells/well (Corning, 3688). After 48 h of incubation, supernatants were removed, HeLa-hACE2 cells were then lysed using luciferase lysis buffer (25 mM Gly-Gly pH 7.8, 15 mM MgSO4, 4 mM EGTA, 1% Triton X-100). Luciferase activity was measured by adding Bright-Glo (Promega, PR-E2620) according to manufacturer's instructions. Plasma or mAbs were tested in duplicate wells. Neutralization ID50 or IC50 titers were calculated using “One-Site Fit Log IC50” regression in GraphPad Prism 8.0. The results from the neutralization assay are reproduced in table 2A, 2B, and 2C below (Table 2C correlates with FIG. 26A-B). In table 2C, the enhanced eL6.P4A3 monoclonal antibody variants neutralize SARS1 and SARS2. Table 1 provides the sequence information relating to each of the clones set forth below. Table 3 depicts the polyreactivity of the PSV between SARS1 and SARS2.

TABLE 2A

| | SARS2 | SARS1 |
|---|---|---|
| L12.bP11A06 | 0.017 | >50 |
| L12.bP11A11 | 0.016 | >50 |
| L12.bP11G07 | 0.013 | >50 |
| L12.bP12D08 | 0.628 | >50 |
| L12.bP10G07 | 0.865 | |
| L12 bP11C10 | 0.092 | >50 |
| L12.bP12E05 | 0.075 | >50 |
| L12.bP12H09 | 0.062 | >50 |
| L12 bP11E12 | 0.025 | |
| L12.bP13A12 | 0.051 | >50 |
| L12.cP7E10 | 0.029 | >50 |
| L12.cP8D07 | 1.81 | >50 |
| L12.cP9B02 | 0.148 | >50 |
| L12.cP8A09 | 0.382 | >50 |
| L12.cP8D10 | 4.30 | >50 |
| L12.cP9C07 | 0.085 | >50 |
| L12.cP9D03 | 0.351 | >50 |
| L12.cP9D05 | 0.008 | >50 |

TABLE 2B

| | | | Maximum |
|---|---|---|---|
| | | | Plateau of |
| mAb Nickname | SARS-CoV-1 | SARS-CoV-2 | Neutralization |
| L12.bP11A06 | >50 | 0.019 | 100% |
| L12.cP09B02 | >50 | 0.224 | 100% |
| L12.bP11G07 | >50 | 0.018 | 100% |
| L12.bP11A11 | >50 | 0.110 | 100% |
| L12.bP10G07 | >50 | 0.328 | 100% |
| L12.bP11C10 | >50 | 0.494 | 100% |
| L12.bP12E05 | >50 | 0.255 | 89% |
| L12.bP12H09 | >50 | 0.106 | 100% |
| L12.cP08D10 | >50 | 23.460 | 60% |
| L12.cP09C07 | >50 | 0.0 [symbol] | 95% |
| L12.cP09D03 | >50 | 0.135 | 85% |
| L12.cP08A09 | >50 | 1.502 | 99% |
| L12.cP07E10 | >50 | 0.100 | 100% |
| L12.cP09D05 | >50 | 0.023 | 100% |
| L12.bP12D08 | >50 | 2.085 | 91% |
| L12.bP13A12 | >50 | >50 | 50% |
| L12.bP11E12 | >50 | >50 | 41% |
| L12.cP08D07 | >50 | 21.850 | 64% |
| L12.bP13A09 | >50 | >50 | 0% |
| L12.eP08E01 | >50 | 0.842 | 100% |
| L6.dP02E05 | >50 | 0.002 | 100% |
| L6.dP03H10 | >50 | 0.001 | 100% |
| L6.dP02A05 | >50 | 0.059 | 100% |
| L6.dP02F01 | 5.559? | 1.087 | 99% |
| L6.dP04A03 | 0.162 | 0.039 | 100% |
| L6.dP02C11 | N/A | N/A | N/A |
| L6.dP04C08 | >50 | >50 | 0% |
| L25.eP04C02 | >50 | 0.155 | 100% |
| L25.dP06E11 | >50 | 34.600 | 55% |

[symbol]

indicates data missing or illegible when filed

TABLE 2C

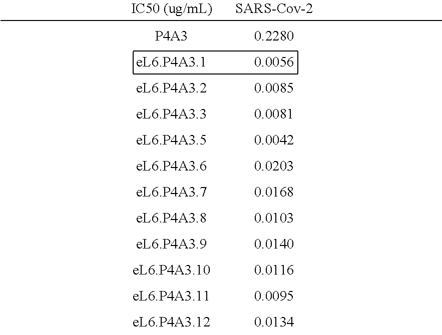

TABLE 3

PSV

| Polyreactivity | SARS1<ug/ml> | SARS2<ug/ml> | MPN |
|---|---|---|---|
| None | >50 | 0.0004 | 100% |

### Exemplary Sequences Used Herein:

RBD-2
(SEQ ID NO: 2)
RVQPTESIVRFPNITNLCPFGEVFNATRFASVYAW
NRKRISNCVADYSVLYNSASFSTFKCYGVSPTKLN
DLCFTNVYADSFVIRGDEVRQIAPGQTGKIADYNY
KLPDDFTGCVIAWNSNNLDSKVGGNYNYLYRLFRK
SNLKPFERDISTEIYQAGSTPCNGVEGFNCYFPLQ
SYGFQPINGVGYQPYRVVVLSFELLHAPATVCGPK
KSTNLVKNKCVNFNFNGLTGTGVLTESNKKFLPFQ
QFGRDIADTTDAVRDPQTLEILDITPCSGSGHHHH
HHHHHHGSGGLNDIFEAQKIEWHE
Legend:
Protein: residues 1-273
His-Tag: residues 277-286
AviTag: residues 290-304
Linker: residues 274-276 and 287-289
RBD-1
(SEQ ID NO: 3)
RVVPSGDVVRFPNITNLCPFGEVFNATKFPSVYAW
ERKKISNCVADYSVLYNSTFFSTFKCYGVSATKLN
DLCFSNVYADSFVVKGDDVRQIAPGQTGVIADYNY
KLPDDFMGCVLAWNTRNIDATSTGNYNYKYRYLRH
GKLRPFERDISNVPFSPDGKPCTPPALNCYWPLND
YGFYTTTGIGYQPYRVVVLSFELLNAPATVCGPKL
STDLIKNQCVNFNFNGLTGTGVLTPSSKRFQPFQQ
FGRDVSDFTDSVRDPKTSEILDISPCSGSGHHHHH
HHHHHGSGGLNDIFEAQKIEWHE
Legend:
Protein: residues 1-272
His-Tag: residues 276-285
AviTag: residues 288-303
Linker: residues 273-275 and 286-289
CoV-2 Spike
(SEQ ID NO: 1)
MFVFLVLLPLVSSQCVNLTTRTQLPPAYTNSFTRG
VYYPDKVFRSSVLHSTQDLFLPFFSNVTWFHAIHV
SGTNGTKRFDNPVLPFNDGVYFASTEKSNIIRGWI
FGTTLDSKTQSLLIVNNATNVVIKVCEFQFCNDPF
LGVYYHKNNKSWMESEFRVYSSANNCTFEYVSQPF
LMDLEGKQGNFKNLREFVFKNIDGYFKIYSKHTPI
NLVRDLPQGFSALEPLVDLPIGINITRFQTLLALH
RSYLTPGDSSSGWTAGAAAYYVGYLQPRTFLLKYN
ENGTITDAVDCALDPLSETKCTLKSFTVEKGIYQT
SNFRVQPTESIVRFPNITNLCPFGEVFNATRFASV
YAWNRKRISNCVADYSVLYNSASFSTFKCYGVSPT
KLNDLCFTNVYADSFVIRGDEVRQIAPGQTGKIAD
YNYKLPDDFTGCVIAWNSNNLDSKVGGNYNYLYRL
FRKSNLKPFERDISTEIYQAGSTPCNGVEGFNCYF
PLQSYGFQPTNGVGYQPYRVVVLSFELLHAPATVC
GPKKSTNLVKNKCVNFNFNGLTGTGVLTESNKKFL
PFQQFGRDIADTIDAVRDPQTLEILDITPCSFGGV
SVITPGTNTSNQVAVLYQDVNCTEVPVAIHADQLT
PTWRVYSTGSNVFQTRAGCLIGAEHVNNSYECDIP
IGAGICASYQTQTNSPGSASSVASQSIIAYTMSLG
AENSVAYSNNSIAIPTNFTISVTTEILPVSMTKTS
VDCTMYICGDSTECSNLLLQYGSFCTQLNRALTGI
AVEQDKNTQEVFAQVKQIYKTPPIKDFGGFNFSQI
LPDPSKPSKRSFIEDLLFNKVTLADAGFIKQYGDC
LGDIAARDLICAQKFNGLTVLPPLLTDEMIAQYTS
ALLAGTITSGWTFGAGAALQIPFAMQMAYRENGIG
VTQNVLYENQKLIANQFNSAIGKIQDSLSSTASAL
GKLQDVVNQNAQALNTLVKQLSSNFGAISSVLNDI
LSRLDPPEAEVQIDRLITGRLQSLQTYVTQQLIRA
AEIRASANLAATKMSECVLGQSKRVDFCGKGYHLM
SFPQSAPHGVVFLHVTYVPAQEKNFTTAPAICHDG
KAHFPREGVFVSNGTHWFVTQRNFYEPQUITTDNT
FVSGNCDVVIGIVNNTVYDPLQPELDSFKEELDKY
FKNHTSPDVDLGDISGINASVVNIQKEIDRLNEVA
KNLNESLIDLQELGKYEQGSGYIPEAPRDGQAYVR
KDGEWVLLSTFLGRSLEVLFQGPGHHHHHHHHSAW
SHPQFEKGGGSGGGGSGGSAWSHPQFEK
Legend:
Protein: residues 1-1208
Foldon trimerization sequence: residues 1211-1249
His-Tag: residues 1250-1257
Strep Tag II: residues 1258-1267 and 1279-1288
Linker: residues 1209-1210 and 1268-1278

### Example: 4

Antigen targets (RBD and CoV-2 spike) were coated on high-binding plates at a concentration of 1 μg/mL and incubated overnight at 4° C. The plates were then washed three times with 100 μL of 1×PBS+0.05% tween and subsequently blocked with 100 ul of 3% BSA for 1 hour at RT. The plates were then washed three times with 100 μL of 1×PBS+0.05% tween and subsequently 50 ul of a dilution series of monoclonal antibodies were added to the plate and incubated at RT for 1 hour. The plates were washed again three times with 100 μL of 1×PBS+0.05% tween before the addition of 50 ul of alkaline phosphatase conjugated goat anti-human Fc antibody (Jackson Immunoresearch 109-055-098) diluted at 1:1000 and incubated at RT for 1 hour. A final wash of 100 μL of 1×PBS+0.05% tween was completed before detection with 50 ul of alkaline phosphatase substrate in buffer (SIGMA-ALDRICH S0942). Non-linear regression curves were analyzed using Prism 8 software to calculate EC50 values.

TABLE 4

| | | ELISA (Binding EC50 in ug/ml) | | | |
|---|---|---|---|---|---|
| | | S protein | RBD | S protein | RBD |
| | Epitope | (SARS- | (SARS- | (SARS- | (SARS- |
| mAB Nickname | Binning | CoV-2) | CoV-2) | CoV-1) | CoV-1) |
| L12.bP11A06 | RBD-A | 0.017 | 0.042 | >50 | >50 |
| L12.cP09B02 | RBD-A | 0.021 | 0.143 | >50 | >50 |
| L12.bP11G07 | RBD-A | 0.018 | 0.025 | >50 | >50 |
| L12.bP11A11 | RBD-A | 0.062 | 1.901 | >50 | >50 |
| L12 bP10G07 | RBD-A | 0.334 | 1.406 | >50 | >50 |
| L12.bP11C10 | RBD-A | 0.355 | 3.121 | >50 | >50 |
| L12.bP12E05 | RBD-A | [symbol] | 1.169 | >50 | >50 |
| L12.bP12H09 | RBD-A | 0.103 | 0.483 | >50 | >50 |
| L12.cP08D10 | RBD-A | 20.030 | >50 | >50 | >50 |
| L12.cP09C07 | RBD-A | 0.013 | 0.160 | >50 | >50 |
| L12.cP09D03 | RBD-A | [symbol] | 1.21 [symbol] | >50 | >50 |
| L12.cP08A09 | RBD-A | 0.146 | 0.666 | >50 | >50 |
| L12.cP07E10 | RBD-A | 0.044 | 0.058 | >50 | >50 |
| L12.cP09D05 | RBD-A | 0.014 | 0.039 | >50 | >50 |
| L12.bP12D08 | RBD-B | 0.020 | 0.044 | >50 | >50 |
| L12.bP13A12 | RBD-B | 0.012 | 0.019 | >50 | >50 |
| L12.bP11E12 | SPIKE-A | >50 | >50 | >50 | >50 |
| L12.cP08D07 | SPIKE-A | 0.014 | >50 | >50 | >50 |
| L12.bP13A09 | SPIKE-B | 0.018 | >50 | 1.091 | >50 |
| L12.eP08E01 | | 0.642 | 0.661 | >50 | >50 |
| L6.dP02E06 | | 0.031 | 0.015 | >50 | >50 |
| L6.dP03H10 | | 0.016 | 0.012 | >50 | >50 |
| L6.dP02A05 | | 0.046 | 0.025 | >50 | >50 |
| L6.dP02F01 | | 0.014 | 0.013 | 0.066 | 0.021 |
| L6.dP04A03 | | 0.373 | [symbol] | 0.202 | 0.915 |
| L6.dP02C11 | | N/A | N/A | N/A | N/A |
| L6.dP04C08 | | >50 | >50 | >50 | >50 |
| L25.eP04C02 | | 0.008 | 0.026 | >50 | >50 |
| L25.dP06E11 | | 0.030 | 0.016 | 0.431 | 0.017 |
| L6-P04E05 | | 0.010 | 0.018 | >10 | >10 |

[symbol]

indicates data missing or illegible when filed

### Example: 5

Antibody heavy and light chain variable genes were amplified by RT-PCR before cloning by homologous recombination into mammalian expression vectors. Across the three donors, a total 1,126 antibodies were cloned and expressed, which represents a 68% PCR recovery of paired variable genes and >86% recovery of fully functional cloned genes. The bulk-transformed ligation product for both the heavy chain and light chain were transfected in 4 ml cultures to screen for functional antibodies, which were then tested for expression, binding to RBD and S protein, and finally for neutralization in the SARS CoV-2 pseudovirus assay using HeLa-ACE2 target cells. On average, 90% of the transfected pairs resulted in IgG expression. Of these, 46% showed binding only to S protein while 7.2% had some specificity to RBD, either solely to RBD (0.4%) or to a combination of RBD and S protein (6.8%). The supernatants were also screened for binding to an unrelated HIV antigen (BG505 SOSIP) to eliminate polyreactive supernatants. Overall, these data highlight the high immunogenicity of the S protein relative to the RBD. These supernatants were subsequently evaluated for neutralization activity using SARS-CoV-2 and SARS-CoV-1 pseudoviruses. Strikingly, a small proportion of the binding antibodies showed neutralization activity, which was equally distributed between RBD+/S+ vs. S+ only binders (˜2% each). These data indicate that while the S protein is highly immunogenic in terms of binding antibodies, only a small proportion of these are capable of neutralizing the virus. In contrast, although there are fewer RBD binding antibodies, a larger proportion of these are capable of neutralizing SARS-CoV-2 pseudovirus. Antibodies that measured positive for neutralization in the high-throughput screening were sequence confirmed and expressed at large scale for additional characterization. Sequencing of the neutralizing heavy and light chain pairs revealed nearly all clones to be from independent lineages. The mAbs varied by gene family and CDR lengths, with members comprising mostly VH1 and VH3-gene families. Interestingly, the somatic hypermutation levels for the neutralizing antibodies are low, with an average of 1.3% mutation from germline in the nucleotide sequence for heavy chain and light chain. A polyreactivity assay with solubilized CHO membrane preps was used to confirm that none of these antibodies are polyreactive.

### Example: 6

Epitope Specificity and Functional Activity of Downselected Antibodies

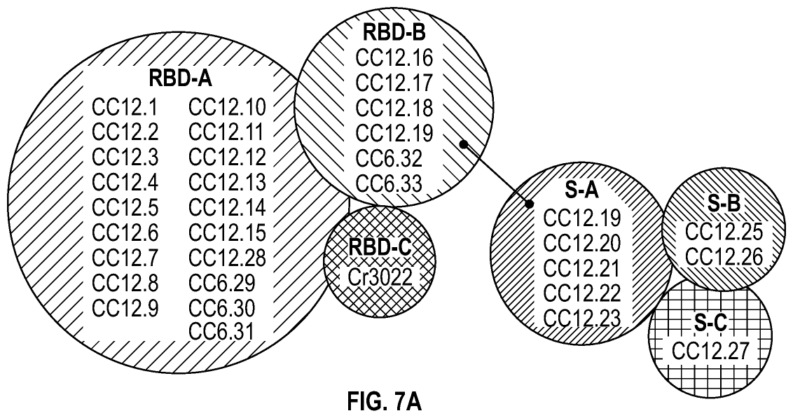

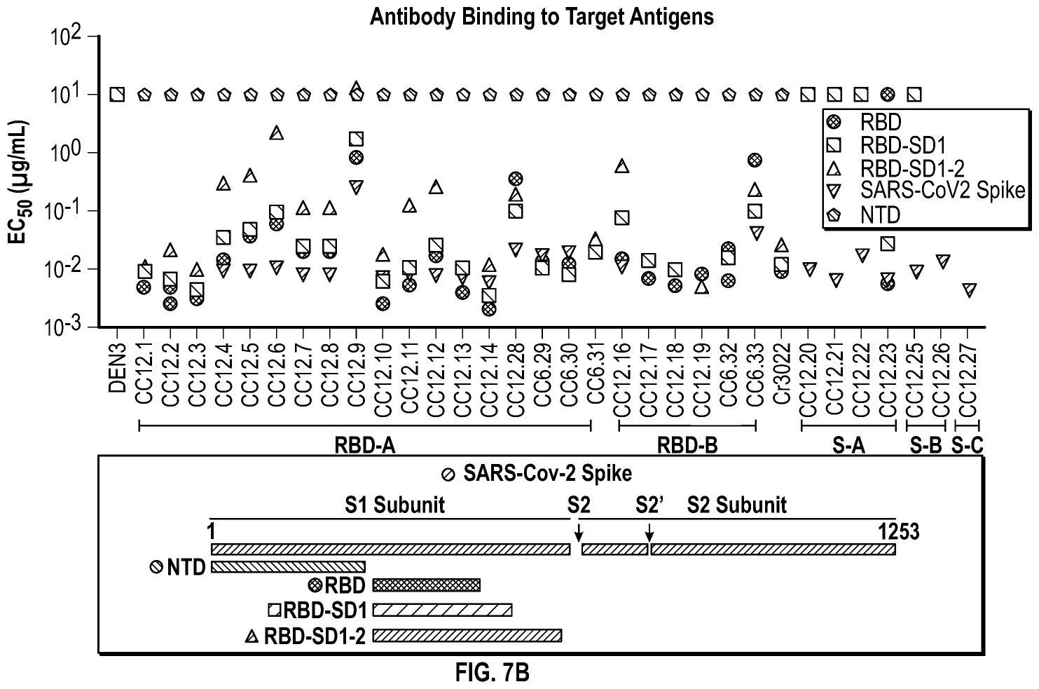

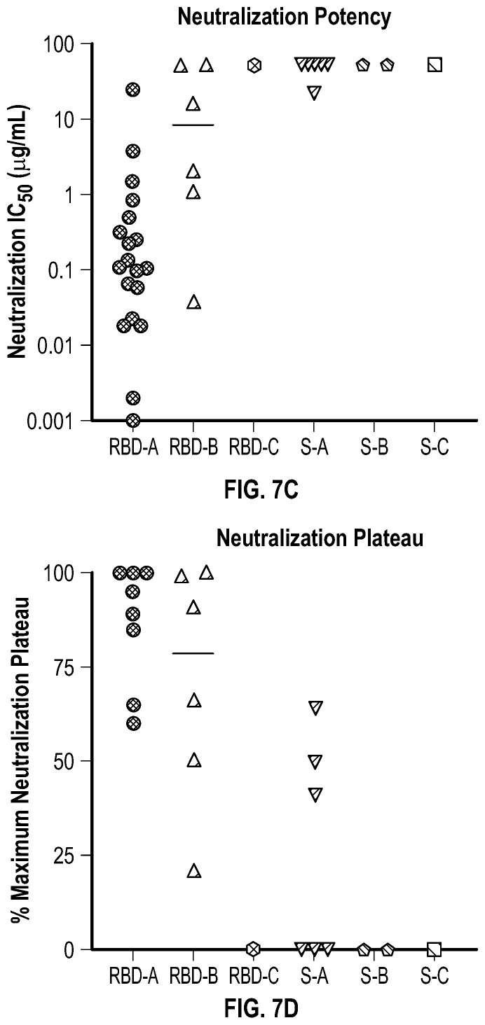

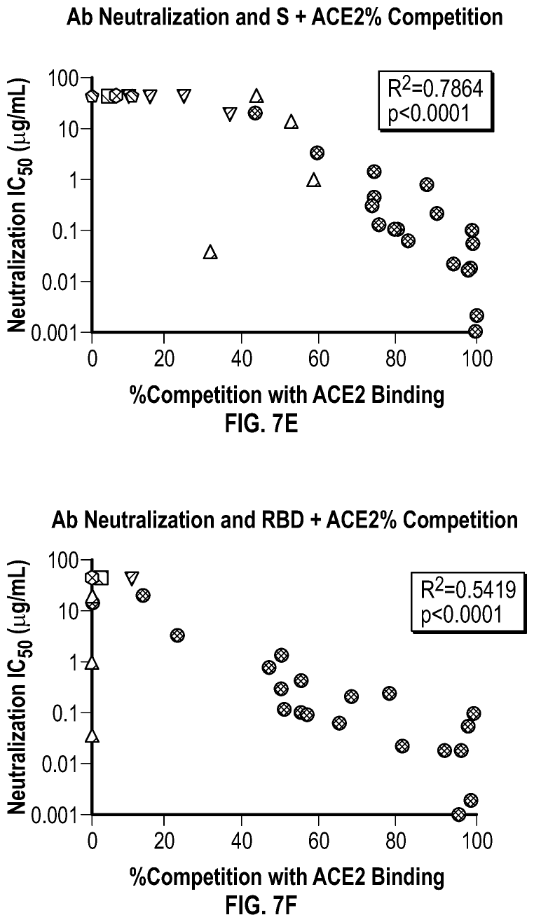

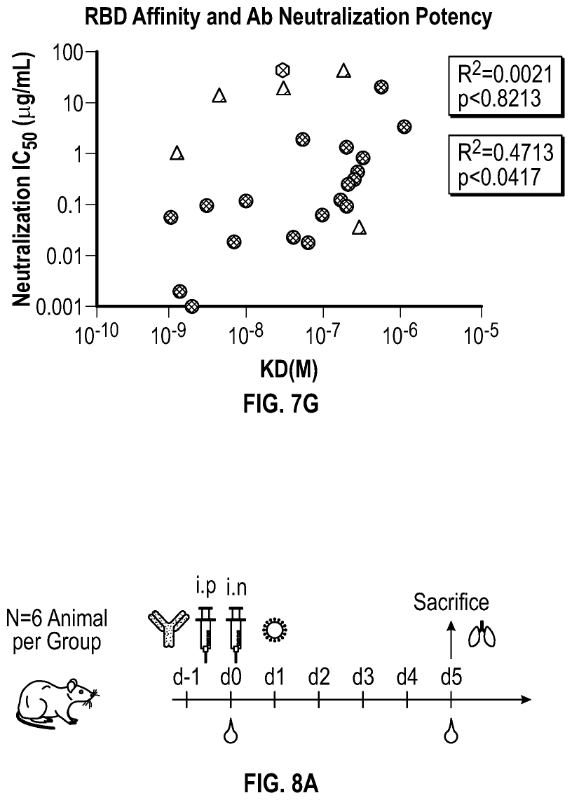

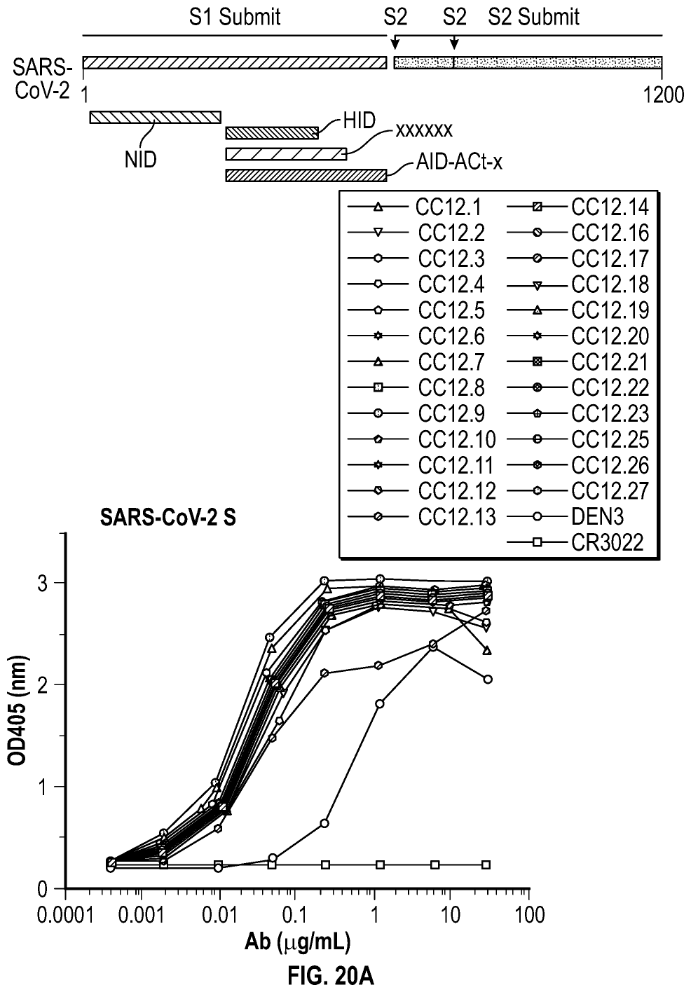

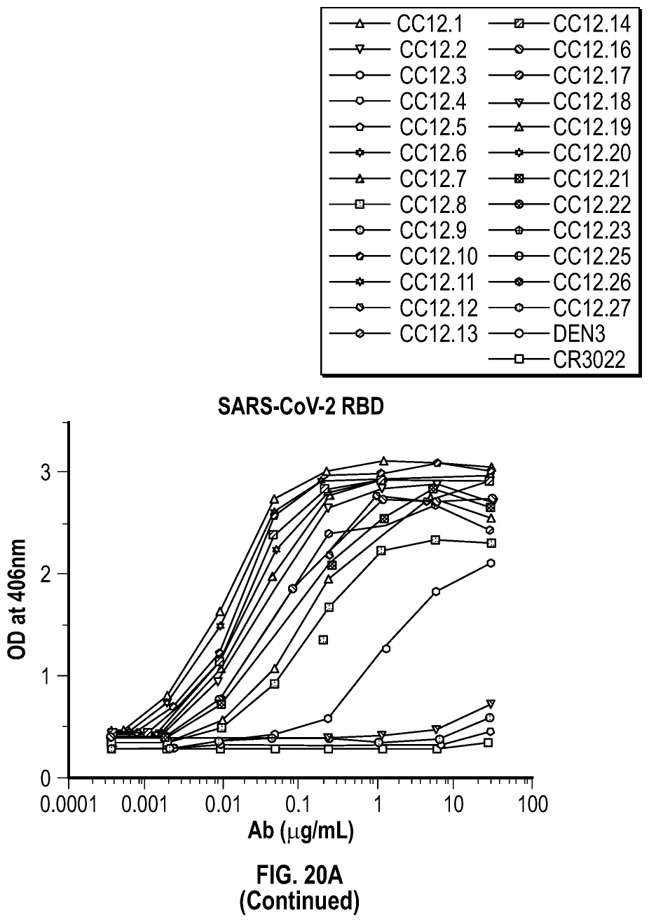

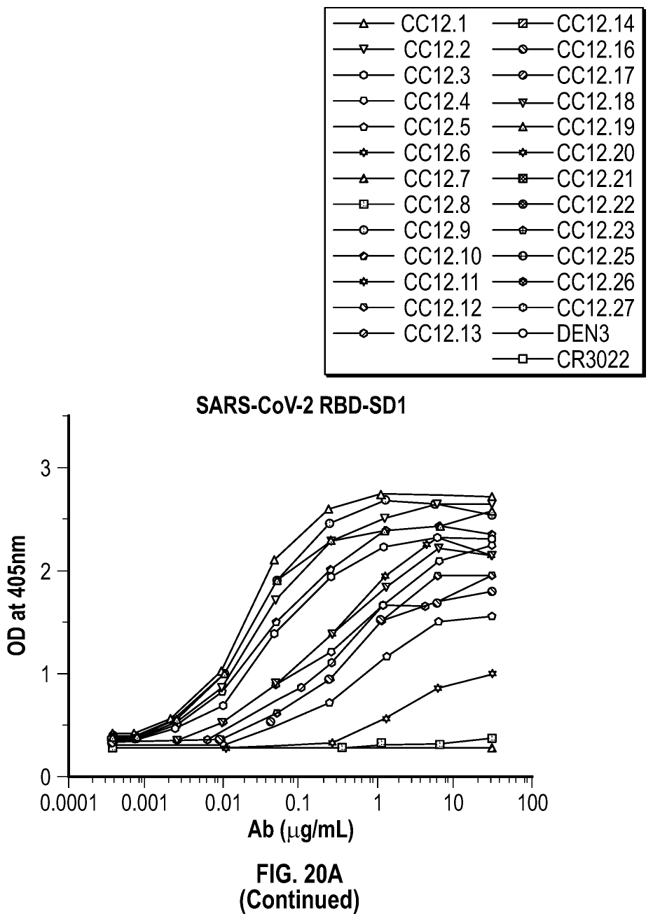

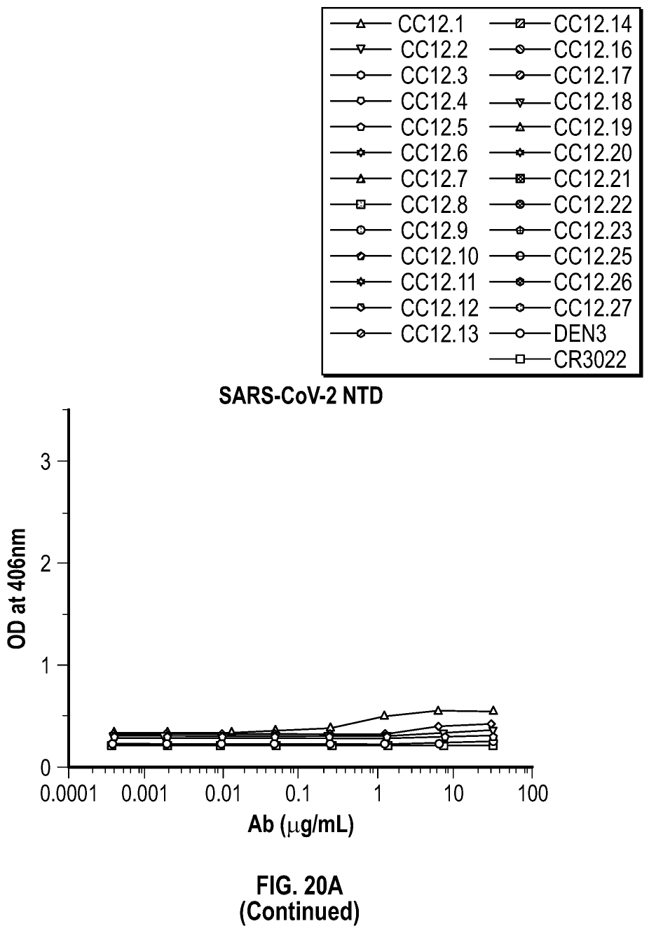

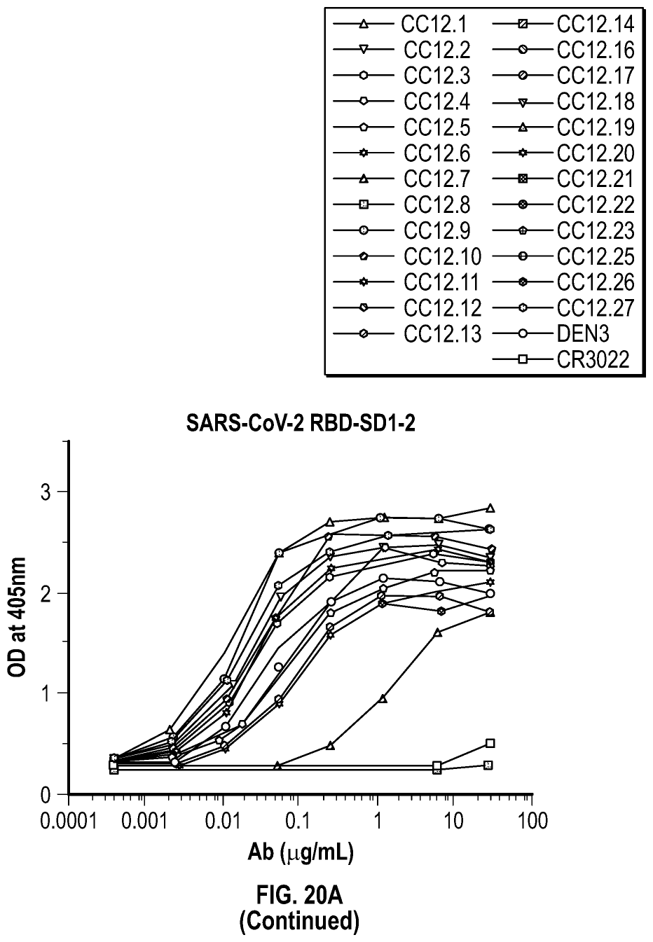

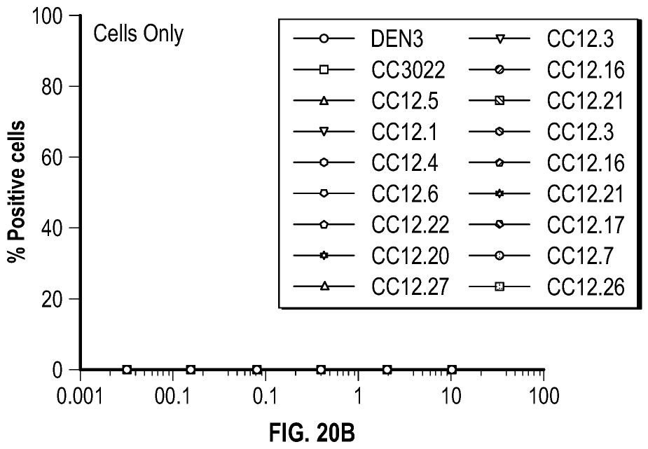

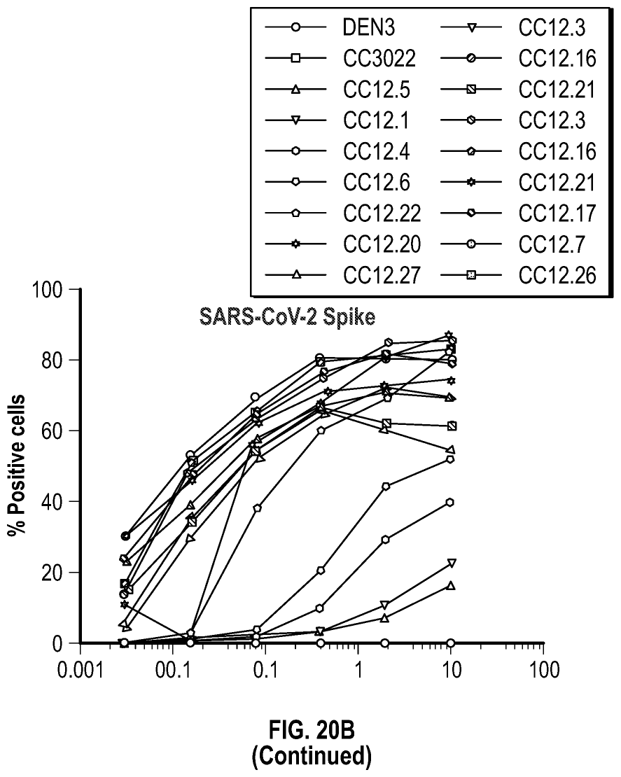

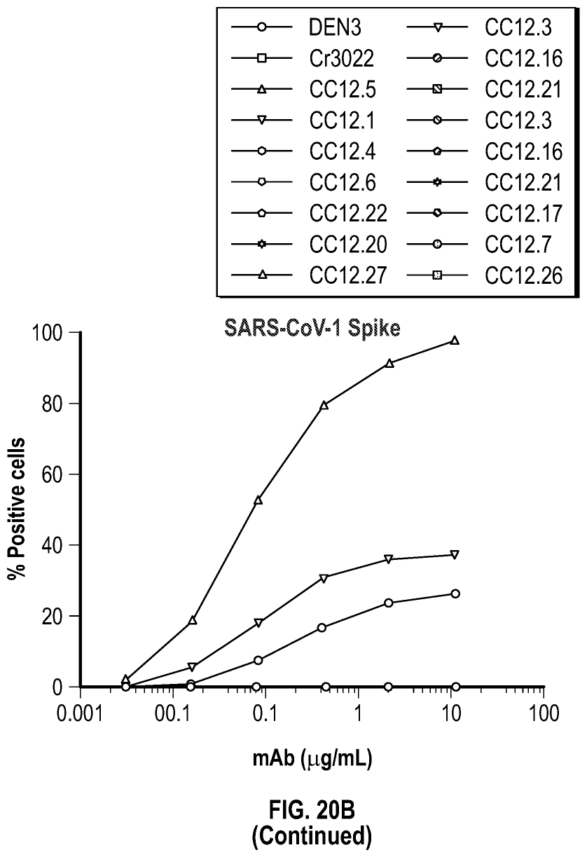

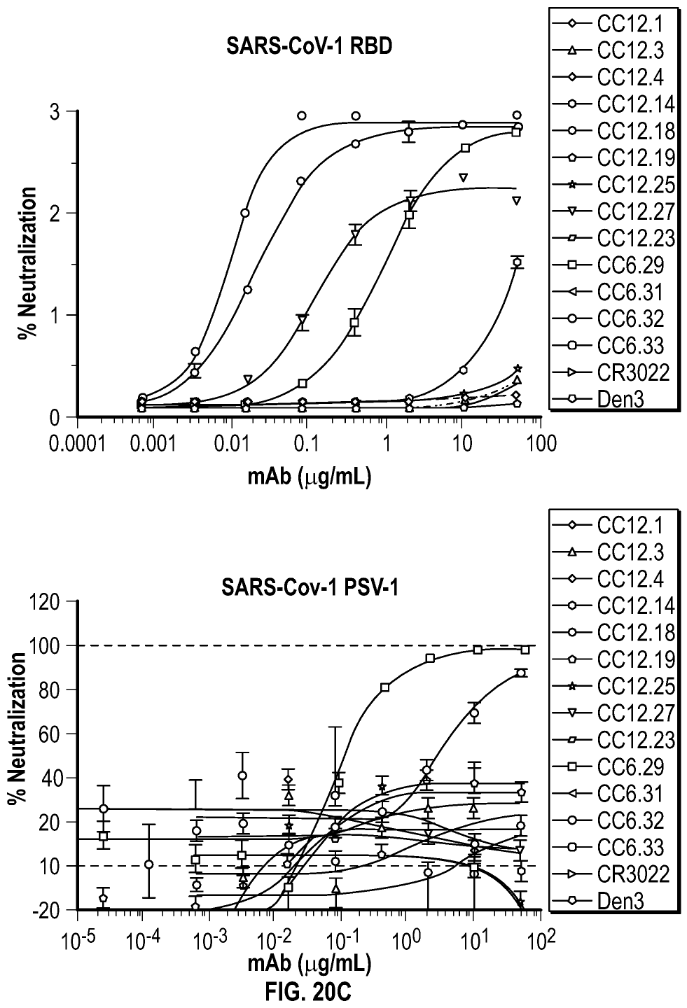

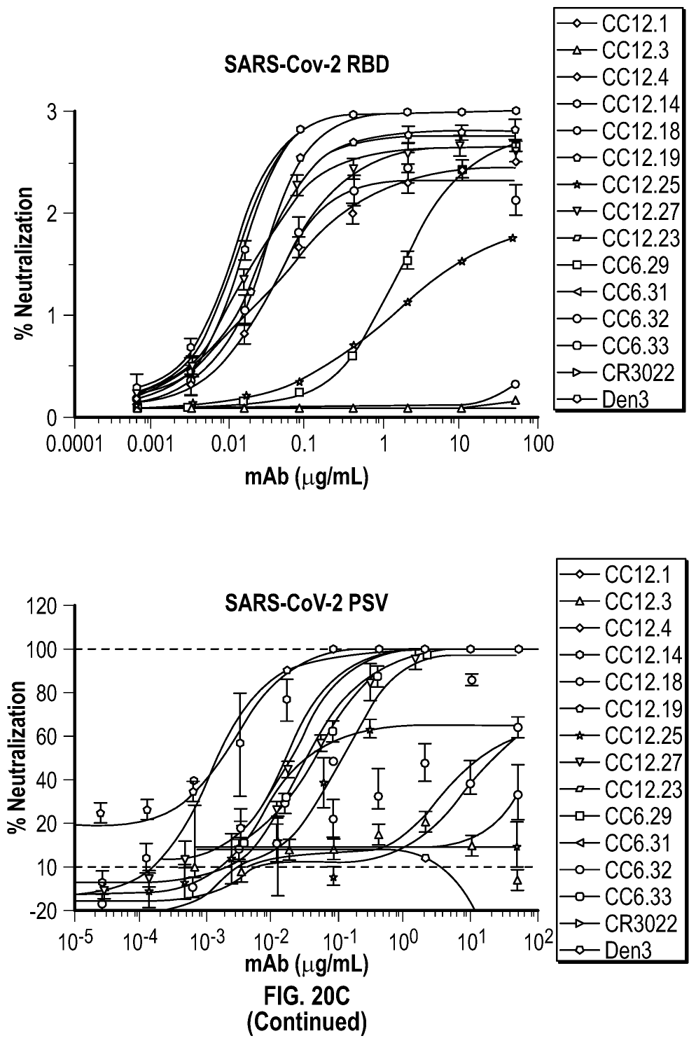

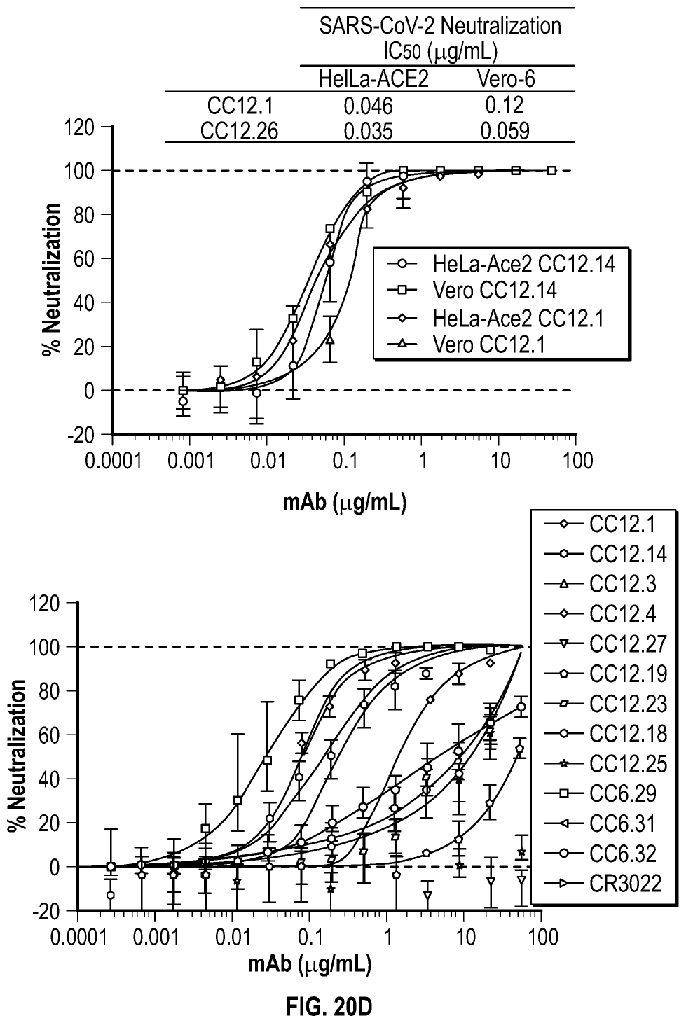

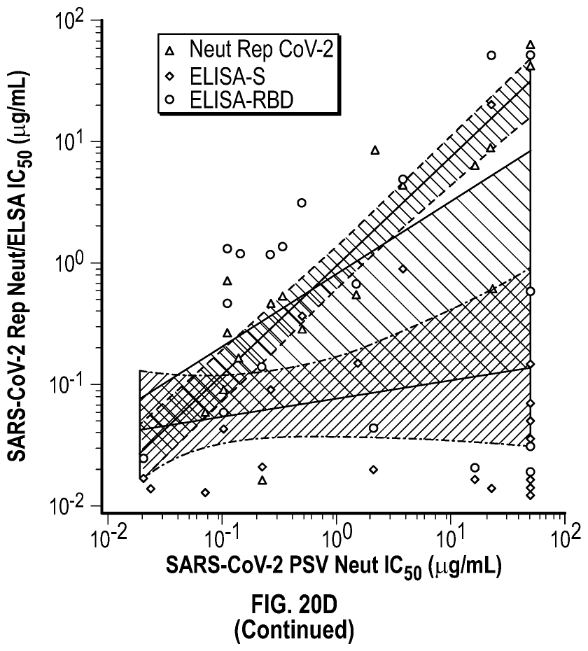

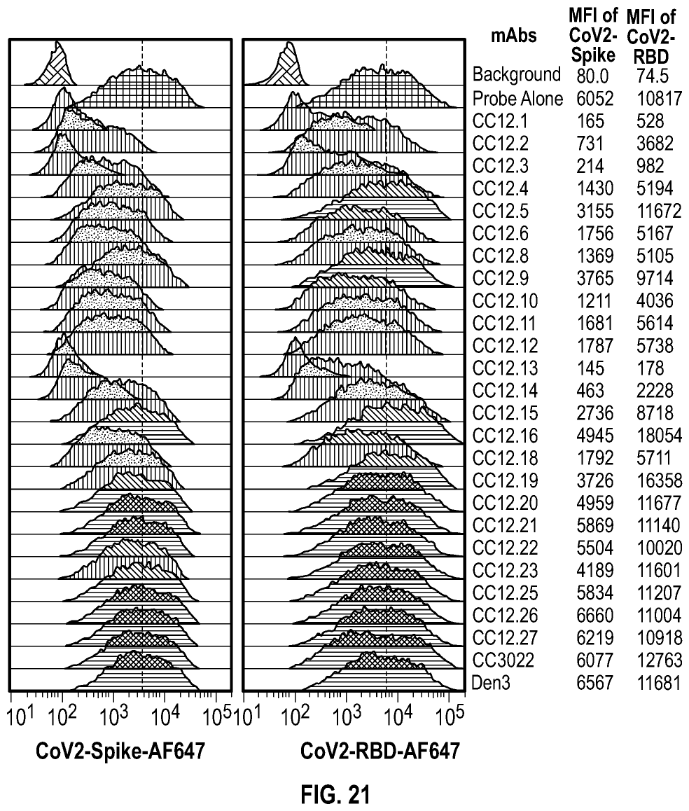

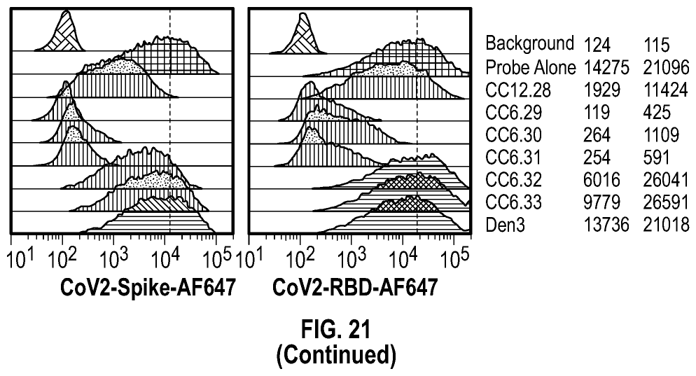

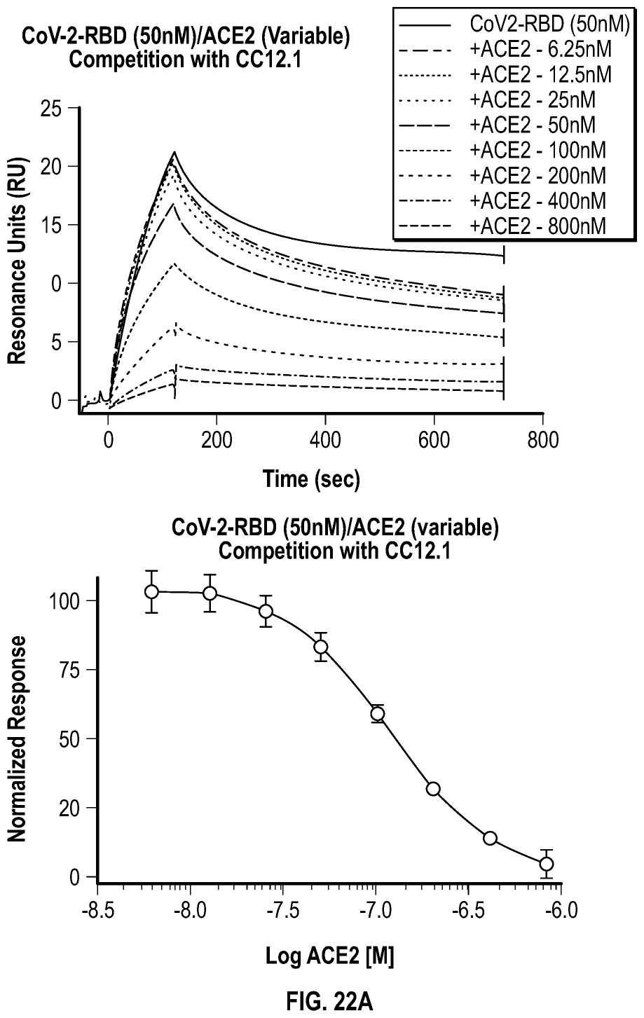

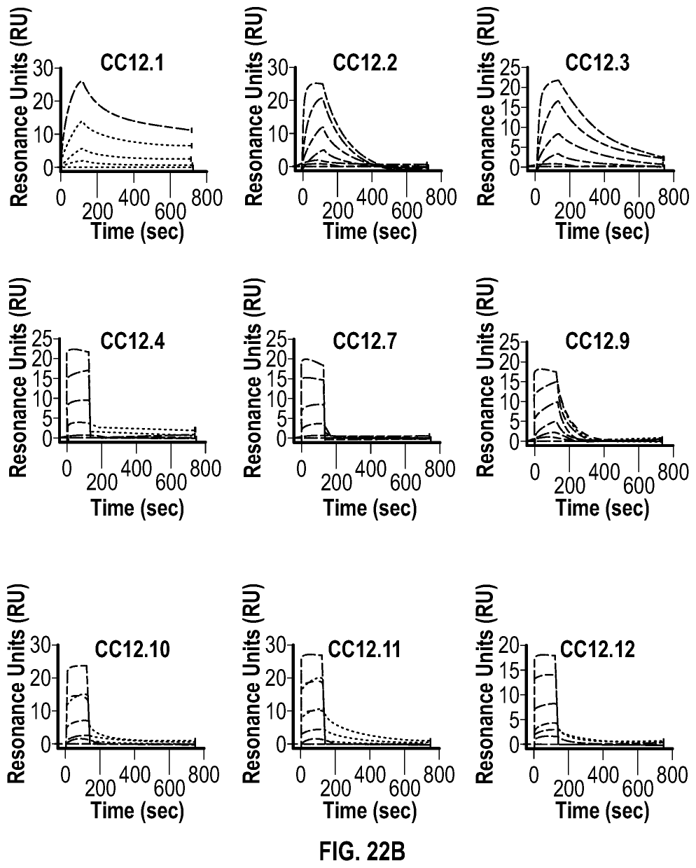

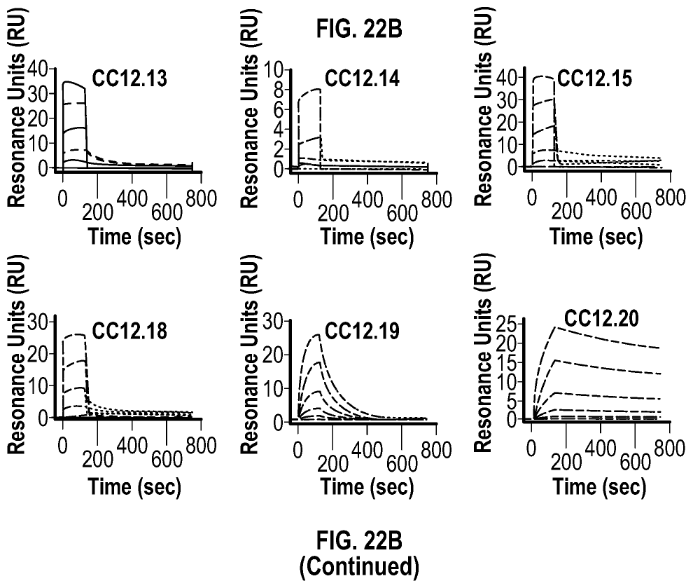

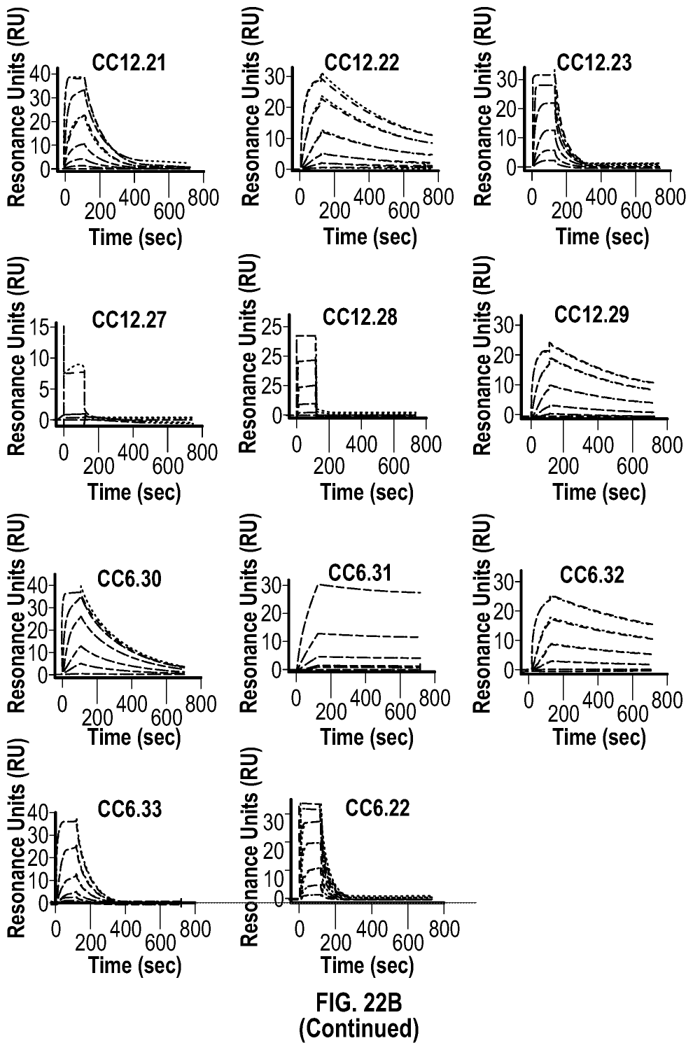

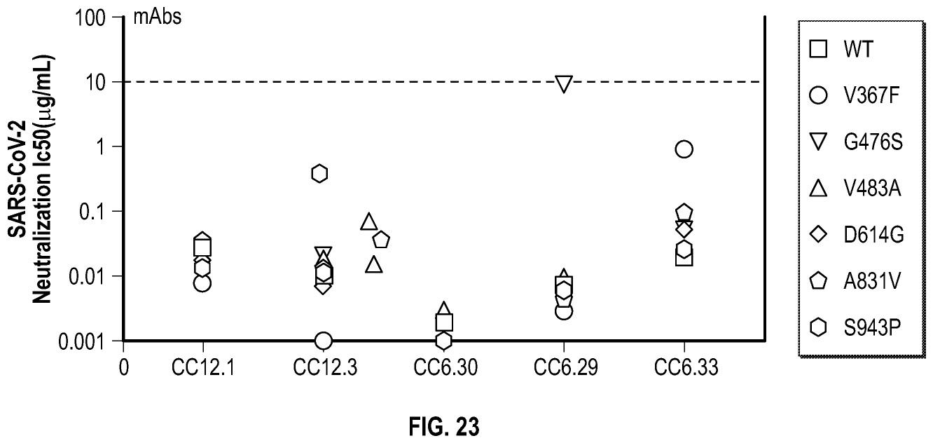

Of the antibodies that tested positive for binding or neutralization in the functional screen, the downselected antibodies were evaluated for epitope specificity by bio-layer interferometry using S and RBD protein as capture antigens. The antigens were captured by HIS-tag and a saturating concentration (100 μg/ml) of antibodies were first added before competing antibodies were added at a lower concentration (25 μg/ml). Accordingly, only antibodies that bind to a non-competing site would be detected in the assay. Among the antibodies evaluated, the results reveal three epitope bins for RBD (designated as RBD-A, RBD-B, and RBD-C) and three epitopes bins for the S protein (designated as S-A, S-B, and S-C). The mAb P13A12 appears to compete with antibodies targeting RBD-A and S-A epitopes, suggesting an interface epitope between the two. To characterize antibodies targeting the S-A epitope further, the antibodies were then evaluated for binding to extended RBD-constructs, including RBD-SD1 and RBD-SD1-2. The mAb P13A12 appears to compete with antibodies targeting two different epitopes, RBD-B and S-A, which might indicate that this mAb targets an epitope spanning RBD-B and S-A. To evaluate epitope specificities further, we next assessed binding of the antibodies to extended RBD-constructs with subdomains (SD) 1 and 2, including the independently folding RBD-SD1 and RBD-SD1-2, and the N-terminal domain (NTD) (FIG. 20A and FIG. 20B). None of the antibodies showed binding to the NTD. P13A12 binds to all the other constructs, which supports the epitope binning data. The other antibodies grouped in the S-A epitope bin that compete with P13A12 show either no binding to RBD or RBD-SD constructs (P11F01 and P12D01) or do show binding to RBD-SD1 and RBD-SD1-2 but not RBD (P08D07). These data suggest two competing epitopes within the S-A epitope bin: one that is confined to the non-RBD region of S protein and the other that includes some element of RBD-SD1-2. The mAbs were next evaluated for neutralization activity against SARS-CoV-2 and SARS-CoV-1 pseudoviruses. The most potent neutralizing antibodies were those directed to RBD-A epitope including two antibodies, P02E05 and P03H10, that neutralize SARS-CoV-2 pseudovirus with an IC50 of 2 ng/mL and 1 ng/mL, respectively (FIG. 7C). In comparison, antibodies directed to RBD-B tended to have higher IC50s and many plateau below 100% neutralization. Despite this trend, CC6.33 is directed against RBD-B and showed complete neutralization of SARS-CoV-2 with an IC50 of 39 ng/mL and also neutralized SARS-CoV-1 with an IC50 of 162 ng/mL. This was the only antibody that showed potent neutralization of both pseudoviruses. The antibodies that do not bind to RBD and are directed to non-RBD epitopes on S protein all show poor neutralization potencies and MPNs well below 100%. To evaluate whether the RBD-A epitope might span the ACE2 binding site, cell surface competition experiments were performed next. Briefly, antibodies were premixed with biotinylated S or RBD proteins at a molar ratio of 4:1 of antibodies to target antigen. The mixture was then incubated with the HeLa-ACE2 cell line and the percent competition against ACE2 receptor was recorded by comparing percent binding of the target antigen with and without antibody present. The antibodies targeting the RBD-A epitope compete best against the ACE2 receptor and the neutralization IC50 correlates well with the percent competition for ACE2 receptor binding for both S protein and for RBD. The affinity of all RBD-specific antibodies to soluble RBD by surface plasmon resonance (SPR) was also assessed a poor correlation between affinity and neutralization potency was found (Table 12). However, the correlation is higher when limited to antibodies targeting the RBD-A epitope. The lack of a correlation between RBD binding and neutralization for mAbs contrasts with the strong correlation described earlier for serum RBD binding and neutralization. Overall, the data highlight epitope RBD-A as the preferred target for eliciting neutralizing antibodies and that corresponding increases in affinity of mAbs to RBD-A will likely result in corresponding increases in neutralization potency. SARS-CoV-2 has shown some propensity for mutation as it has circulated worldwide as evidenced for example in the emergence of the D614G variant. We investigated the activity of 5 nAbs against 6 viral variants that have been reported. All 5 nAbs neutralized the D614G variant. However, one variant with a mutation in the ACE2 binding site (G476S) did show effectively complete resistance to one of the nAbs and another variant (V367F) showed a 10-fold higher IC50 than the WA-1 strain (FIG. 23).

### Example: 7

Functional Activity of Antibodies after Epitope Binning

The mAbs were then evaluated for neutralization activity against SARS-CoV-2 pseudovirus. The neutralization IC50 potencies of these antibodies are shown in herein and their associated maximum plateaus of neutralization. The most potent antibodies that also neutralize virus to completion are those directed to epitope RBD-A. In comparison, antibodies directed to RBD-B do not neutralize potently and also plateau below 100%. The antibodies that do not bind to RBD and are directed to epitopes on S protein all show poor neutralization potencies.

### Example: 8

RBD-A Epitope Binding Studies with Antibodies

Cell surface competition experiments were performed to evaluate whether the RBD-A epitope may span the ACE2 binding site. Briefly, antibodies were premixed with Streptavidin-conjugated biotinylated S or RBD proteins at a molar ratio of 4:1 of antibodies to target antigen. The mixture was then incubated with the HeLa-ACE2 cell line and the % competition against ACE2 receptor was recorded by comparing percent binding of the target antigen with and without antibody present. The data indicate that the antibodies targeting the RBD-A epitope compete best against the ACE2 receptor and that the neutralization IC50 correlates well with the % competition for ACE2 receptor binding for both S protein and for RBD. Similarly, the RBD-binding antibodies were evaluated for affinity to ACE2 by surface plasmon resonance. A summary of these values are plotted compared to neutralization IC50 potency. The correlation between affinity for RBD and neutralization potency is generally poor (R2=0.02), but the correlation is high when limited to antibodies targeting the RBD-A epitope (R2=0.77). These data highlight epitope RBD-A as the preferred target for eliciting neutralizing antibodies and that corresponding increases in affinity of mAbs to RBD-A will likely result in corresponding increases in neutralization potency.

### Example: 9

Passive Transfer of Neutralizing Antibodies and SARS-CoV-2 Challenge in Syrian Hamsters

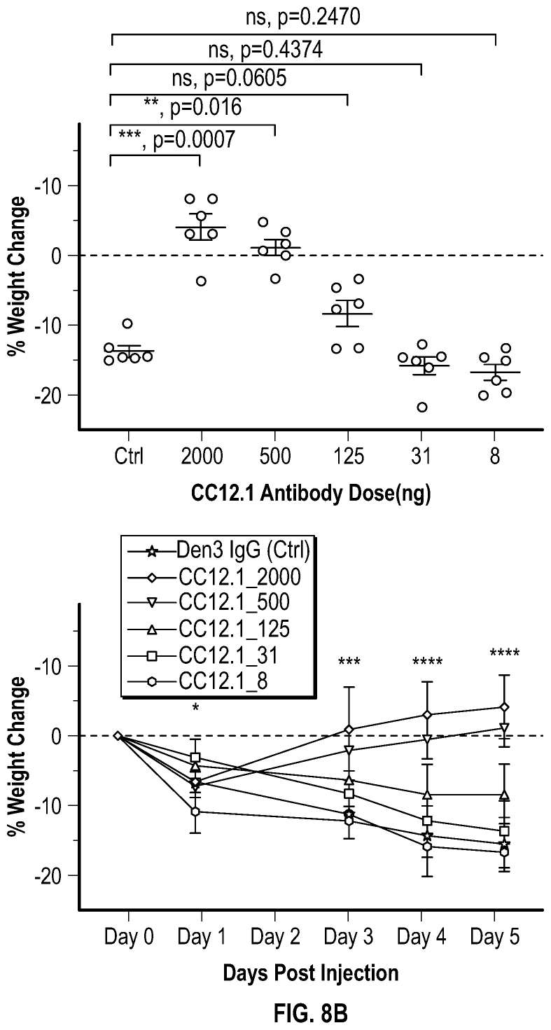

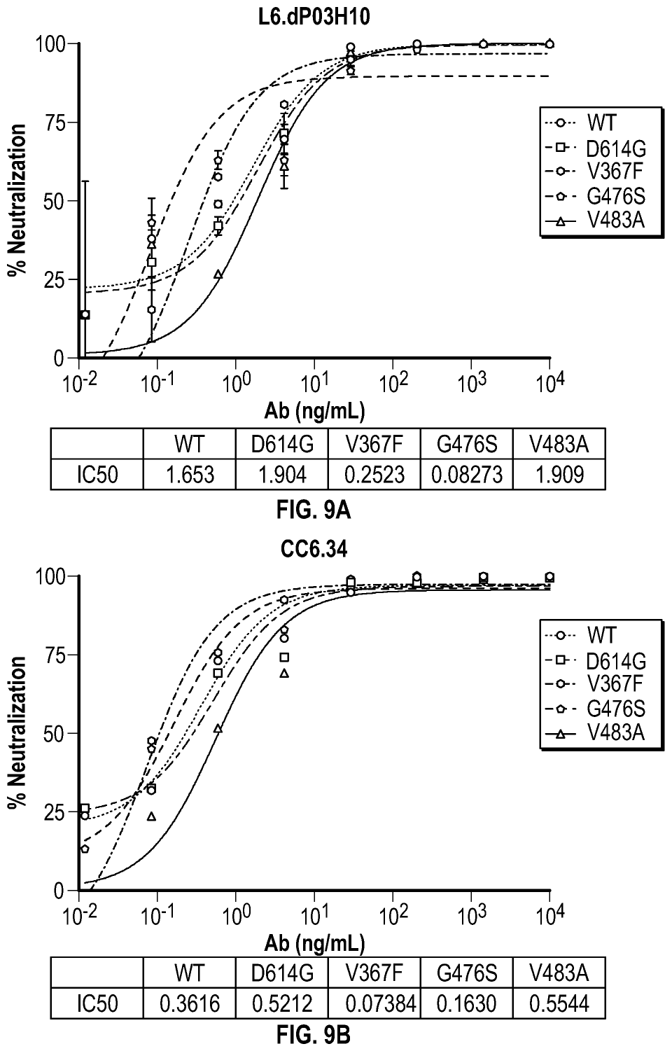

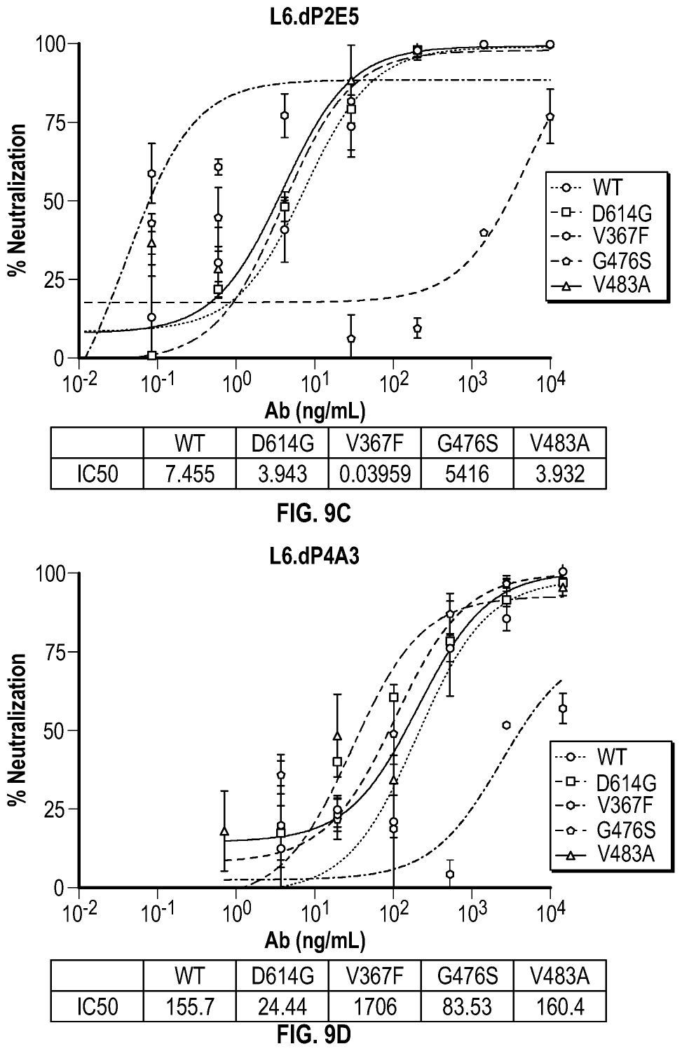

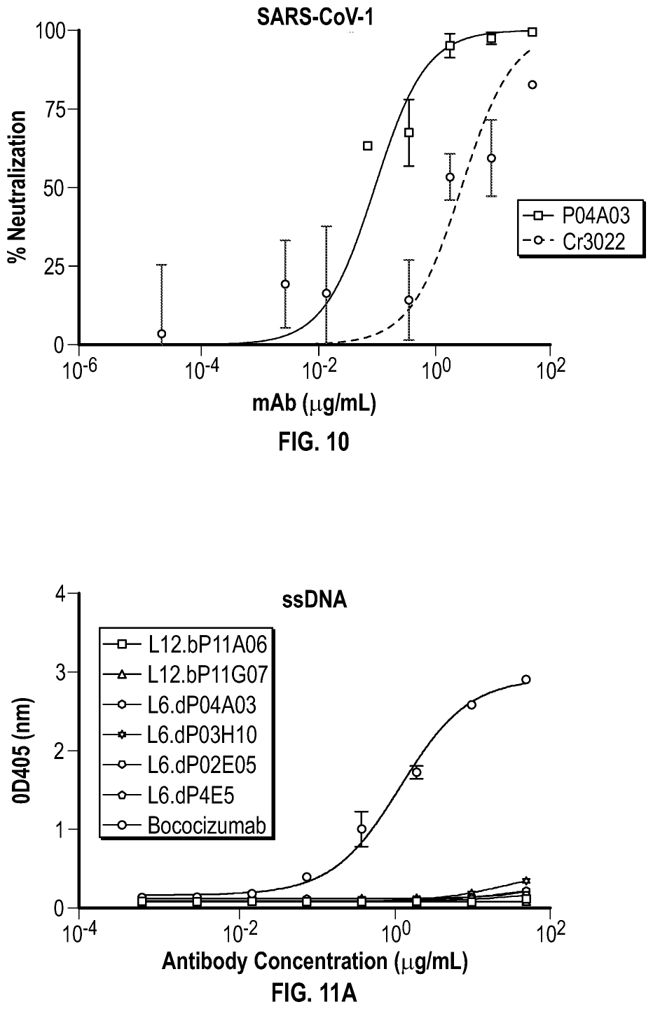

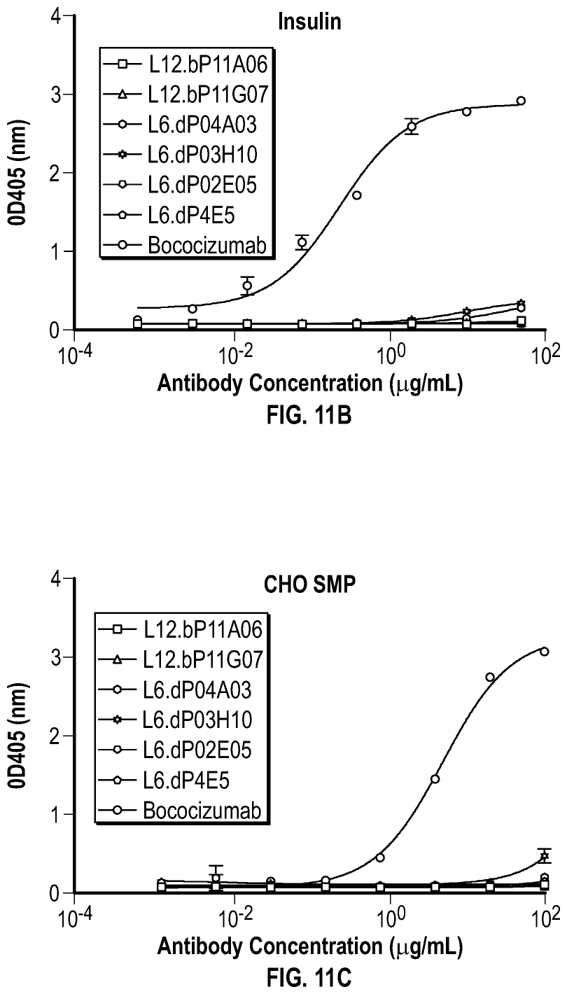

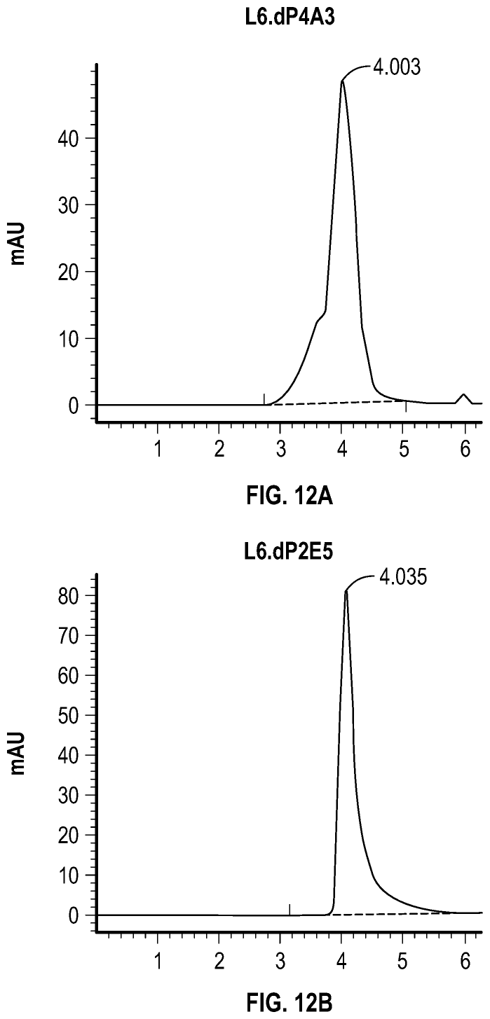

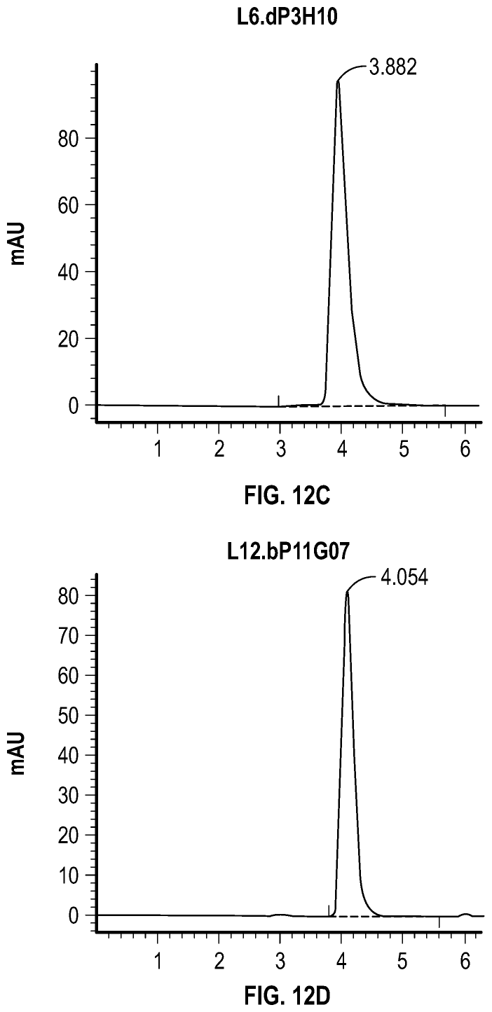

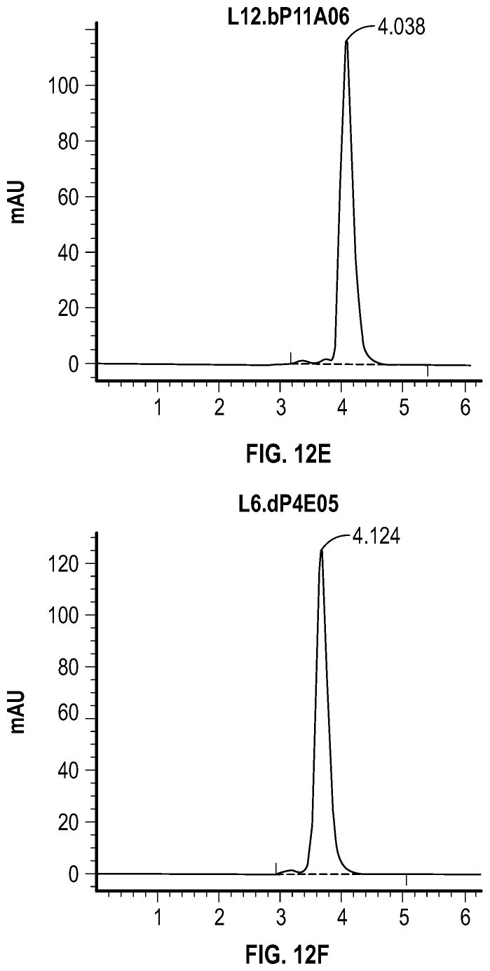

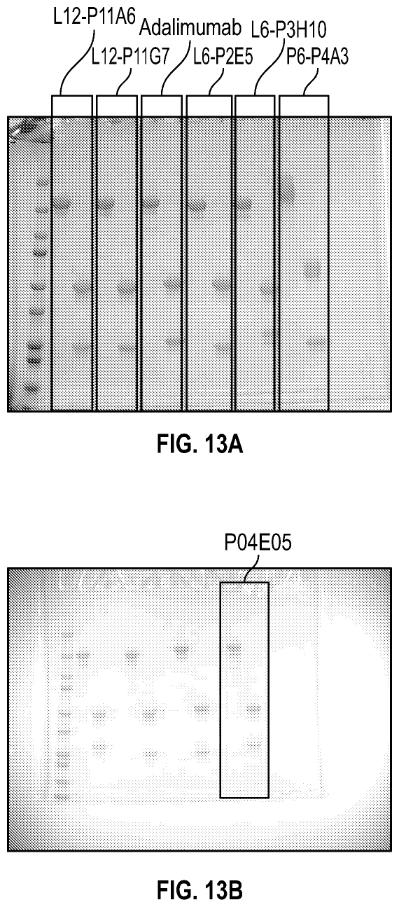

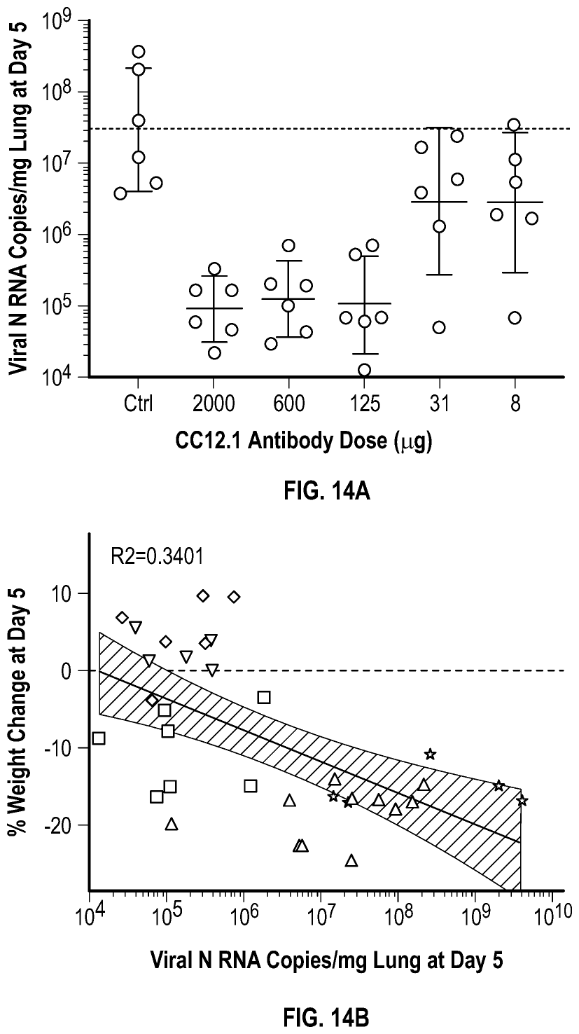

To translate the observed in vitro antibody neutralization potency to in vivo protection against SARS-CoV-2, two monoclonal antibodies were then selected for passive transfer experiments in a Syrian hamster animal model. A total of three groups of 6 animals were given antibodies by intraperitoneal route. Group 1 received an antibody targeting the RBD-A epitope, Group 2 received an antibody targeting the S-B epitope, and Group 3 received an unrelated antibody to Dengue called DEN3. For Groups 1 and 2, the antibodies were delivered at 5 different concentrations to evaluate dose-dependent protection starting at 2 mg/animal (14 mg/kg) at the highest dose and 8 ng/animal at the lowest dose. The DEN3 control antibody was delivered at a single dose of 2 mg/animal (0.06 mg/kg). Sera were collected from each animal post IP infusion of the antibody and all animals were subsequently challenged with a dose of 1×106 PFU of SARS-CoV-2 (USA-WA1/2020) by intranasal route 12 hours post antibody infusion. Syrian hamsters typically clear virus within one week after SARS-CoV-2 infection. Accordingly, the hamsters were weighed as a measure of disease due to infection. Lung tissues were also collected to measure viral load at day 5 following termination of the study and culling of the animals. A data summary is presented herein for animals that received an antibody targeting the RBD-A epitope. The control animals that received DEN3 on average lost nearly ˜15% of body weight at 5 days post virus challenge. In comparison, the animals that received the neutralizing RBD-A antibody at a dose of 2 mg (14 mg/kg) or 0.5 mg (3.6 mg/kg) had no changes in body weight or gained weight, which were both statistically significant. However, animals that received a dose of 0.125 mg (0.9 mg/kg) had on average 8% loss of body weight, while animals that received a dose of 31 ng/ml (0.2 mg/kg) and 8 ng/ml (0.06 mg/kg) lost more weight than the control group. This enhanced weight loss is not statistically significant using a one-way-ANOVA test, but might suggest an antibody-mediated enhanced disease phenotype. This observation would require larger animal sizes to confirm definitively. These data are further corroborated by the viral load data measured by real-time PCR. These data indicate comparable viral loads between the three higher doses (2 mg, 0.5 mg, and 125 ng) of neutralizing antibodies (FIG. 8B). In contrast, equivalent viral loads were observed between the control group receiving DEN3 and the low dose groups receiving 31 and 8 ng of neutralizing antibody. In contrast to the neutralizing antibody to RBD-A, the poorly neutralizing antibody to the S-B epitope showed no evidence of protection at any concentration compared to the control animals. No evidence or trend for enhanced disease was observed for this antibody as well. To determine the antibody serum concentrations that are required for protection against SARS-CoV-2, the antibody serum concentrations were also measured by retro-orbital bleed prior to intranasal virus challenge. The data highlight that the data highlight that the antibodies disclosed herein are 2 fold, 3 fold, 4 fold, 5 fold, 6 fold, or 7 fold more potent than reference antibodies.

### Example: 10

Flow Cytometry-Based Cell Surface ACE2 Binding Inhibition Assay.

MAb inhibition of SARS-CoV-2 S or RBD binding to cell surface hACE2 was performed by flow cytometry as follows. Purified mAbs were mixed with biotinylated SARS-CoV-2 S or RBD in the molar ratio of 4:1 on ice for 1 h. In the meantime, HeLa-ACE2 cells were washed once with DPBS then detached by incubation with DPBS supplemented with 5 mM EDTA. The detached HeLa-ACE2 cells were washed and resuspended in FACS buffer (2% FBS and 1 mM EDTA in DPBS). 0.5 million Hela-ACE2 cells were added to mAb/S or RBD mixture and incubated at 4° C. for 0.5 h. HeLa-ACE2 cells were then washed once with FACS buffer, resuspended FACS buffer with 1 μg/ml streptavidin-AF647 (Thermo, S21374) and incubated for another 0.5 h. After washing, HeLa-ACE2 cells were resuspended in FACS buffer in the presence of 2 μg/ml propidium iodide (Sigma, P4170-100MG) for live/dead staining. HeLa and HeLa-ACE2 cells stained with SARS-CoV-2 S or RBD alone were used as background and positive control separately. The AF647 mean fluorescence intensity (MFI) was determined from the gate of singlet and PI negative cells. The percentage of ACE2 binding inhibition was calculated using the following equation.

$$100 * \text{?}$$

$$\text{?} \text{indicates text missing or illegible when filed}$$

TABLE 5

% Inhibition of CoV2 Spike
binding to cell surface hACE2

| | mAb:CoV2- | mAb:CoV2- |
|---|---|---|
| | S = 4:1 | S = 2:1 |
| L12-bP11A06 | 98.6 | 98.3 |
| L12-cP09B02 | 89.8 | 86.0 |
| L12-bP11G07 | 97.9 | 97.0 |
| L12-bP11A11 | 78.8 | 68.5 |
| L12-bP10G07 | 73.2 | 62.6 |
| L12-bP11C10 | 73.6 | 63.2 |
| L12-bP12E05 | NA | NA |
| L12-bP12H09 | 79.8 | 71.8 |
| L12-cP08D10 | 42.2 | 35.8 |
| L12-cP09C07 | 82.2 | 76.8 |
| L12-cP09D03 | 74.9 | 66.9 |
| L12-cP08A09 | 73.3 | 65.9 |
| L12-cP07E10 | 98.9 | 98.7 |
| L12-cP09D05 | 94.0 | 91.1 |
| L12-cP07H01 | 58.4 | 49.6 |
| L12-bP11G10 | 23.8 | 20.6 |
| L12-bP12D08 | NA | NA |
| L12-bP12G07 | 51.9 | 45.8 |
| L12-bP13A12 | 42.8 | 41.2 |
| L12-bP11F01 | 23.6 | 26.2 |
| L12-bP12D01 | 9.4 | 6.3 |
| L12-bP11E12 | 15.0 | 10.8 |
| L12-cP08D07 | 35.7 | 30.0 |
| L12-cP07F10 | NA | NA |
| L12-bP13A09 | 10.0 | 11.2 |
| L12-bP12E07 | −3.0 | 0.6 |
| L12-bP11F04 | 3.9 | 6.0 |
| L12-eP08E01 | 77.3 | |

TABLE 6

| | Inhibition of CoV2 Spike binding to cell surface | | |
|---|---|---|---|
| | <MFI of Spike> | | |
| | ka [1/Ms] | kd [1/s] | KD [M] |
| L12.bP11A06 | 6.95E+05 | 4.12E−03 | 5.92E−09 |
| L12.cP09B02 | 1.48E+05 | 8.03E−03 | 5.43E−08 |
| L12.bP11G07 | 3.99E+05 | 3.42E−03 | 8.59E−09 |
| L12.bP11A11 | 3.88E+05 | 6.60E−02 | 1.70E−07 |
| L12.bP10G07 | 4.36E+05 | 9.61E−02 | 2.20E−07 |
| L12.bP11C10 | 4.93E+05 | 1.26E−01 | 2.55E−07 |
| L12.bP12E05 | 7.82E+05 | 1.43E−01 | 1.83E−07 |
| L12.bP12H09 | 7.73E+05 | 1.20E−01 | 1.55E−07 |
| L12.cP08D10 | 5.94E+04 | 3.12E−01 | >5.00E−07 |
| L12.cP09C07 | 7.86E+05 | 6.91E−02 | 8.79E−08 |
| L12.cP09D03 | 5.75E+05 | 8.43E−02 | 1.47E−07 |
| L12.cP08A09 | 4.04E+05 | 7.07E−02 | 1.75E−07 |
| L12.CP07E10 | 1.40E+05 | 3.64E−04 | 2.60E−09 |
| L12.cP09D05 | 4.35E+05 | 1.57E−02 | 3.62E−08 |
| L12.bP12D08 | 3.64E+05 | 1.70E−02 | 4.66E−08 |
| L12.bP13A12 | 4.43E+05 | 1.15E−02 | 2.60E−08 |
| L12.bP11E12 | No Binding | No Binding | No Binding |
| L12.cP08D07 | 6.36E+05 | 1.70E−02 | 2.68E−08 |
| L12.bP13A09 | No Binding | No Binding | No Binding |

TABLE 7

| | Inhibition of CoV2 Spike binding to | | % Inhibition of CoV2 Spike binding to | |
|---|---|---|---|---|
| | cell surface <MFI of Spike> | | cell surface hACE2 | |
| | mAb:CoV2- | mAb:CoV2- | mAb:CoV2- | mAb:CoV2- |
| | S = 4:1 | S = 2:1 | S = 4:1 | S = 2:1 |
| L12.bP11A06 | 165 | 185 | 98.6 | 98.3 |
| L12.cP09B02 | 731 | 974 | 89.8 | 86.0 |
| L12.bP11G07 | 214 | 266 | 97.9 | 97.0 |
| L12.bP11A11 | 1430 | 2088 | 78.8 | 68.5 |
| L12.bP10G07 | 1792 | 2467 | 73.2 | 62.6 |
| L12.bP11C10 | 1762 | 2432 | 73.6 | 63.2 |
| L12.bP12E05 | | | NA | NA |
| L12.bP12H09 | 1369 | 1882 | 79.8 | 71.8 |
| L12.cP08D10 | 3770 | 4182 | 42.2 | 35.8 |
| L12.cP09C07 | 1216 | 1561 | 82.2 | 76.8 |
| L12.cP09D03 | 1681 | 2191 | 74.9 | 66.9 |
| L12.cP08A09 | 1786 | 2260 | 73.3 | 65.9 |
| L12.cP07E10 | 145 | 158 | 98.9 | 98.7 |
| L12.cP09D05 | 463 | 644 | 94.0 | 91.1 |
| L12.bP12D08 | | | NA | NA |
| L12.bP13A12 | 3736 | 3835 | 42.8 | 41.2 |
| L12.bP11E12 | 5509 | 5777 | 15.0 | 10.8 |
| L12.cP08D07 | 4189 | 4551 | 35.7 | 30.0 |
| L12 bP13A09 | 5834 | 5754 | 10.0 | 11.2 |

TABLE 8

| | Inhibition of CoV2-RBD binding | | % Inhibition of CoV2-RBD binding | |
|---|---|---|---|---|
| | to cell surface <MFI of Spike> | | to cell surface hACE2 | |
| | mAb:CoV2- | mAb:CoV2- | mAb:CoV2- | mAb:CoV2- |
| | RBD = 4:1 | RBD = 2:1 | RBD = 4:1 | RBD = 2:1 |
| L12.bP11A06 | 528 | 817 | 95.9 | 93.3 |
| L12.cP09B02 | 3682 | 5050 | 67.5 | 55.2 |
| L12.bP11G07 | 982 | 1548 | 91.9 | 86.8 |
| L12.bP11A11 | 5194 | 6332 | 53.9 | 43.6 |
| L12.bP10G07 | 5711 | 7118 | 49.2 | 36.6 |
| L12.bP11C10 | 5167 | 6899 | 54.1 | 38.5 |
| L12.bP12E05 | | | NA | NA |
| L12.bP12H09 | 5105 | 6562 | 54.7 | 41.6 |
| L12.cP08D10 | 9714 | 10503 | 13.2 | 6.1 |
| L12.cP09C07 | 4036 | 5131 | 64.3 | 54.5 |
| L12.cP09D03 | 5615 | 6640 | 50.1 | 40.9 |
| L12.cP08A09 | 5738 | 6803 | 49.0 | 39.4 |
| L12.cP07E10 | 178 | 237 | 99.1 | 98.6 |
| L12.cP09D05 | 2228 | 2813 | 80.6 | 75.4 |
| L12.bP12D08 | | | NA | NA |
| L12.bP13A12 | 16358 | 16079 | −46.7 | −44.2 |
| L12.bP11E12 | 10020 | 10322 | 10.4 | 7.7 |
| L12.cP08D07 | 11606 | 10845 | −3.9 | 3.0 |
| L12.bP13A09 | 11207 | 11002 | −0.3 | 1.6 |

### Example: 11

SARS-CoV-2 Focus Reduction Neutralization Test (FRNT)

HeLa-ACE2 cells were plated in 12 μL complete DMEM at a density of 2×10³ cells per well. In a dilution plate, plasma or mAb was diluted in series with a final volume of 12.5 μL, 12.5 μL of SARS-CoV-2 was added to the dilution plate at a concentration of 1.2×10⁴ pfu/mL. After 1 h incubation, the media remaining on the 384-well cell plate was removed and 25 μL of the virus/mAb mixture was added to the 384-well cell plate. The plate was incubated for 20 h after which the plate was fixed for 1 h. The plate was then washed three times with 100 μL of 1×PBS 0.05% tween, 12.5 μL of human polyclonal sera diluted 1:500 in Perm/Wash buffer (BD Biosciences 554723) were added to the plate and incubated at RT for 2 h. The plate was washed three times and peroxidase goat anti-human Fab (Jackson Scientific) were diluted 1:200 in Perm/Wash buffer then added to the plate and incubated at RT for 2 h. The plate was then washed three times and 12.5 μL of Perm/Wash buffer was added to the plate then incubated at RT for 5 min. The Perm/Wash buffer was removed and TrueBlue peroxidase substrate was immediately added (Sera Care 5510-0030). Infected cell non-linear regression curves were analyzed using Prism 8 software to calculate EC₅₀ values.

### Example: 12

Pseudovirus (PSV) Assay

MLV-gag/pol and MLV-CMV-Luciferase plasmids were co-transfected with full-length or truncated SARS-CoV-2 and SARS-CoV-2 plasmid, respectively, with transfection reagent Lipotransfectmine 2000 in HEK293T cells. After 48 h of transfection, supernatants containing pseudotyped virus were collected and frozen at −80° C. for long-term storage. Serially diluted plasma or mAbs were incubated with pseudovirus at 37° C. for 1 h, then transferred onto HeLa-hACE2 cells in 96-well plates at 10,000 cells/well (Corning, 3688). After 48 h of incubation, supernatants were removed, HeLa-hACE2 cells were then lysed in luciferase lysis buffer (25 mM Gly-Gly pH 7.8, 15 mM MgSO₄, 4 mM EGTA, 1% Triton X-100). Luciferase activity was measured by adding Bright-Glo (Promega, PR-E2620) according to the manufacturer's instructions. Plasma or mAbs were tested in duplicate wells. Neutralization ID₅₀ or IC₅₀ titers were calculated using “One-Site Fit Log IC₅₀” regression in GraphPad Prism 8.0.

### Example: 13

Cohort Information

De-identified PBMC and plasma were provided through the “Collection of Biospecimens from Persons Under Investigation for 2019-Novel Coronavirus Infection to Understand Viral Shedding and Immune Response Study” UCSD IRB #200236. Protocol was approved by the UCSD Human Research Protection Program.

### Example: 14

Whole Virus ELISA

High binding plates (Corning 3700) were coated with 12.5 μL of Galanthus nivalis Lectin (GNL; Vector Laboratories L-1240-5) at 10 μg/mL and incubated overnight at 4° C. The GNL was removed and 12.5 μL of SARS-CoV-2 was added to the plate at a concentration of 2×10⁶ pfu/mL then incubated for 24 h at 4° C. 12.5 μl of 8% Formaldehyde was added to a final concentration of 4% then incubated at RT for 1 h. The plate was then washed three times with 100 μL of 1×PBS supplemented with 0.05% tween. 50 μL of 3% BSA were added to the plate and incubated at RT for 2 h. The BSA was removed and 12.5 μL of plasma or mAb diluted in series was added to the plate then incubated at RT for 1.5 h. The plate was then washed three times with 100 μL of 1×PBS supplemented with 0.05% tween. 12.5 μL of alkaline phosphatase conjugated goat anti-human Fc antibody (Jackson Immunoresearch 109-055-098) diluted 1:2000 was added to the plate and incubated for 1 h at RT. The plate was then washed three times with 100 μL of 1×PBS supplemented with 0.05% tween. 12.5 μL of phosphatase substrate (SIGMA-ALDRICH S0942) were added to the plate. Non-linear regression curves were analyzed using Prism 8 software to calculate EC₅₀ values.

### Example: 15

Plasmid Construction for Fill-Length and Recombinant Soluble Proteins

To generate full-length SARS-CoV-1 (1255 amino acids; GenBank: AAP13567) and SARS-CoV-2 (1273 amino acids; GenBank: MN908947) spike genes were synthesized by GeneArt (Life Technologies) and cloned into the mammalian expression vector phCMV3 (Genlantis. USA) using PstI and BamH restriction sites. Expression plasmids for soluble S ectodomain protein SARS-CoV-1 (residue 1-1190) and SARS-CoV-2 (residue 1-1208) were constructed by PCR amplification and Gibson assembly cloning into vector phCMV3. To stabilize soluble S proteins in the prefusion state and to improve trimerization, the following changes were made: double proline substitutions in the S2 subunit, replacement of the furin cleavage site in SARS-CoV-2 (residues 682-685), and S2 cleavage site in SARS-CoV-1 (residues 664-667) with “GSAS” and incorporation of a C-terminal T4 fibritin trimerization motif (16, 17). Additionally, a HRV-3C protease cleavage site, 6×HisTag, and AviTag spaced by GS-linkers were added to the C-terminus to aid purification strategies. To generate gene fragments encoding SARS-CoV-2 N-terminal domain-NTD (residue 1-290), receptor-binding domain-RBD (residue 332-527), RBD-SD1 (residue 320-591), and RBD-SD1-2 (residue 320-681) subdomains, PCR-amplifications were carried out from the SARS-CoV-2 plasmid and gene fragments were cloned in frame with the original secretion signal or the Tissue Plasminogen Activator (TPA) leader sequence. A similar design strategy was used to construct the SARS-CoV-1-RBD (residue 319-513) gene encoding plasmid.

### Example: 16

Flow Cytometry Based Cell Surface SARS-CoV-1/CoV-2 Spike Binding Assay

Binding of mAbs/sera to the HEK293T cell-surface expressed SARS-CoV-1 and SARS-CoV-2 spikes was performed as described below. Briefly, HEK293T cells were transfected with plasmids encoding full-length SARS-CoV-1 or SARS-CoV-2 spikes and incubated for 36-48 h at 37° C. Post incubation cells were trypsinized to prepare a single cell suspension and were distributed into 96-well plates. 50 μl/well of 3-fold serial titrations of mAbs starting at 10 μg/ml or serum samples starting at 1:30 dilution were added to transfected cells. The Abs were incubated with cells for 1 h on ice. The plates were washed twice in FACS buffer (1×PBS, 2% FBS, 1 mM EDTA) and stained with 50 μl/well of 1:200 dilution of R-phycoerythrin (PE)-conjugated mouse anti-human IgG Fc antibody (SouthernBiotech) and 1:1000 dilution of Zombie-NIR viability dye (BioLegend). After another two washes, stained cells were analyzed using flow cytometry (BD Lyrics cytometers), and the binding data were generated by calculating the percent (%) PE-positive cells for antigen binding using FlowJo 10 software. CR3022, a SARS-CoV-1 and SARS-CoV-2 spike-binding antibody, and dengue antibody. Den3, were respectively positive and negative controls, respectively, for the assay.

### Example: 17

Protein Expression and Purification

To express the soluble S ectodomain proteins from SARS-CoV-1, SARS-CoV-2 and their truncated protein versions, protein-encoding plasmids were transfected into FreeStyle293F cells (Thermo Fisher) at a density of approximately 1 million cells/mL. For large-scale production, we mixed 350 μg plasmids with 16 mL Transfectagro™ (Corning) in a conical tube and filtered with 0.22 μm Steriflip™ Sterile Disposable Vacuum Filter Units (MilliporeSigma™). In another conical tube, we added 1.8 mL 40K PEI (1 mg/mL) into 16 mL Transfectagro™ and mixed briefly. The premixed 40K PEI-transfectagro™ solution was gently poured into the filtered plasmid solution. The solution was thoroughly mixed by inverting the tube several times. The mixture rested at room temperature for 30 min and was poured into 1 L FreeStyle293F cell culture. After 5 days, the cells were removed from the supernatant by centrifuging at 3500 rpm for 15 min. The supernatant was filtered in a glass bottle with a 0.22 μm membrane and kept in 4° C. storage before loading into the columns. The His-tagged proteins were purified with the HisPur Ni-NTA Resin (Thermo Fisher). To eliminate nonspecific binding proteins, each column was washed with at least 3 bed volumes of wash buffer (25 mM Imidazole, pH 7.4). To elute the purified proteins from the column, we loaded 25 mL of the elution buffer (250 mM Imidazole, pH 7.4) at slow gravity speed (˜4 sec/drop). By using Amicon tubes, we buffer exchanged the solution with PBS and concentrated the proteins. The proteins were further purified by size-exclusion chromatography using Superdex 200 (GE Healthcare). The selected fractions were pooled and concentrated again for further use.

### Example: 18

Recombinant Protein ELISAs

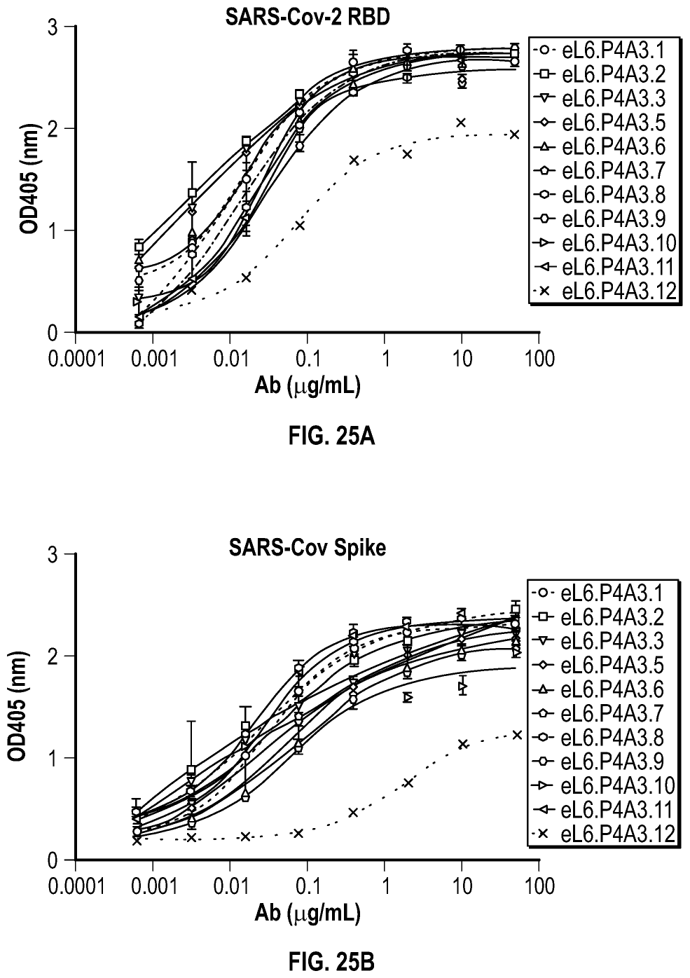

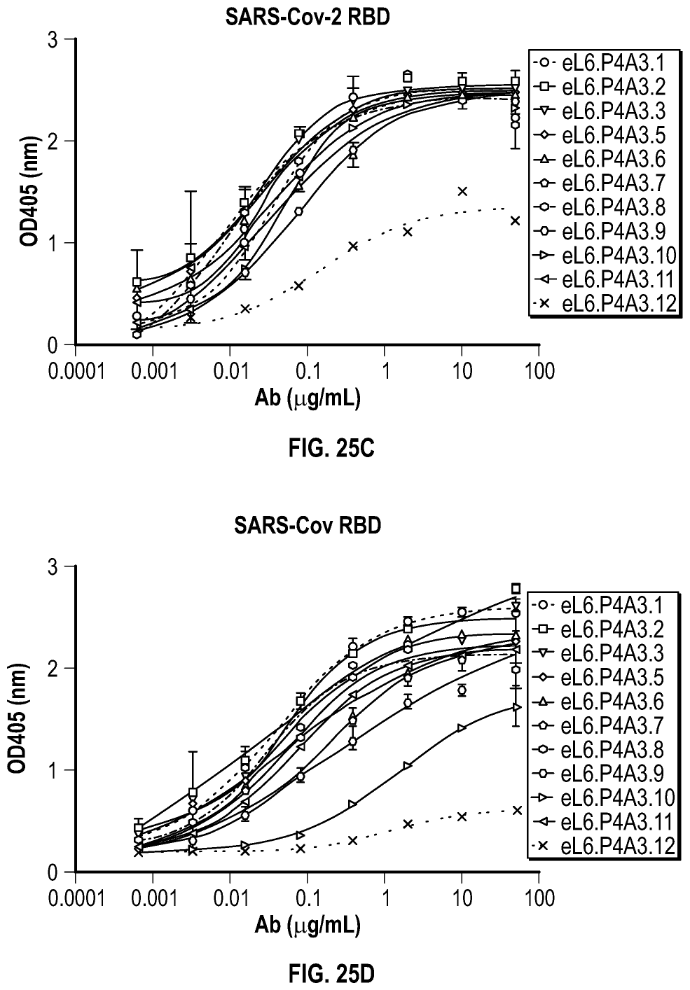

6×-His tag monoclonal antibody (Invitrogen. UA280087) was coated onto high-binding 96-well plates (Corning, 3690) at 2 μg/mL overnight at 4° C. After washing, plates were blocked with 3% BSA in PBS for 1 h. Then his-tag recombinant RBD and Spike protein were captured at 1 μg/mL in 1% BSA and incubated for 1 h at RT. After washing, serially diluted mAbs or sera were added into wells and incubated for 1 h at RT. Detection was measured with alkaline phosphatase-conjugated goat anti-human IgG Fey (Jackson ImmunoResearch) at 1:1000 dilution for 1 h. After the final wash, phosphatase substrate (Sigma-Aldrich) was added into wells. Absorption was measured at 405 nm. Non-linear regression curves were analyzed using Prism 8 software to calculate EC₅₀ values, eL6.P4A3 enhanced IgG1 monoclonal antibodies were evaluated for binding to SARS-Cov-2 and SARS-Cov antigens in the same method above. Data indicate that all the enhanced antibodies bind to the target antigens at a higher apparent affinity than the parental antibody (P4A3), which is highlighted as a black square. (FIG. 25A-D).

### Example: 19

Isolation of SARS-2 S-Specific mAbs

The process for sorting antigen-specific memory B cells was adapted for high-throughput such that each step could be performed in a 96-well format. Fluorescent-labeled antibodies recognizing cell surface markers were purchased from BD Biosciences. Avil-tagged SARS-2 S and RBD proteins were produced, purified, labeled with biotin (Avidity), and coupled to streptavidin-AF647, streptavidin-AF488 (Thermo Fisher), and streptavidin-BV421 (BD Biosciences), as previously described (20) at 2:1 and 4:1 molecular ratio respectively 30 min prior to staining. Cells were first labeled with antibodies for surface markers together with biotinylated probes (200 nM final) for 30 min in sort-buffer (PBS 1% FBS, 2.5M EDTA, 25 mM Hepes) on ice. Cells were then stained with the Live/Dead Fixable Aqua Dead Cell Stain (Thermo Fisher) for 15 min on ice according to the manufacturer's instructions. Single antigen-specific (S+ and RBD+) memory B cells (CD3-CD4-CD8-CD14-CD19+IgD-IgG+) were sorted into individual empty wells of a 96-well plate using a BD FACSAria Fusion sorter. Plates were immediately sealed and stored at −80° C.

cDNA was generated from cells sorted using Superscript IV Reverse Transcriptase (Thermo Fisher), dNTPs (Thermo Fisher), random hexamers (Gene Link) and Ig gene-specific primers in a lysis buffer containing Igepal (Sigma), DTT and RNAseOUT (Thermo Fisher). Nested PCR amplification of heavy- and light-chain variable regions was performed using Hot Start DNA Polymerases (Qiagen, Thermo Fisher), and previously described primer sets (21, 22). Second round PCR primers were modified to include additional nucleotides overlapping with the expression vectors. PCR efficiency was assessed using 96w E-gels (Thermo Fisher). Paired wells were picked individually, re-arrayed into new 96w plates and cloned in-frame into expression vectors encoding the human IgG1, Ig kappa or Ig lambda constant domains using the Gibson Assembly Enzyme mix (New England BioLabs) according to the manufacturer's instructions. Ligation reactions were transformed into DH5a competent E-coli, transferred into 1 mL Plasmid+ media (Thomson Instrument Company) supplemented with antibiotic and grown overnight at 37° C. under agitation. The next day the cultures were used to inoculate duplicate cultures before being lysed for plasmid DNA extraction using NucleoSpin 96 miniprep kit (Macherey-Nagel, Takara). Cloned heavy- and light-chain variable regions were sequenced (Genewiz) and subsequently analyzed using the IMGT (International ImMunoGeneTics Information System, www.imgt.org) V-quest webserver.

### Example: 20

Antibody Expression and Purification

Antibodies HC and LC constructs were transiently expressed with the Expi293 Expression System (Thermo fisher). After 4 days, 24-deep well culture supernatants were harvested to be directly tested for binding and neutralization. Selected mAbs showing neutralizing activity in the HTP screening were re-expressed in small to medium scale cultures using individual colony plasmid DNA, and IgG purified on Protein A sepharose (GE Healthcare).

### Example: 21

Epitope Binning by Bio-Layer Interferometry

The antibody hits that were identified in the high-throughput screening were next evaluated for epitope specificity by bio-layer interferometry (BLI) using S and RBD proteins as capture antigens. The antigens were captured on anti-HIS biosensors before addition of saturating concentrations (100 μg/ml) of antibodies that were then followed by competing antibodies at a lower concentration (25 μg/ml) for 5 minutes. Accordingly, only antibodies that bind to a non-competing site would be detected in the assay. Among the antibodies evaluated, the results reveal three epitope bins for RBD (designated as RBD-A. RBD-B, and RBD-C) and three epitope bins for the S protein (designated as S-A. S-B, and S-C). Interestingly, the mAb CC12.19 appears to compete with antibodies targeting two different epitopes, RBD-B and S-A, which might indicate that this mAb targets an epitope spanning RBD-B and S-A.

### Example: 22

Surface Plasmon Resonance Methods

SPR measurements were carried out on a Biacore 8K instrument at 25° C. All experiments were carried out with a flow rate of 30 μL/min in a mobile phase of HBS-EP+ [0.01 M HEPES (pH 7.4), 0.15 M NaCl, 3 mM EDTA, 0.0005% (v/v) Surfactant P20]. Anti-Human IgG (Fc) antibody (Cytiva) was immobilized to a density of ˜7000-10000 RU via standard NHS/EDC coupling to a Series S CM-5 (Cytiva) sensor chip. A reference surface was generated through the same method. For conventional kinetic/dose-response, listed antibodies were captured to ˜50-100 RU via Fc-capture on the active flow cell prior to analyte injection. A concentration series of SARS-CoV-2 RBD was injected across the antibody and control surface for 2 min, followed by a 5 min dissociation phase using a multi-cycle method. Regeneration of the surface in between injections of SARS-CoV-2 RBD was achieved with a single, 120 s injection of 3M MgCl₂. Kinetic analysis of each reference subtracted injection series was performed using the BIAEvaluation software (Cytiva). All sensorgram series were fit to a 1:1 (Langmuir) binding model of interaction.

A nAb SPR assay was also used to assess the competition between SARS-CoV-2 RBD and ACE2 for binding to CC12.1. CC12.1 was captured to the surface of 3 flow cells to ˜100 RU via Fc-capture. SARS-CoV-2 RBD was injected to each flow cell at a concentration of 50 nM to establish a basal level of SARS-CoV-2 RBD binding. This concentration was held constant for the competition experiments, which were carried out by varying the ACE2 concentration over eight points from 800 to 6.25 nM. To calculate residual SARS-CoV-2 RBD binding, the sensorgram responding to the corresponding ACE2 injection alone was subtracted from the SARS-CoV-2 RBD plus ACE2 injection series. The average response for the 5 s preceding the injection stop was plotted against the concentration of ACE2 and fit to a dose-response inhibition curve by nonlinear regression [log(inhibitor) vs. response−variable slope (4 parameters)] using GraphPad Prism. Regeneration between injections was carried out as noted above.

### Example: 23

Animal Study

SARS-CoV-2 infection of 8-week old Syrian hamsters was achieved through the intranasal installation of 10⁶ total pfu per animal in 100 ul of PBS. Animal weights were obtained during the study as a measure of disease progression. Treatment groups included the intraperitoneal injection of varying doses of monoclonal antibody. After 12 h, serum was obtained to quantify mAb titers and animals were infected as described above. At day-5 post-infection, lungs were harvested for analysis.

### Example: 24

Viral Load Measurements

Viral RNA was isolated from lung tissue and subsequently amplified and quantified in a RT-qPCR reaction. Lung tissue was extracted at day 5 post infection. The lung tissue was divided into sections approximately 100-300 mg in size. Samples were placed in 1 mL of TRIzol-LS reagent (Invitrogen). Samples for virus load were then subjected to tissue homogenization using disposable pestles in 15 mL conical tubes (Corning). Tissue homogenates were then spun down to remove any remaining cellular debris and the supernatant was added to a RNA purification column (Qiagen). Purified RNA was eluted in 80 μL of DNase-, RNase-, endotoxin-free molecular biology grade water (Millipore) and quantified using a nanodrop (Thermo Fisher). RNA was then subjected to reverse transcription and quantitative PCR using the CDC's N1 primer sets (Forward 5′-GAC CCC AAA ATC AGC GAA AT-3′; Reverse 5′-TCT GGT TAC TGC CAG TTG AAT CTG-3′) and a double-quenched (ZEN/Iowa Black FQ) and fluorescently labeled (FAM) probe (5′-FAM-ACC CCG CAT TAC GTT TGG TGG ACC-BHQ1-3′) (Integrated DNA Technologies) on an BioRad CFX96 Real-Time instrument. For quantification, a standard curve was generated by diluting 1-1010 RNA copies SARS-CoV-2 genome/mL, 10-fold in water (Virapur). Every run utilized eight to ten-fold serial dilutions of the standard. SARS-CoV-2-positive and negative samples were included.

### Example: 25

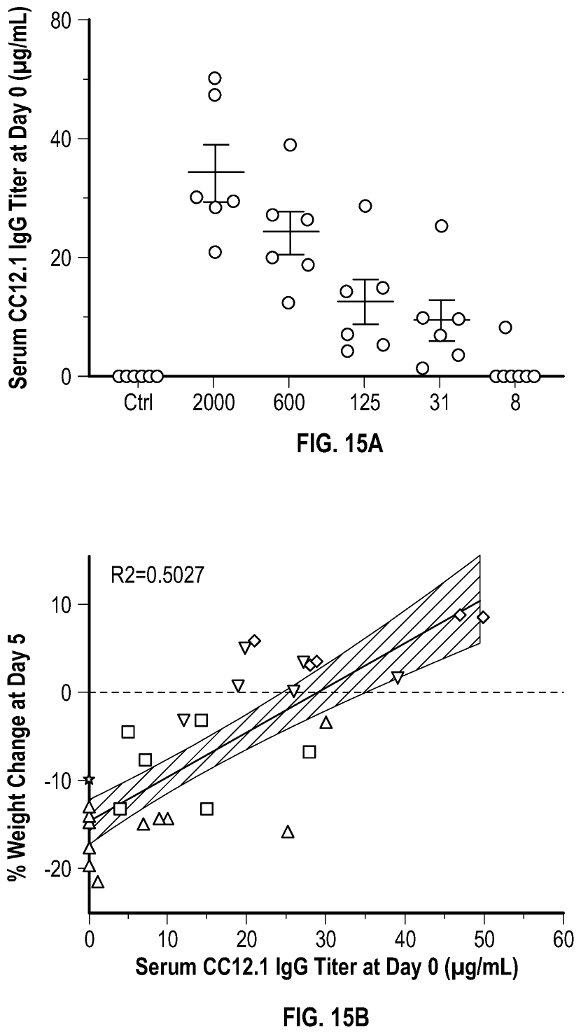

The weight loss data from Example 9 is further corroborated by quantification of lung viral load measured by real-time PCR and showed a moderate correlation to weight loss. The data indicate comparable viral loads between the three higher doses (2 mg, 500 μg, and 125 μg) of nAbs. To determine the antibody serum concentrations that may be required for protection against disease from SARS-CoV-2 infection, the antibody serum concentrations were also measured just prior to intranasal virus challenge (FIGS. 15A and B). The data highlight that an antibody serum concentration of approximately 22 μg/ml of nAb (1160×PSV neutralization IC₅₀) enables full protection and a serum concentration of 12 μg/ml (630×PSV neutralization IC₅₀) is adequate for 50% reduced disease as measured by weight loss. The effective antibody concentration required at the site of infection to protect from disease remains to be determined. Sterilizing immunity at serum concentrations that represent a large multiplier of the in vitro neutralizing IC₅₀ is observed for many viruses.

### Example: 26

mAb Sensitivity to Different CoV-2 Mutants

L6.dP04E05 is ˜50-fold more potent than L12.bP11A6, which was used in passive transfer and challenge experiment in Syrian hamsters. The potent antibodies in Table 9A were screened against different virus variants to down-select a lead. L6.P4E5 in Table 9A and eL6.P4A3 variants in Table 9B and are capable of neutralizing resistant RBD variant (V367F). Additionally, enhanced monoclonal antibodies were evaluated for neutralization against SARS-Cov-2 mutants. Values shown are neutralization IC50 in ug/ml. “Escaped viral mutants” were generated by site-directed mutagenesis using mutations reported in the literature. Values >10 or >50 indicate no neutralization activity was observed at an antibody concentration of 10 ug/ml and 50 ug/ml, respectively. Data indicate that all the enhanced antibodies neutralize circulating SARS-Cov-2 mutants. While the V367F mutant is completely resistant to neutralization by the parental antibody, the enhanced antibodies are able to neutralization at ˜0.02 ug/ml.

The details of the spike mutation of the viral variants and the geographical sampling information can be found in Table 9C.

TABLE 9A

| | SARS-CoV-2 strain: USA-WA 1/2020 (WT) | | | | |
|---|---|---|---|---|---|
| | WT | D614G | V367F | G476S | V483A |
| L6.dP04E05 | 0.36 | 0.52 | 0.07 | 0.16 | 0.55 |
| L6.dP03H10 | 1.7 | 1.9 | 0.25 | 0.08 | 1.9 |
| L6.dP02E05 | 7.5 | 3.9 | 0.04 | KO | 3.9 |
| L6.dP04A03 | 168 | 24 | KO | 84 | 160 |

TABLE 9B

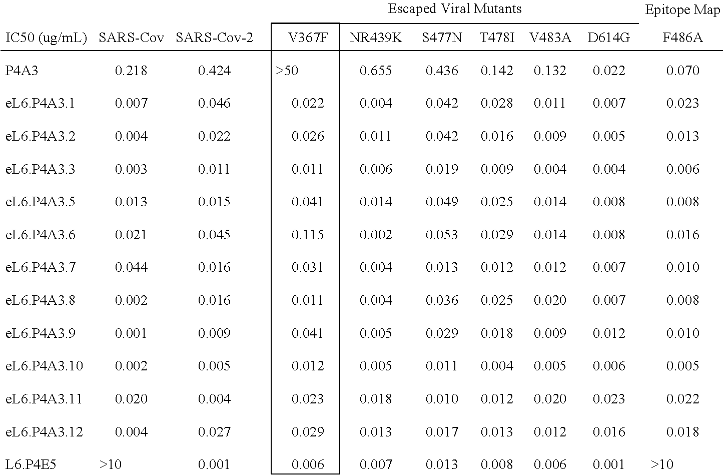

TABLE 9C

| | | Apr. 13, | | |
|---|---|---|---|---|
| Spike | Spike Location | 2020 | Geographic | Phylogenetic |
| Mutation | Possible Impact | Count | Sampling | Pattern |
| D614G | SARS-CoV epitope | 3577 | Global | One main lineage |
| | Interprotomer stabilization | | | & recurrent emergence |
| L5F | Signal Peptide | 37 | 13 countries | Recurrent emergence |
| L8V/W | Signal Peptide | 18 | Hong Kong | |
| H49Y | S1 NTD domain | 12 | China | |
| Y145H/del | S1 NTD domain | 10 | 6 countries | |
| Q239K | S1 NTD domain | 8 | Europe | |
| V367F | Up/Down conformations | 12 | Europe/Hong Kong | |
| G476S | Directly in the RBD | 8 | Washington, USA | |
| V483A | Up/Down conformations | 21 | Washington, USA | |
| V615I/F | In SARS-CoV ADE epitope | 13 | Wales | |
| A831V | Potential fusion peptide in S2 | 28 | Iceland | One lineage |
| D839Y/N/E | S2 subunit | 27 | Europe | |
| P1263L | Cytoplasmic Tail | 49 | UK/Iceland/Australia | One lineage |
| Mutational | Fusion core of HR1 | 65 | Different forms in 9 countries | |
| cluster | | | | |
| 934-940: | | | | |

### Example: 27

MARS-CoV-2 nAb Functional Summary

Table 10A summarizes the SARS-Cov-1 and SARS-CoV-2 binding affinities and neutralization potencies of the indicated SARS-CoV-2 specific mAbs isolated from CC12. Neutralization was tested against pseudotyped (PSV) and live replicating SARS-CoV-1 and SARS-CoV-2 viruses. MPN: Maximum Neutralization Plateau. Table 10A also correlates the monoclonal antibody ID number with the name designations used herein. Table 10B demonstrates that the enhanced antibody, eL6.P4A3.1 (from parental eL6.P4A3), neutralizes both pseudotype and live SARS-Cov-2 virus at very high potencies.

TABLE 10A

| | | Predicted | ELISA IC [symbol] (μg/mL) | | | | | PSV Neutralization IC [symbol] (μg/mL) | | | |
|---|---|---|---|---|---|---|---|---|---|---|---|
| | | Unique | SARS- | SARS- | SARS- | SARS- | Poly | SARS- | SARS- | | [symbol] |
| mAb ID | mAb name | Epitope | CoV-2 [symbol] | CoV-2 [symbol] | CoV-1 [symbol] | CoV-1 [symbol] | [symbol] | CoV-1 | CoV-2 | [symbol] | SARS-CoV-2 |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |

mAb ID
eLC6.P4A3.1
mAb name
eCC6.33.1 [symbol]
indicates data missing or illegible when filed

TABLE 10B

| | PSV in HeLa-ACE2 cells IC50(ug/ml) | | | | |
|---|---|---|---|---|---|
| | | | SARS COV-2 | SARS COV-2 live virus IC 50(ug/ml) | |
| Abs | SARS COV1 | SARS COV-2 | D614G | Vero | HeLa-ACE2 |
| REGN10987 | >25 | 0.093 | 0.019 | 0.001 | 0.005 |
| REGN10933 | >25 | 0.006 | 0.009 | 0.001 | 0.002 |
| eL6.P4A3.1 | 0.049 | 0.005 | 0.013 | 0.001 | 0.008 |
| L6.P4E5 | >25 | 0.006 | 0.008 | 0.006 | 0.003 |

### Example: 28

Neutralization Assay Development

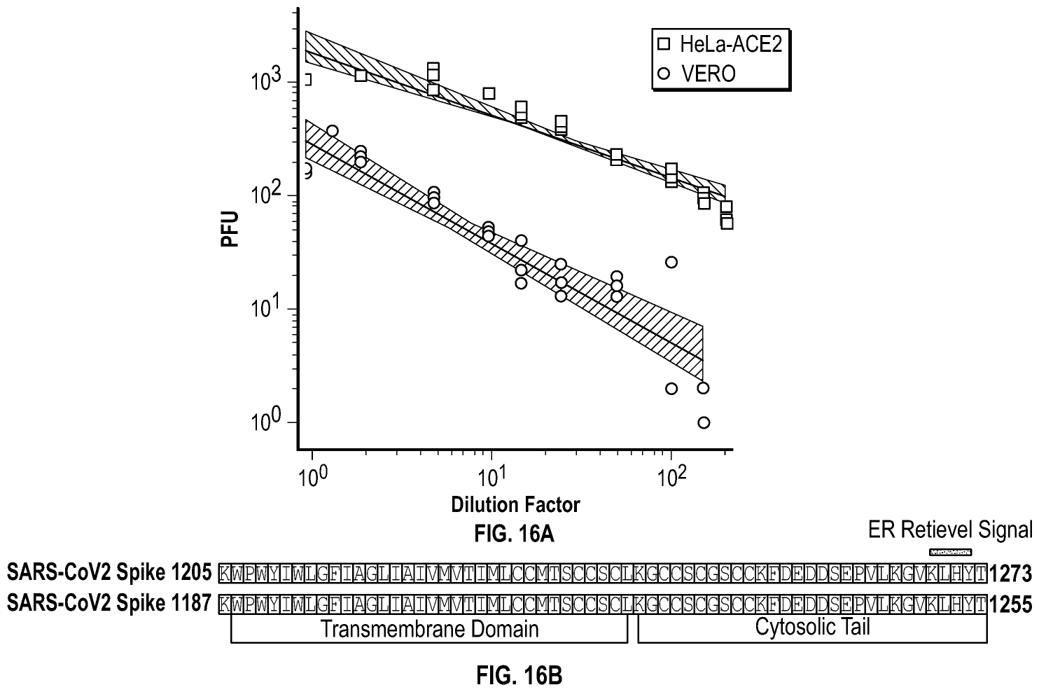

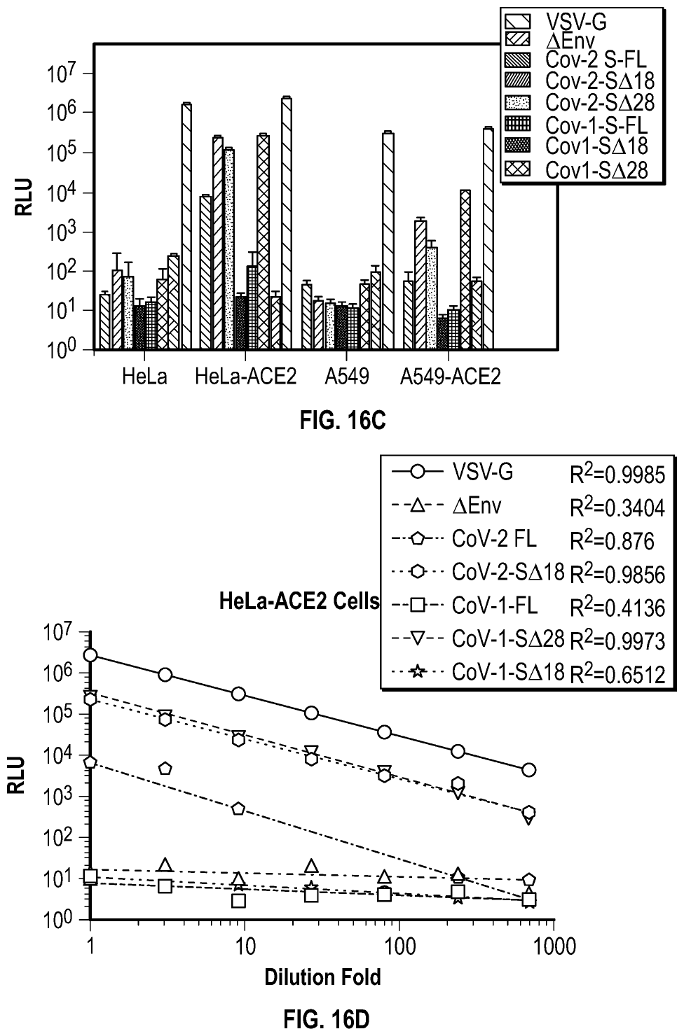

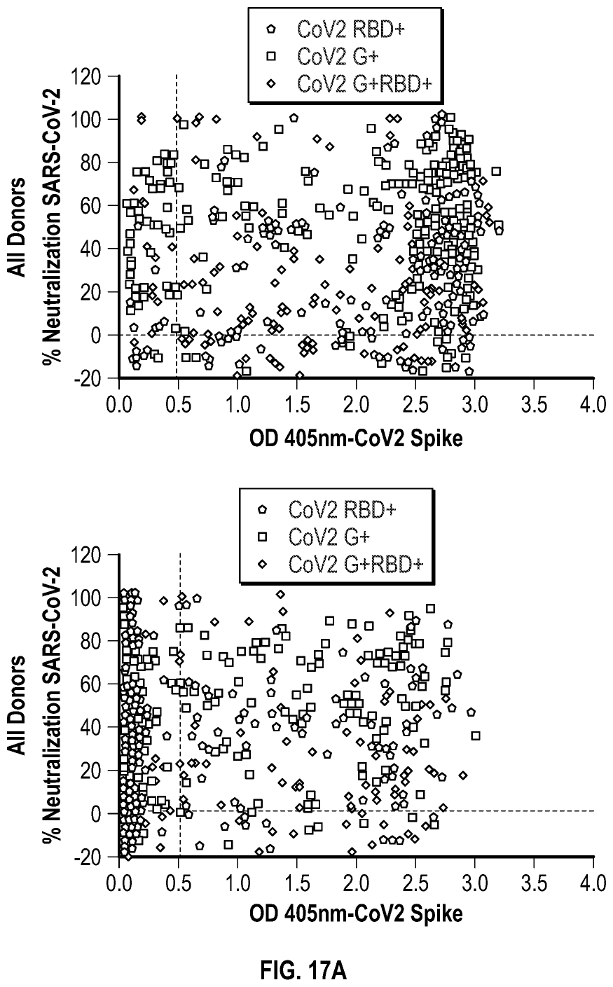

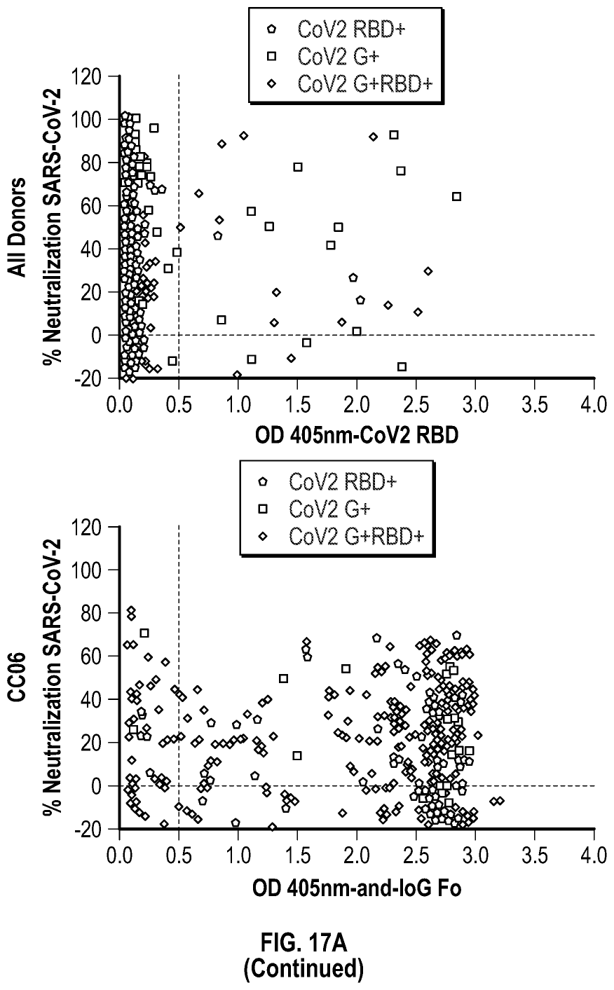

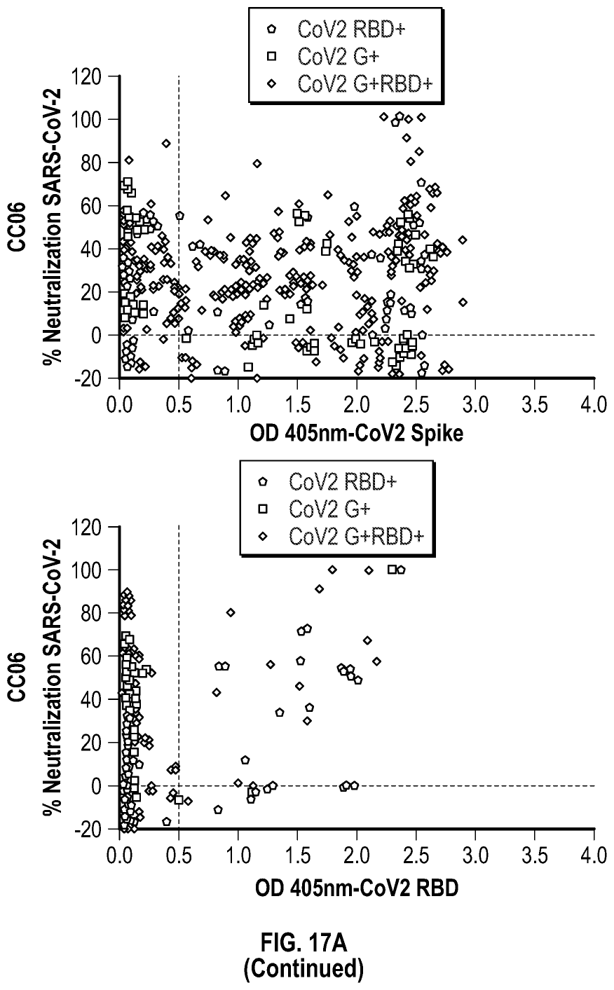

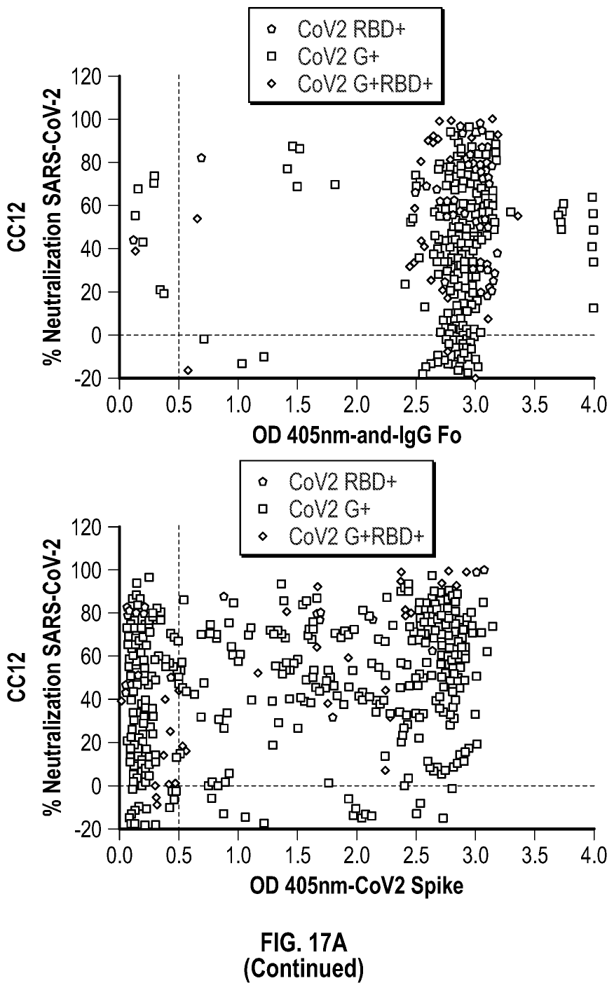

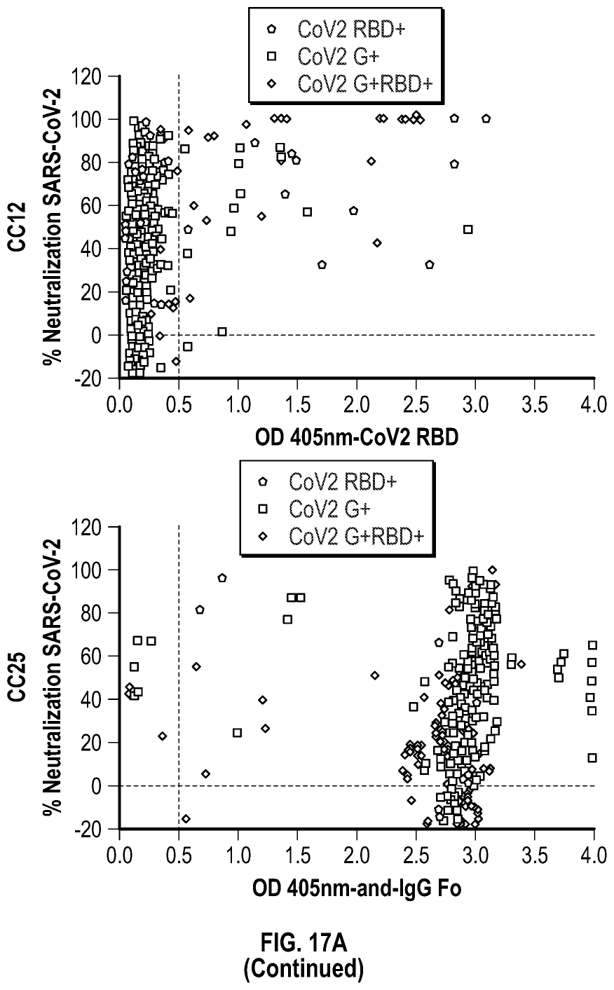

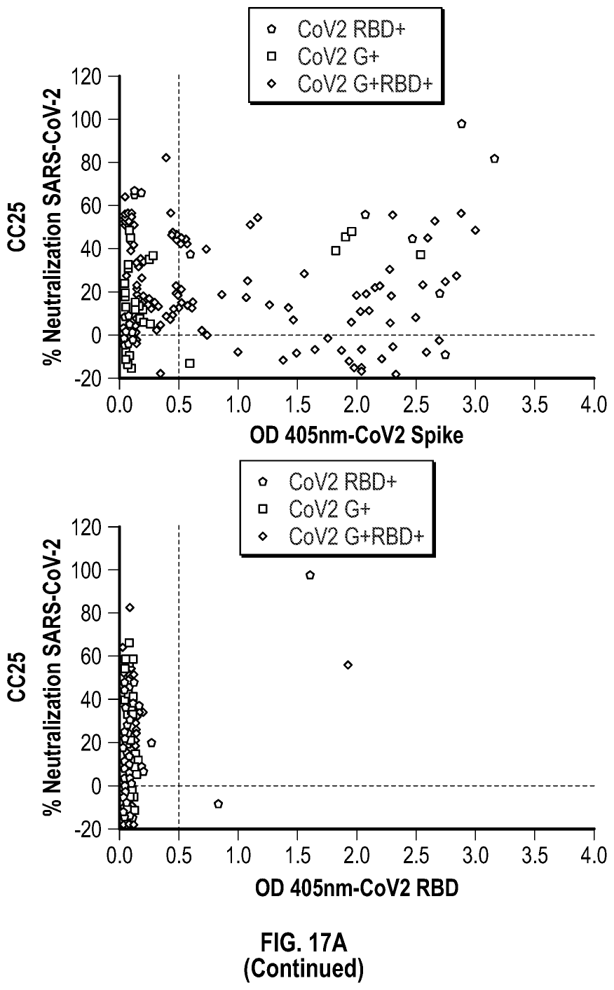

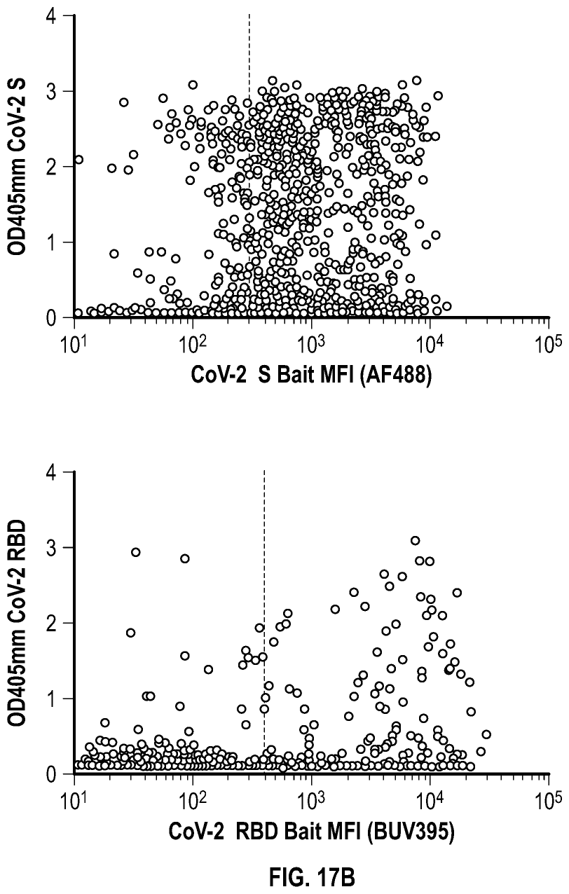

Vero and HeLa-ACE2 cells were infected with serially diluted SARS-CoV-2. Both cell types were plated at 1000 cells/well. The HeLa-ACE2 cell line showed 100% infection at a dilution factor of 2 (FIG. 16A). SARS-CoV-1 and SARS-CoV-2 transmembrane domain and cytoplasmic tail were aligned (FIG. 16B). The infectious efficiency of MLV viral particles pseudotyped with the indicated spike (S) proteins: FL indicates full-length spike, D18 and D28 denote C-terminal truncations were compared (FIG. 16C). Also included were control VSV-G-pseudotyped virions and empty vector control (D Env). Target cells were HeLa cells, A549 cells, HeLa or A549 cells overexpressing human ACE2. After 48 h of infection, luciferase expression was assessed. Pseudovirions were titrated in HeLa-ACE2 cells in 3-fold serial dilutions. Relative luciferase and fold-dilution were plotted (FIG. 16D). Correlation coefficient (R2) was calculated using linear regression in Prism.

### Example: 29

Preliminary Functional Screens for Downselection

Across the 3 donors, a total of 1043 antibodies were cloned and expressed, which represents, on average, a 65% PCR recovery of paired variable genes and >86% recovery of fully functional cloned genes (Table 11). The bulk-transformed ligation products for both the heavy chain and light chain were transfected and tested for binding to RBD and S protein, and for neutralization in the SARS-CoV-2 pseudovirus assay using HeLa-ACE2 target cells.

TABLE 11

| | Heavy Chain | Kappa chain | Lambda chain | H + K + L |
|---|---|---|---|---|
| | (H) Colonies | (K) Colonies | (L) Colonies | All Colonies |
| Total Colonies Screened | 819 | 819 | 819 | 2457 |
| Off-frame insert | 2 | 2 | 0 | 0.2% |
| IMGT: No Results | 45 | 4 | 9 | 2.4% |
| IMGT: Unproductive | [symbol] | 20 | 12 | 2.6% |
| IMGT: No rearrangement found | 7 | 0 | [symbol] | [symbol] % |
| Total Cloning Reactions | 273 | 273 | 273 | 819 |
| Overall Cloning Failures | 12 | 10 | 11 | 5.0% |

| | | |
|---|---|---|
| Total Cloned Pairs | 273 | 273 |
| Predicted Unproductive Pairs | [symbol] | [symbol] % |

[symbol]

indicates data missing or illegible when filed

### Example: 30

Functional Screening of Ab H+L Pairs Rescued from &ARS-CoV-2-Specific Single B-Cell Sorting

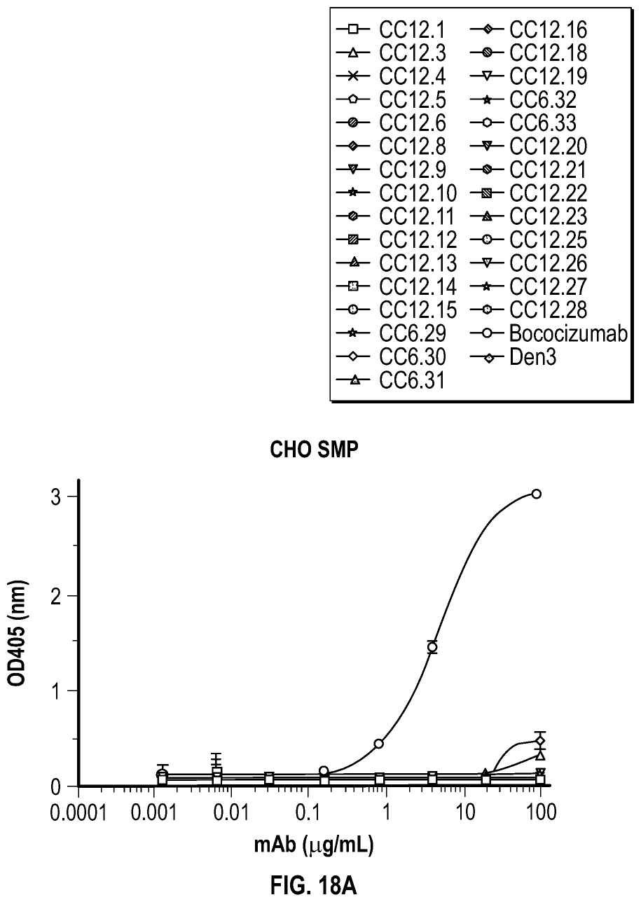

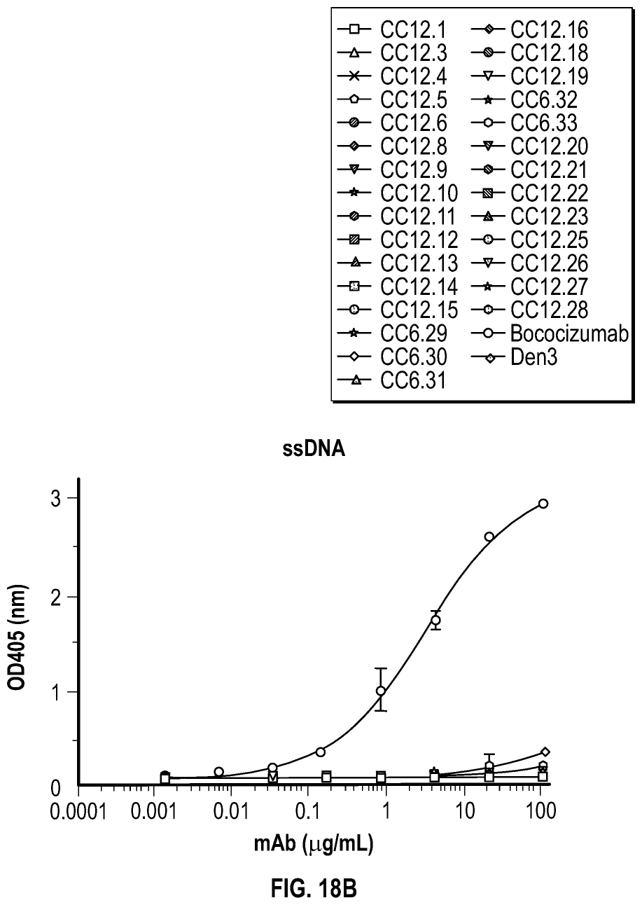

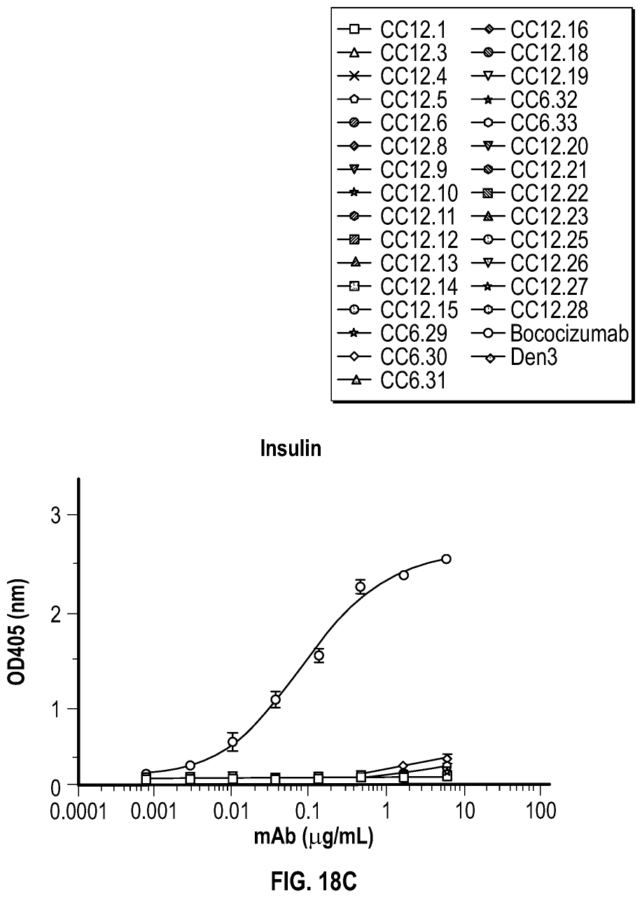

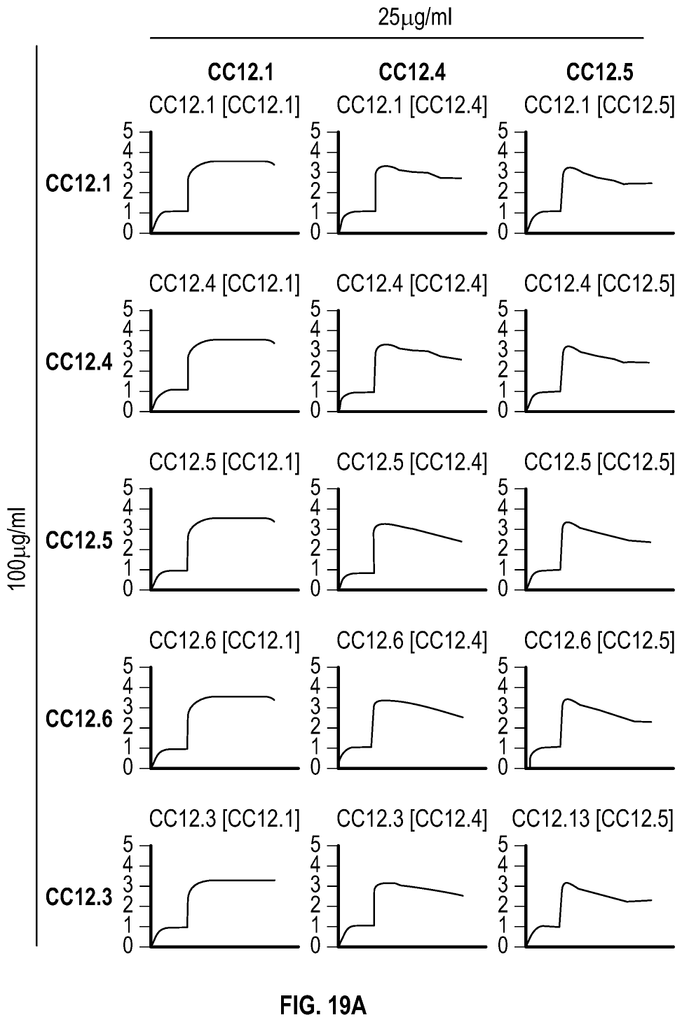

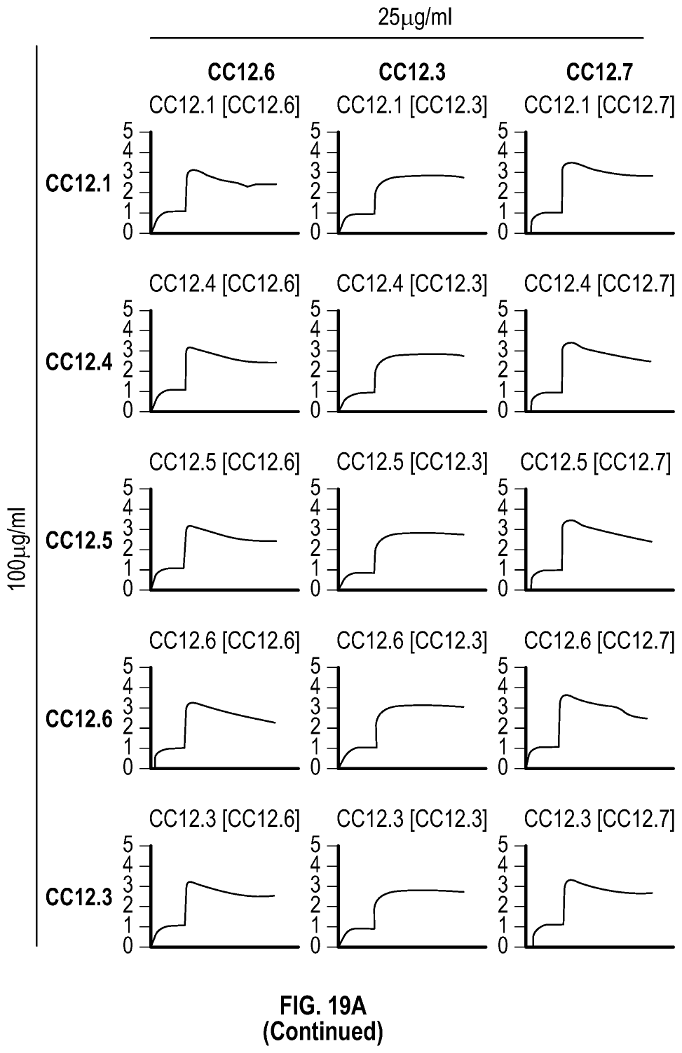

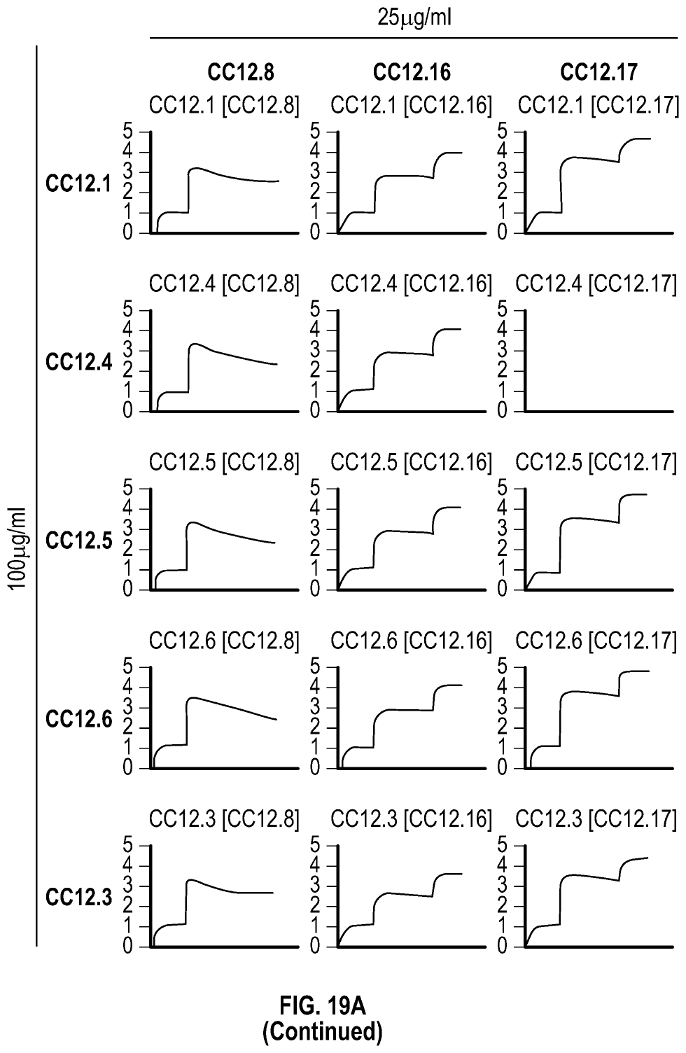

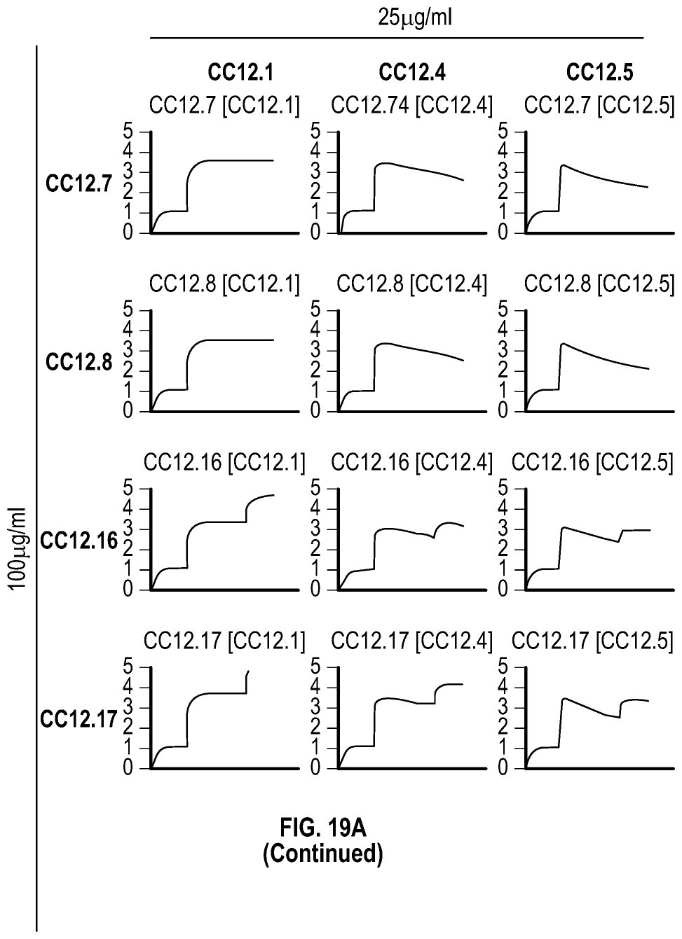

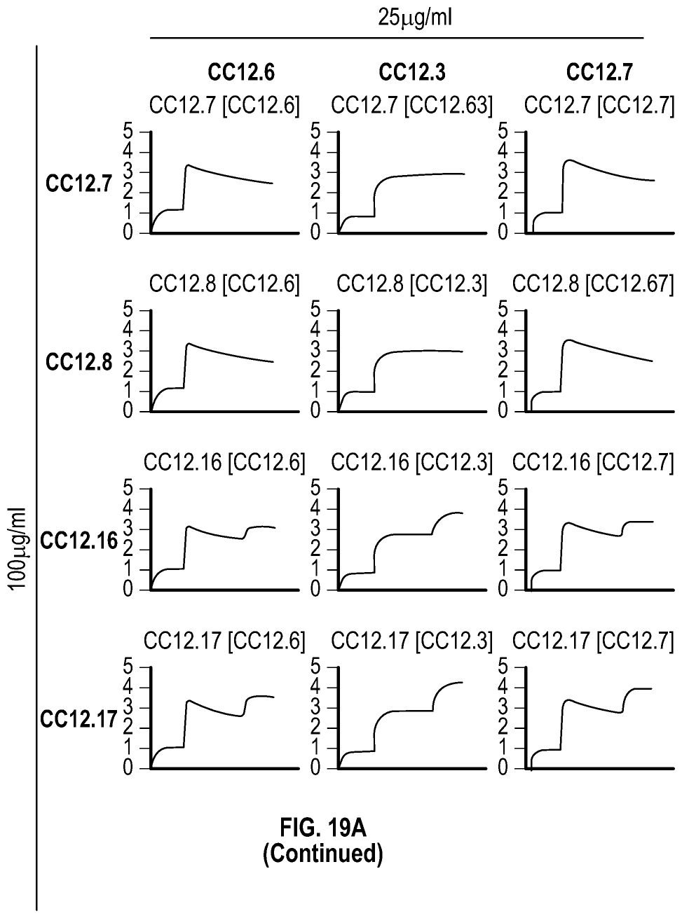

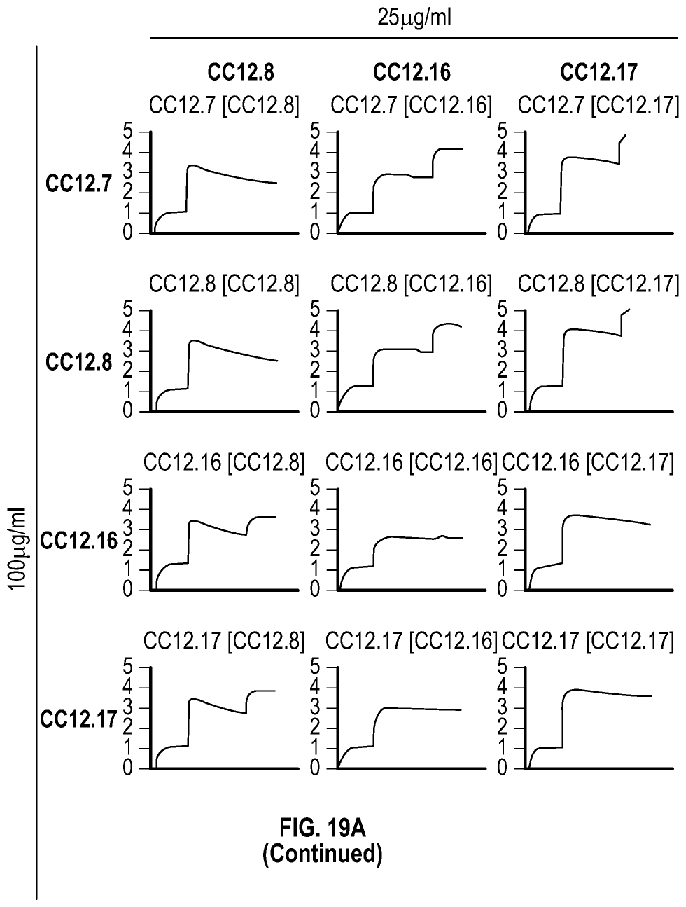

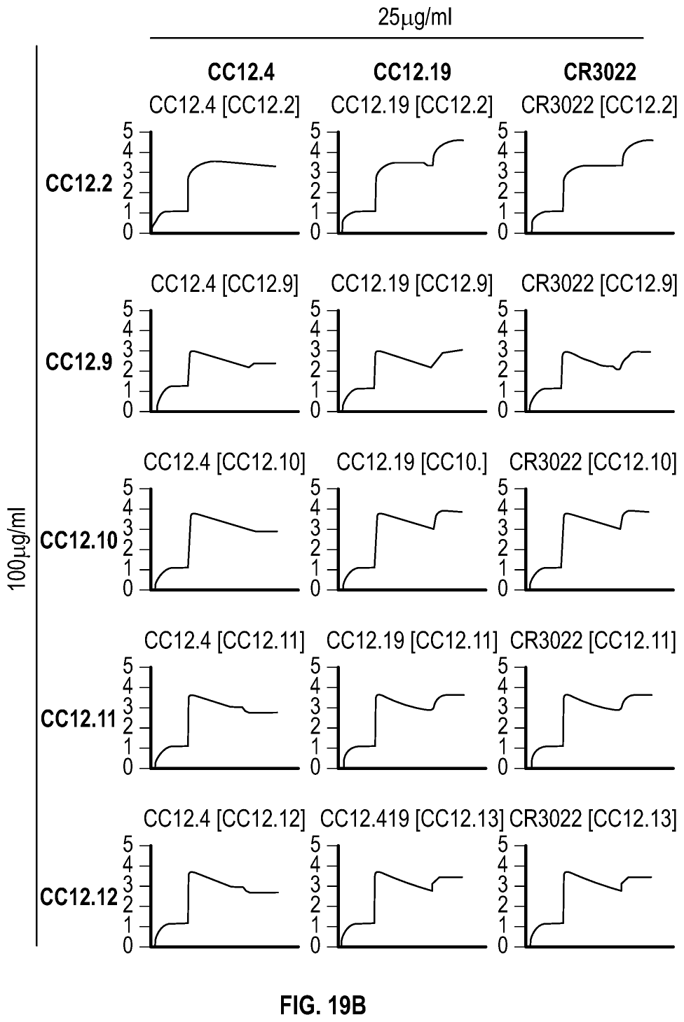

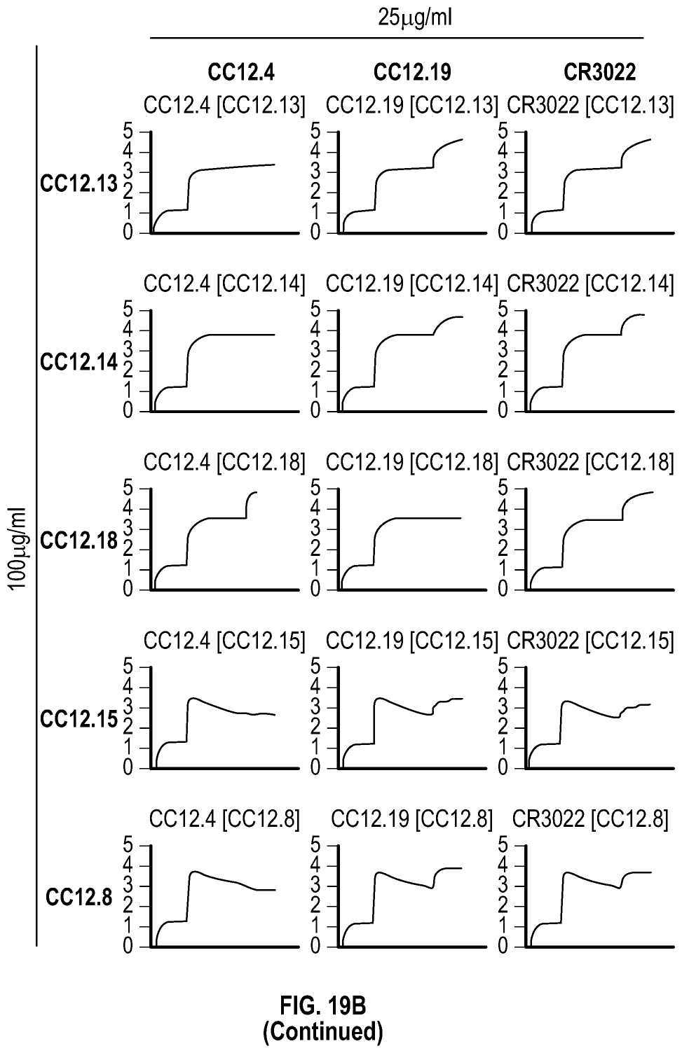

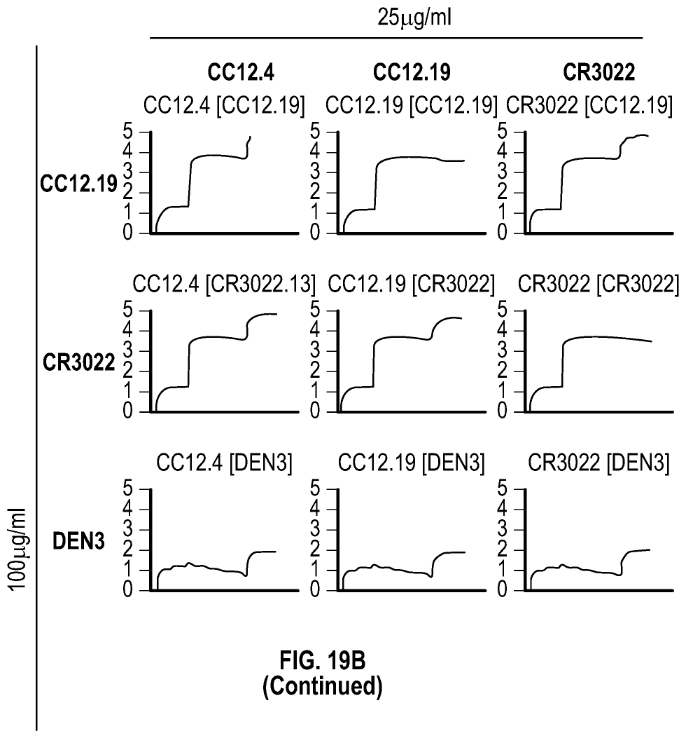

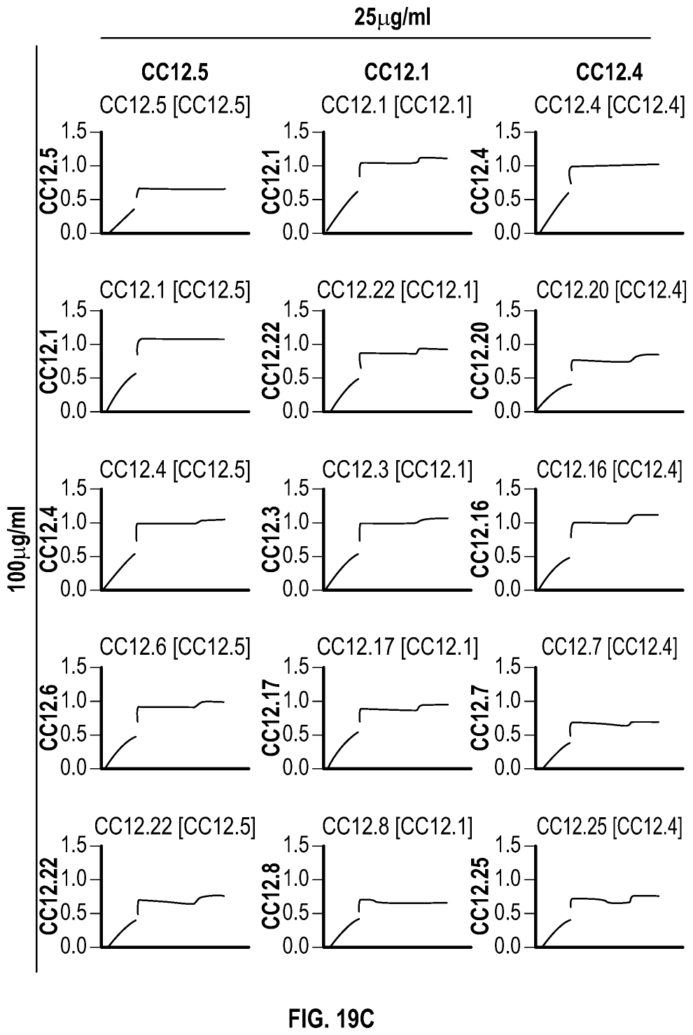

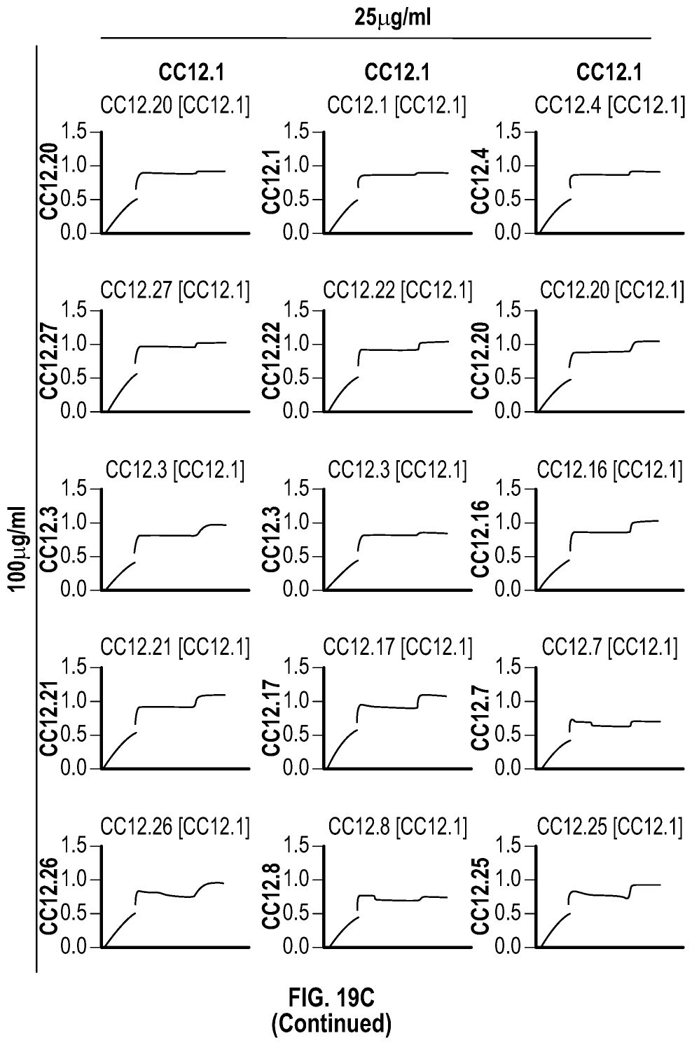

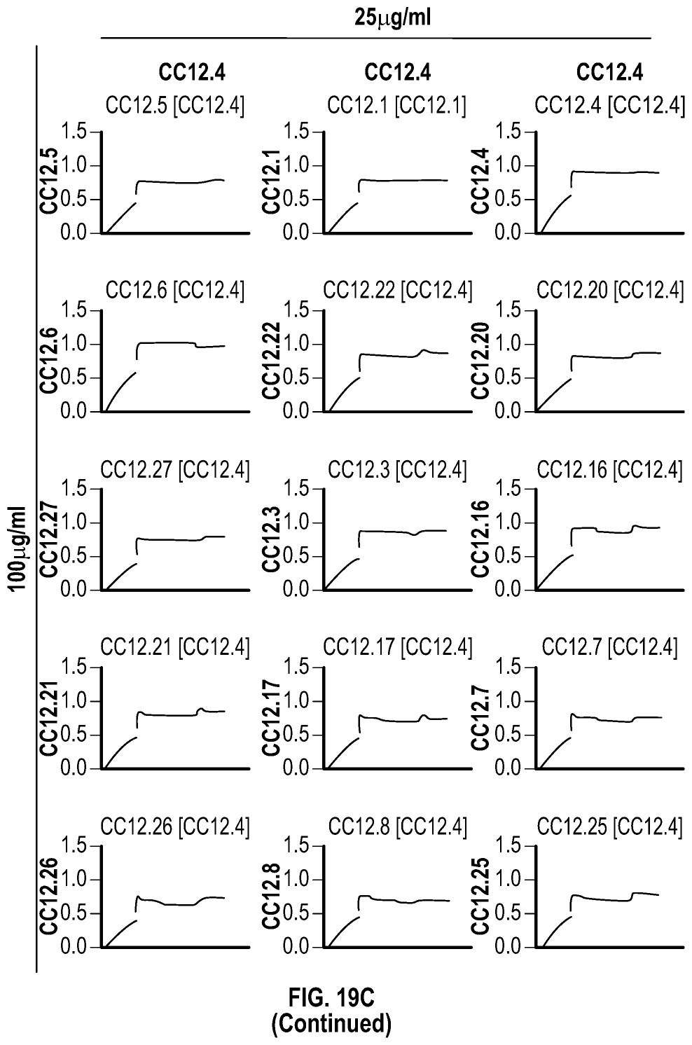

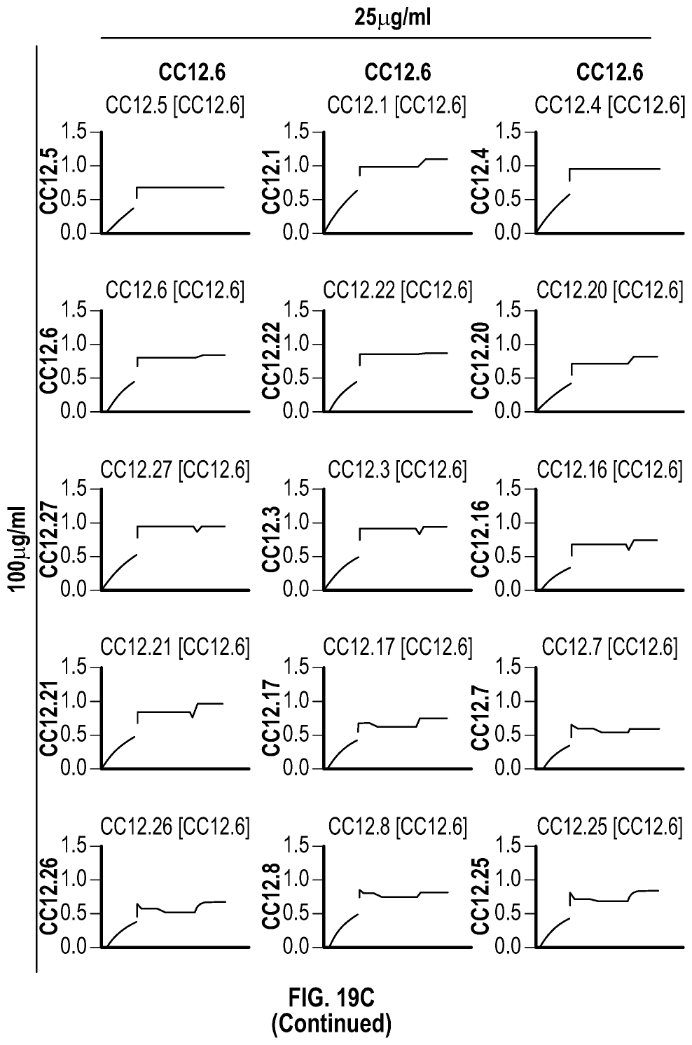

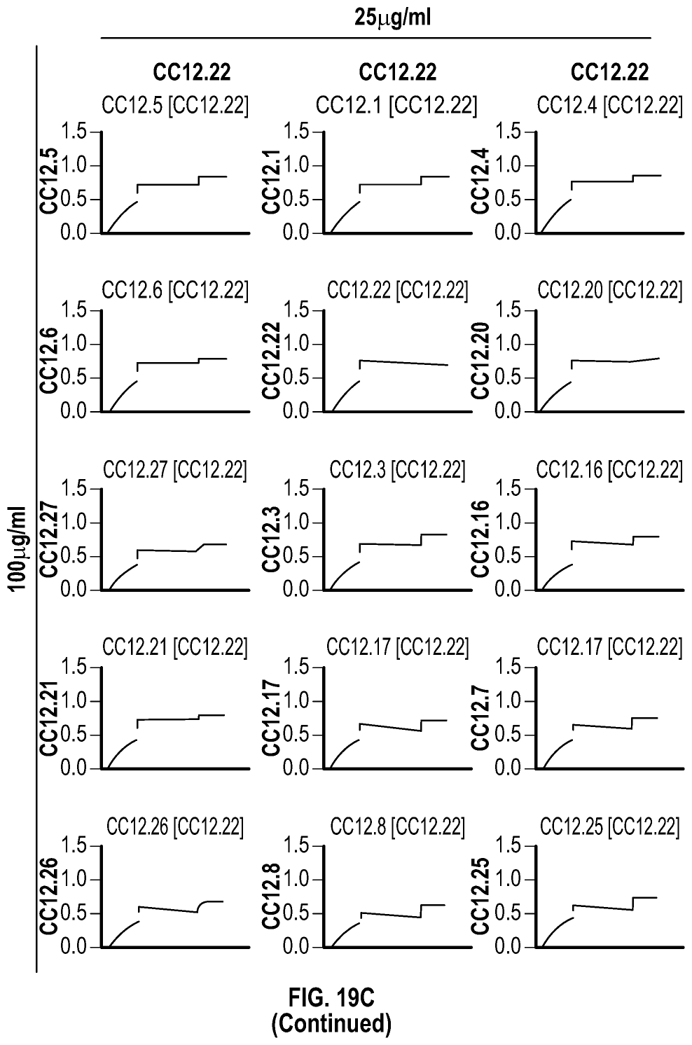

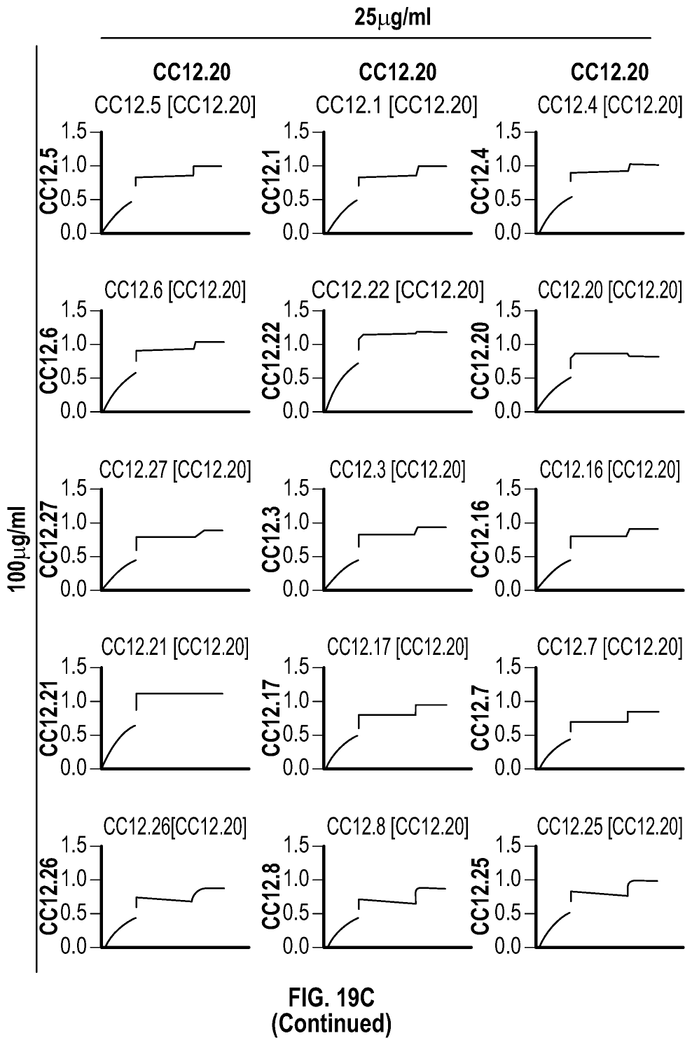

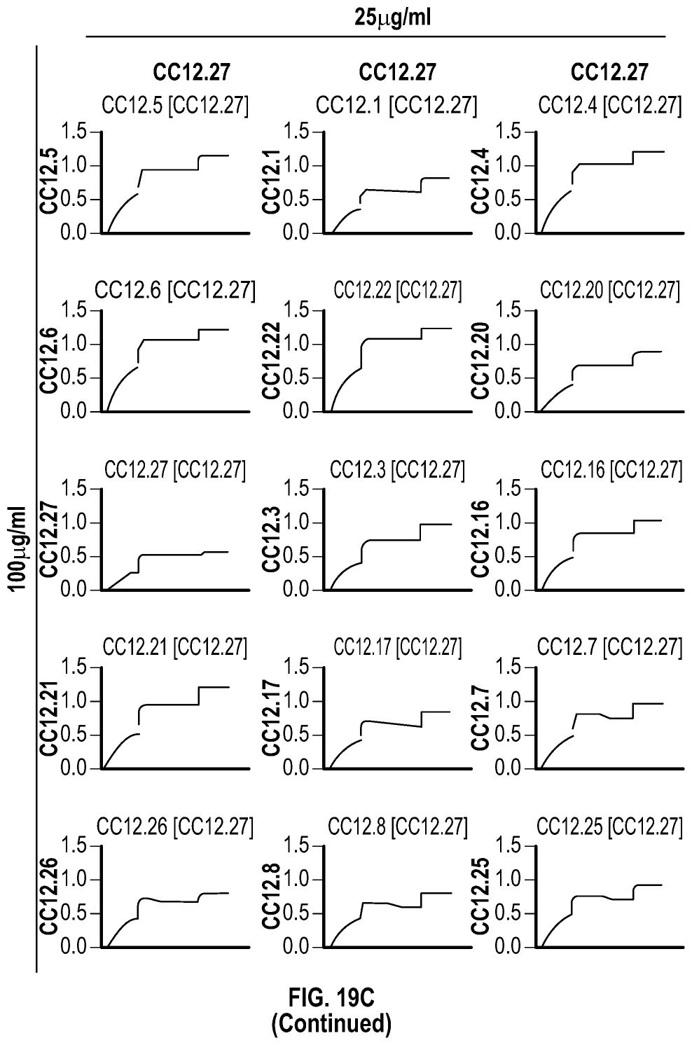

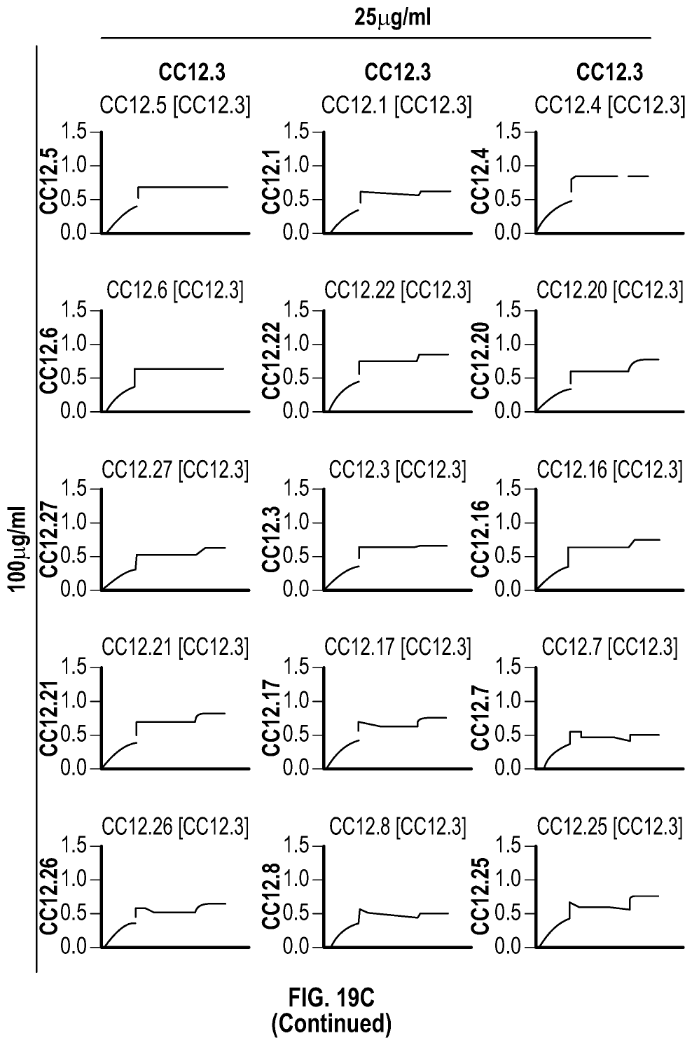

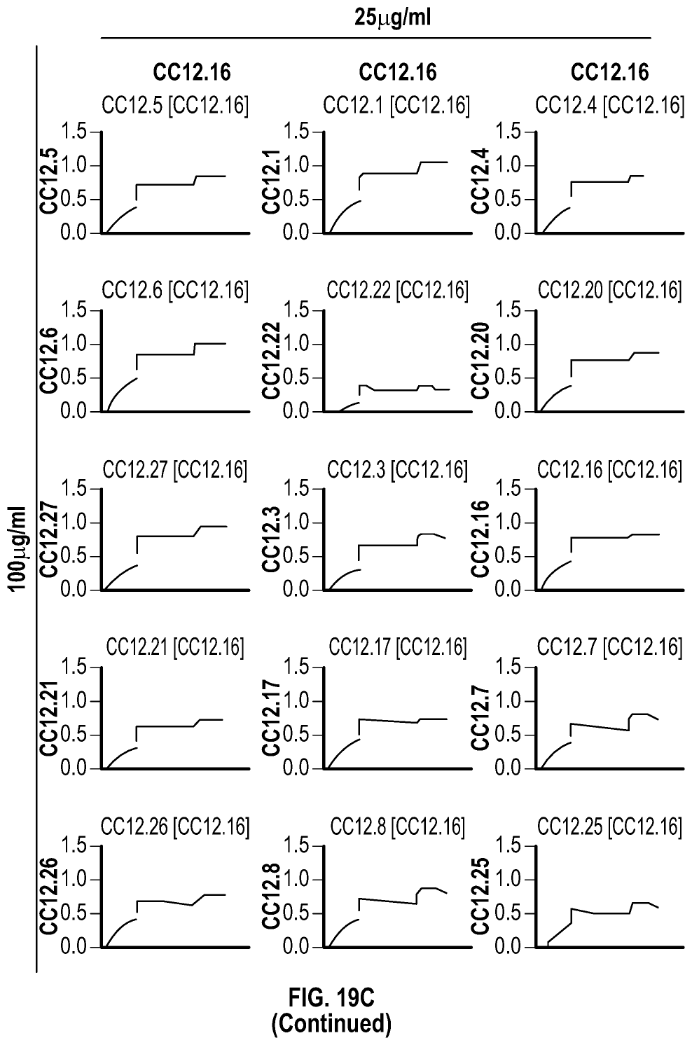

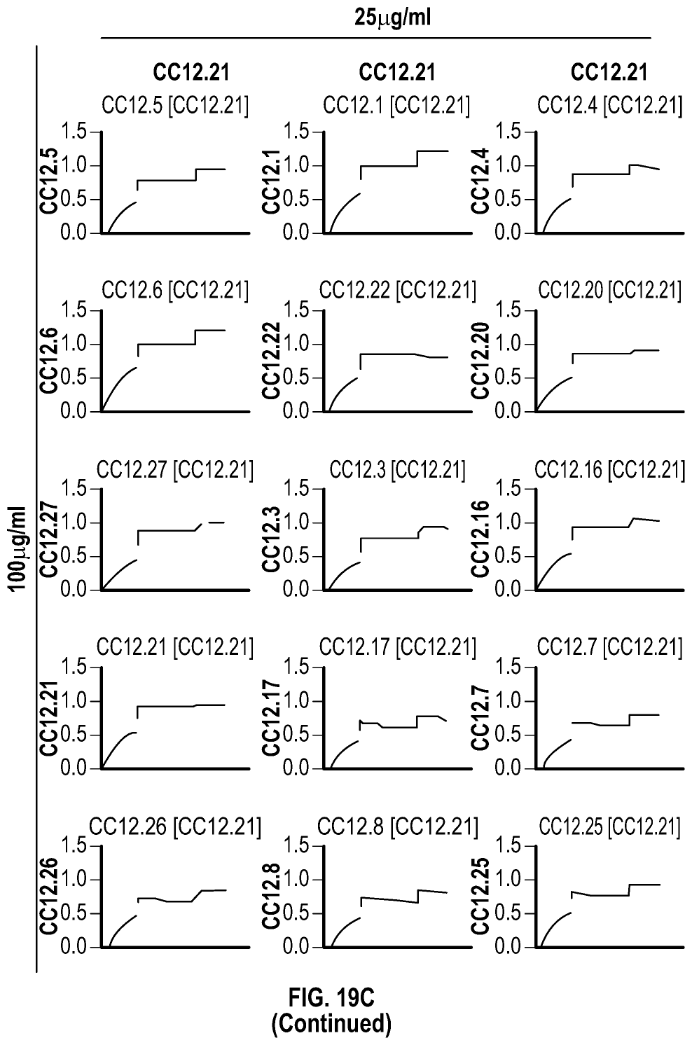

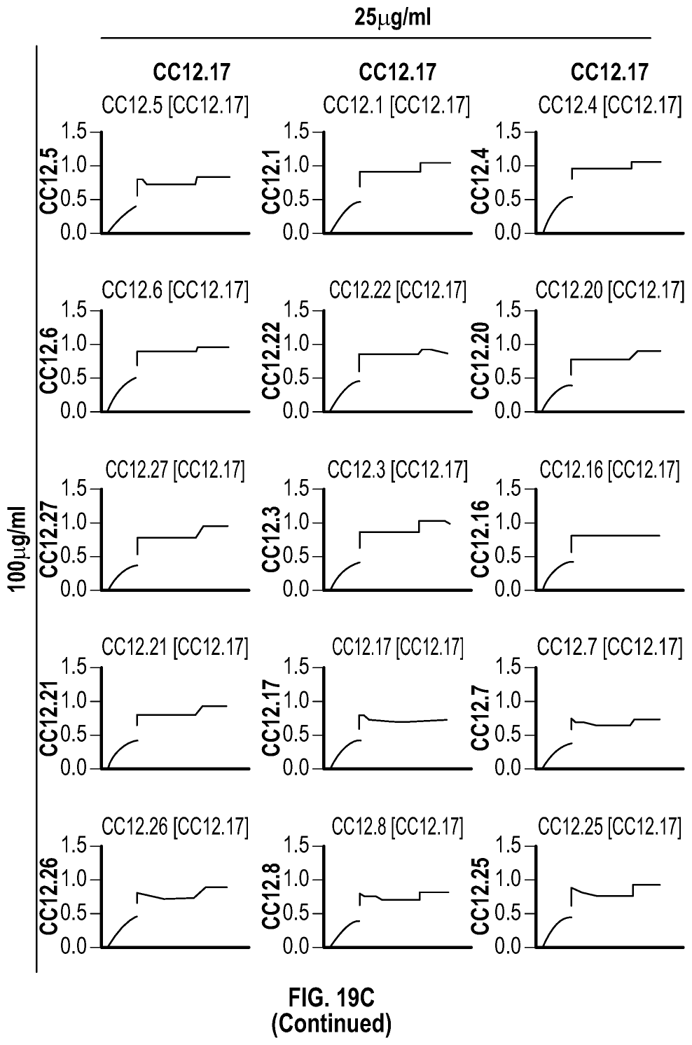

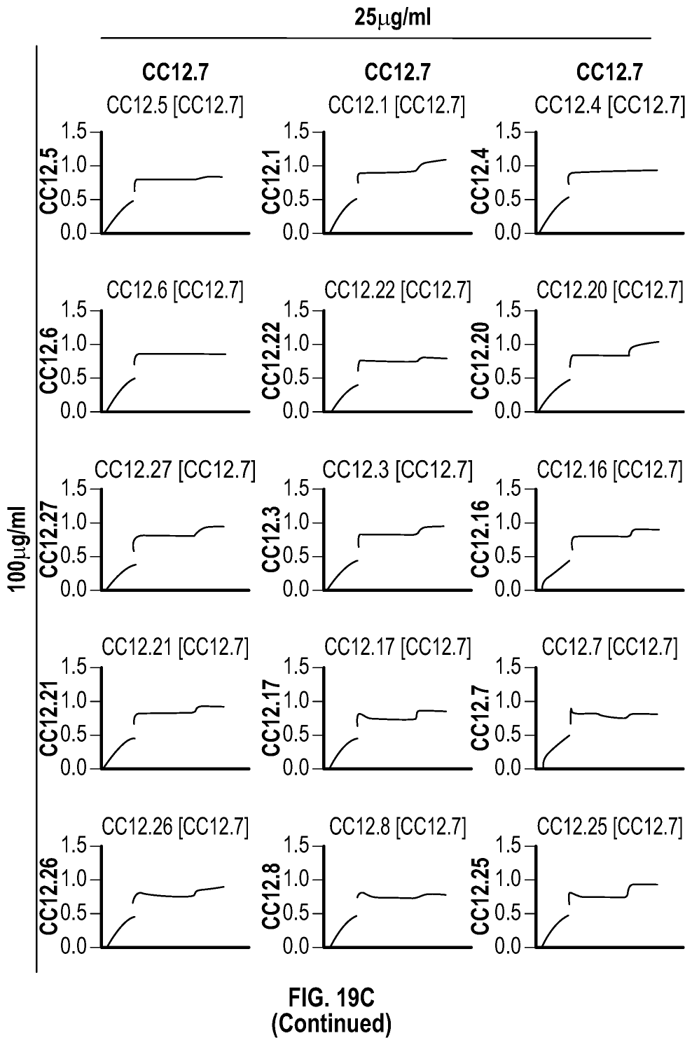

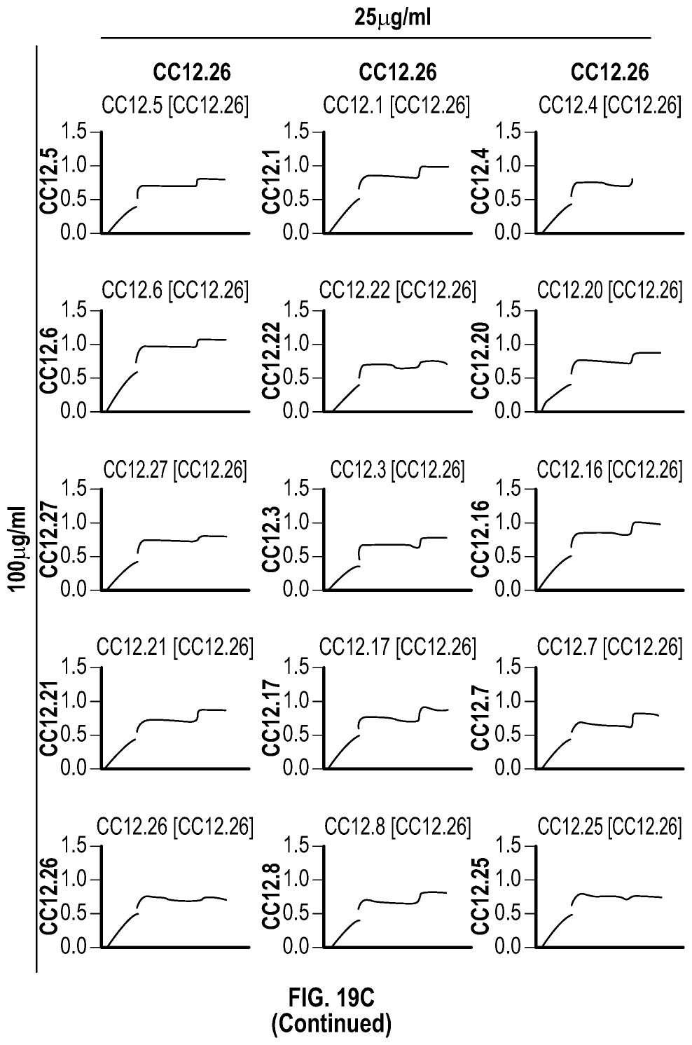

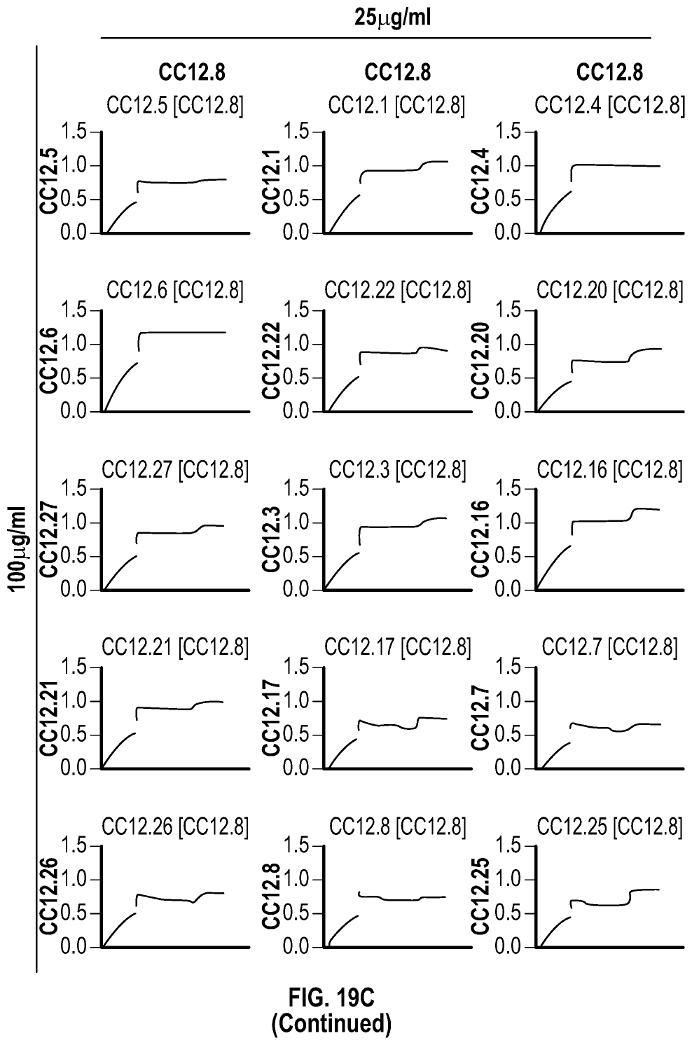

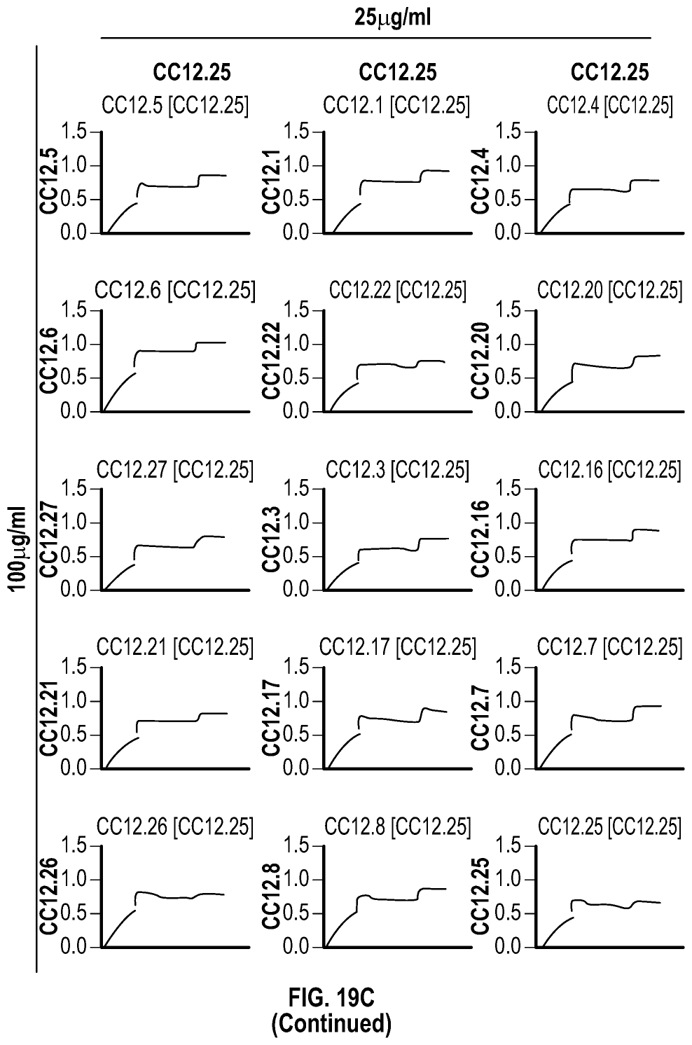

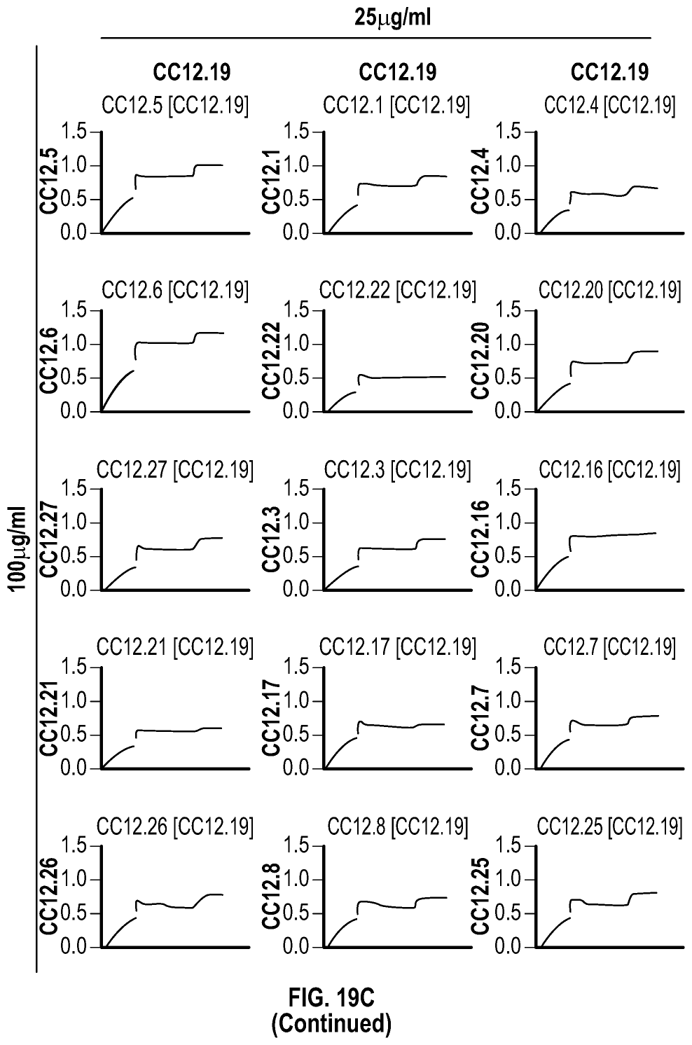

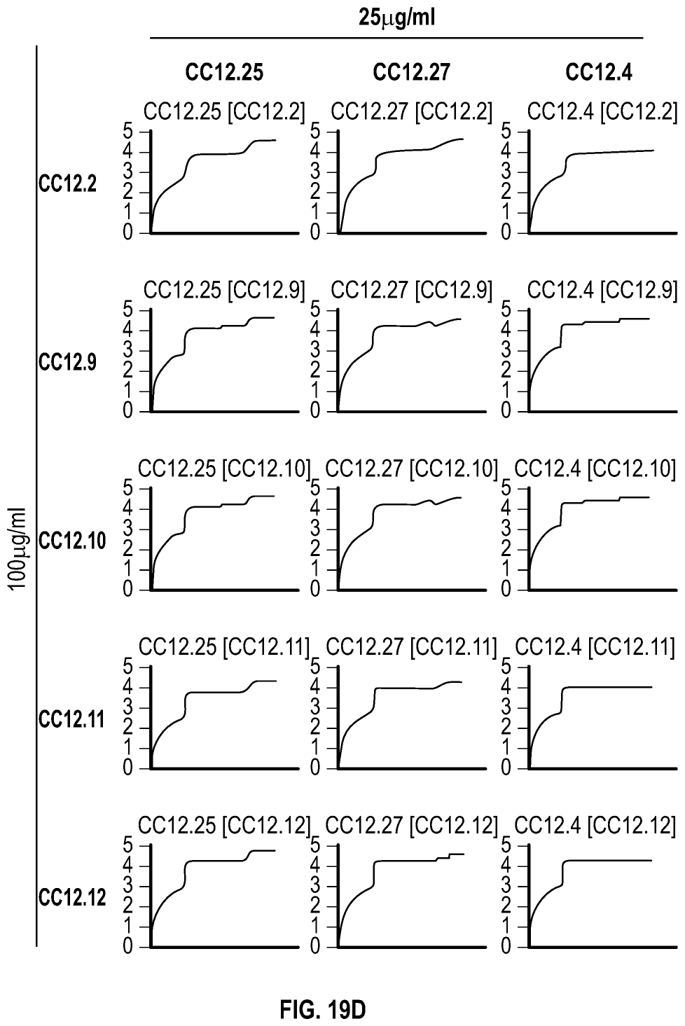

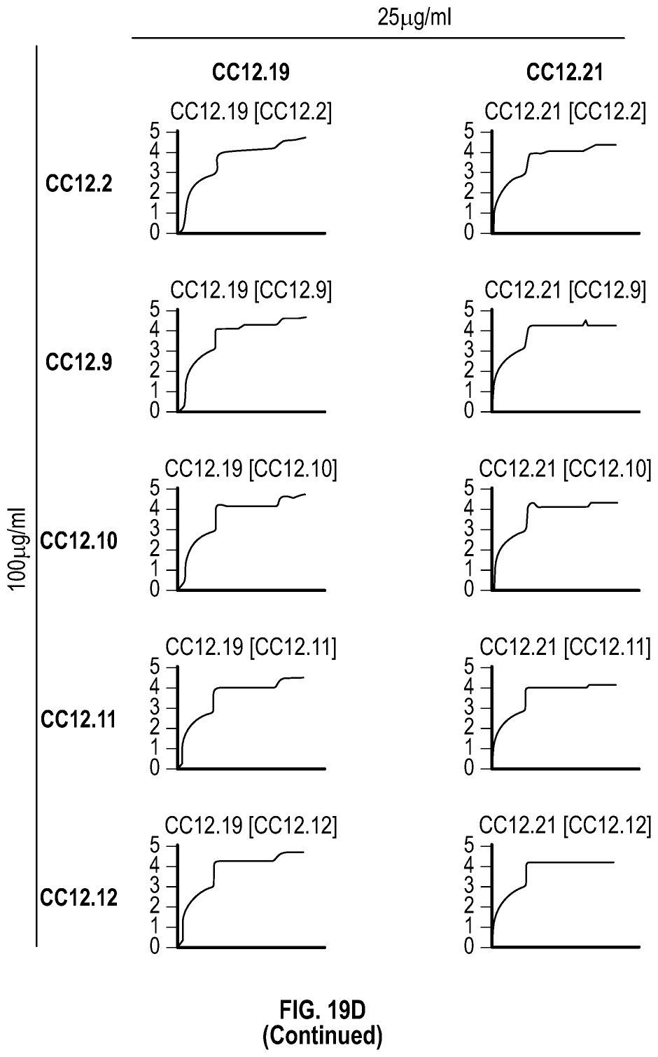

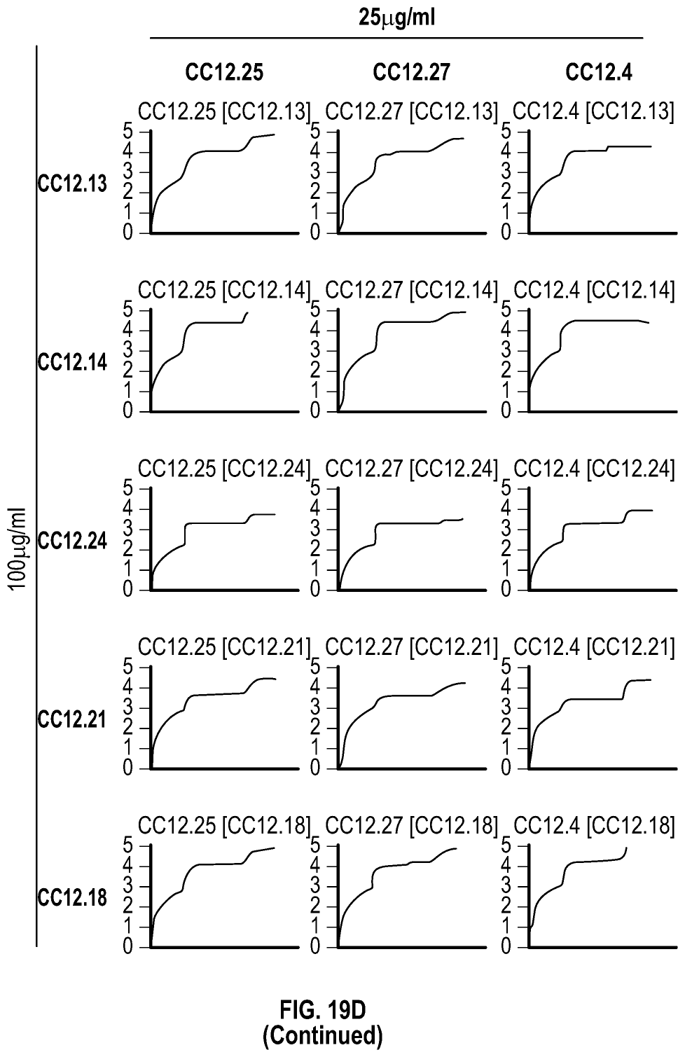

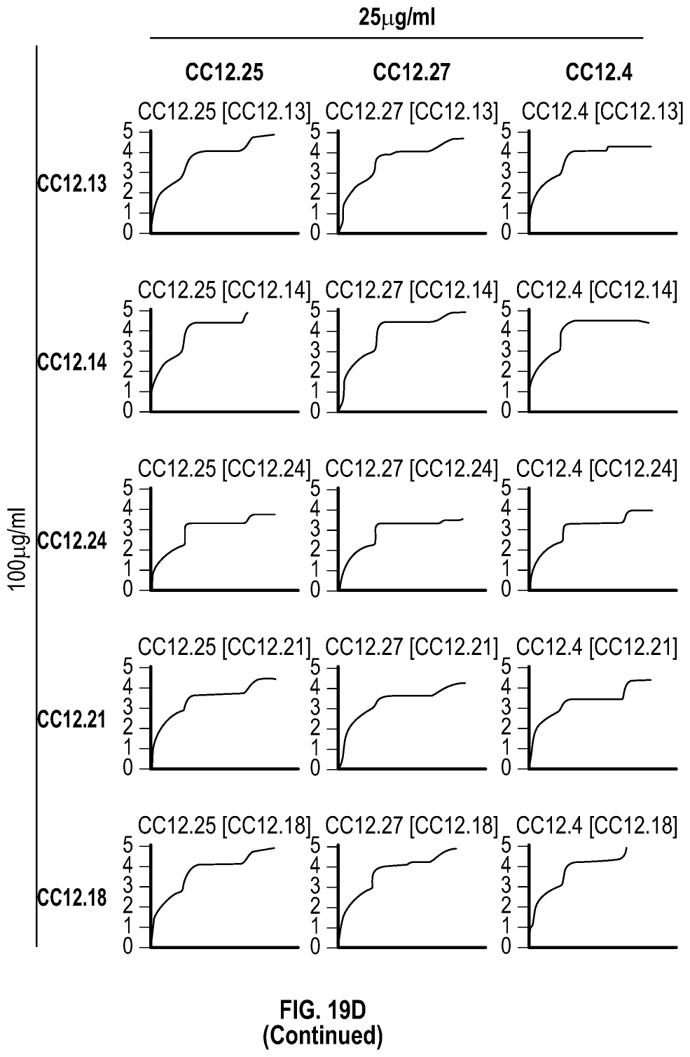

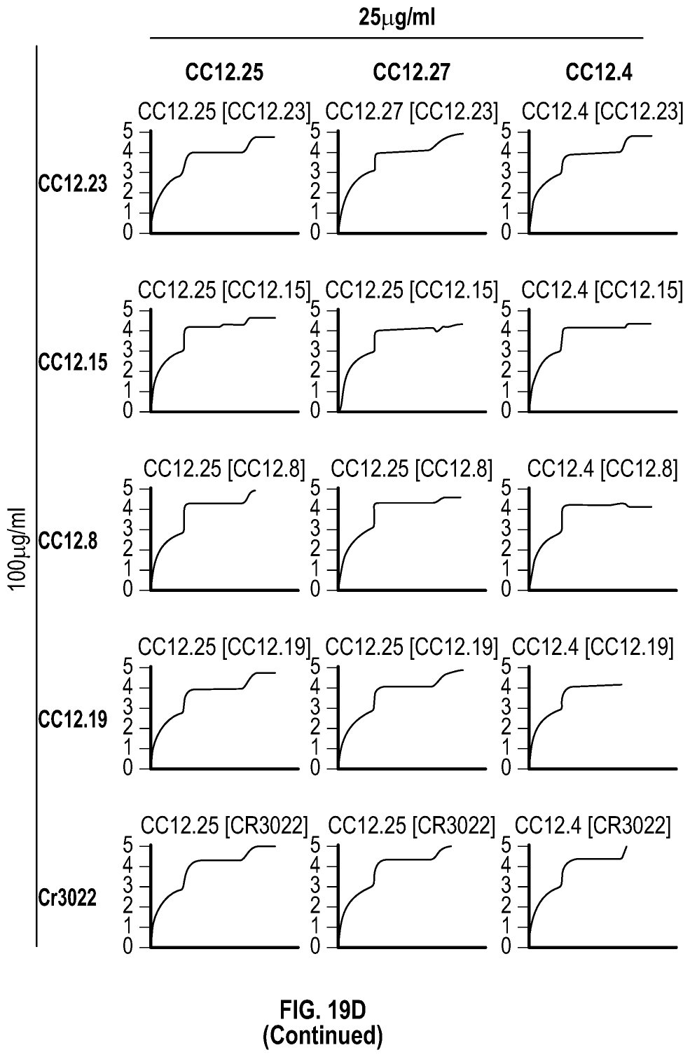

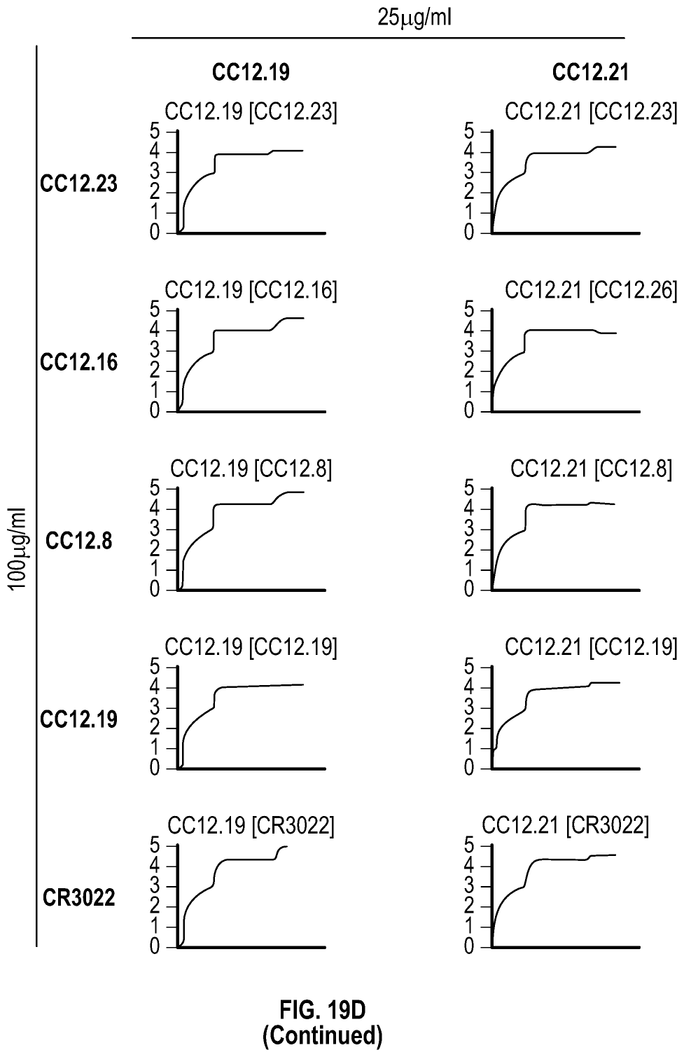

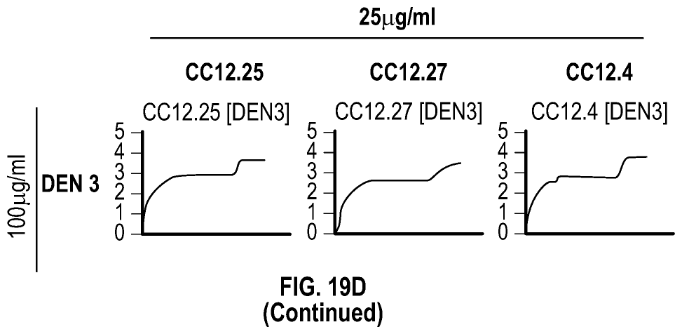

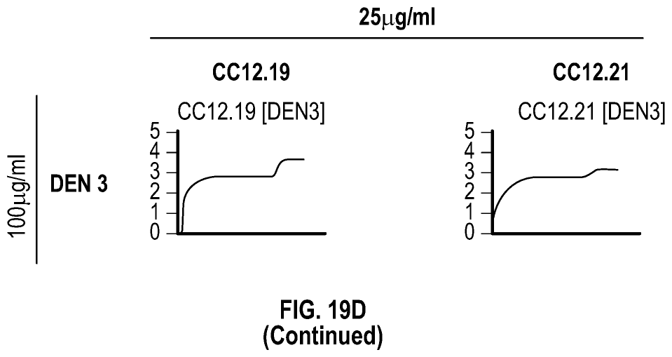

Cloned H+L chain pairs isolated from SARS-2 specific single B-cells were transfected into a high efficiency expression cell line. Small-scale culture supernatants were harvested at day 5-post transfection and evaluated for the presence of IgG, binding to recombinant SARS-CoV-2 S-protein and RBD subunit as well as for pseudotyped SARS-CoV-2 neutralization. ELISA considered positive when OD405 nm was >0.5 (dotted line). Results are plotted to show the proportion of expressed, binding and neutralizing pairs. Correlation between ELISA binding signal (OD 405 nm) and corresponding sorted cell staining level (MFI) for each antigenic bait (SARS-CoV-2 S-protein or RBD) (FIG. 18).

### Example: 31

Statistical Methods

To compute neutralization IC50 and binding EC50 values, 5-parameter logistic regression (sigmoidal) curves were fit using Python and the SciPy package or with Graphpad Prism. For neutralization data, curve fits were bounded at 0 and 100. For pairwise feature comparisons (binding versus neutralization, for example), linear regressions were calculated in Python using the statsmodels package or in Graphpad Prism. Confidence intervals of the regression were computed by bootstrap resampling, with associated R2 and p-values computed in Python using the SciPy package or computed using Graphpad Prism. For animal protection studies, significance between the groups was evaluated with Mann-Whitney U-tests using a 95% confidence interval.

### Example: 32

SARS-CoV-2-RBD Binding to Antibodies Via a Fccapture Multi-Cycle Method

The antibodies targeting the RBD-A epitope compete best against the ACE2 receptor and the neutralization IC50 correlates well with the percent competition for ACE2 receptor binding for both S protein and for RBD. The affinity of all RBD-specific antibodies to soluble RBD by surface plasmon resonance (SPR) was also assessed a poor correlation between affinity and neutralization potency was found (Table 12). However, the correlation is higher when limited to antibodies targeting the RBD-A epitope.

TABLE 12

| mAb ID | Method | Analyte | Fit Model | ka [1/Ms] | kd [1/s] | KD [M] |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | — | — | ND |
| [symbol] | [symbol] | [symbol] | [symbol] | — | — | ND |
| [symbol] | [symbol] | [symbol] | [symbol] | [symbol] | [symbol] | [symbol] |
| [symbol] | [symbol] | [symbol] | [symbol] | — | — | ND |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | — | — | ND |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | — | — | ND |
| [symbol] | [symbol] | [symbol] | [symbol] | — | — | ND |
| [symbol] | [symbol] | [symbol] | [symbol] | — | — | ND |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

[symbol]

indicates data missing or illegible when filed

### Example: 33

Sequencing of these antibodies identified 25 distinct lineages, with 23 containing a single member (Table 13). VH1 and VH3-gene families were notably prominent in these antibodies and there was a diversity of CDR3 lengths.

TABLE 13

Sequence characteristics

| | | % SH [symbol] | | %SHM |
|---|---|---|---|---|
| mAD ID | VHG [symbol] | (n [symbol] ) | VL Gene | (n [symbol] ) |
| CC12.1 | IGHV [symbol] | 1.05 | [symbol] | [symbol] |
| CC12.2 | IGHV [symbol] | 0.70 | [symbol] | [symbol] |
| CC12.3 | IGHV [symbol] | 1.40 | [symbol] | [symbol] |
| CC12.4 | IGHV [symbol] | 1.74 | [symbol] | 1.74 |
| CC12.5 | IGHV [symbol] | [symbol] | [symbol] | 0.47 |
| CC12.6 | IGHV [symbol] | 4.17 | [symbol] | 1.74 |
| CC12.7 | IGHV [symbol] | [symbol] | [symbol] | [symbol] |
| CC12.8 | IGHV [symbol] | 3.12 | [symbol] | 1.04 |
| CC12.9 | IGHV [symbol] | 3.47 | [symbol] | [symbol] |
| CC12.10 | IGHV [symbol] | 4.51 | [symbol] | 4.17 |
| CC12.11 | IGHV [symbol] | 3.47 | [symbol] | 1.74 |
| CC12.12 | IGHV [symbol] | 3.47 | [symbol] | [symbol] |
| CC12.13 | IGHV [symbol] | 0.35 | [symbol] | [symbol] |
| CC12.14 | IGHV [symbol] | 3.47 | [symbol] | [symbol] |
| CC12.15 | IGHV [symbol] | 1.04 | [symbol] | 1.04 |
| CC12.16 | IGHV [symbol] | 1.74 | [symbol] | [symbol] |
| CC12.17 | IGHV [symbol] | 1.39 | [symbol] | [symbol] |
| CC12.18 | IGHV [symbol] | 0.00 | [symbol] | [symbol] |
| CC12.19 | IGHV [symbol] | 1.04 | [symbol] | [symbol] |
| CC12.20 | IGHV [symbol] | 1.04 | [symbol] | [symbol] |
| CC12.21 | IGHV [symbol] | [symbol] | [symbol] | [symbol] |
| CC12.22 | IGHV [symbol] | [symbol] | [symbol] | [symbol] |
| CC12.23 | IGHV [symbol] | [symbol] | [symbol] | [symbol] |
| CC12.24 | IGHV [symbol] | 1.04 | [symbol] | [symbol] |
| CC12.25 | IGHV [symbol] | 0.35 | [symbol] | [symbol] |
| CC12.26 | IGHV [symbol] | 0.35 | [symbol] | 1.04 |
| CC12.27 | IGHV [symbol] | 1.04 | [symbol] | 0.35 |
| CC6.29 | IGHV [symbol] | [symbol] | [symbol] | [symbol] |
| CC6.30 | IGHV [symbol] | [symbol] | [symbol] | [symbol] |
| CC6.31 | IGHV [symbol] | [symbol] | [symbol] | 0.72 |
| CC6.32 | IGHV [symbol] | [symbol] | [symbol] | 2.15 |
| CC6.33 | IGHV [symbol] | 1.74 | [symbol] | 0.71 |

[symbol]

indicates data missing or illegible when filed

### Example: 34

Hamster Passive Immunization Study Summary

To investigate the relationship between in vitro neutralization and protection in vivo against SARS-CoV-2, we selected two mAbs for passive transfer/challenge experiments in a Syrian hamster animal model based on a summary of the nAb data (Table 14A and Table 14B). The experimental design for the passive transfer study is shown in FIG. 8A. In the first experiment, nAb CC12.1 was tested, which targets the RBD-A epitope and has an in vitro IC50 neutralization of 0.019 μg/mL against pseudovirus and in the second nAb C12.23 was tested, which targets the S-B epitope with an IC50 neutralization of 22 Ag/mL. In both experiments an unrelated antibody to dengue virus, Den3, was used as a control. The anti-SARS-CoV-2 nAbs were delivered at 5 different concentrations to evaluate dose-dependent protection starting at 2 mg/animal (average of 16.5 mg/kg) at the highest dose and 8 μg/animal at the lowest dose. The Den3 control antibody was delivered at a single dose of 2 mg/animal. Sera were collected from each animal 12 hours post IP infusion of the antibody and all animals were subsequently challenged with a dose of 1×106 PFU of SARS-CoV-2 (USA-WA1/2020) by intranasal administration 12 h post antibody infusion

TABLE 14A

| | [symbol] | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] |

[symbol]

indicates data missing or illegible when filed

TABLE 14B

| | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |
| [symbol] | [symbol] | [symbol] | | [symbol] | [symbol] | | [symbol] | | | | [symbol] |

[symbol]

indicates data missing or illegible when filed

### Example: 35

Hamster Passive Immunization Study Statistics

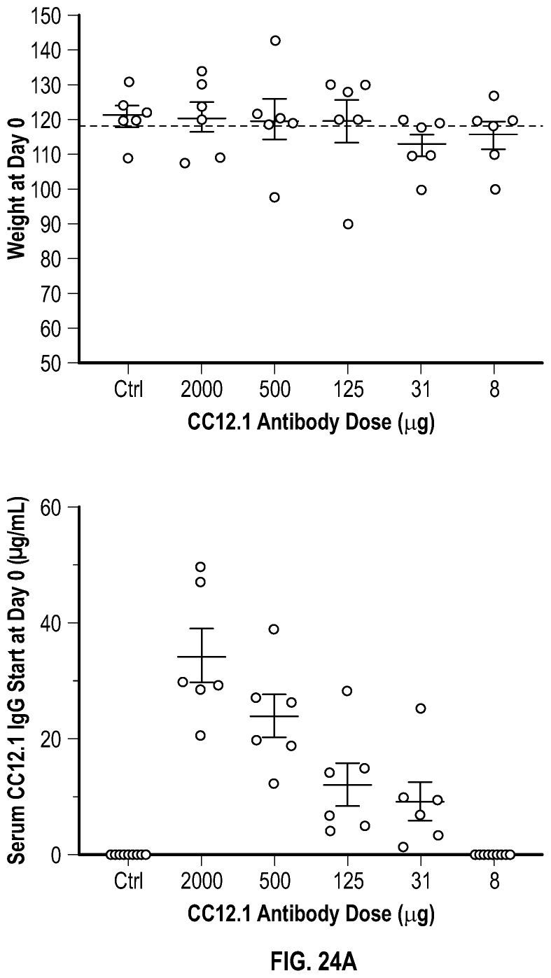

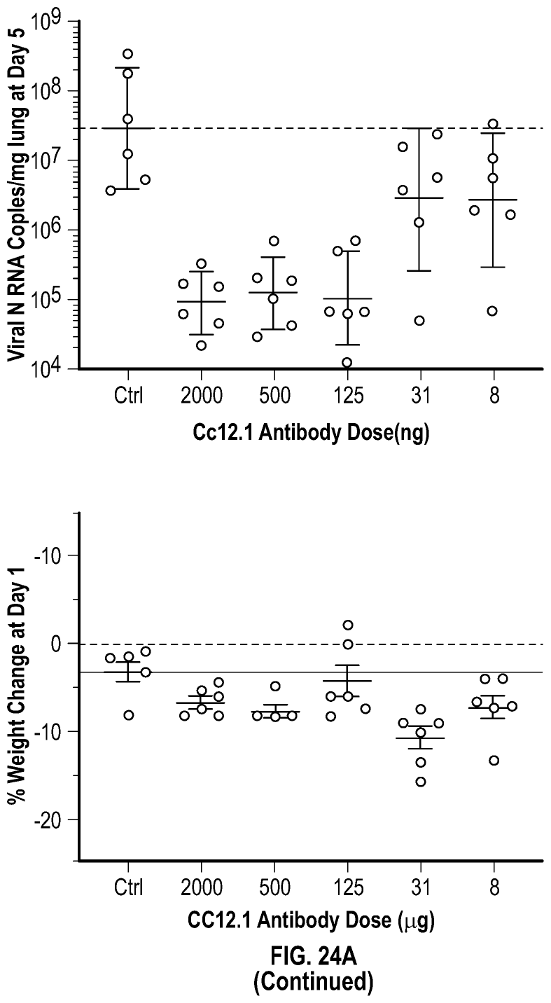

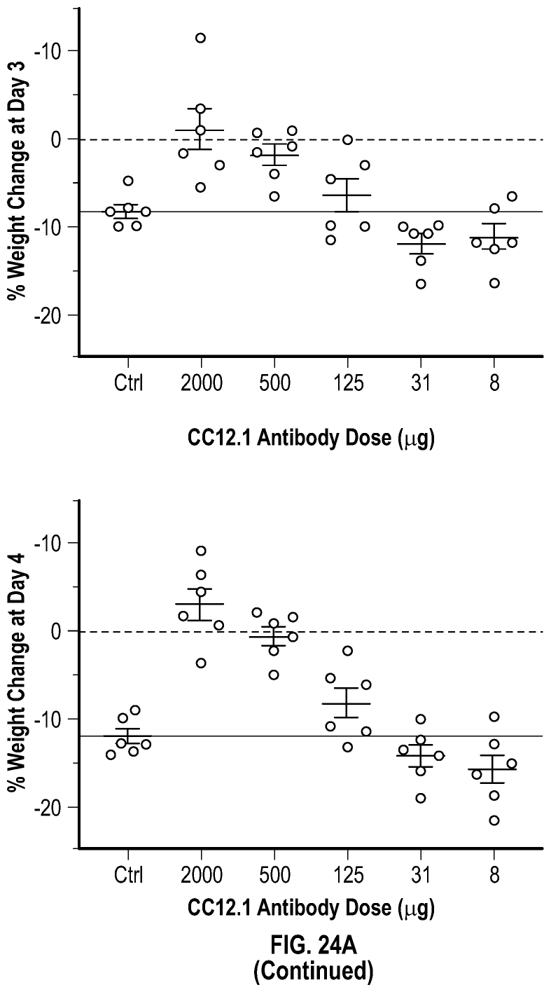

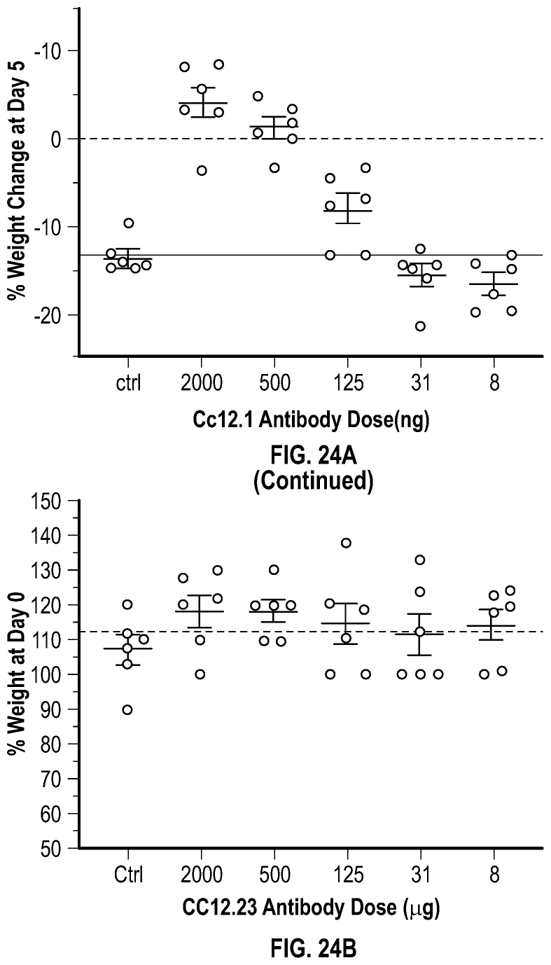

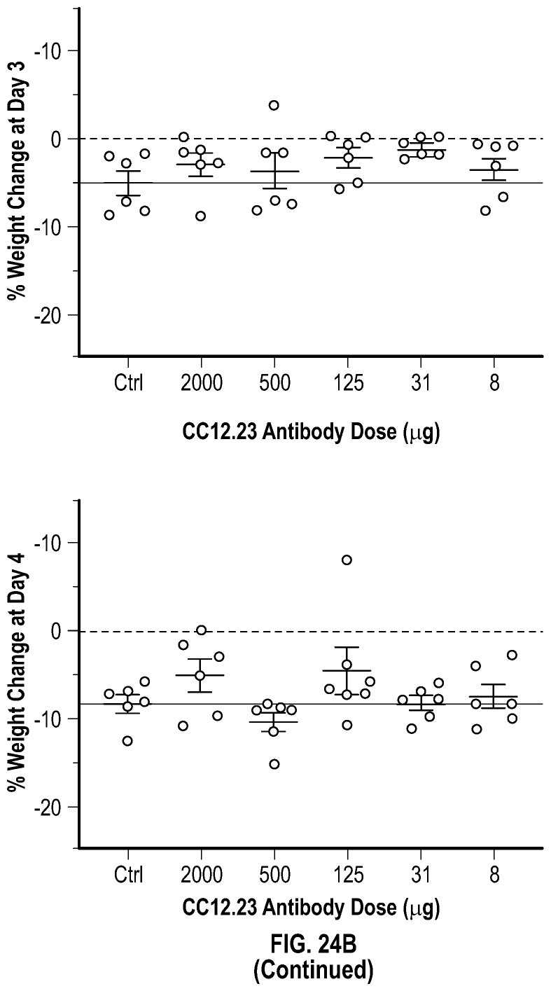

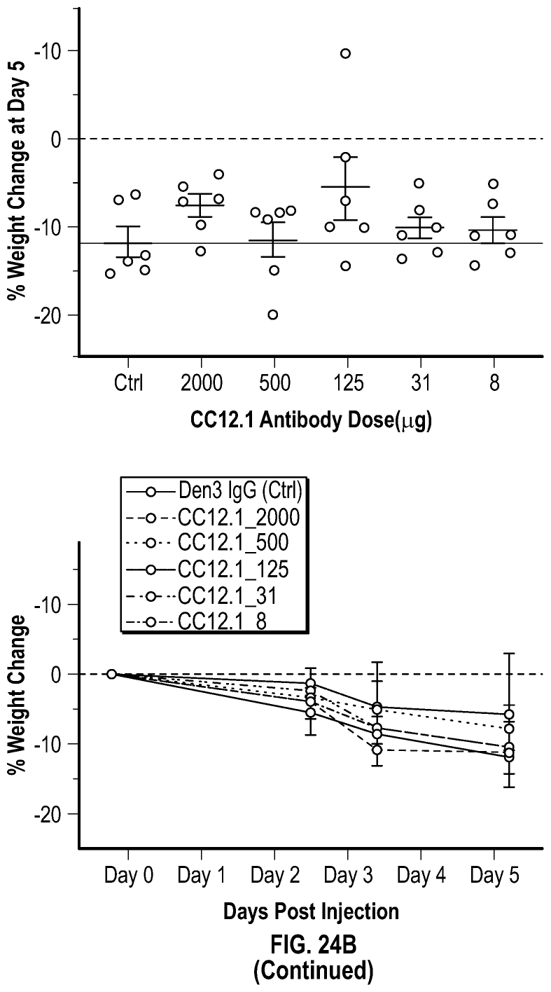

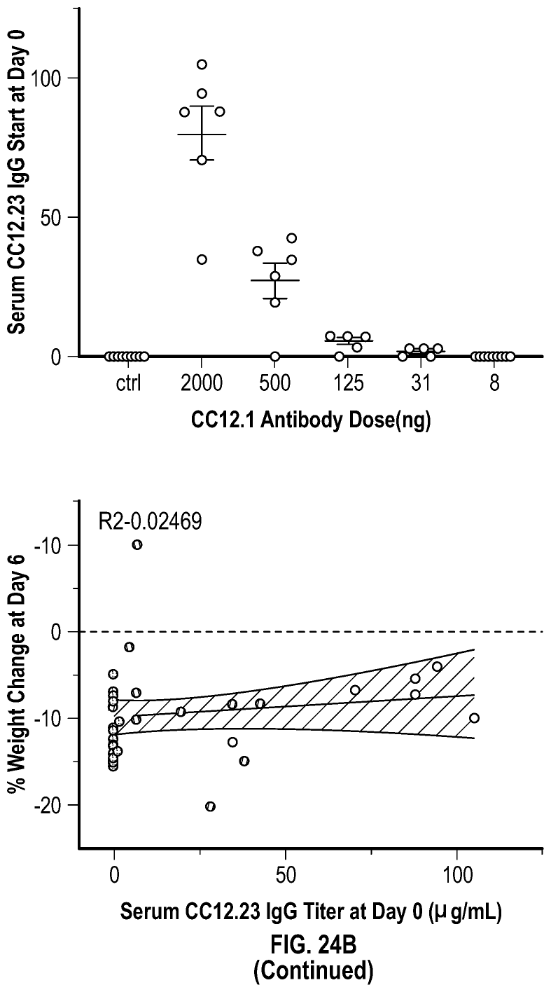

Syrian hamsters typically clear virus within one week after SARS-COV-1 infection. Accordingly, the hamsters were weighed as a measure of disease due to infection. Lung tissues were collected to measure viral load on day 5 (FIG. 24). A data summary is presented herein for animals that received CC12.1, which targets the RBD-A epitope. The control animals that received Den3 lost on average 13.6% of body weight at 5 days post virus challenge. In comparison, the animals that received the neutralizing RBD-A antibody at a dose of 2 mg (average of 16.5 mg/kg) or 500 μg (average of 4.2 mg/kg) exhibited no weight loss. However, animals that received a dose of 125 μg (average of 0.9 mg/kg) had an average 8% loss of body weight, while animals that received a dose of 31 μg/ml (0.2 mg/kg) and $ μg/ml (0.06 mg/kg) lost 15.8% and 16.7% of body weight, respectively. We note these animals showed a trend for greater weight loss than control animals but this did not achieve statistical significance (Table 15A and Table 15B).

TABLE 15A

| | | | [symbol] | | | | | | | | | | | | | |
|---|---|---|---|---|---|---|---|---|---|---|---|---|---|---|---|---|
| | | | [symbol] | | [symbol] | | [symbol] | | [symbol] | | [symbol] | | [symbol] | | [symbol] | |
| [symbol] | | [symbol] | | [symbol] | | [symbol] | | [symbol] | | [symbol] | | [symbol] | | [symbol] | | [symbol] |
| [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

TABLE 15B

| | | | [symbol] | | | | | | | | | |
|---|---|---|---|---|---|---|---|---|---|---|---|---|
| | | | [symbol] | | [symbol] | | [symbol] | | [symbol] | | [symbol] | |
| [symbol] | | [symbol] | | [symbol] | | [symbol] | | [symbol] | | [symbol] | | [symbol] |
| [symbol] | | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] |

[symbol]

indicates data missing or illegible when filed

### Example: 36

Autoreactivity Staining Assay

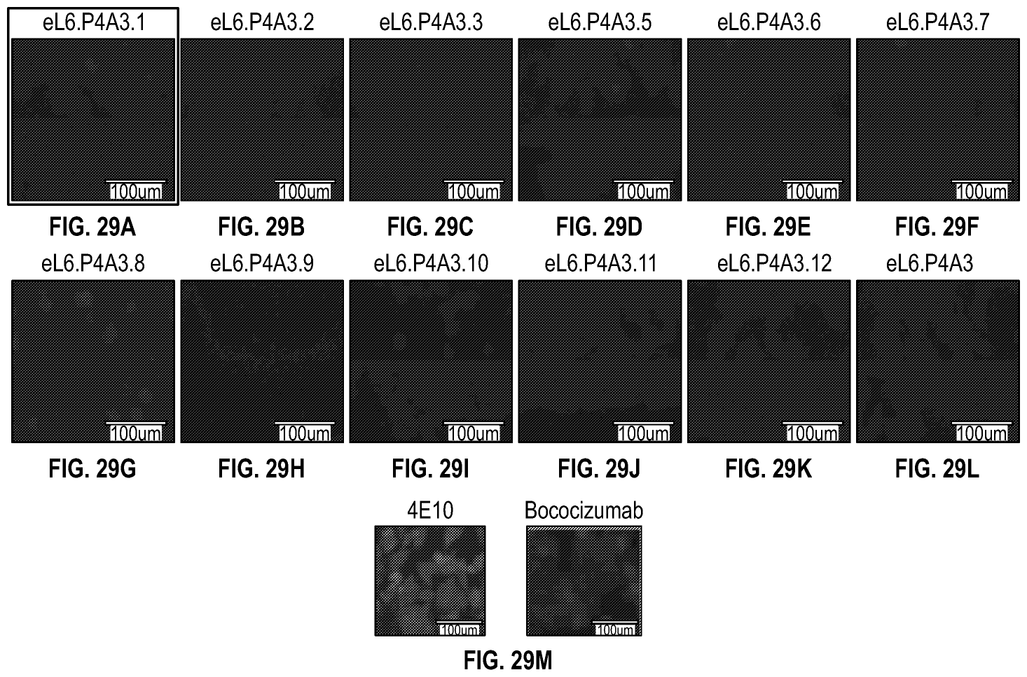

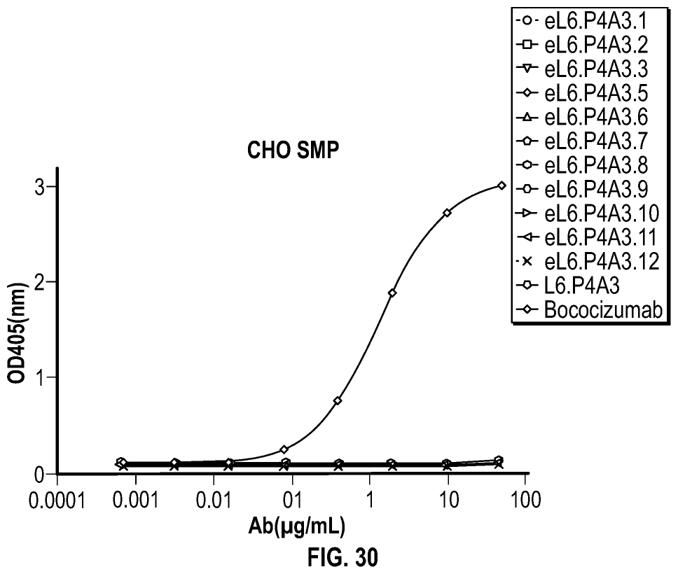

Autoreactivity staining assays were performed on human epithelial type 2 (HEp-2) cells per the manufacturer recommendations (Aesku Diagnostics, Oakland, CA). These Aesku slides use optimally fixed human epithelial (HEp-2) cells (ATCC) as substrate and affinity purified, FITC-conjugated goat anti-human IgG for the detection. Briefly, 2.5 μg or 25 μl of 100 μg/ml mAb and controls were added to wells and incubated on HEp-2 slides in a moist chamber at room temperature for 30 min. Slides were then rinsed and submerged in PBS and 25 μl of FITC-conjugated goat anti-human IgG was immediately applied to each well. Slides were allowed to incubate at room temperature in a moist chamber for another 30 min. Slides were then washed in the same manner as above and then mounted on coverslips using the provided mounting medium. Slides were viewed at 20× magnification and photographed on an EVOS f1 fluorescence microscope at a 250 ms exposure with 100% intensity. Antibodies 4E10 and Bococizumab were included as positive control. Data indicate antibodies are not polyreactive in the HEp-2 assay relative to 4E10 positive controls (FIG. 29A-L).

### Example: 37

Affinity Maturation of Antibodies

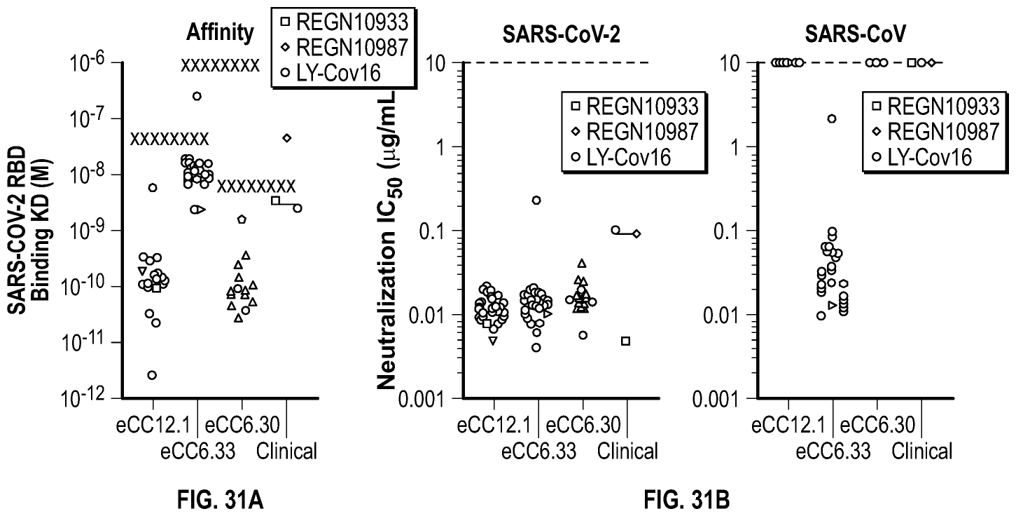

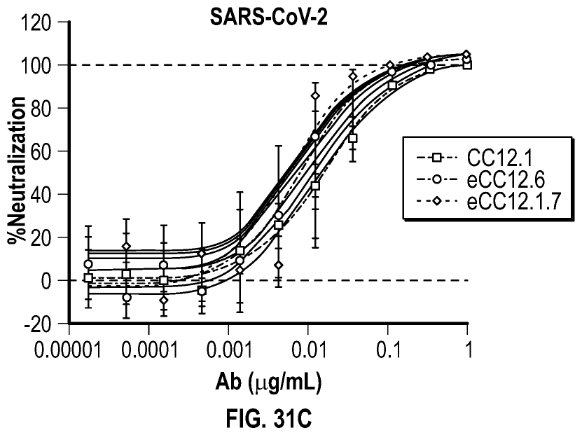

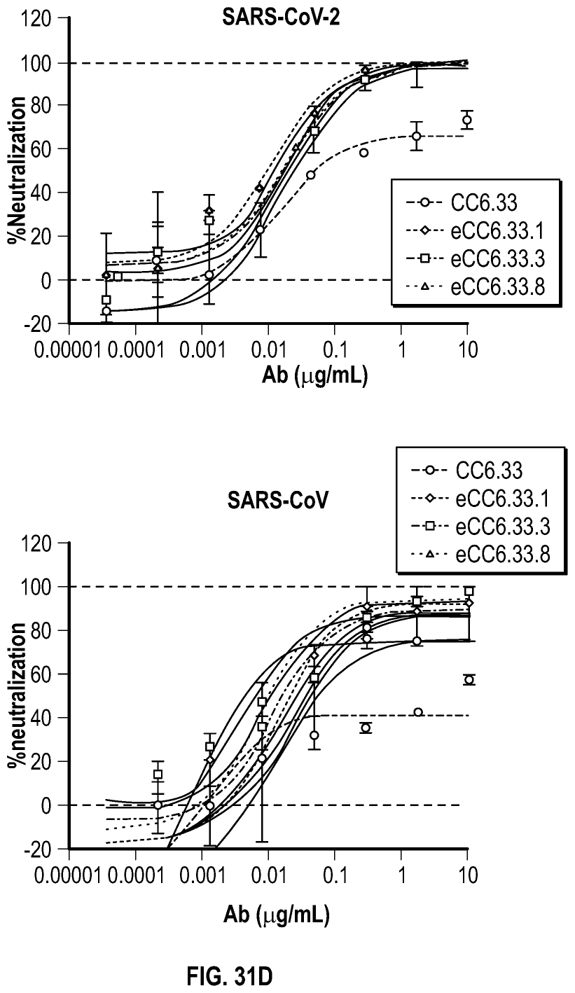

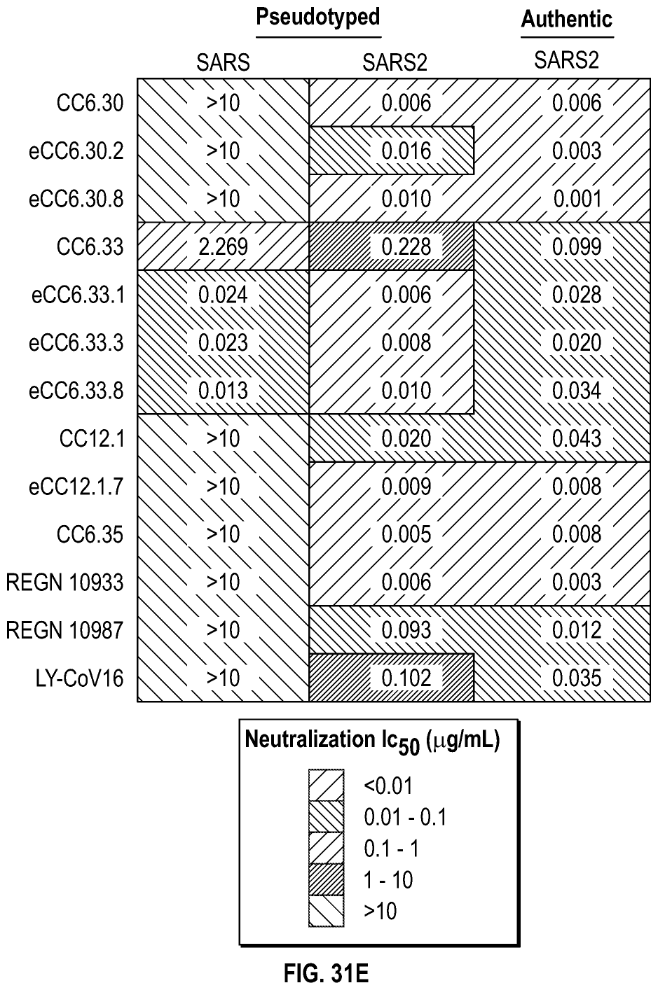

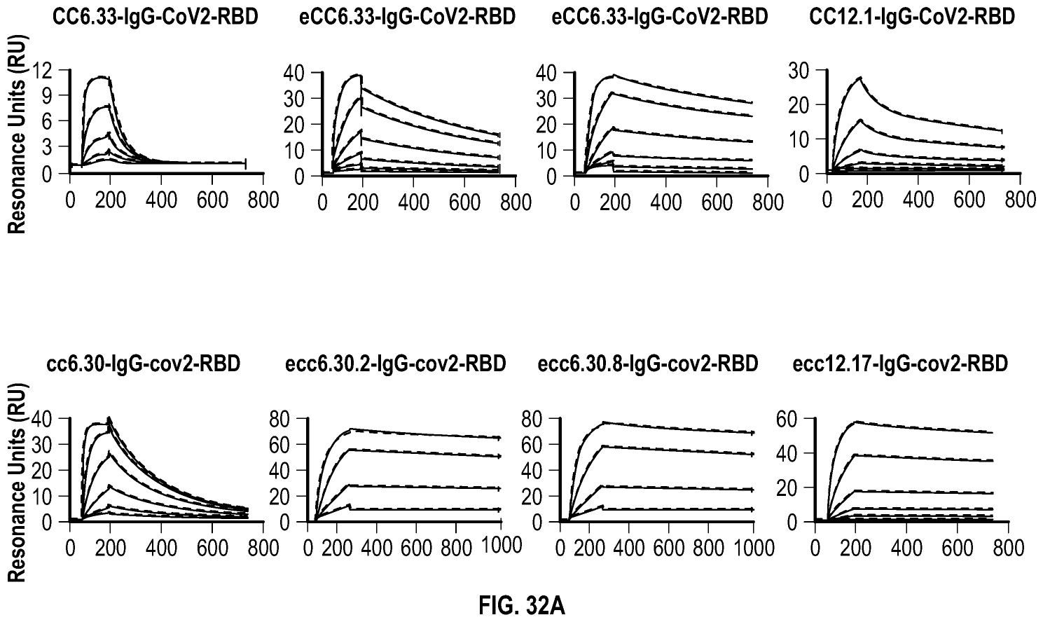

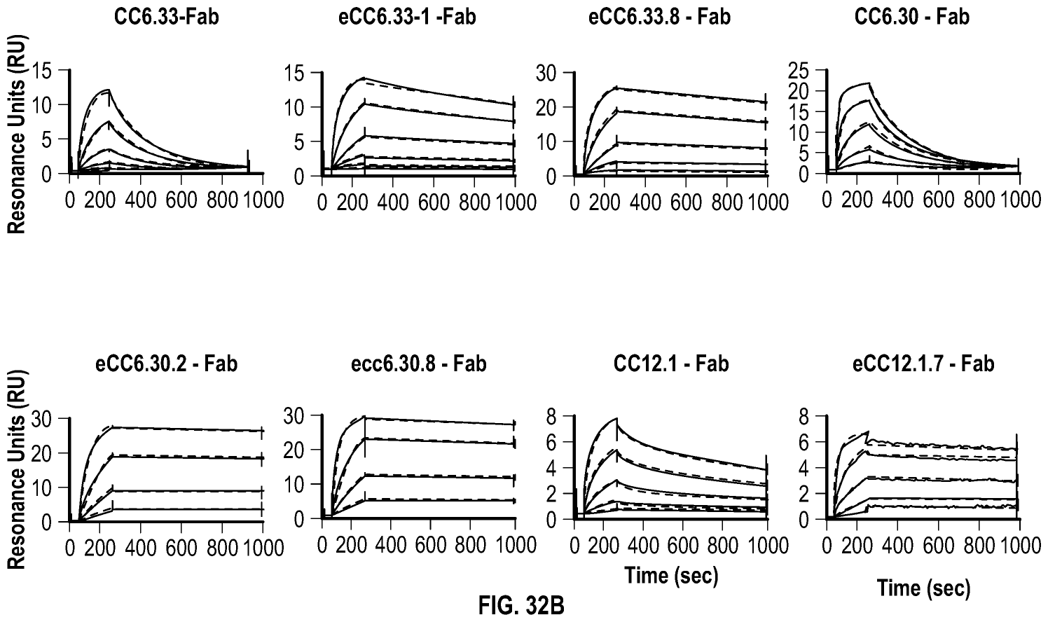

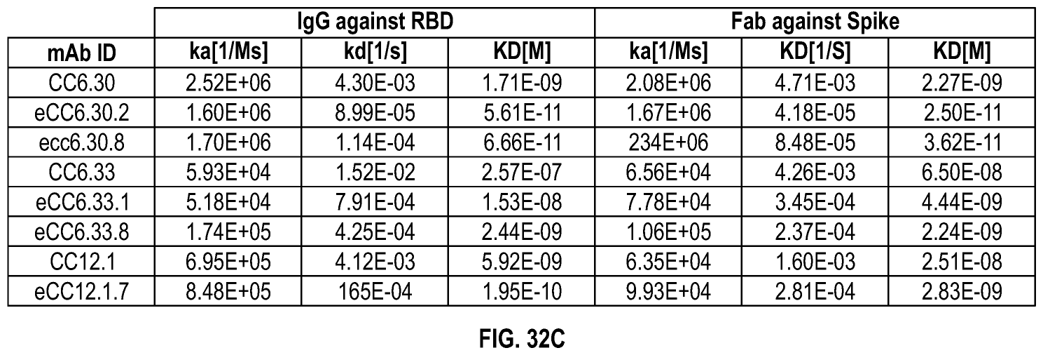

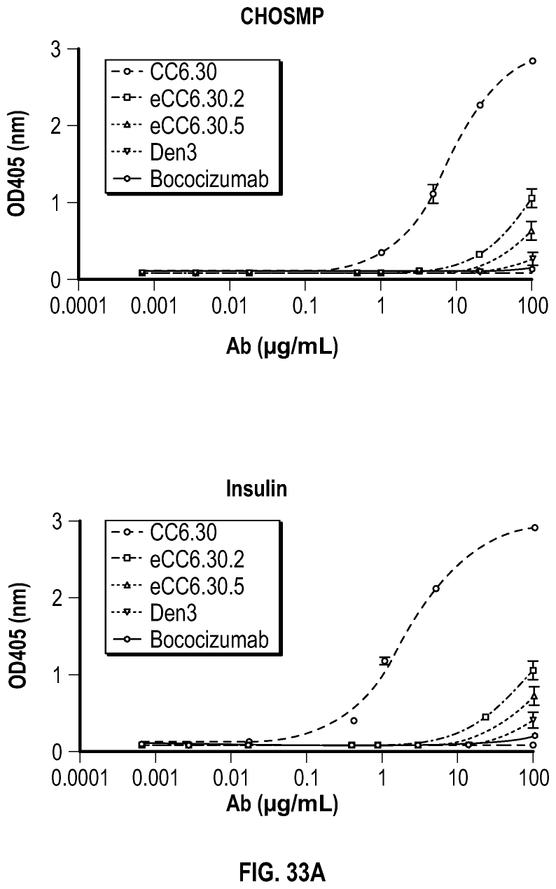

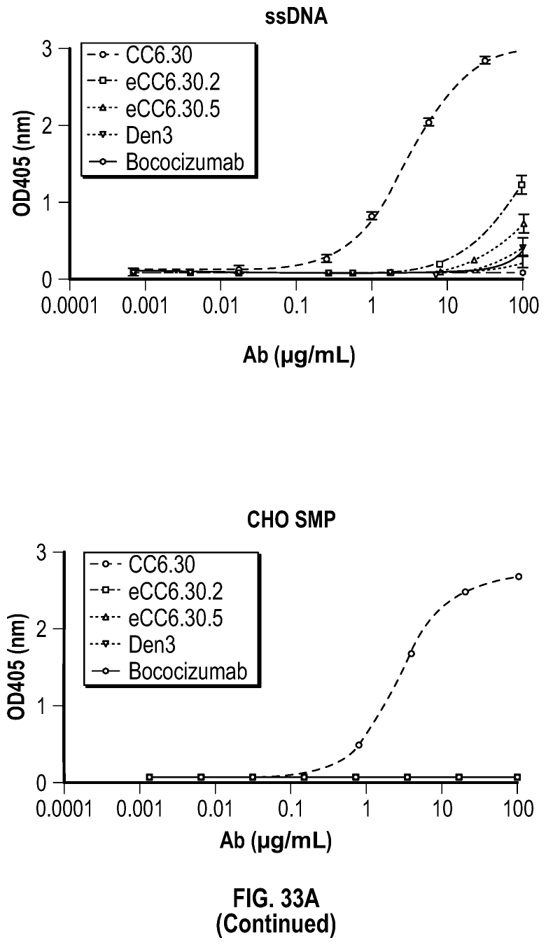

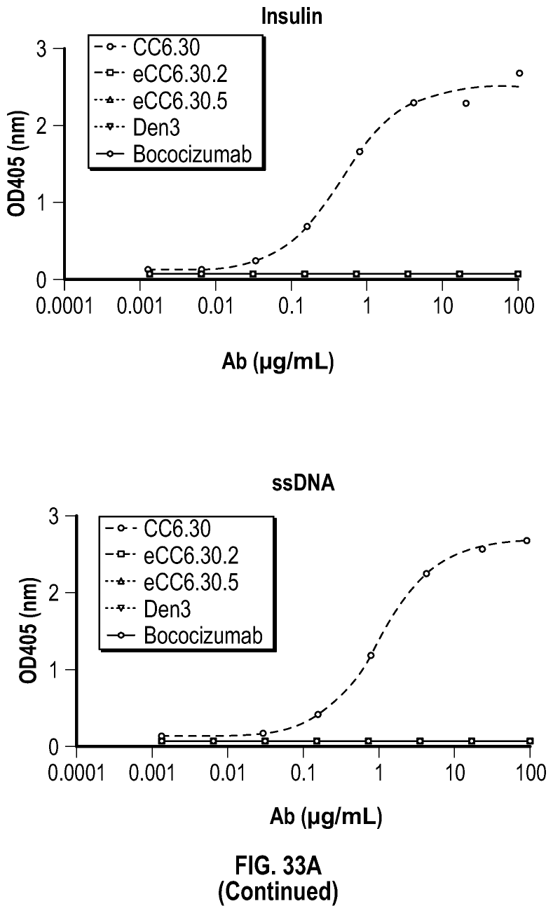

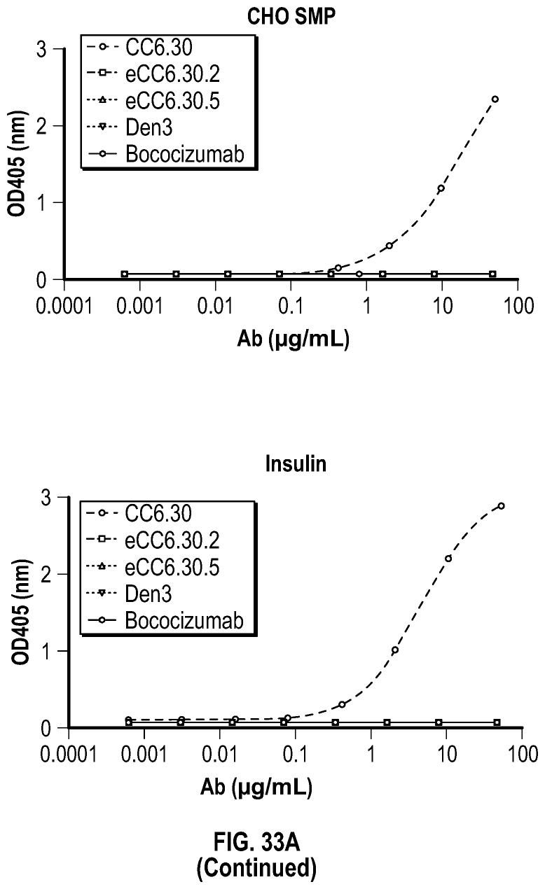

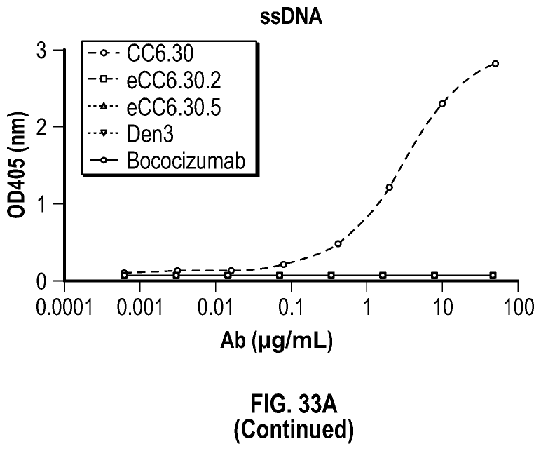

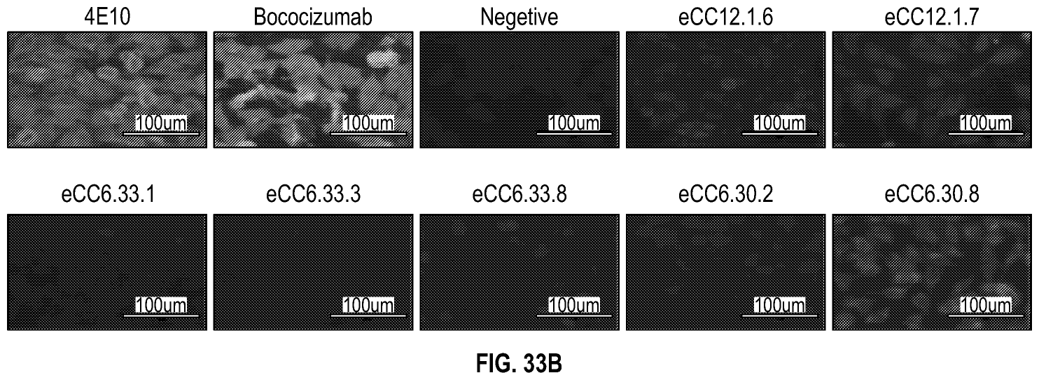

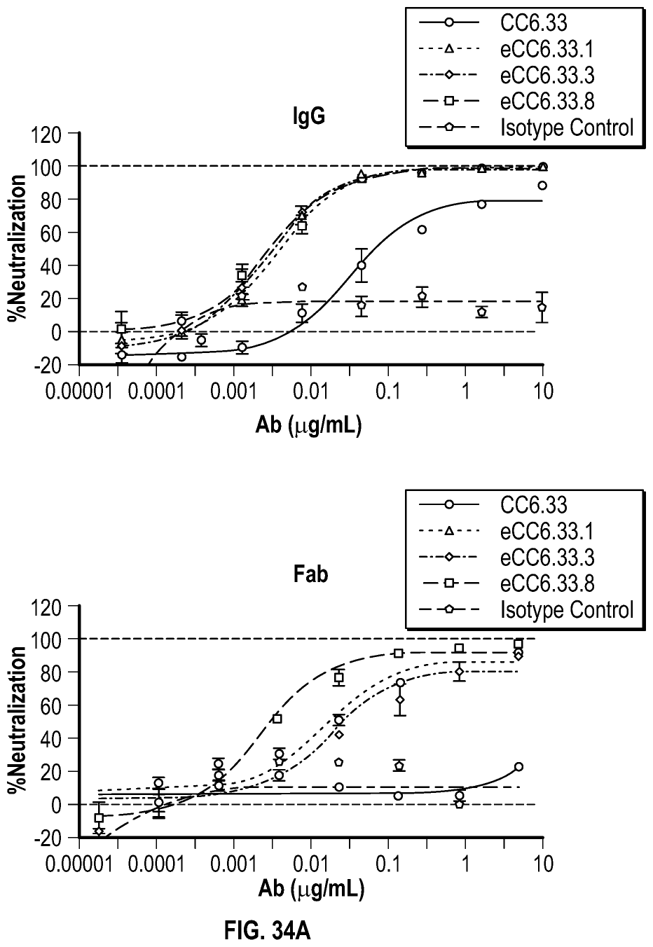

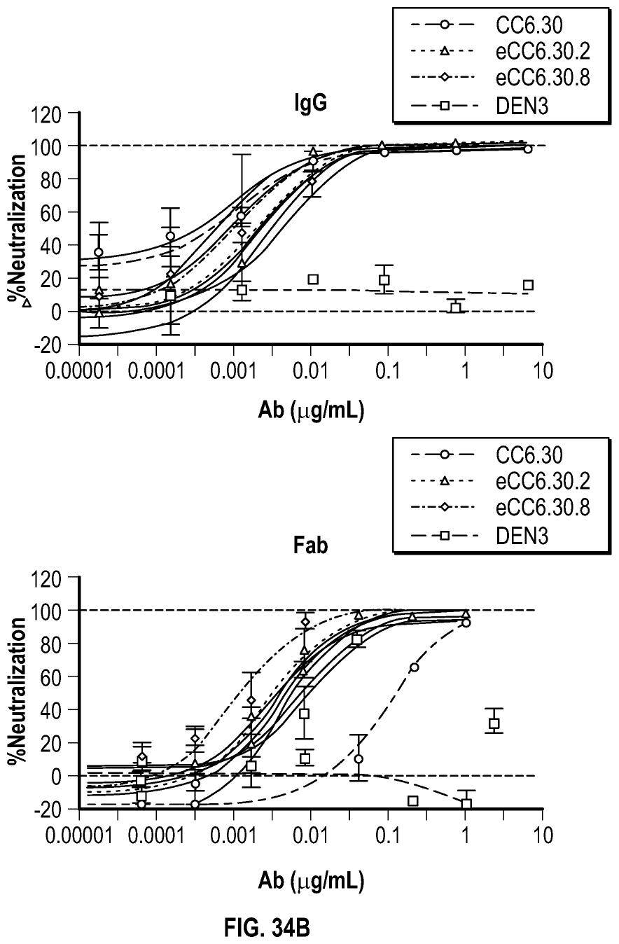

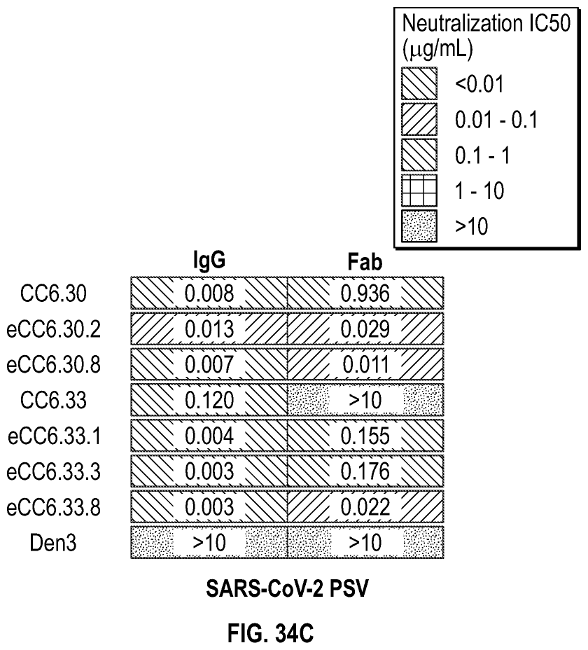

To explore the relationship between binding affinity, in vitro neutralization, and in vivo protection againsts SARS-CoV-2, neutralizing antibodies against SARS-CoV-2 were affinity matured using a rapid maturation strategy. Briefly, rationally designed heavy chain and light chain libraries were synthesized containing one mutation per CDR loop from the starting sequence, for up to three mutations per chain. Potential liabilities were informatically filtered from the library process and an N-linked glycan at in the CDR-L1 of CC6.30 was removed by a mutation, reverting that position to the original amino acid, so that any improved CC6.30 variant would not contain that glycan. The heavy chain and light chain library were displayed on the surface of yeast and iterative rounds of selections were used to enrich for clones with higher affinity for SARS-CoV-2 RBD or S. The sort process also included a round of negative selection, where clones with low binding to a polyclonal preparation of detergent solubilized HEK293 cell membrane proteins were enriched to remove polyreactive variants. The enriched clones were then combined into a heavy/light combinatorial library and screened again with the same four round selection strategy to identify the optimal heavy/light pairs. At the conclusion of the selection process, sequences of the antibodies were recovered and 12 improved variants from each library were selected to be reformatted and expressed as IgG for characterization. All enhanced CC12.1 (eCC12.1) and enhanced CC6.30 (eCC6.30) variants that recognize the RBD-A epitope bound to SARS-CoV-2 RBD with monovalent equilibrium dissociation constants (K_{D}s) in picomolar affinity relative to their parental clones (1.7 nM and 5.9 nM respectively) by surface plasmon resonance (SPR) (FIG. 31A and FIG. 32). The affinities of eCC6.33 variants to SARS-CoV-2 RBD increased up to 100 fold more than parental CC6.33 (K_{D} of 257 nM), ranging from 2.4 nM to 17.3 nM (FIG. 31A). ELISA binding to SARS-CoV-2 RBD and S of CC12.1 and CC6.30 parental and engineered nAbs was comparable, however, a large difference in EC₅₀ and the maximum plateau was observed for eCC6.33 variants compared to the CC6.33 parental (FIG. 32). None of the eCC12.1 or eCC6.33 variants bound to antigens in the polyreactive panel (Chinese hamster ovary cell solubilized membrane proteins, single-stranded DNA, and insulin) or stained HEp2 epithelial cells (FIG. 33). Several of the CC6.30 variants showed low levels of binding to one or more of the antigens in the polyreactivity panel or stained HEp2 cells, but the majority of engineered variants were clean in all assays, highlighting the importance of expressing and validating multiple variants. Taken together, several variants of each antibody with improved binding affinity and no off-target specificities.

Pseudovirus (PSV) Assay with Enhanced Antibodies

To evaluate the relationship between improved binding affinity and in vitro neutralization potency of the enhanced antibodies, the murine leukemia virus (MLV) pseudovirus system was used. All eCC6.33 variants showed improved neutralization potency against both SARS-CoV and SARS-CoV-2 pseudotyped viruses, neutralizing both with an IC50 of around 10 ng/mL and achieving complete neutralization (FIG. 31B). In contrast to the improvements observed with the eCC6.33 variants, the affinity matured eCC12.1 and eCC6.30 variants showed no significant change compared to the parental, with IC₅₀s again remaining at around 10 ng/mL (FIG. 31B). The fragment antigen binding (Fab) variants showed remarkably increased neutralization activity, as the most potent eCC6.30.8 Fab neutralized SARS-CoV-2 with 85-fold improved IC₅₀ (0.01 ug/mL) than parental CC6.30 Fab (0.94 ug/mL) (FIG. 32). The eCC6.33.8 Fab neutralized SARS-CoV-2 with an IC₅₀ of 0.02 ug/mL, whereas parental CC6.33 Fab failed to neutralize virus at the concentration of 50 ug/mL (FIG. 32). These data suggest that the neutralization potency of first generation SARS-CoV-2 nAbs is likely mediated by avidity, therefore displaying Fab on the surface of yeast results in enhanced binding affinity. Overall, the neutralization potency of eCC6.33, eCC12.1 and eCC6.30 variants was comparable to or better than clinical-stage nAbs: REGN10933, REGN10987, and LY-CoV16. In the authentic SARS-CoV-2 neutralization assay, engineered antibody variants also reached 100% neutralization on HeLa-hACE2 cells with higher potency than parental mAbs (FIG. 31E).

Antibody Neutralization of Circulating Variants

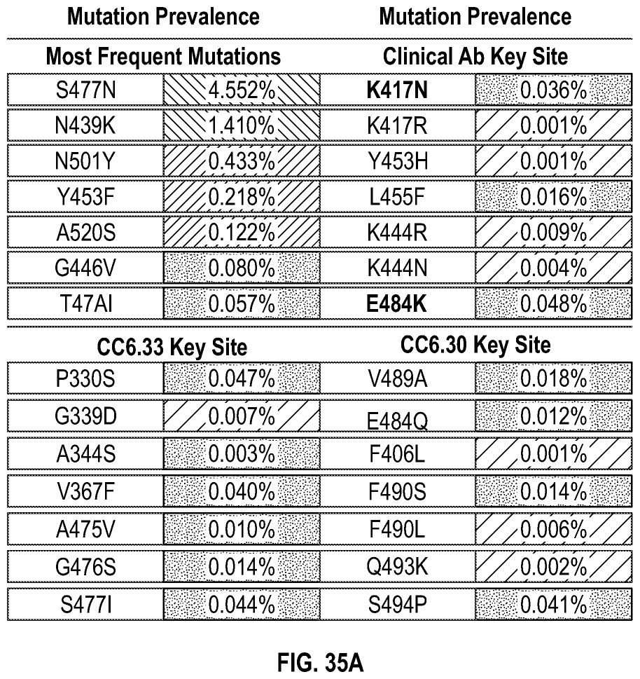

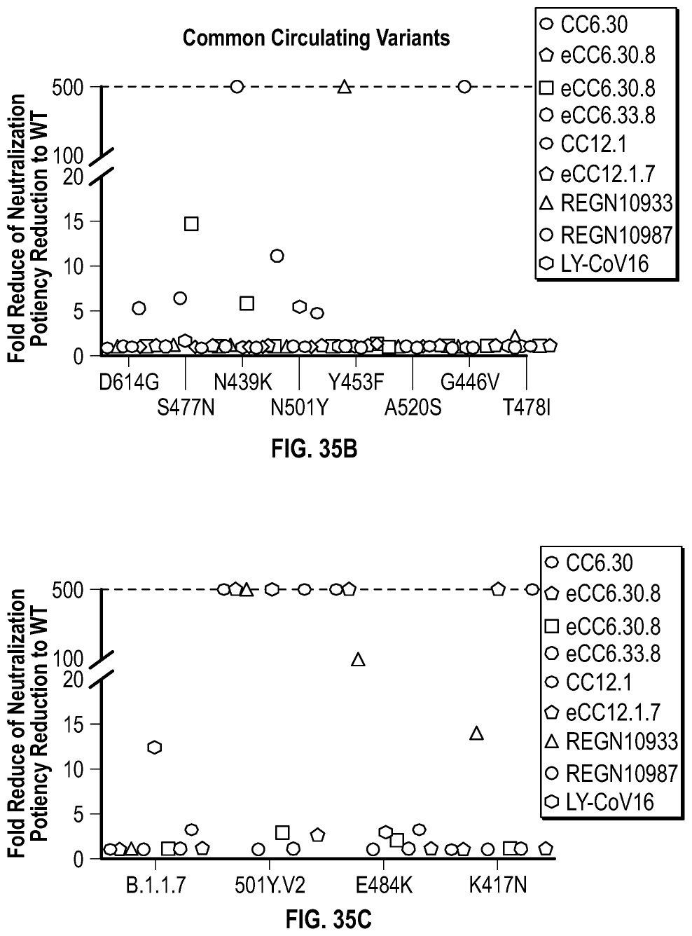

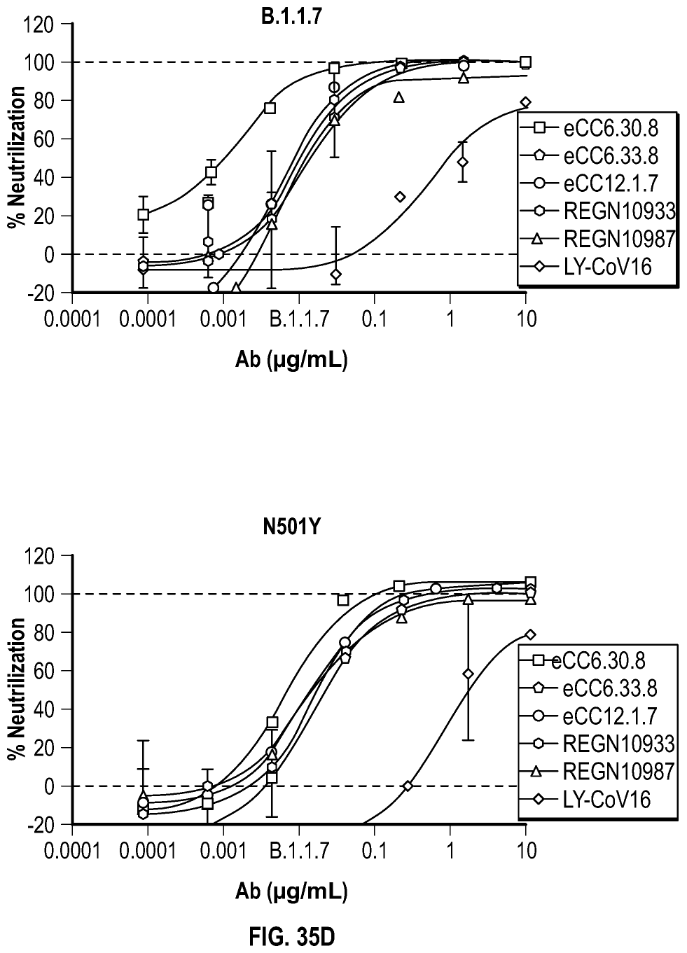

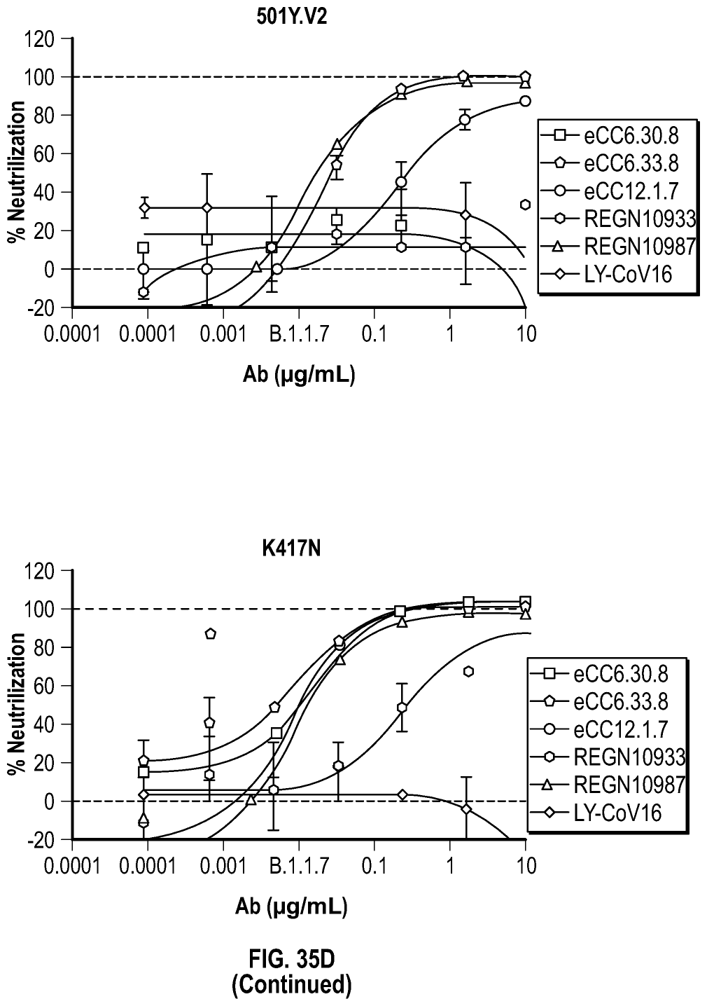

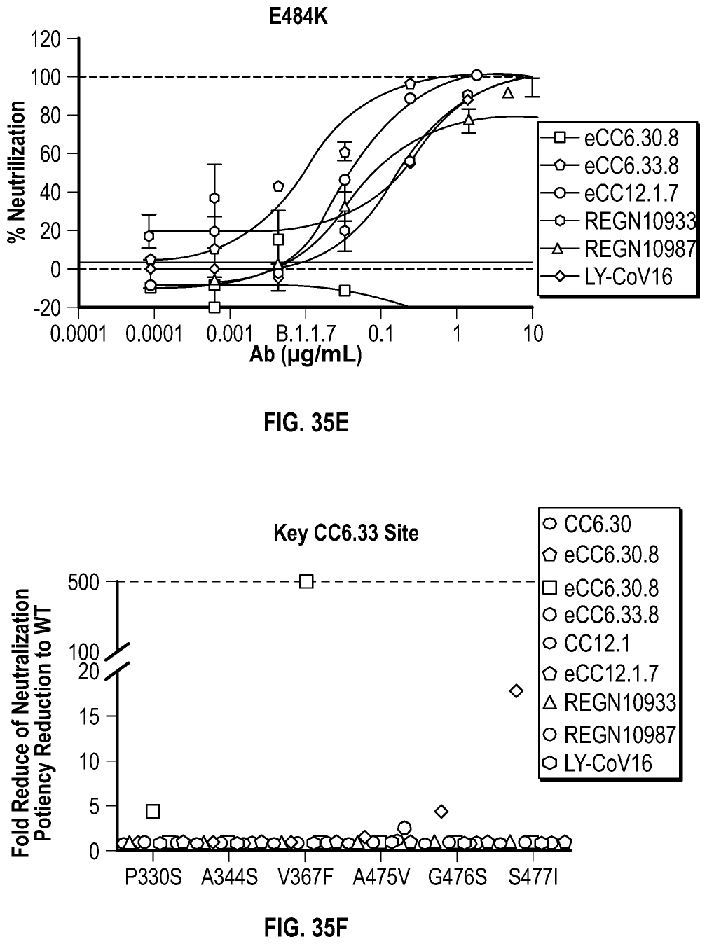

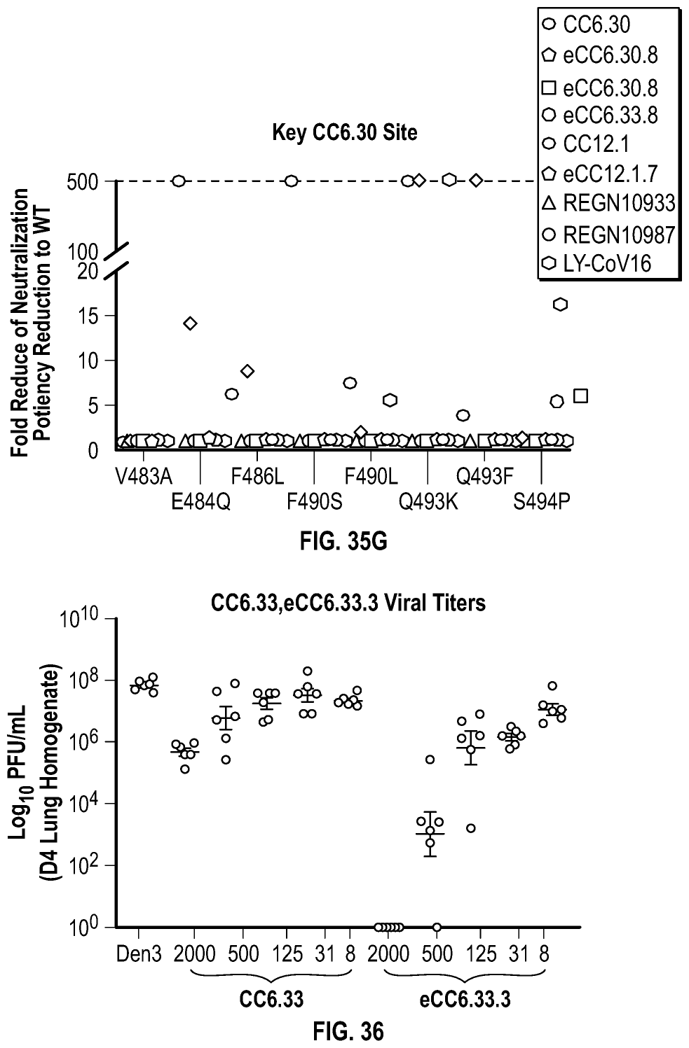

The evolution of SARS-CoV-2 with mutations in RBD could impair nAb recognition, which raises concerns for monoclonal antibody therapy and vaccine efficacy. Specifically, the most recent B.1.1.7 viral lineage emerging in UK (with N501Y mutation in RBD) and the novel variant 501Y.V2 collected in South Africa (with three mutations in RBD: N501Y, E484K, K417N) could potentially escape from antibody neutralization. The neutralization profile of parental and enhanced SARS-CoV-2 nAbs was examined in parallel with clinical-stage nAbs against pseudotyped SARS-CoV-2 variants. Seven of the most prevalent circulating variants from the GISAID database: S477N (4.522%), N439K (1.410%), N501Y (0.433%), Y453F (0.218%), A520S (0.122%), G446V (0.080%), and T4781 (0.057%) (FIG. 35A). The most dominant strain, D614G, was included as a control because the substitution is outside of SARS-CoV-2 RBD. As expected, the affinity-matured clones and clinical-stage nAbs neutralized D614G with equivalent potency as wild-type (WT) SARS-CoV-2 pseudovirus (FIG. 35B). No circulating variant with single amino acid mutation was able to escape all nAbs that recognize the same specificities. However, REGN10933 completely lost neutralizing ability against the Y453F variant while REGN10987 failed to neutralize the N439K and G446V variants (FIG. 35B). The neutralization potency of CC6.33 against N439K and S477N mutations reduced 5 to 15 fold respectively, but that of eCC6.33.8 remained unchanged. Next, the antibody neutralization activity against B.1.1.7 strain, 501Y.V2 strain, including E484K and K417N single mutation was investigated. The N501Y and 501Y.V2 variants didn't affect neutralization activity of nAbs (FIGS. 35B and 35C), with the exception of CC12.1 and LY-CoV16. This suggests that the N501Y mutation mostly improves transmissibility via increasing binding affinity against human ACE2. However, consistent with previous studies, 501Y.V2 strain completely eliminated neutralization from multiple RBD-A-targeting nAbs including LY-CoV-16, REGN10933. CC6.30, eCC6.30.8 and CC12.1 (FIGS. 35C and 35D). Particularly, the K417N (0.036% prevalence) mutation was resistant to CC12.1 and LY-CoV-16 while the E484K (0.048% prevalence) mutation was resistant to CC6.30 and REGN10933 (FIGS. 35D and 35E). The second generation CC12.1 variant eCC12.1.7, potently neutralized K417N and 501Y.V2, suggesting that the affinity matured SARS-CoV-2 nAb with improved affinity was resistant to potential escaped viruses. The neutralization activity against a panel of SARS-CoV-2 circulating variants with mutations within the key binding site of CC6.30, CC6.33 and clinical stage nAbs was further assessed (FIG. 35A). Parental antibody, CC6.33, failed to neutralize V367F (0.040% 5 prevalence) and G339D (0.007% prevalence) variants, but eCC6.33.8 maintained neutralization potency (FIG. 35F). Additionally, parental CC6.30 failed to neutralize E484Q (0.012%), F490S (0.014%), and Q493K (0.002%) variants, but neutralized F486L (0.008%) and F490L (0.006%) variants with 7 to 9 fold reduced potency relative to WT virus (FIG. 35G). The eCC6.30.8 nAb with improved affinity maintained potent neutralization against most of the escaped variants (FIGS. 35D and 35F), except variants having the E484K mutation, which has been observed in South Africa and Brazil. These variants also reduced REGN10933 neutralization potency.

Several mutations such as F486L, 0493K, Q493F, and S494P, albeit with low to medium prevalence, affected neutralization from RBD-A nAbs such as CC6.30, REGN10933 and LY-CoV16 but not RBD-B nAbs (FIG. 35G), indicating the requirement of antibody cocktail therapy to avoid viral escape. Similarly, Y453H and L455F mutations were resistant to REGN10933 while K444R and K444N mutations were resistant to REGN10987 (FIG. 35). In contrast, eCC12.1.7 and eCC6.33.8 variants were likely to neutralize all viral mutants.

These findings were confirmed in a subsequent neutralization experiment using the parental and enhanced nAbs against known SARS-CoV-2 variants of concern (Table 16).

TABLE 16

| mAb ID | D614G | B.1.1.7 | B.1.351 | N501Y | E484K | K417N | L452R | E484Q |
|---|---|---|---|---|---|---|---|---|
| L12-bP11A06 | 0.065 | 1.061 | >10 | 0.433 | 0.165 | >10 | 0.040 | 0.023 |
| L6-dP04A03 | 0.0757 | 0.435 | 0.443 | 0.075 | 0.128 | 0.019 | 0.225 | 0.174 |
| eL6-dP04A03.1 | 0.0172 | 0.047 | 0.017 | 0.031 | 0.007 | 0.005 | 0.026 | 0.012 |
| eL6-dP04A03.8 | 0.0201 | 0.035 | 0.026 | 0.014 | 0.008 | 0.002 | 0.027 | 0.014 |
| L6-dP03H10 | 0.004 | 0.009 | >10 | >10 | >10 | 0.001 | >10 | >10 |
| eL6-dP03H10.2 | 0.008 | 0.004 | >10 | 0.005 | >10 | 0.020 | 0.141 | 0.029 |
| eL6-dP03H10.8 | 0.006 | 0.003 | >10 | 0.006 | >10 | 0.003 | 0.052 | 0.057 |

Together this data suggest that engineered SARS-CoV-2 nAbs with improved binding affinity neutralize many potential escaped circulating strains as well as the emerging B.1.1.7 and 501Y.V2 lineages.

Animal Study

Similar to the study in Example 9, the ability of the enhanced neutralizing antibodies was assessed using a Syrian hamster animal model. Groups of six hamsters received an intraperitoneal infusion 2 mg, 500 μg, 125 μg, 31 μg or 8 μg of CC6.33 or eCC6.33.3. A control group received 2 mg of Den3 isotope matched control antibody. Three days post infusion animals were challenged with 1×10⁵ plaque forming units (PFU) of SARS-CoV-2 (USA-WA1/2020) by intranasal administration. Lung tissue was collected 4 days post challenge and viral load was measured by live virus plaque assay on Vero E6 cells from lung tissue homogenate. There was a dose-dependent decrease in the viral titer in animals given either the parental or enhanced antibody, as the control group (FIG. 36). Error bars represent geometric standard deviations of the geometric mean.

We claim:

1. A recombinant monoclonal antibody or antigen-binding fragment thereof that binds a Spike protein of SARS-CoV-2, SARS-CoV-1, SARS-CoV-2 and SARS-CoV-1, and variants thereof.

2. The recombinant monoclonal antibody or antigen-binding fragment of claim 1, wherein the SARS-CoV-2 Spike protein comprises an amino acid sequence that is at least 97%, 98%, or 99% identical to SEQ ID Nos: 1 or 194.

3. The recombinant monoclonal antibody or antigen-binding fragment of claim 2, wherein the SARS-CoV-2 Spike protein comprises an amino acid sequence of SEQ ID Nos: 1 or 194.

4. The recombinant monoclonal antibody or antigen-binding fragment of claim 1, wherein the antibody or antigen-binding fragment binds the S1 region of the Spike protein and variants thereof.

5. The recombinant monoclonal antibody or antigen-binding fragment of claim 4, wherein the antibody or antigen-binding fragment binds a receptor binding domain (RBD) of the Spike protein.

6. The recombinant monoclonal antibody or antigen-binding fragment of claim 5, wherein the RBD comprises an amino acid sequence that is at least 97%, 98%, or 99% identical to SEQ ID Nos: 2 and 195.

7. The recombinant monoclonal antibody or antigen-binding fragment of claim 5, wherein the RBD comprises an amino acid sequence of SEQ ID Nos: 2 and 195.

8. The recombinant monoclonal antibody or antigen-binding fragment of claim 1, wherein the SARS-CoV-1 Spike protein comprises an amino acid sequence that is at least 97%, 98%, or 99% identical to SEQ ID Nos: 3 or 196.

9. The recombinant monoclonal antibody or antigen-binding fragment of claim 8, wherein the SARS-CoV-1 Spike protein comprises an amino acid sequence of SEQ ID Nos: 3 or 196.

10. The recombinant monoclonal antibody or antigen-binding fragment of any of the preceding claims, wherein the antibody or antigen-binding fragment inhibits binding of SARS-CoV-2 and/or SARS-CoV-1 to an ACE-2 receptor.

11. The recombinant monoclonal antibody or antigen-binding fragment of any of the preceding claims, wherein the antibody or antigen-binding fragment has a binding affinity of 10⁻⁶ to 10⁻⁹ kD.

12. The recombinant monoclonal antibody or antigen-binding fragment of any of the preceding claims, wherein the antibody or antigen-binding fragment has a binding affinity of 10⁻⁹ to 10⁻¹² kD.

13. The recombinant monoclonal antibody or antigen-binding fragment of any preceeding claims, wherein the antibody is at least 2 fold, 3, 4 fold, 5 fold, 6 fold more potent than a reference antibody.

14. The recombinant monoclonal antibody or antigen-binding fragment of claim 11, wherein potency is measured by a neutralizing assay.

15. The recombinant monoclonal antibody or antigen-binding fragment of any of the preceding claims, wherein the antibody or antigen-binding fragment does not cross-react with human antigen.

16. The recombinant monoclonal binding fragment of claim 1 wherein the antibody or antigen binding fragment does not cross react with a SARS-CoV-1 Spike protein.

17. The recombinant monoclonal binding fragment of claim 1 or claim 16 wherein the antibody or antigen binding fragment does not cross react with the RBD region SARS-CoV-1 Spike protein.

18. The recombinant monoclonal binding fragment of claim 17 wherein the antibody or antigen binding fragment wherein in the RBD portion of S1 comprises an amino acid sequence of SEQ ID Nos: 3 or 196.

19. The recombinant monoclonal antibody or antigen-binding fragment of any of the preceding claims, comprising:
(a) a variable heavy chain (VH) selected from the group comprising:
1) a VH-CDR1 comprising an amino acid sequence of SEQ ID NO: 6, a VH-CDR2 comprising an amino acid sequence of SEQ ID NO: 7, and a VH-CDR3 comprising an amino acid sequence of SEQ ID NO: 8;
2) a VH-CDR1 comprising an amino acid sequence of SEQ ID NO: 16, a VH-CDR2 comprising an amino acid sequence of SEQ ID NO: 17, and a VH-CDR3 comprising an amino acid sequence of SEQ ID NO: 18;
3) a VH-CDR1 comprising an amino acid sequence of SEQ ID NO: 26, a VH-CDR2 comprising an amino acid sequence of SEQ ID NO: 27, and a VH-CDR3 comprising an amino acid sequence of SEQ ID NO: 28;
4) a VH-CDR1 comprising an amino acid sequence of SEQ ID NO: 36, a VH-CDR2 comprising an amino acid sequence of SEQ ID NO: 37, and a VH-CDR3 comprising an amino acid sequence of SEQ ID NO: 38;
5) a VH-CDR1 comprising an amino acid sequence of SEQ ID NO: 46, a VH-CDR2 comprising an amino acid sequence of SEQ ID NO: 47, and a VH-CDR3 comprising an amino acid sequence of SEQ ID NO: 48;
6) a VH-CDR1 comprising an amino acid sequence of SEQ ID NO: 56, a VH-CDR2 comprising an amino acid sequence of SEQ ID NO: 57, and a VH-CDR3 comprising an amino acid sequence of SEQ ID NO: 58;
7) a VH-CDR1 comprising an amino acid sequence of SEQ ID NO: 66, a VH-CDR2 comprising an amino acid sequence of SEQ ID NO: 67, and a VH-CDR3 comprising an amino acid sequence of SEQ ID NO: 68;
8) a VH-CDR1 comprising an amino acid sequence of SEQ ID NO: 76, a VH-CDR2 comprising an amino acid sequence of SEQ ID NO: 77, and a VH-CDR3 comprising an amino acid sequence of SEQ ID NO: 78;
9) a VH-CDR1 comprising an amino acid sequence of SEQ ID NO: 86, a VH-CDR2 comprising an amino acid sequence of SEQ ID NO: 87, and a VH-CDR3 comprising an amino acid sequence of SEQ ID NO: 88;
10) a VH-CDR1 comprising an amino acid sequence of SEQ ID NO: 96, a VH-CDR2 comprising an amino acid sequence of SEQ ID NO: 97, and a VH-CDR3 comprising an amino acid sequence of SEQ ID NO: 98;
11) a VH-CDR1 comprising an amino acid sequence of SEQ ID NO: 106, a VH-CDR2 comprising an amino acid sequence of SEQ ID NO: 107, and a VH-CDR3 comprising an amino acid sequence of SEQ ID NO: 108;
12) a VH-CDR1 comprising an amino acid sequence of SEQ ID NO: 116, a VH-CDR2 comprising an amino acid sequence of SEQ ID NO: 117, and a VH-CDR3 comprising an amino acid sequence of SEQ ID NO: 118;
13) a VH-CDR1 comprising an amino acid sequence of SEQ ID NO: 126, a VH-CDR2 comprising an amino acid sequence of SEQ ID NO: 127, and a VH-CDR3 comprising an amino acid sequence of SEQ ID NO: 128;
14) a VH-CDR1 comprising an amino acid sequence of SEQ ID NO: 136, a VH-CDR2 comprising an amino acid sequence of SEQ ID NO: 137, and a VH-CDR3 comprising an amino acid sequence of SEQ ID NO: 138;
15) a VH-CDR1 comprising an amino acid sequence of SEQ ID NO: 146, a VH-CDR2 comprising an amino acid sequence of SEQ ID NO: 147, and a VH-CDR3 comprising an amino acid sequence of SEQ ID NO: 148;
16) a VH-CDR1 comprising an amino acid sequence of SEQ ID NO: 156, a VH-CDR2 comprising an amino acid sequence of SEQ ID NO: 157, and a VH-CDR3 comprising an amino acid sequence of SEQ ID NO: 158;
17) a VH-CDR1 comprising an amino acid sequence of SEQ ID NO: 166, a VH-CDR2 comprising an amino acid sequence of SEQ ID NO: 167, and a VH-CDR3 comprising an amino acid sequence of SEQ ID NO: 168;
18) a VH-CDR1 comprising an amino acid sequence of SEQ ID NO: 176, a VH-CDR2 comprising an amino acid sequence of SEQ ID NO: 177, and a VH-CDR3 comprising an amino acid sequence of SEQ ID NO: 178;
19) a VH-CDR1 comprising an amino acid sequence of SEQ ID NO: 186, a VH-CDR2 comprising an amino acid sequence of SEQ ID NO: 187, and a VH-CDR3 comprising an amino acid sequence of SEQ ID NO: 188;
20) a VH-CDR1 comprising an amino acid sequence of SEQ ID NO: 199, a VH-CDR2 comprising an amino acid sequence of SEQ ID NO: 200, and a VH-CDR3 comprising an amino acid sequence of SEQ ID NO: 201;
21) a VH-CDR1 comprising an amino acid sequence of SEQ ID NO: 209, a VH-CDR2 comprising an amino acid sequence of SEQ ID NO: 210, and a VH-CDR3 comprising an amino acid sequence of SEQ ID NO: 211;
22) a VH-CDR1 comprising an amino acid sequence of SEQ ID NO: 219, a VH-CDR2 comprising an amino acid sequence of SEQ ID NO: 220, and a VH-CDR3 comprising an amino acid sequence of SEQ ID NO: 221;
23) a VH-CDR1 comprising an amino acid sequence of SEQ ID NO: 229, a VH-CDR2 comprising an amino acid sequence of SEQ ID NO: 230, and a VH-CDR3 comprising an amino acid sequence of SEQ ID NO: 231;
24) a VH-CDR1 comprising an amino acid sequence of SEQ ID NO: 239, a VH-CDR2 comprising an amino acid sequence of SEQ ID NO: 240, and a VH-CDR3 comprising an amino acid sequence of SEQ ID NO: 241;
25) a VH-CDR1 comprising an amino acid sequence of SEQ ID NO: 249, a VH-CDR2 comprising an amino acid sequence of SEQ ID NO: 250, and a VH-CDR3 comprising an amino acid sequence of SEQ ID NO: 251;
26) a VH-CDR1 comprising an amino acid sequence of SEQ ID NO: 259, a VH-CDR2 comprising an amino acid sequence of SEQ ID NO: 260, and a VH-CDR3 comprising an amino acid sequence of SEQ ID NO: 261;
27) a VH-CDR1 comprising an amino acid sequence of SEQ ID NO: 269, a VH-CDR2 comprising an amino acid sequence of SEQ ID NO: 270, and a VH-CDR3 comprising an amino acid sequence of SEQ ID NO: 271;
28) a VH-CDR1 comprising an amino acid sequence of SEQ ID NO: 279, a VH-CDR2 comprising an amino acid sequence of SEQ ID NO: 280, and a VH-CDR3 comprising an amino acid sequence of SEQ ID NO: 281;
29) a VH-CDR1 comprising an amino acid sequence of SEQ ID NO: 289, a VH-CDR2 comprising an amino acid sequence of SEQ ID NO: 290, and a VH-CDR3 comprising an amino acid sequence of SEQ ID NO: 291;
30) a VH-CDR1 comprising an amino acid sequence of SEQ ID NO: 299, a VH-CDR2 comprising an amino acid sequence of SEQ ID NO: 300, and a VH-CDR3 comprising an amino acid sequence of SEQ ID NO: 301;
31) a VH-CDR1 comprising an amino acid sequence of SEQ ID NO: 309, a VH-CDR2 comprising an amino acid sequence of SEQ ID NO: 310, and a VH-CDR3 comprising an amino acid sequence of SEQ ID NO: 311;
32) a VH-CDR1 comprising an amino acid sequence of SEQ ID NO: 319, a VH-CDR2 comprising an amino acid sequence of SEQ ID NO: 320, and a VH-CDR3 comprising an amino acid sequence of SEQ ID NO: 321;
33) a VH-CDR1 comprising an amino acid sequence of SEQ ID NO: 329, a VH-CDR2 comprising an amino acid sequence of SEQ ID NO: 330, and a VH-CDR3 comprising an amino acid sequence of SEQ ID NO: 331;
34) a VH-CDR1 comprising an amino acid sequence of SEQ ID NO: 339, a VH-CDR2 comprising an amino acid sequence of SEQ ID NO: 340, and a VH-CDR3 comprising an amino acid sequence of SEQ ID NO: 341;
35) a VH-CDR1 comprising an amino acid sequence of SEQ ID NO: 349, a VH-CDR2 comprising an amino acid sequence of SEQ ID NO: 350, and a VH-CDR3 comprising an amino acid sequence of SEQ ID NO: 351;
36) a VH-CDR1 comprising an amino acid sequence of SEQ ID NO: 359, a VH-CDR2 comprising an amino acid sequence of SEQ ID NO: 360, and a VH-CDR3 comprising an amino acid sequence of SEQ ID NO: 361;
37) a VH-CDR1 comprising an amino acid sequence of SEQ ID NO: 369, a VH-CDR2 comprising an amino acid sequence of SEQ ID NO: 370, and a VH-CDR3 comprising an amino acid sequence of SEQ ID NO: 371;
38) a VH-CDR1 comprising an amino acid sequence of SEQ ID NO: 379, a VH-CDR2 comprising an amino acid sequence of SEQ ID NO: 380, and a VH-CDR3 comprising an amino acid sequence of SEQ ID NO: 381;
39) a VH-CDR1 comprising an amino acid sequence of SEQ ID NO: 389, a VH-CDR2 comprising an amino acid sequence of SEQ ID NO: 390, and a VH-CDR3 comprising an amino acid sequence of SEQ ID NO: 391;
40) a VH-CDR1 comprising an amino acid sequence of SEQ ID NO: 399, a VH-CDR2 comprising an amino acid sequence of SEQ ID NO: 400, and a VH-CDR3 comprising an amino acid sequence of SEQ ID NO: 401;
41) a VH-CDR1 comprising an amino acid sequence of SEQ ID NO: 409, a VH-CDR2 comprising an amino acid sequence of SEQ ID NO: 410, and a VH-CDR3 comprising an amino acid sequence of SEQ ID NO: 411;
42) a VH-CDR1 comprising an amino acid sequence of SEQ ID NO: 419, a VH-CDR2 comprising an amino acid sequence of SEQ ID NO: 420, and a VH-CDR3 comprising an amino acid sequence of SEQ ID NO: 421;
43) a VH-CDR1 comprising an amino acid sequence of SEQ ID NO: 429, a VH-CDR2 comprising an amino acid sequence of SEQ ID NO: 430, and a VH-CDR3 comprising an amino acid sequence of SEQ ID NO: 431;
44) a VH-CDR1 comprising an amino acid sequence of SEQ ID NO: 439, a VH-CDR2 comprising an amino acid sequence of SEQ ID NO: 440, and a VH-CDR3 comprising an amino acid sequence of SEQ ID NO: 441;
45) a VH-CDR1 comprising an amino acid sequence of SEQ ID NO: 449, a VH-CDR2 comprising an amino acid sequence of SEQ ID NO: 450, and a VH-CDR3 comprising an amino acid sequence of SEQ ID NO: 451;
46) a VH-CDR1 comprising an amino acid sequence of SEQ ID NO: 459, a VH-CDR2 comprising an amino acid sequence of SEQ ID NO: 460, and a VH-CDR3 comprising an amino acid sequence of SEQ ID NO: 461;
47) a VH-CDR1 comprising an amino acid sequence of SEQ ID NO: 469, a VH-CDR2 comprising an amino acid sequence of SEQ ID NO: 470, and a VH-CDR3 comprising an amino acid sequence of SEQ ID NO: 471;
48) a VH-CDR1 comprising an amino acid sequence of SEQ ID NO: 479, a VH-CDR2 comprising an amino acid sequence of SEQ ID NO: 480, and a VH-CDR3 comprising an amino acid sequence of SEQ ID NO: 481;
wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody; and
(b) a variable light chain (VL)) selected from the group comprising:
1) a VL-CDR1 comprising an amino acid sequence of SEQ ID NO: 11, a VL-CDR2 comprising an amino acid sequence of SEQ ID NO: 12, and a VL-CDR3 comprising an amino acid sequence of SEQ ID NO: 13;
2) a VL-CDR1 comprising an amino acid sequence of SEQ ID NO: 21, a VL-CDR2 comprising an amino acid sequence of SEQ ID NO: 22, and a VL-CDR3 comprising an amino acid sequence of SEQ ID NO: 23;
3) a VL-CDR1 comprising an amino acid sequence of SEQ ID NO: 31, a VL-CDR2 comprising an amino acid sequence of SEQ ID NO: 32, and a VL-CDR3 comprising an amino acid sequence of SEQ ID NO: 33;
4) a VL-CDR1 comprising an amino acid sequence of SEQ ID NO: 41, a VL-CDR2 comprising an amino acid sequence of SEQ ID NO: 42, and a VL-CDR3 comprising an amino acid sequence of SEQ ID NO: 43;
5) a VL-CDR1 comprising an amino acid sequence of SEQ ID NO: 51, a VL-CDR2 comprising an amino acid sequence of SEQ ID NO: 52, and a VL-CDR3 comprising an amino acid sequence of SEQ ID NO: 53;
6) a VL-CDR1 comprising an amino acid sequence of SEQ ID NO: 61, a VL-CDR2 comprising an amino acid sequence of SEQ ID NO: 62, and a VL-CDR3 comprising an amino acid sequence of SEQ ID NO: 63;
7) a VL-CDR1 comprising an amino acid sequence of SEQ ID NO: 71, a VL-CDR2 comprising an amino acid sequence of SEQ ID NO: 72, and a VL-CDR3 comprising an amino acid sequence of SEQ ID NO: 73;
8) a VL-CDR1 comprising an amino acid sequence of SEQ ID NO: 81, a VL-CDR2 comprising an amino acid sequence of SEQ ID NO: 82, and a VL-CDR3 comprising an amino acid sequence of SEQ ID NO: 83;
9) a VL-CDR1 comprising an amino acid sequence of SEQ ID NO: 91, a VL-CDR2 comprising an amino acid sequence of SEQ ID NO: 92, and a VL-CDR3 comprising an amino acid sequence of SEQ ID NO: 93;
10) a VL-CDR1 comprising an amino acid sequence of SEQ ID NO: 101, a VL-CDR2 comprising an amino acid sequence of SEQ ID NO: 102, and a VL-CDR3 comprising an amino acid sequence of SEQ ID NO: 103;
11) a VL-CDR1 comprising an amino acid sequence of SEQ ID NO: 111, a VL-CDR2 comprising an amino acid sequence of SEQ ID NO: 112, and a VL-CDR3 comprising an amino acid sequence of SEQ ID NO: 113; 12) a VL-CDR1 comprising an amino acid sequence of SEQ ID NO: 121, a VL-CDR2 comprising an amino acid sequence of SEQ ID NO: 122, and a VL-CDR3 comprising an amino acid sequence of SEQ ID NO: 123;
13) a VL-CDR1 comprising an amino acid sequence of SEQ ID NO: 131, a VL-CDR2 comprising an amino acid sequence of SEQ ID NO: 132, and a VL-CDR3 comprising an amino acid sequence of SEQ ID NO: 133;
14) a VL-CDR1 comprising an amino acid sequence of SEQ ID NO: 141, a VL-CDR2 comprising an amino acid sequence of SEQ ID NO: 142, and a VL-CDR3 comprising an amino acid sequence of SEQ ID NO: 143;
15) a VL-CDR1 comprising an amino acid sequence of SEQ ID NO: 151, a VL-CDR2 comprising an amino acid sequence of SEQ ID NO: 152, and a VL-CDR3 comprising an amino acid sequence of SEQ ID NO: 153;
16) a VL-CDR1 comprising an amino acid sequence of SEQ ID NO: 161, a VL-CDR2 comprising an amino acid sequence of SEQ ID NO: 162, and a VL-CDR3 comprising an amino acid sequence of SEQ ID NO: 163;
17) a VL-CDR1 comprising an amino acid sequence of SEQ ID NO: 171, a VL-CDR2 comprising an amino acid sequence of SEQ ID NO: 172, and a VL-CDR3 comprising an amino acid sequence of SEQ ID NO: 173;
18) a VL-CDR1 comprising an amino acid sequence of SEQ ID NO: 181, a VL-CDR2 comprising an amino acid sequence of SEQ ID NO: 182, and a VL-CDR3 comprising an amino acid sequence of SEQ ID NO: 183;
19) a VL-CDR1 comprising an amino acid sequence of SEQ ID NO: 191, a VL-CDR2 comprising an amino acid sequence of SEQ ID NO: 192, and a VL-CDR3 comprising an amino acid sequence of SEQ ID NO: 193;
20) a VL-CDR1 comprising an amino acid sequence of SEQ ID NO: 204, a VL-CDR2 comprising an amino acid sequence of SEQ ID NO: 205, and a VL-CDR3 comprising an amino acid sequence of SEQ ID NO: 206;
21) a VL-CDR1 comprising an amino acid sequence of SEQ ID NO: 214, a VL-CDR2 comprising an amino acid sequence of SEQ ID NO: 215, and a VL-CDR3 comprising an amino acid sequence of SEQ ID NO: 216;
22) a VL-CDR1 comprising an amino acid sequence of SEQ ID NO: 224, a VL-CDR2 comprising an amino acid sequence of SEQ ID NO: 225, and a VL-CDR3 comprising an amino acid sequence of SEQ ID NO: 226;
23) a VL-CDR1 comprising an amino acid sequence of SEQ ID NO: 234, a VL-CDR2 comprising an amino acid sequence of SEQ ID NO: 235, and a VL-CDR3 comprising an amino acid sequence of SEQ ID NO: 236;
24) a VL-CDR1 comprising an amino acid sequence of SEQ ID NO: 244, a VL-CDR2 comprising an amino acid sequence of SEQ ID NO: 245, and a VL-CDR3 comprising an amino acid sequence of SEQ ID NO: 246;
25) a VL-CDR1 comprising an amino acid sequence of SEQ ID NO: 254, a VL-CDR2 comprising an amino acid sequence of SEQ ID NO: 255, and a VL-CDR3 comprising an amino acid sequence of SEQ ID NO: 256;
26) a VL-CDR1 comprising an amino acid sequence of SEQ ID NO: 264, a VL-CDR2 comprising an amino acid sequence of SEQ ID NO: 265, and a VL-CDR3 comprising an amino acid sequence of SEQ ID NO: 266;
27) a VL-CDR1 comprising an amino acid sequence of SEQ ID NO: 274, a VL-CDR2 comprising an amino acid sequence of SEQ ID NO: 275, and a VL-CDR3 comprising an amino acid sequence of SEQ ID NO: 276;
28) a VL-CDR1 comprising an amino acid sequence of SEQ ID NO: 284, a VL-CDR2 comprising an amino acid sequence of SEQ ID NO: 285, and a VL-CDR3 comprising an amino acid sequence of SEQ ID NO: 286;
29) a VL-CDR1 comprising an amino acid sequence of SEQ ID NO: 294, a VL-CDR2 comprising an amino acid sequence of SEQ ID NO: 295, and a VL-CDR3 comprising an amino acid sequence of SEQ ID NO: 296;
30) a VL-CDR1 comprising an amino acid sequence of SEQ ID NO: 304, a VL-CDR2 comprising an amino acid sequence of SEQ ID NO: 305, and a VL-CDR3 comprising an amino acid sequence of SEQ ID NO: 306;
31) a VL-CDR1 comprising an amino acid sequence of SEQ ID NO: 314, a VL-CDR2 comprising an amino acid sequence of SEQ ID NO: 315, and a VL-CDR3 comprising an amino acid sequence of SEQ ID NO: 316;
32) a VL-CDR1 comprising an amino acid sequence of SEQ ID NO: 324, a VL-CDR2 comprising an amino acid sequence of SEQ ID NO: 325, and a VL-CDR3 comprising an amino acid sequence of SEQ ID NO: 326;
33) a VL-CDR1 comprising an amino acid sequence of SEQ ID NO: 334, a VL-CDR2 comprising an amino acid sequence of SEQ ID NO: 335, and a VL-CDR3 comprising an amino acid sequence of SEQ ID NO: 336;
34) a VL-CDR1 comprising an amino acid sequence of SEQ ID NO: 344, a VL-CDR2 comprising an amino acid sequence of SEQ ID NO: 345, and a VL-CDR3 comprising an amino acid sequence of SEQ ID NO: 346;
35) a VL-CDR1 comprising an amino acid sequence of SEQ ID NO: 354, a VL-CDR2 comprising an amino acid sequence of SEQ ID NO: 355, and a VL-CDR3 comprising an amino acid sequence of SEQ ID NO: 356;
36) a VL-CDR1 comprising an amino acid sequence of SEQ ID NO: 364, a VL-CDR2 comprising an amino acid sequence of SEQ ID NO: 365, and a VL-CDR3 comprising an amino acid sequence of SEQ ID NO: 366;
37) a VL-CDR1 comprising an amino acid sequence of SEQ ID NO: 374, a VL-CDR2 comprising an amino acid sequence of SEQ ID NO: 375, and a VL-CDR3 comprising an amino acid sequence of SEQ ID NO: 376;
38) a VL-CDR1 comprising an amino acid sequence of SEQ ID NO: 384, a VL-CDR2 comprising an amino acid sequence of SEQ ID NO: 385, and a VL-CDR3 comprising an amino acid sequence of SEQ ID NO: 386;
39) a VL-CDR1 comprising an amino acid sequence of SEQ ID NO: 394, a VL-CDR2 comprising an amino acid sequence of SEQ ID NO: 395, and a VL-CDR3 comprising an amino acid sequence of SEQ ID NO: 396;
40) a VL-CDR1 comprising an amino acid sequence of SEQ ID NO: 404, a VL-CDR2 comprising an amino acid sequence of SEQ ID NO: 405, and a VL-CDR3 comprising an amino acid sequence of SEQ ID NO: 406;
41) a VL-CDR1 comprising an amino acid sequence of SEQ ID NO: 414, a VL-CDR2 comprising an amino acid sequence of SEQ ID NO: 415, and a VL-CDR3 comprising an amino acid sequence of SEQ ID NO: 416;
42) a VL-CDR1 comprising an amino acid sequence of SEQ ID NO: 424, a VL-CDR2 comprising an amino acid sequence of SEQ ID NO: 425, and a VL-CDR3 comprising an amino acid sequence of SEQ ID NO: 426;
43) a VL-CDR1 comprising an amino acid sequence of SEQ ID NO: 434, a VL-CDR2 comprising an amino acid sequence of SEQ ID NO: 435, and a VL-CDR3 comprising an amino acid sequence of SEQ ID NO: 436;
44) a VL-CDR1 comprising an amino acid sequence of SEQ ID NO: 444, a VL-CDR2 comprising an amino acid sequence of SEQ ID NO: 445, and a VL-CDR3 comprising an amino acid sequence of SEQ ID NO: 446;
45) a VL-CDR1 comprising an amino acid sequence of SEQ ID NO: 454, a VL-CDR2 comprising an amino acid sequence of SEQ ID NO: 455, and a VL-CDR3 comprising an amino acid sequence of SEQ ID NO: 456;
46) a VL-CDR1 comprising an amino acid sequence of SEQ ID NO: 464, a VL-CDR2 comprising an amino acid sequence of SEQ ID NO: 465, and a VL-CDR3 comprising an amino acid sequence of SEQ ID NO: 466;
47) a VL-CDR1 comprising an amino acid sequence of SEQ ID NO: 474, a VL-CDR2 comprising an amino acid sequence of SEQ ID NO: 475, and a VL-CDR3 comprising an amino acid sequence of SEQ ID NO: 476;
48) a VL-CDR1 comprising an amino acid sequence of SEQ ID NO: 484, a VL-CDR2 comprising an amino acid sequence of SEQ ID NO: 485, and a VL-CDR3 comprising an amino acid sequence of SEQ ID NO: 486;
wherein the VL-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody.

20. The recombinant monoclonal antibody or antigen-binding fragment of any of the preceding claims, comprising:
a) a variable heavy chain (VH) selected from the group comprising:
1) a VH-CDR1 comprising an amino acid sequence of SEQ ID NO: 6, a VH-CDR2 comprising an amino acid sequence of SEQ ID NO: 7, and a VH-CDR3 comprising an amino acid sequence of SEQ ID NO: 8;
2) a VH-CDR1 comprising an amino acid sequence of SEQ ID NO: 219, a VH-CDR2 comprising an amino acid sequence of SEQ ID NO: 220, and a VH-CDR3 comprising an amino acid sequence of SEQ ID NO: 221;
3) a VH-CDR1 comprising an amino acid sequence of SEQ ID NO: 249, a VH-CDR2 comprising an amino acid sequence of SEQ ID NO: 250, and a VH-CDR3 comprising an amino acid sequence of SEQ ID NO: 251;
4) a VH-CDR1 comprising an amino acid sequence of SEQ ID NO: 369, a VH-CDR2 comprising an amino acid sequence of SEQ ID NO: 370, and a VH-CDR3 comprising an amino acid sequence of SEQ ID NO: 371;
5) a VH-CDR1 comprising an amino acid sequence of SEQ ID NO: 379, a VH-CDR2 comprising an amino acid sequence of SEQ ID NO: 380, and a VH-CDR3 comprising an amino acid sequence of SEQ ID NO: 381;
6) a VH-CDR1 comprising an amino acid sequence of SEQ ID NO: 389, a VH-CDR2 comprising an amino acid sequence of SEQ ID NO: 390, and a VH-CDR3 comprising an amino acid sequence of SEQ ID NO: 391;
7) a VH-CDR1 comprising an amino acid sequence of SEQ ID NO: 399, a VH-CDR2 comprising an amino acid sequence of SEQ ID NO: 400, and a VH-CDR3 comprising an amino acid sequence of SEQ ID NO: 401;
8) a VH-CDR1 comprising an amino acid sequence of SEQ ID NO: 409, a VH-CDR2 comprising an amino acid sequence of SEQ ID NO: 410, and a VH-CDR3 comprising an amino acid sequence of SEQ ID NO: 411;
9) a VH-CDR1 comprising an amino acid sequence of SEQ ID NO: 419, a VH-CDR2 comprising an amino acid sequence of SEQ ID NO: 420, and a VH-CDR3 comprising an amino acid sequence of SEQ ID NO: 421;
10) a VH-CDR1 comprising an amino acid sequence of SEQ ID NO: 429, a VH-CDR2 comprising an amino acid sequence of SEQ ID NO: 430, and a VH-CDR3 comprising an amino acid sequence of SEQ ID NO: 431;
11) a VH-CDR1 comprising an amino acid sequence of SEQ ID NO: 439, a VH-CDR2 comprising an amino acid sequence of SEQ ID NO: 440, and a VH-CDR3 comprising an amino acid sequence of SEQ ID NO: 441;
12) a VH-CDR1 comprising an amino acid sequence of SEQ ID NO: 449, a VH-CDR2 comprising an amino acid sequence of SEQ ID NO: 450, and a VH-CDR3 comprising an amino acid sequence of SEQ ID NO: 451;
13) a VH-CDR1 comprising an amino acid sequence of SEQ ID NO: 459, a VH-CDR2 comprising an amino acid sequence of SEQ ID NO: 460, and a VH-CDR3 comprising an amino acid sequence of SEQ ID NO: 461;
14) a VH-CDR1 comprising an amino acid sequence of SEQ ID NO: 469, a VH-CDR2 comprising an amino acid sequence of SEQ ID NO: 470, and a VH-CDR3 comprising an amino acid sequence of SEQ ID NO: 471; and
15) a VH-CDR1 comprising an amino acid sequence of SEQ ID NO: 479, a VH-CDR2 comprising an amino acid sequence of SEQ ID NO: 480, and a VH-CDR3 comprising an amino acid sequence of SEQ ID NO: 481;
wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody; and
(b) a variable light chain (VL)) selected from the group comprising:
1) a VL-CDR1 comprising an amino acid sequence of SEQ ID NO: 11, a VL-CDR2 comprising an amino acid sequence of SEQ ID NO: 12, and a VL-CDR3 comprising an amino acid sequence of SEQ ID NO: 13;
2) a VL-CDR1 comprising an amino acid sequence of SEQ ID NO: 224, a VL-CDR2 comprising an amino acid sequence of SEQ ID NO: 225, and a VL-CDR3 comprising an amino acid sequence of SEQ ID NO: 226;
3) a VL-CDR1 comprising an amino acid sequence of SEQ ID NO: 254, a VL-CDR2 comprising an amino acid sequence of SEQ ID NO: 255, and a VL-CDR3 comprising an amino acid sequence of SEQ ID NO: 256;
4) a VL-CDR1 comprising an amino acid sequence of SEQ ID NO: 374, a VL-CDR2 comprising an amino acid sequence of SEQ ID NO: 375, and a VL-CDR3 comprising an amino acid sequence of SEQ ID NO: 376;
5) a VL-CDR1 comprising an amino acid sequence of SEQ ID NO: 384, a VL-CDR2 comprising an amino acid sequence of SEQ ID NO: 385, and a VL-CDR3 comprising an amino acid sequence of SEQ ID NO: 386;
6) a VL-CDR1 comprising an amino acid sequence of SEQ ID NO: 394, a VL-CDR2 comprising an amino acid sequence of SEQ ID NO: 395, and a VL-CDR3 comprising an amino acid sequence of SEQ ID NO: 396;
7) a VL-CDR1 comprising an amino acid sequence of SEQ ID NO: 404, a VL-CDR2 comprising an amino acid sequence of SEQ ID NO: 405, and a VL-CDR3 comprising an amino acid sequence of SEQ ID NO: 406;
8) a VL-CDR1 comprising an amino acid sequence of SEQ ID NO: 414, a VL-CDR2 comprising an amino acid sequence of SEQ ID NO: 415, and a VL-CDR3 comprising an amino acid sequence of SEQ ID NO: 416;
9) a VL-CDR1 comprising an amino acid sequence of SEQ ID NO: 424, a VL-CDR2 comprising an amino acid sequence of SEQ ID NO: 425, and a VL-CDR3 comprising an amino acid sequence of SEQ ID NO: 426;
10) a VL-CDR1 comprising an amino acid sequence of SEQ ID NO: 434, a VL-CDR2 comprising an amino acid sequence of SEQ ID NO: 435, and a VL-CDR3 comprising an amino acid sequence of SEQ ID NO: 436;
11) a VL-CDR1 comprising an amino acid sequence of SEQ ID NO: 444, a VL-CDR2 comprising an amino acid sequence of SEQ ID NO: 445, and a VL-CDR3 comprising an amino acid sequence of SEQ ID NO: 446;
12) a VL-CDR1 comprising an amino acid sequence of SEQ ID NO: 454, a VL-CDR2 comprising an amino acid sequence of SEQ ID NO: 455, and a VL-CDR3 comprising an amino acid sequence of SEQ ID NO: 456;
13) a VL-CDR1 comprising an amino acid sequence of SEQ ID NO: 464, a VL-CDR2 comprising an amino acid sequence of SEQ ID NO: 465, and a VL-CDR3 comprising an amino acid sequence of SEQ ID NO: 466;
14) a VL-CDR1 comprising an amino acid sequence of SEQ ID NO: 474, a VL-CDR2 comprising an amino acid sequence of SEQ ID NO: 475, and a VL-CDR3 comprising an amino acid sequence of SEQ ID NO: 476;
15) a VL-CDR1 comprising an amino acid sequence of SEQ ID NO: 484, a VL-CDR2 comprising an amino acid sequence of SEQ ID NO: 485, and a VL-CDR3 comprising an amino acid sequence of SEQ ID NO: 486;
wherein the VL-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody.

21. The recombinant monoclonal antibody or antigen-binding fragment of any of the preceding claims, comprising:
a) a variable heavy chain (VH) comprising a VH-CDR1 comprising an amino acid sequence of SEQ ID NO: 219, a VH-CDR2 comprising an amino acid sequence of SEQ ID NO: 220, and a VH-CDR3 comprising an amino acid sequence of SEQ ID NO: 221;
wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody
(b) a variable light chain (VL)) selected from the group comprising a VL-CDR1 comprising an amino acid sequence of SEQ ID NO: 224, a VL-CDR2 comprising an amino acid sequence of SEQ ID NO: 225, and a VL-CDR3 comprising an amino acid sequence of SEQ ID NO: 226;
wherein the VL-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody.

22. The recombinant monoclonal antibody or antigen-binding fragment of any of the preceding claims, comprising:
a) a variable heavy chain (VH) comprising a VH-CDR1 comprising an amino acid sequence of SEQ ID NO: 249, a VH-CDR2 comprising an amino acid sequence of SEQ ID NO: 250, and a VH-CDR3 comprising an amino acid sequence of SEQ ID NO: 251;
wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody
(b) a variable light chain (VL)) selected from the group comprising a VL-CDR1 comprising an amino acid sequence of SEQ ID NO: 254, a VL-CDR2 comprising an amino acid sequence of SEQ ID NO: 255, and a VL-CDR3 comprising an amino acid sequence of SEQ ID NO: 256:
wherein the VL-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody.

23. The recombinant monoclonal antibody or antigen-binding fragment of any of the preceding claims, comprising:
a) a variable heavy chain comprising a VH-CDR1 comprising an amino acid sequence of SEQ ID NO: 369, a VH-CDR2 comprising an amino acid sequence of SEQ ID NO: 370, and a VH-CDR3 comprising an amino acid sequence of SEQ ID NO: 371;
wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody;
(b) a variable light chain (VL) comprising a VL-CDR1 comprising an amino acid sequence of SEQ ID NO: 374, a VL-CDR2 comprising an amino acid sequence of SEQ ID NO: 375, and a VL-CDR3 comprising an amino acid sequence of SEQ ID NO: 376:
wherein the VL-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody.

24. The recombinant monoclonal antibody or antigen-binding fragment of any of the preceding claims, comprising:
a) a variable heavy chain (VH) comprising a VH-CDR1 comprising an amino acid sequence of SEQ ID NO: 439, a VH-CDR2 comprising an amino acid sequence of SEQ ID NO: 440, and a VH-CDR3 comprising an amino acid sequence of SEQ ID NO: 441:
wherein the VH-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody
(b) a variable light chain (VL)) selected from the group comprising a VL-CDR1 comprising an amino acid sequence of SEQ ID NO: 444, a VL-CDR2 comprising an amino acid sequence of SEQ ID NO: 445, and a VL-CDR3 comprising an amino acid sequence of SEQ ID NO: 446;
wherein the VL-CDR sequences optionally comprise 1, 2, 3, 4, or 5 amino acid substitutions, deletions or insertions, and wherein the amino acid substitutions, deletions or insertions reduce the binding affinity by no more than 2, 3, 4, 5, or 10-fold as compared to a reference antibody.

25. The recombinant monoclonal antibody of any one of claims 19 to 24, wherein the amino acid substitutions, deletions or insertions comprises a homologous substitution.

26. The recombinant monoclonal antibody or antigen-binding fragment of any of claims 1-18, wherein the VH chain comprises an amino acid sequence that is at least 80%, 85%, 90%, 92%, 93%, 95%, 97%, 98%, 99% or 100% identical to a sequence selected from the group consisting of: SEQ ID NOs: 4, 14, 24, 34, 44, 54, 64, 74, 84, 94, 104, 114, 124, 134, 144, 154, 164, 174, 184, 197, 207, 217, 227, 237, 247, 257, 267, 277, 287, 297, 307, 317, 327, 337, 347, 357, 367, 377, 387, 397, 407, 417, 427, 437, 447, 457, 467, and 477.

27. The recombinant monoclonal antibody or antigen-binding fragment of any of claims 1-18, wherein the VL chain comprises an amino acid sequence that is at least 80%, 85%, 90%, 92%, 93%, 95%, 97%, 98%, 99% or 100% identical to a sequence selected from the group consisting of: SEQ ID NOs: 9, 19, 29, 39, 49, 59, 69, 79, 89, 99, 109, 119, 129, 139, 149, 159, 169, 179, 189, 202, 212, 222, 232, 242, 252, 262, 272, 282, 292, 302, 312, 322, 332, 342, 352, 362, 372, 382, 392, 402, 412, 422, 432, 442, 452, 462, 472, and 482.

28. The recombinant antibody or antigen-binding fragment of any one of the preceding claims, wherein the antibody or antigen-binding fragment is an antigen-binding fragment.

29. The recombinant antibody or antigen-binding fragment of any one of the preceding claims, wherein the antigen-binding fragment is an scFv.

30. The recombinant antibody or antigen-binding fragment of any one of the preceding claims, wherein the antigen-binding fragment is a Fab′.

31. The recombinant antibody or antigen-binding fragment of any one of the preceding claims, wherein the antibody or antigen-binding fragment is an antibody.

32. The recombinant antibody or antigen-binding fragment of any one of the preceding claims, wherein the antibody is an IgG antibody.

33. The recombinant antibody or antigen-binding fragment thereof in any one of the preceding claims, wherein in the antibody or antigen-binding fragment is a neutralizing antibody.

34. The recombinant antibody or antigen-binding fragment thereof in any one of the preceding claims, wherein in the antibody or antigen-binding fragment is an isolated antibody.

35. The recombinant antibody or antigen-binding fragment thereof in any one of the preceding claims, wherein in the antibody or antigen-binding fragment is capable of binding and neutralizing one or more of SARS-CoV-2 variants designated as: D614G, N501Y, E484K, E484Q, K417N and/or L452R.

36. The recombinant antibody or antigen-binding fragment thereof in any one of the preceding claims, wherein in the antibody or antigen-binding fragment is capable of binding and neutralizing one or more of SARS-CoV-2 variants designated as: B.1.1.7, B.1.351, 501YV2.

37. A composition comprising an antibody or antigen-binding fragment of any one of the preceding claims and a pharmaceutically acceptable carrier.

38. A nucleic acid encoding the antibody or antigen-binding fragment of any one of the preceding claims.

39. A nucleic acid molecule encoding a VH chain comprising an amino acid sequence that is at least 80%, 85%, 90%, 92%, 93%, 95%, 97%, 98%, 99% or 100% identical to an amino acid sequence selected from the group consisting of: SEQ ID NOs: 4, 14, 24, 34, 44, 54, 64, 74, 84, 94, 104, 114, 124, 134, 144, 154, 164, 174, 184, 197, 207, 217, 227, 237, 247, 257, 267, 277, 287, 297, 307, 317, 327, 337, 347, 357, 367, 377, 387, 397, 407, 417, 427, 437, 447, 457, 467, and 477.

40. The nucleic acid molecule of claim 38, wherein the nucleic acid sequence is at least 80%, 85%, 90%, 92%, 93%, 95%, 97%, 98%, 99% or 100% identical to a nucleic acid sequence selected from the group consisting of SEQ ID NOs: 5, 15, 25, 35, 45, 55, 65, 75, 85, 95, 105, 115, 125, 135, 145, 155, 165, 175, 185, 198, 208, 218, 228, 238, 248, 258, 268, 278, 288, 298, 308, 318, 328, 338, 348, 358, 368, 378, 388, 398, 408, 418, 428, 438, 448, 458, 468, and 478.

41. A nucleic acid molecule that encodes a VL chain comprising an amino acid sequence that is at least 80%, 85%, 90%, 92%, 93%, 95%, 97%, 98%, 99% or 100% identical to an amino acid sequence selected from the group consisting of: SEQ ID NOs: 9, 19, 29, 39, 49, 59, 69, 79, 89, 99, 109, 119, 129, 139, 149, 159, 169, 179, 189, 202, 212, 222, 232, 242, 252, 262, 272, 282, 292, 302, 312, 322, 332, 342, 352, 362, 372, 382, 392, 402, 412, 422, 432, 442, 452, 462, 472, and 482.

42. The nucleic acid molecule of claim 38, wherein the nucleic acid sequence is at least 80%, 85%, 90%, 92%, 93%, 95%, 97%, 98%, 99% or 100% identical to a nucleic acid sequence selected from the group consisting of SEQ ID NOs: 10, 20, 30, 40, 50, 60, 70, 80, 90, 100, 110, 120, 130, 140, 150, 160, 170, 180, 190, 203, 213, 223, 233, 243, 253, 263, 273, 283, 293, 303, 313, 323, 333, 343, 353, 363, 373, 383, 393, 403, 413, 423, 433, 443, 453, 463, 473, and 483.

43. A vector comprising the nucleic acid of any one of claims 38-42.

44. A host cell comprising the vector of claim 43.

45. A lyophilized composition comprising the antibody or antigen-binding fragment thereof of any of the preceding claims.

46. A reconstituted lyophilized composition comprising the antibody or antigen-binding fragment thereof of any one of the preceding claims.

47. The composition of any one of the preceding claims, wherein the composition is formulated for administration by lozenge, spray, oral administration, delayed release or sustained release, transmucosal administration, syrup, mucoadhesive, buccal formulation, mucoadhesive tablet, topical administration, parenteral administration, injection, subdermal administration, oral solution, rectal administration, subcutaneous administration, buccal administration or transdermal administration.

48. A method of treating or preventing a SARS-CoV-2 infection comprising administering a therapeutically effective amount of the antibody or antigen-binding fragment of the preceding claims.

49. A method of producing the antibody or antigen-binding fragment of the preceding claims comprising the steps of expressing the nucleic acid or set of nucleic acids of claims 38-42 in a cultured cell, purifying the antibody or antigen-binding fragment.

50. A method of treating or preventing a SARS-CoV-2 infection comprising administering a therapeutically effective amount of a nucleic acid that encodes the antibody or antigen-binding fragment of the preceding claims.
